(19) United States
(12) Patent Application Publication
(10) Pub. No.: US 2011/0306597 A1
(43) Pub. Date: Dec. 15, 2011

(54) Nicotinamide Derivatives

(76) Inventors: James Michael Crawforth, Kent (GB); Paul Alan Glossop, Kent (GB); Bruce Cameron Hamper, Kirkwood, MO (US); Wei Huang, Wildwood, MO (US); Bradley Elwood Neal, Saint Louis, MO (US); Simon John Mantell, Kent (GB); Kirk Olson, Canton, MI (US); Atli Thorarensen, Stow, MA (US); Steve Ronald Turner, Chesterfield, MO (US)

(21) Appl. No.: 12/997,987

(22) PCT Filed: Jun. 12, 2009
(86) PCT No.: PCT/IB2009/052516

§ 371 Date: Mar. 7, 2011

Related U.S. Application Data
(60) Provisional application No. 61/073,884, filed on Jun. 19, 2008.

(30) Foreign Application Priority Data
Jun. 18, 2008 (EP) 08158516.8

(51) Int. Cl. A61K 31/55; A61K 31/437; C07D 417/12; A61K 31/4439; C07D 401/12; A61K 31/444; C07D 487/04; A61K 31/519; C07D 413/14; A61K 31/5377; C07D 213/82; A61K 31/44; C07D 413/12; A61K 31/5355; C07D 451/02; A61K 31/46; A61P 11/00; A61P 37/08; A61P 11/06; C07D 471/04

(52) U.S. Cl. 514/212.07; 546/121; 514/300; 546/269.7; 514/342; 546/262; 514/332; 546/275.1; 514/341; 546/268.4; 514/338; 544/281; 514/259.1; 544/131; 514/236.2; 546/316; 514/355; 544/96; 514/228.8; 514/230.8; 544/120; 540/523; 546/125; 514/304

(57) ABSTRACT

The present invention relates to compounds of the formula (I) and pharmaceutically acceptable salts and solvates thereof, wherein the substituents are defined herein, to compositions containing such compounds and to the uses of such compounds for the treatment of allergic and respiratory conditions.

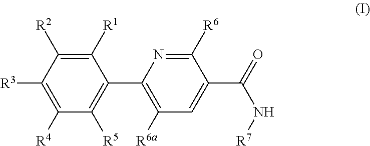

The present invention relates to nictonamide derivatives, pharmaceutical compositions comprising such derivatives and their use as medicaments. More particularly, the present invention provides N-cycloalkyl-3-phenylnicotinamide derivatives which are hematopoietic prostaglandin D₂ synthase inhibitors and useful for the treatment of allergic and respiratory conditions and diseases.

Prostaglandin D₂ (PGD₂) is a metabolite of arachidonic acid. PGD₂ promotes sleep, inhibits platelet aggregation, relaxes smooth muscle contraction, induces bronchoconstriction and attracts inflammatory cells including Th2 cells, eosinophils and basophils. Both lipocalin-type PGD synthase (L-PGDS) and hematopoietic PGDS (H-PGDS) convert PGH₂ to PGD₂.

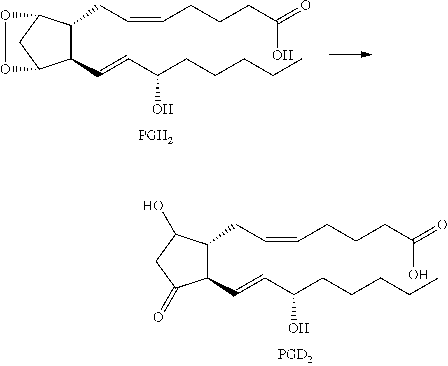

L-PGDS, also known as glutathione-independent PGDS or brain PGDS, is a 26 kDa secretory protein that is expressed by meningeal cells, epithelial cells of the choroid plexus and oligodendrocytes in the brain. L-PGDS secreted into cerebrospinal fluid is thought to be the source of PGD₂ in the central nervous system. In addition, epithelial cells in the epididymis and Leydig cells in the testis express L-PGDS and are thought to be the source of PGD₂ found in the seminal fluid. L-PGDS belongs to the lipocalin superfamily that consists of lipophilic ligand carrier proteins such as retinol- and retinoic acid-binding proteins.

In contrast, H-PGDS is a 26 kDa cytosolic protein that is responsible for the synthesis of PGD₂ in immune and inflammatory cells including mast cells, antigen-presenting cells and Th2 cells. H-PGDS is the only vertebrate member of the sigma class of glutathione S-transferases (GSTs). While both H- and L-PGDS convert PGH₂ to PGD₂, the mechanism of catalysis and specific activity of the enzymes are quite different.

The production of PGD₂ by H-PGDS is thought to play a pivotal role in airway allergic and inflammatory processes and induces vasodilatation, bronchoconstriction, pulmonary eosinophil and lymphocyte infiltration, and cytokine release in asthmatics. PGD₂ levels increase dramatically in bronchoalveolar lavage fluid following allergen challenge and the observation that patients with asthma exhibit bronchoconstriction upon inhalation of PGD₂ underscores the pathologic consequences of high levels of PGD₂ in the lung. Treatment with PGD₂ produces significant nasal congestion and fluid secretion in man and dogs, and PGD₂ is 10 times more potent than histamine and 100 times more potent than bradykinin in producing nasal blockage in humans, demonstrating a role for PGD₂ in allergic rhinitis.

Several lines of evidence suggest that PGDS is an excellent target for allergic and respiratory diseases or conditions. H-PGDS overexpresssing transgenic mice show increased allergic reactivity accompanied by elevated levels of Th2 cytokines and chemokines as well as enhanced accumulation of eosinophils and lymphocytes in the lung. In addition, PGD₂ binds to two GPCR receptors, DP1 and CRTH2. Antigen-induced airway and inflammatory responses are strongly decreased in DP1-receptor null mice and recent evidence shows that PGD₂ binding to CRTH2 mediates cell migration and the activation of Th2 cells, eosinophils, and basophils in vitro and likely promotes allergic disease in vivo. Finally, several published reports link H-PGDS gene polymorphisms with atopic asthma. For example, Aritake et al., Structural and Functional Characterization of HQL-79, and Orally Selective inhibitor of Human Hematopoietic Prostaglandin D Synthase, Journal of Biological Chemistry 2006, 281(22), pp. 15277-15286, provides a rational basis for believing that inhibition of H-PGDS is an effective way of treating several allergic and non-allergic diseases.

There is a need to provide new inhibitors of H-PDGS that are suitable as drug candidates. Such compounds should be potent, selective inhibitors of H-PGDS with appropriate metabolic stability and pharmacokinetic properties. Compounds have now been found that are inhibitors of H-PGDS, and at expected efficacious doses, do not significantly inhibit L-PGDS or kinases.

The invention therefore provides, as embodiment E1, a compound of formula (I):

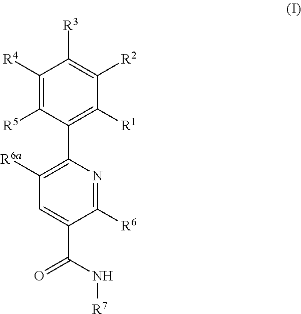

or a pharmaceutically acceptable salt thereof, or a pharmaceutically acceptable solvate of said compound or salt, wherein:
R¹, R², R³, R⁴ and R⁵ are each independently H, F, Cl, —CN, —NH₂, —CH₃, —CH₂F, —CHF₂, —CF₃, —OH, —OCH₃, —OCH₂F, —OCHF₂ or —OCF₃;

### R⁶ is H, —NH₂, —OH or —CH₃;

### R^{6a} is H, F or Cl;

R⁷ is C₁-C₆ alkyl, phenyl, Het¹, Het², Het³ or Het⁴, said C₁-C₆ alkyl, phenyl, Het¹, Het², Het³ or Het⁴ being (a) optionally substituted by 1-3 substituents selected from R^{a}, —OR^{b}, —S(O)ₙR^{b}, —COR^{b}, —NR^{x}R^{b}, —OCOR^{b}, —COOR^{b}, —NR^{x}COR^{b}, —CONR^{x}R^{b} —NR^{x}SO₂R^{b}, —SO₂NR^{x}R^{b}, —NR^{x}SO₂NR^{x}R^{b}, —NR^{x}COOR^{b}, —NR^{x}CONR^{x}R^{b}, —OCONR^{x}R^{b}, —OCOOR^{b}, —CONR^{x}SO₂R^{b}, oxo and —CN, and (b) optionally substituted by one or more halo atoms;
R^{a} is in each instance independently selected from C₁-C₆ alkyl, C₃-C₈ cycloalkyl, C₆-C₁₂ bicycloalkyl, Aryl¹, Het⁵, Het⁶, Het⁷ and Het⁸, said C₁-C₆ alkyl, C₃-C₈ cycloalkyl, C₆-C₁₂ bicycloalkyl, Aryl¹, Het⁵, Het⁶, Het⁷ and Het⁸ each being optionally substituted by 1-3 substituents selected from R^{c}, —OR^{d}, —S(O)ₙR^{d}, —COR^{d}, —NR^{x}R^{d}, —OCOR^{d}, —COOR^{d}, —NR^{x}COR^{d}, —CONR^{x}R^{d} —NR^{x}SO₂R^{d}, —SO₂NR^{x}R^{d}, —NR^{x}SO₂NR^{x}R^{d}, —NR^{x}COOR^{d}, —NR^{x}CONR^{x}R^{d}, —OCONR^{x}R^{d}, —OCOOR^{d}, —CONR^{x}SO₂R^{d}, oxo and —CN and one or more halo atoms;
R^{b} is in each instance independently selected from H, C₁-C₆ alkyl, C₃-C₈ cycloalkyl, C₆-C₁₂ bicycloalkyl, Aryl¹, Het⁵, Het⁶, Het⁷ and Het⁸, said C₁-C₆ alkyl, C₃-C₈ cycloalkyl, C₆-C₁₂ bicycloalkyl, Aryl¹, Het⁵, Het⁶, Het⁷ and Het⁸ each being optionally substituted by 1-3 substituents selected from R^{c}, —OR^{d}, —S(O)ₙR^{d}, —COR^{d}, —NR^{x}R^{d}, —OCOR^{d}, —COOR^{d}, —NR^{x}COR^{d}, —CONR^{x}R^{d} —NR^{x}SO₂R^{d}, —SO₂NR^{x}R^{d}, —NR^{x}SO₂NR^{x}R^{d}, —NR^{x}COOR^{d}, —NR^{x}CONR^{x}R^{d}, —OCONR^{x}R^{d}, —OCOOR^{d}, —CONR^{x}SO₂R^{d}, oxo and —CN and one or more halo atoms;
n is 0, 1 or 2;
R^{x} is in each instance independently H, C₁-C₆ alkyl or C₃-C₈ cycloalkyl, said C₁-C₆ alkyl or C₃-C₈ cycloalkyl being optionally substituted by one or more halo atoms;
Aryl¹ is phenyl or naphthyl;
Het¹ is a 3 to 8-membered saturated or partially unsaturated monocyclic heterocycle, containing 1 or 2 heteroatoms selected from O and N, with the proviso that Het¹ is not piperidinyl, pyrrolidinyl and azetidinyl;
Het² is a 6 to 12-membered saturated or partially unsaturated multicyclic heterocycle containing 1 or 2 heteroatoms selected from O and N, with the proviso that Het² is not a bridged piperidinyl, pyrrolidinyl or azetidinyl ring;
Het³ is (i) a 6-membered aromatic heterocycle containing 1-3 N atoms or (ii) a 5-membered aromatic heterocycle containing either (a) 1-4 N atoms or (b) 1 O or S atom and 0-3 N atoms;
Het⁴ is (i) a 10-membered bicyclic aromatic heterocycle containing 1-4 N atoms or (ii) a 9-membered bicyclic aromatic heterocycle containing either (a) 1-4 N atoms or (b) 1 O or S atom and 0-3 N atoms;
Het⁵ is a 3 to 8-membered saturated or partially unsaturated monocyclic heterocycle, containing 1 or 2 heteroatoms selected from O and N;
Het⁶ is a 6 to 12-membered saturated or partially unsaturated multicyclic heterocycle containing 1 or 2 heteroatoms selected from O and N;
Het⁷ is (i) a 6-membered aromatic heterocycle containing 1-3 N atoms or (ii) a 5-membered aromatic heterocycle containing either (a) 1-4 N atoms or (b) 1 O or S atom and 0-3 N atoms;
Het⁸ is (i) a 10-membered bicyclic aromatic heterocycle containing 1-4 N atoms or (ii) a 9-membered bicyclic aromatic heterocycle containing either (a) 1-4 N atoms or (b) 1 O or S atom and 0-3 N atoms;
R^{c} is in each instance independently selected from C₁-C₆ alkyl, C₃-C₈ cycloalkyl, C₆-C₁₂ bicycloalkyl, Aryl², Het⁹, Het¹⁰, Het¹¹ and Het¹², said C₁-C₆ alkyl, C₃-C₈ cycloalkyl, C₆-C₁₂ bicycloalkyl, Aryl², Het⁹, Het¹⁰, Het¹¹ and Het¹² each being optionally substituted by 1-3 substituents selected from R^{e} and one or more halo atoms;
R^{d} is in each instance independently selected from H, C₁-C₆ alkyl, C₃-C₈ cycloalkyl, C₆-C₁₂ bicycloalkyl, Aryl², Het⁹, Het¹⁰, Het¹¹ and Het¹², said C₁-C₆ alkyl, C₃-C₈ cycloalkyl, C₆-C₁₂ bicycloalkyl, Aryl², Het⁹, Het¹⁰, Het¹¹ and Het¹² each being optionally substituted by 1-3 substituents selected from R^{e} and one or more halo atoms;
Aryl² is phenyl or naphthyl;
Het⁹ is a 3 to 8-membered saturated or partially unsaturated monocyclic heterocycle, containing 1 or 2 heteroatoms selected from O and N;
Het¹⁰ is a 6 to 12-membered saturated or partially unsaturated multicyclic heterocycle containing 1 or 2 heteroatoms selected from O and N;
Het¹¹ is (i) a 6-membered aromatic heterocycle containing 1-3 N atoms or (ii) a 5-membered aromatic heterocycle containing either (a) 1-4 N atoms or (b) 1 O or S atom and 0-3 N atoms;
Het¹² is (i) a 10-membered bicyclic aromatic heterocycle containing 1-4 N atoms or (ii) a 9-membered bicyclic aromatic heterocycle containing either (a) 1-4 N atoms or (b) 1 O or S atom and 0-3 N atoms; and
R^{e} is —OR^{x}, —S(O)ₙR^{x}, —COR^{x}, —NR^{x}R^{x}, —OCOR^{x}, —COOR^{x}, —NR^{x}COR^{x}, —CONR^{x}R^{x} —NR^{x}SO₂R^{x}, —SO₂NR^{x}R^{x}, —NR^{x}SO₂NR^{x}NR^{x}, —NR^{x}COOR^{x}, —NR^{x}CONR^{x}R^{x}, —OCONR^{x}R^{x}, —OCOOR^{x}, —CONR^{x}SO₂R^{x}, oxo or —CN;
with the proviso that the compound of formula (I) is not:
2-hydroxy-N,6-diphenyl-3-pyridinecarboxamide,
N,6-diphenyl-3-pyridinecarboxamide,
6-(2-chlorophenyl)-N-phenyl-3-pyridinecarboxamide,
6-(2-fluorophenyl)-N-phenyl-3-pyridinecarboxamide,
6-(2-methylphenyl)-N-phenyl-3-pyridinecarboxamide,
2-methyl-N,6-diphenyl-3-pyridinecarboxamide,
N-(5-butyl-1,3,4-thiadiazol-2-yl)-2-methyl-6-phenyl-3-pyridinecarboxamide,
N-(4-acetyl-2-thiazolyl)-2-methyl-6-phenyl-3-pyridinecarboxamide,
5-[[(2-methyl-6-phenyl-3-pyridinyl)carbonyl]amino]-2-thiophenecarboxylic acid, methyl ester,
N-[4-(1,1-dimethylethyl)-2-thiazolyl]-2-methyl-6-phenyl-3-pyridinecarboxamide,
N-[4-[5-[(acetylamino)methyl]-2-thienyl]-2-thiazolyl]-2-methyl-6-phenyl-3-pyridinecarboxamide,
N-[4-[4-[(methylsulphonyl)(methyl)amino]phenyl]-2-thiazolyl]-2-methyl-6-phenyl-3-pyridinecarboxamide,
N-[4-[4-(acetylamino)-2-fluorophenyl]-2-thiazolyl]-2-methyl-6-phenyl-3-pyridinecarboxamide,
N-[4-[(2,6-dimethyl-4-morpholinyl)methyl]-2-thiazolyl]-2-methyl-6-phenyl-3-pyridinecarboxamide,
N-[5-[1-(difluoromethyl)-1H-imidazol-2-yl]-4-methyl-2-thiazolyl]-2-methyl-6-phenyl-3-pyridinecarboxamide,
N-[5-(1-ethylpropyl)-1,3,4-thiadiazol-2-yl]-2-methyl-6-phenyl-3-pyridinecarboxamide,
N-(3,5-dimethyl-1-phenyl-1H-pyrazol-4-yl)-2-methyl-6-phenyl-3-pyridinecarboxamide,
N-antipyrinyl-2-methyl-6-phenyl-nicotinamide,
1,2-dihydro-2-oxo-6-phenyl-N-1H-tetrazol-5-yl-3-pyridinecarboxamide,
2-methyl-6-phenyl-N-2-thiazolyl-3-pyridinecarboxamide,
2-methyl-N-(5-methyl-2-thiazolyl)-6-phenyl-3-pyridinecarboxamide,
2-methyl-N-(4-methyl-2-pyridinyl)-6-phenyl-3-pyridinecarboxamide,
N-(5-ethyl-1,3,4-thiadiazol-2-yl)-2-methyl-6-phenyl-3-pyridinecarboxamide,
N-[4-(2-amino-2-oxoethyl)-2-thiazolyl]-2-methyl-6-phenyl-3-pyridinecarboxamide, or
N-[5-(ethylthio)-1,3,4-thiadiazol-2-yl]-2-methyl-6-phenyl-3-pyridinecarboxamide;
6-(2-methylphenyl)-N-[2-[[[1-phenyl-3-(trifluoromethyl)-1H-pyrazol-4-yl]carbonyl]amino]ethyl]-3-pyridinecarboxamide,
N-[2-(5-methoxy-1H-indol-3-yl)ethyl]-6-phenyl-3-pyridinecarboxamide,
N-[4-[4-[1-(2-amino-2-oxoethoxy)-5,6,7,8-tetrahydro-2-naphthalenyl]-1-piperidinyl]butyl]-6-(4-chlorophenyl)-3-pyridinecarboxamide,
N-[4-[4-[1-(2-amino-2-oxoethoxy)-5,6,7,8-tetrahydro-2-naphthalenyl]-1-piperidinyl]butyl]-6-(4-cyanophenyl)-3-pyridinecarboxamide,
6-(4-chlorophenyl)-N-[4-[4-(5,6,7,8-tetrahydro-1-methoxy-2-naphthalenyl]-1-piperidinyl]butyl]-3-pyridinecarboxamide,
6-(4-chlorophenyl)-N-[4-[4-(5,6,7,8-tetrahydro-1-methoxy-2-naphthalenyl]-1-piperidinyl]butyl]-3-pyridinecarboxamide,
6-(2-chlorophenyl)-N-[(1S)-2-[(cyanomethyl)amino]-1-[(2,6-difluorophenyl)methyl]-2-oxoethyl]-3-pyridinecarboxamide,
6-(2-chlorophenyl)-N-[(1S)-2-[(cyanomethyl)amino]-1-[(2,6-difluoro-4-methoxyphenyl)methyl]-2-oxoethyl]-3-pyridinecarboxamide,
6-(2-chlorophenyl)-N-[(1S)-2-[(4-cyano-1-ethyl-4-piperidinyl)amino]-1-[(2,6-difluorophenyl)methyl]-2-oxoethyl]-3-pyridinecarboxamide,
6-(2-chlorophenyl)-N-[(1S)-2-[(cyanomethyl)amino]-2-oxo-1-(2-thiazolylmethyl)ethyl]-3-pyridinecarboxamide,
6-(2-chlorophenyl)-N-[(1S,3S)-1-[[(4-cyano-1-ethyl-4-piperidinyl)amino]carbonyl]-3-phenyl)butyl]-3-pyridinecarboxamide,
N-[[6-(2-chlorophenyl)-3-pyridinyl]carbonyl]-2,6-difluoro-L-phenylalanine,
6-(2-chlorophenyl)-N-[(1S)-2-[(cyanomethyl)amino]-1-[(2,6-difluorophenyl)methyl]-2-oxoethyl]-3-pyridinecarboxamide,
6-(2-chlorophenyl)-N-[(1S)-1-[[(cyanomethyl)amino]carbonyl]-3-methylbutyl]-3-pyridinecarboxamide,
6-(4-methoxyphenyl)-N-[2-[4-(1-pyrrolidinylmethyl)phenyl]ethyl]-3-pyridinecarboxamide,
6-(4-fluorophenyl)-N-[2-[4-(1-pyrrolidinylmethyl)phenyl]ethyl]-3-pyridinecarboxamide,
α-[[[6-(3,4-dimethoxyphenyl)-1,2-dihydro-2-oxo-3-pyridinyl]carbonyl]amino]-4-hydroxybenzeneacetic acid,
N-[4-[4-(2,4-dimethoxyphenyl)-1-piperazinyl]butyl]-6-phenyl-3-pyridinecarboxamide,
5-[[2-(4-fluorophenyl)-1,1-dimethylethylamino]-4-[[[6-(3-methoxyphenyl)-3-pyridinyl]carbonyl]amino]-5-oxo-pentanoic acid,
5-[[2-(4-fluorophenyl)-1,1-dimethylethylamino]-5-oxa-4-[[(6-phenyl)-3-pyridinyl]carbonyl]amino]-(4S)-pentanoic acid,
5-[(1,1-dimethyl-2-phenylethyl)amino]-5-oxo-4-[[(6-phenyl)-3-pyridinyl]carbonyl]amino]-pentanoic acid,
5-[[2-(4-chlorophenyl)-1,1-dimethylethyl]amino]-5-oxo-4-[[(6-phenyl-3-pyridinyl)carbonyl]amino]-(4S)-pentanoic acid,
5-oxo-5-[(phenylmethyl)amino]-4-[[(6-phenyl-3-pyridinyl)carbonyl]amino]-(4S)-pentanoic acid 1,1-dimethylethyl ester,
5-oxo-5-[(phenylmethyl)amino]-4-[[(6-phenyl-3-pyridinyl)carbonyl]amino]-pentanoic acid,
5-[[(3-methoxyphenyl)methyl]amino]-5-oxo-4-[[(6-phenyl-3-pyridinyl)carbonyl]amino]-(4S)-pentanoic acid 1,1-dimethylethyl ester,
5-[[(3-methoxyphenyl)methyl]amino]-5-oxo-4-[[(6-phenyl-3-pyridinyl)carbonyl]amino]-(4S)-pentanoic acid,
N-(2-furanylmethyl)-2-methyl-6-phenyl-3-pyridinecarboxamide,
N-methyl-6-phenyl-3-pyridinecarboxamide, or
6-(4-methoxyphenyl)-N-[[3-[(8-methyl-8-azabicyclo[3.2.1]oct-3-yl)phenyl]methyl]-3-pyridinecarboxamide;
   and with the proviso that when R¹, R², R³, R⁴ and R⁵ are each H, and R⁷ is optionally substituted C₁-C₆ alkyl, R⁶ is not CH₃ or OH;
   and with the proviso that when R¹, R², R⁴ and R⁵ are each H, R³ is trifluoromethyl, R⁶ is CH₃ and R⁷ is methyl or ethyl substituted by R^{a}, R^{a} is not an optionally substituted phenyl ring or an optionally substituted phenyoxy group;
   and with the proviso that when R¹, R², R⁴ and R⁵ are each H, R³ is F, R⁶ is H and R⁷ is methyl substituted by R^{a}, R^{a} is not an optionally substituted quinolinyl group;
   and with the proviso that when one of R¹ and R⁵ is Cl and the other of R¹ and R⁵ is H, R² is H, R³ is H, R⁴ is H, R⁷ is methyl substituted by —CONR^{x}R^{b} and R^{b} is propyl, R^{b} is not substituted by —COHet³ or —COHet⁴;
   and with the proviso that when R⁶ is H, R^{6a} is H, and R⁷ is methyl substituted by R^{a}, R^{a} is not a substituted phenyl group;
   and with the proviso that when R⁶ is H and R^{6a} is H, R⁷ is not (CH₃)₂CHCH₂CH₂—.

In a preferred embodiment E2, R¹, R², R³, R⁴ and R⁵ are each independently H, F, —CH₃, —OH or —OCH₃ and R⁶, R^{6a} and R⁷ are as defined in embodiment E1 above.

In a preferred embodiment E3, R¹ is H, R², R³, R⁴ and R⁵ are each independently H, F, —CH₃, —OH or —OCH₃ and R⁶, R^{6a} and R⁷ are as defined in embodiment E1 above.

In a preferred embodiment E4, R¹, R³, R⁴ and R⁵ are H and R² is F; or R¹, R³, R⁴ and R⁵ are H and R² is —CH₃; or R¹, R³, R⁴ and R⁵ are H and R² is —OCH₃; or R¹, R², R⁴ and R⁵ are H and R³ is F; or R¹, R³ and R⁵ are H and R² and R⁴ are both F; or R¹, R², R³, R⁴ and R⁵ are each H; or R¹, R³ and R⁵ are H, R² is F and R⁴ is —OCH₃; or R¹, R³ and R⁴ are H, R² is F and R⁵ is —OH; and R⁶, R^{6a} and R⁷ are as defined in embodiment E1 above.

In a preferred embodiment E5, R¹, R³, R⁴ and R⁵ are H, R² is F and R⁶, R^{6a} and R⁷ are as defined in embodiment E1 above.

In a preferred embodiment E6, R⁶ is H and R¹, R², R³, R⁴, R⁵, R^{6a} and R⁷ are as defined in embodiment E1 above.

In a preferred embodiment E7, R^{6a} is H or Cl and R¹, R², R³, R⁴, R⁵, R⁶ and R⁷ are as defined in embodiment E1 above.

In a preferred embodiment E8, R^{6a} is H and R¹, R², R³, R⁴, R⁵, R⁶ and R⁷ are as defined in embodiment E1 above.

In a preferred embodiment E9, R⁷ is C₁-C₆ alkyl optionally substituted by 1-3 substituents selected from R^{a}, —OR^{b}, —S(O)ₙR^{b}, —COR^{b}, —NR^{x}R^{b}, —OCOR^{b}, —COOR^{b}, —NR^{x}COR^{b}, —CONR^{x}R^{b} —NR^{x}SO₂R^{b}, —SO₂NR^{x}R^{b}, —NR^{x}SO₂NR^{x}R^{b}, —NR^{x}COOR^{b}, —NR^{x}CONR^{x}R^{b}, —OCONR^{x}R^{b}, —OCOOR^{b}, —CONR^{x}SO₂R^{b}, oxo and —CN, and optionally substituted by one or more halo atoms; and R¹, R², R³, R⁴, R⁵, R⁶ and R^{6a} are as defined in embodiment E1 above.

In a preferred embodiment E9a, R⁷ is C₁-C₆ alkyl and R¹, R², R³, R⁴, R⁵, R⁶ and R^{6a} are as defined in embodiment E1 above.

In a preferred embodiment E9b, R⁷ is C₁-C₆ alkyl optionally substituted 1-3 substituents selected from —OH, —N(C₁-C₆ alkyl)(C₁-C₆ alkyl), —O(C₁-C₆ alkyl), —CO₂H, —NH—(C₁-C₆ alkylene)-O(C₁-C₆ alkyl), —COO(C₁-C₆ alkyl), —CN, —SO₂(C₁-C₆ alkyl), —CON(C₁-C₆ alkyl)(C₁-C₆ alkyl), —CONH—(C₁-C₆ alkylene)-COO(C₁-C₆ alkyl), —O—(C₁-C₆ alkylene)-OH, —NH₂, —NHCOO—(C₁-C₆ alkylene)-phenyl, —CO(C₁-C₆ alkyl) and C₁-C₆ alkyl; and R¹, R², R³, R⁴, R⁵, R⁶ and R^{6a} are as defined in embodiment E1 above.

In a preferred embodiment E9c, R⁷ is methyl optionally substituted by 1-3 substituents selected from R^{a}, —OR^{b}, —S(O)ₙR^{b}, —COR^{b}, —NR^{x}R^{b}, —OCOR^{b}, —COOR^{b}, —NR^{x}COR^{b}, —CONR^{x}R^{b} —NR^{x}SO₂R^{b}, —SO₂NR^{x}R^{b}, —NR^{x}SO₂NR^{x}R^{b}, —NR^{x}COOR^{b}, —NR^{x}CONR^{x}R^{b}, —OCONR^{x}R^{b}, —OCOOR^{b}, —CONR^{x}SO₂R^{b}, oxo and —CN, and optionally substituted by one or more halo atoms; and R¹, R², R³, R⁴, R⁵, R⁶ and R^{6a} are as defined in embodiment E1 above.

In a preferred embodiment E9d, R⁷ is methyl optionally substituted by 1-3 substituents selected from phenyl, —CN, —OH, —COO(C₁-C₆ alkyl), C₃-C₈ cycloalkyl, —COO—(C₁-C₆ alkylene)-phenyl, Het⁵, Het⁶, Het⁷ and Het⁸, said phenyl, C₃-C₈ cycloalkyl, Het⁵, Het⁶, Het⁷ and Het⁸ being optionally substituted by 1-3 substituents selected from C₁-C₆ alkyl, C₃-C₈ cycloalkyl —CO(C₁-C₆ alkyl), C₁-C₆ alkoxy, (C₁-C₆ alkoxy)C₁-C₆ alkyl, halo, C₁-C₆ haloalkyl, —S(C₁-C₆ alkyl), —SO₂NH₂, —COO(C₁-C₆ alkyl), —SO₂(C₁-C₆ alkyl), phenyl, phenyl(C₁-C₆ alkyl), (C₁-C₆ alkoxy)phenyl, ((C₁-C₆ alkoxy)phenyl)C₁-C₆ alkyl, —(C₁-C₆ alkylene)-SO₂—(C₁-C₆ alkyl), halophenyl, Het⁹, Het¹⁰, Het¹¹, —COHet⁹, —(C₁-C₆ alkylene)-Het⁹, —(C₁-C₆ alkylene)-Het¹¹, —SO₂NH(C₁-C₆ alkyl), —(C₁-C₆ alkylene)-COO(C₁-C₆ alkyl), —OH and oxo, said Het⁹, Het¹⁰ and Het¹¹ being optionally substituted by 1-3 substituents selected from C₁-C₆ alkyl, C₃-C₈ cycloalkyl, C₁-C₆ haloalkyl, C₁-C₆ alkoxy(C₁-C₆ alkyl), —OH and oxo.

In a preferred embodiment E9e, R⁷ is ethyl optionally substituted by 1-3 substituents selected from R^{a}, —OR^{b}, —S(O)ₙR^{b}, —COR^{b}, —NR^{x}R^{b}, —OCOR^{b}, —COOR^{b}, —NR^{x}COR^{b}, —CONR^{x}R^{b} —NR^{x}SO₂R^{b}, —SO₂NR^{x}R^{b}, —NR^{x}SO₂NR^{x}R^{b}, —NR^{x}COOR^{b}, —NR^{x}CONR^{x}R^{b}, —OCONR^{x}R^{b}, —OCOOR^{b}, —CONR^{x}SO₂R^{b}, oxo and —CN, and optionally substituted by one or more halo atoms; and R¹, R², R³, R⁴, R⁵, R⁶ and R^{6a} are as defined in embodiment E1 above.

In a preferred embodiment E9f, R⁷ is ethyl optionally substituted by 1-3 substituents selected from phenyl, Het⁵, Het⁷, Het⁸, —NHHet⁷, —NHHet⁸, —O—(C₁-C₆ alkylene)-Het⁸, —CN, —OH, —CONH₂, —CONH—(C₁-C₆ alkylene)-Het⁵, —COO(C₁-C₆ alkyl), C₃-C₈ cycloalkyl, —NH(phenyl), —N(C₁-C₆ alkyl)(C₁-C₆ alkyl), —O(phenyl) and —NHCOO—(C₁-C₆ alkylene)-phenyl, said phenyl, Het⁵, Het⁷ and Het⁸ being optionally substituted by 1-3 substituents selected from —OH, halo, C₁-C₆ alkyl, C₁-C₆ haloalkyl C₃-C₈ cycloalkyl, C₁-C₆ alkoxy, hydroxy(C₁-C₆ alkyl), oxo, phenyl, halophenyl, (C₁-C₆ alkyl)phenyl, phenyl(C₁-C₆ alkyl), (hydroxyphenyl)C₁-C₆ alkyl, (C₁-C₆ alkoxy)phenyl, Het¹¹, —(C₁-C₆ alkylene)-Het⁹, (C₁-C₆ alkoxy)C₁-C₆ alkyl and —(C₁-C₆ alkylene)-Het¹¹, said Het⁹ and Het¹¹ being optionally substituted by 1-3 substituents selected from C₁-C₆ alkyl, C₁-C₆ alkoxy(C₁-C₆ alkyl) and oxo.

In a preferred embodiment E9g, R⁷ is propyl optionally substituted by 1-3 substituents selected from R^{a}, —OR^{b}, —S(O)ₙR^{b}, —COR^{b}, —NR^{x}R^{b}, —OCOR^{b}, —COOR^{b}, —NR^{x}COR^{b}, —CONR^{x}R^{b} —NR^{x}SO₂R^{b}, —SO₂NR^{x}R^{b}, —NR^{x}SO₂NR^{x}R^{b}, —NR^{x}COOR^{b}, —NR^{x}CONR^{x}R^{b}, —OCONR^{x}R^{b}, —OCOOR^{b}, —CONR^{x}SO₂R^{b}, oxo and —CN, and optionally substituted by one or more halo atoms; and R¹, R², R³, R⁴, R⁵, R⁶ and R^{6a} are as defined in embodiment E1 above.

In a preferred embodiment E9h, R⁷ is propyl optionally substituted by 1-3 substituents selected from Het⁵, Het⁷, Het⁸, —NHHet⁷, —NH₂, C₃-C₈ cycloalkyl, —OH, oxo, —O(phenyl) and —O—(C₁-C₆ alkylene)-phenyl, said phenyl, Het⁵, Het⁷ and Het⁸ being optionally substituted by 1-3 substituents selected from C₁-C₆ alkyl, C₁-C₆ alkoxy and oxo.

In a preferred embodiment E9i, R⁷ is C₁-C₃ alkyl optionally substituted by 1-3 substituents selected from phenyl, —CN, —OH, —NH₂, oxo, —COO(C₁-C₆ alkyl), C₃-C₈ cycloalkyl, —COO—(C₁-C₆ alkylene)-NHHet⁷, —NHHet⁸, —O—(C₁-C₆ alkylene)-Het⁸, —O—(C₁-C₆ alkylene)-phenyl, —CONH₂, —CONH—(C₁-C₆ alkylene)-Het⁹, —NH(phenyl), phenyl, —N(C₁-C₆ alkyl)(C₁-C₆ alkyl), —O(phenyl), —NHCOO—(C₁-C₆ alkylene)-phenyl, Het⁵, Het⁶, Het⁷ and Het⁸, said phenyl, C₃-C₈ cycloalkyl, Het⁵, Het⁶, Het⁷ and Het⁸ being optionally substituted by 1-3 substituents selected from C₁-C₆ alkyl, C₃-C₈ cycloalkyl —CO(C₁-C₆ alkyl), C₁-C₆ alkoxy, (C₁-C₆ alkoxy)C₁-C₆ alkyl, hydroxyl(C₁-C₆ alkyl), hydroxylphenyl(C₁-C₆ alkyl), halophenyl, (C₁-C₆ alkyl)phenyl, halo, C₁-C₆ haloalkyl, —S(C₁-C₆ alkyl), —SO₂NH₂, —COO(C₁-C₆ alkyl), —SO₂(C₁-C₆ alkyl), phenyl, phenyl(C₁-C₆ alkyl), (C₁-C₆ alkoxyphenyl), ((C₁-C₆ alkoxy)phenyl)C₁-C₆ alkyl, —(C₁-C₆ alkylene)-SO₂(C₁-C₆ alkyl), halophenyl, Het⁹, Het¹⁰, Het¹¹, —COHet⁹, —(C₁-C₆ alkylene)-Het⁹, —(C₁-C₆ alkylene)-Het¹¹, —SO₂NH(C₁-C₆ alkyl), —(C₁-C₆ alkylene)-COO(C₁-C₆ alkyl), —OH and oxo, said Het⁹, Het¹⁰ and Het¹¹ being optionally substituted by 1-3 substituents selected from C₁-C₆ alkyl, C₃-C₈ cycloalkyl, C₁-C₆ haloalkyl, C₁-C₆ alkoxy(C₁-C₆ alkyl), —OH and oxo.

In a preferred embodiment E10, R⁷ is phenyl optionally substituted by 1-3 substituents selected from R^{a}, —OR^{b}, —S(O)ₙR^{b}, —COR^{b}, —OCOR^{b}, —COOR^{b}, —NR^{x}COR^{b}, —CONR^{x}R^{b} —NR^{x}SO₂R^{b}, —SO₂NR^{x}R^{b}, —NR^{x}SO₂NR^{x}R^{b}, —NR^{x}COOR^{b}, —NR^{x}CONR^{x}R^{b}, —OCONR^{x}R^{b}, —OCOOR^{b}, —CONR^{x}SO₂R^{b}, oxo and —CN, and optionally substituted by one or more halo atoms; and R¹, R², R³, R⁴, R⁵, R⁶ and R^{6a} are as defined in embodiment E1 above.

In a preferred embodiment E10a, R⁷ is phenyl optionally substituted by 1-2 substituents selected from R^{a} and —OR^{b}, and optionally substituted by one or more halo atoms; and R¹, R², R³, R⁴, R⁵, R⁶ and R^{6a} are as defined in embodiment E1 above.

In a preferred embodiment E10b, R⁷ is phenyl optionally substituted by 1-3 substituents selected from C₁-C₆ alkyl, C₁-C₆ alkoxy and halo; and R¹, R², R³, R⁴, R⁵, R⁶ and R^{6a} are as defined in embodiment E1 above.

In a preferred embodiment E11, R⁷ is Het¹ optionally substituted by 1-3 substituents selected from R^{a}, —S(O)ₙR^{b}, —COR^{b}, —NR^{x}R^{b}, —OCOR^{b}, —COOR^{b}, —NR^{x}COR^{b}, —CONR^{x}R^{b} —NR^{x}SO₂R^{b}, —SO₂NR^{x}R^{b}, —NR^{x}SO₂NR^{x}R^{b}, —NR^{x}COOR^{b}, —NR^{x}CONR^{x}R^{b}, —OCONR^{x}R^{b}, —OCOOR^{b}, —CONR^{x}SO₂R^{b}, oxo and —CN, and optionally substituted by one or more halo atoms; and R¹, R², R³, R⁴, R⁵, R⁶ and R^{6a} are as defined in embodiment E1 above.

In a preferred embodiment E11a, R⁷ is a 5- or 6-membered saturated heterocycle comprising one O or N atom, said heterocycle being optionally substituted by 1-3 substituents selected from R^{a}, —OR^{b}, —S(O)ₙR^{b}, —COR^{b}, —NR^{x}R^{b}, —OCOR^{b}, —COOR^{b}, —NR^{x}COR^{b}, —CONR^{x}R^{b} —NR^{x}SO₂R^{b}, —SO₂NR^{x}R^{b}, —NR^{x}SO₂NR^{x}R^{b}, —NR^{x}COOR^{b}, —NR^{x}CONR^{x}R^{b}, —OCONR^{x}R^{b}, —OCOOR^{b}, —CONR^{x}SO₂R^{b}, oxo and —CN, and optionally substituted by one or more halo atoms; and R¹, R², R³, R⁴, R⁵, R⁶ and R^{6a} are as defined in embodiment E1 above.

In a preferred embodiment E11b, R⁷ is a 5- or 6-membered saturated heterocycle comprising one O or N atom, said heterocycle being optionally substituted by 1-3 substituents selected from R^{a}, —OR^{b}, —COOR^{b}, oxo, —NR^{x}R^{b}; and R¹, R², R³, R⁴, R⁵, R⁶ and R^{6a} are as defined in embodiment E1 above.

In a preferred embodiment E11c, R⁷ is tetrahydropyranyl, pyrrolidinyl, azepinyl or tetrahydrofuranyl, each being optionally substituted by 1-3 substituents selected from R^{a}, —OR^{b}, —COOR^{b}, —COR^{b}, oxo, —NR^{x}R^{b}; and R¹, R², R³, R⁴, R⁵, R⁶ and R^{6a} are as defined in embodiment E1 above.

In a preferred embodiment E11d, R⁷ is tetrahydropyranyl, pyrrolidinyl, azepinyl or tetrahydrofuranyl, each being optionally substituted by 1-3 substituents selected from C₁-C₆ alkyl, —OH, —COO(C₁-C₆ alkyl), —CO(C₁-C₆ alkyl), Het⁶, Het⁷, Het⁸, oxo, —N(C₁-C₆ alkyl)(C₁-C₆ alkyl), —(C₁-C₆ alkyl)Aryl¹, said Het⁶, Het⁷, Het⁸ and Aryl¹ being optionally substituted by 1-3 substituents selected from C₁-C₆ alkyl, —CN and halo; and R¹, R², R³, R⁴, R⁵, R⁶ and R^{6a} are as defined in embodiment E1 above.

In a preferred embodiment E12, R⁷ is Het² optionally substituted by 1-3 substituents selected from R^{a}, —OR^{b}, —S(O)ₙR^{b}, —COR^{b}, —OCOR^{b}, —COOR^{b}, —NR^{x}COR^{b}, —CONR^{x}R^{b} —NR^{x}SO₂R^{b}, —SO₂NR^{x}R^{b}, —NR^{x}SO₂NR^{x}R^{b}, —NR^{x}COOR^{b}, —NR^{x}CONR^{x}R^{b}, —OCONR^{x}R^{b}, —OCOOR^{b}, —CONR^{x}SO₂R^{b}, oxo and —CN, and optionally substituted by one or more halo atoms; and R¹, R², R³, R⁴, R⁵, R⁶ and R^{6a} are as defined in embodiment E1 above.

In a preferred embodiment E12a, R⁷ is Het² optionally substituted by 1-3 substituents selected from R^{a}, —COOR^{b}, —SO₂R^{b}, —COR^{b} and oxo; and R¹, R², R³, R⁴, R⁵, R⁶ and R^{6a} are as defined in embodiment E1 above.

In a preferred embodiment E12b, R⁷ is an 8- to 11-membered saturated or partially unsaturated heterocycle containing 1 oxygen atom, 1 nitrogen atom or 1 oxygen and 1 nitrogen atom, said heterocycle being optionally substituted by 1-3 substituents selected from R^{a}, —COOR^{b}, —SO₂R^{b}, —COR^{b} and oxo; and R¹, R², R³, R⁴, R⁵, R⁶ and R^{6a} are as defined in embodiment E1 above.

In a preferred embodiment E12c, R⁷ is an 8- to 11-membered saturated or partially unsaturated heterocycle containing 1 oxygen atom, 1 nitrogen atom or 1 oxygen and 1 nitrogen atom, said heterocycle being optionally substituted by 1-3 substituents selected from C₁-C₆ alkyl, —COO(C₁-C₆ alkyl), —SO₂(C₁-C₆ alkyl), —CO(C₁-C₆ alkyl), Het⁷, Het⁸, —(C₁-C₆ alkylene)-Het⁷, (C₁-C₆ alkoxy)C₁-C₆ alkyl and oxo, wherein Het⁷ and Het⁸ may optionally be substituted by a C₁-C₆ alkyl, hydroxyl(C₁-C₆ alkyl) or morpholinylcarbonyl group; and R¹, R², R³, R⁴, R⁵, R⁶ and R^{6a} are as defined in embodiment E1 above.

In a preferred embodiment E12d, R⁷ is 8-azabicyclo[3.2.1]octyl, 3,4-dihydro-2H-chromenyl, azabicyclo[3.1.0]hex-6-yl] or 1-oxa-8-azaspiro[4.5]decyl, each being optionally substituted by 1-3 substituents selected from C₁-C₆ alkyl, —COO(C₁-C₆ alkyl), —SO₂(C₁-C₆ alkyl), —CO(C₁-C₆ alkyl), Het⁷, Het⁸, —(C₁-C₆ alkylene)-Het⁷, (C₁-C₆ alkoxy)C₁-C₆ alkyl and oxo, wherein Het⁷ and Het⁸ may optionally be substituted by a C₁-C₆ alkyl, hydroxyl(C₁-C₆ alkyl) or morpholinylcarbonyl group; and R¹, R², R³, R⁴, R⁵, R⁶ and R^{6a} are as defined in embodiment E1 above.

In a preferred embodiment E12e, R⁷ is 8-azabicyclo[3.2.1]octyl (preferably 8-azabicyclo[3.2.1]oct-3-yl) optionally substituted by 1-3 substituents selected from R^{a}, —OR^{b}, —S(O)ₙR^{b}, —COR^{b}, —NR^{x}R^{b}, —COOR^{b}, —COOR^{b}, —NR^{x}COR^{b}, —CONR^{x}R^{b} —NR^{x}SO₂R^{b}, —SO₂NR^{x}R^{b}, —NR^{x}SO₂NR^{x}R^{b}, —NR^{x}COOR^{b}, —NR^{x}CONR^{x}R^{b}, —OCONR^{x}R^{b}, —OCOOR^{b}, —CONR^{x}SO₂R^{b}, oxo and —CN, and optionally substituted by one or more halo atoms; and R¹, R², R³, R⁴, R⁵, R⁶ and R^{6a} are as defined in embodiment E1 above.

In a preferred embodiment E12f, R⁷ is 8-azabicyclo[3.2.1]octyl (preferably 8-azabicyclo[3.2.1]oct-3-yl) optionally substituted by 1-3 substituents selected from C₁-C₆ alkyl, —COO(C₁-C₆ alkyl), —SO₂(C₁-C₆ alkyl), —CO(C₁-C₆ alkyl), Het⁷, Het⁸, —(C₁-C₆ alkylene)-Het⁷, (C₁-C₆ alkoxy)C₁-C₆ alkyl and oxo, wherein Het⁷ and Het⁸ may optionally be substituted by a C₁-C₆ alkyl, hydroxyl(C₁-C₆ alkyl) or morpholinylcarbonyl group; and R¹, R², R³, R⁴, R⁵, R⁶ and R^{6a} are as defined in embodiment E1 above.

In a preferred embodiment E13, R⁷ is Het³ optionally substituted by 1-3 substituents selected from R^{a}, —OR^{b}, —S(O)ₙR^{b}, —COR^{b}, —NR^{x}R^{b}, —COOR^{b}, —COOR^{b}, —NR^{x}COR^{b}, —CONR^{x}R^{b} —NR^{x}SO₂R^{b}, —SO₂NR^{x}R^{b}, —NR^{x}SO₂NR^{x}R^{b}, —NR^{x}COOR^{b}, —NR^{x}CONR^{x}R^{b}, —OCONR^{x}R^{b}, —OCOOR^{b}, —CONR^{x}SO₂R^{b}, oxo and —CN, and optionally substituted by one or more halo atoms; and R¹, R², R³, R⁴, R⁵, R⁶ and R^{6a} are as defined in embodiment E1 above.

In a preferred embodiment E13a, R⁷ is Het³ optionally substituted by 1-3 substituents R^{a} and optionally substituted by one or more halo atoms; and R¹, R², R³, R⁴, R⁵, R⁶ and R^{6a} are as defined in embodiment E1 above.

In a preferred embodiment E13b, R⁷ is pyridyl or pyrid-2-onyl optionally substituted by 1-3 substituents R^{a} and optionally substituted by one or more halo atoms; and R¹, R², R³, R⁴, R⁵, R⁶ and R^{6a} are as defined in embodiment E1 above.

In a preferred embodiment E13c, R⁷ is pyridyl or pyrid-2-onyl optionally substituted by one C₁-C₆ alkyl group, said C₁-C₆ alkyl group being optionally substituted by R^{c}; and R¹, R², R³, R⁴, R⁵, R⁶ and R^{6a} are as defined in embodiment E1 above.

In a preferred embodiment E14, the compound of formula (I) is a compound of formula (Ia):

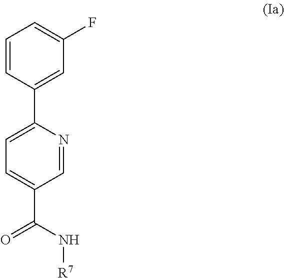

or a pharmaceutically acceptable salt thereof, or a pharmaceutically acceptable solvate of said compound or salt, wherein R⁷ is as defined above in any one of embodiments E1, E9, E9a, E9b, E9c, E9d, E9e, E9f, E9g, E9h, E9i, E10, E10a, E10b, E11, E11a, E11b, E11c, E11d, E12, E12a, E12b, E12c, E12d, E12e, E12f, E13, E13a, E13b or E13c.

Further preferred embodiments of the invention are created by combining the definitions given for R¹-R⁵ in any one of embodiments E1, E2, E3, E4 or E5 with the definition given for R⁶ in embodiment E1 or E6, the definition given for R^{6a} in any one of embodiments E1, E7 or E8 and the definition given for R⁷ in any one of embodiments E1, E9, E9a, E9b, E9c, E9d, E9e, E9f, E9g, E9h, E9i, E10, E10a, E10b, E11, E11a, E11b, E11c, E11d, E12, E12a, E12b, E12c, E12d, E12e, E12f, E13, E13a, E13b or E13c.

The present invention also provides: a method of treating a disease or condition mediated at least in part by prostaglandin D₂ produced by H-PGDS, in a subject in need of such treatment, comprising administering to the subject a therapeutically effective amount of a compound of formula (I), or a pharmaceutically acceptable salt or solvate thereof; the use of a compound of formula (I), or a pharmaceutically acceptable salt or solvate thereof, for the manufacture of a medicament for treating a disease or condition mediated at least in part by prostaglandin D₂ produced by H-PGDS; a compound of formula (I), or a pharmaceutically acceptable salt or solvate thereof, for use as a medicament; a compound of formula (I), or a pharmaceutically acceptable salt or solvate thereof, for use in the treatment of a disease or condition mediated at least in part by prostaglandin D₂ produced by H-PGDS; a pharmaceutical composition comprising a compound of formula (I), or a pharmaceutically acceptable salt or solvate thereof, and a pharmaceutically acceptable excipient; a pharmaceutical composition for the treatment of a disease or condition mediated at least in part by prostaglandin D₂ produced by H-PGDS comprising a compound of formula (I), or a pharmaceutically acceptable salt or solvate thereof.

It is to be noted that in embodiment E1, defined above, several compounds and groups of compounds have been disclaimed, since these compounds are already known per se. However, such compounds are not known in relation to the method and uses described above and the disclaimers may therefore be omitted when the invention is claimed in terms of the use of such compounds. For example, the invention provides as embodiment E1a, a method of treating a disease or condition mediated at least in part by prostaglandin D2 produced by H-PGDS, in a subject in need of such treatment, comprising administering to the subject a therapeutically effective amount of a compound of formula (I):

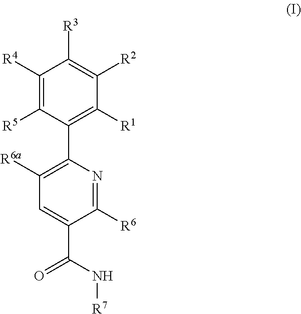

or a pharmaceutically acceptable salt thereof, or a pharmaceutically acceptable solvate of said compound or salt, wherein:
R¹, R², R³, R⁴ and R⁵ are each independently H, F, Cl, —CN, —NH₂, —CH₃, —CHF, —CHF₂, —CF₃, —OH, —OCH₃, —OCH₂F, —OCHF₂ or —OCF₃;

### R⁶ is H, —NH₂, —OH or —CH₃;

### R^{6a} is H, F or Cl;

R⁷ is C₁-C₆ alkyl, phenyl, Het¹, Het², Het³ or Het⁴, said C₁-C₆ alkyl, phenyl, Het¹, Het², Het³ or Het⁴ being (a) optionally substituted by 1-3 substituents selected from R^{a}, —OR^{b}, —S(O)ₙR^{b}, —COR^{b}, —OCOR^{b}, —COOR^{b}, —NR^{x}COR^{b}, —CONR^{x}R^{b} —NR^{x}SO₂R^{b}, —SO₂NR^{x}R^{b}, —NR^{x}SO₂NR^{x}R^{b}, —NR^{x}COOR^{b}, —NR^{x}CONR^{x}R^{b}, —OCONR^{x}R^{b}, —OCOOR^{b}, —CONR^{x}SO₂R^{b}, oxo and —CN, and (b) optionally substituted by one or more halo atoms;
R^{a} is in each instance independently selected from C₁-C₆ alkyl, C₃-C₈ cycloalkyl, C₆-C₁₂ bicycloalkyl, Aryl¹, Het⁵, Het⁶, Het⁷ and Het⁸, said C₁-C₆ alkyl, C₃-C₈ cycloalkyl, C₆-C₁₂ bicycloalkyl, Aryl¹, Het⁵, Het⁶, Het⁷ and Het⁸ each being optionally substituted by 1-3 substituents selected from R^{c}, —OR^{d}, —S(O)ₙR^{d}, —COR^{d}, —NR^{x}R^{d}, —OCOR^{d}, —COOR^{d}, —NR^{x}COR^{d}, —CONR^{x}R^{d} —NR^{x}SO₂R^{d}, —SO₂NR^{x}R^{d}, —NR^{x}SO₂NR^{x}R^{d}, —NR^{x}COOR^{d}, —NR^{x}CONR^{x}R^{d}, —OCONR^{x}R^{d}, —OCOOR^{d}, —CONR^{x}SO₂R^{d}, oxo and —CN and one or more halo atoms;
R^{b} is in each instance independently selected from H, C₁-C₆ alkyl, C₃-C₈ cycloalkyl, C₆-C₁₂ bicycloalkyl, Aryl¹, Het⁵, Het⁶, Het⁷ and Het⁸, said C₁-C₆ alkyl, C₃-C₈ cycloalkyl, C₆-C₁₂ bicycloalkyl, Aryl¹, Het⁵, Het⁶, Het⁷ and Het⁸ each being optionally substituted by 1-3 substituents selected from R^{c}, OR^{d}, —S(O)ₙR^{d}, —COR^{d}, —NR^{x}R^{d}, —OCOR^{d}, —COOR^{d}, —NR^{x}COR^{d}, —CONR^{x}R^{d} —NR^{x}SO₂R^{d}, —SO₂NR^{x}R^{d}, —NR^{x}SO₂NR^{x}R^{d}, —NR^{x}COOR^{d}, —NR^{x}CONR^{x}R^{d}, —OCONR^{x}R^{d}, —OCOOR^{d}, —CONR^{x}SO₂R^{d}, oxo and —CN and one or more halo atoms;
n is 0, 1 or 2;
R^{x} is in each instance independently H, C₁-C₆ alkyl or C₃-C₈ cycloalkyl, said C₁-C₆ alkyl or C₃-C₈ cycloalkyl being optionally substituted by one or more halo atoms;
Aryl¹ is phenyl or naphthyl;
Het¹ is a 3 to 8-membered saturated or partially unsaturated monocyclic heterocycle, containing 1 or 2 heteroatoms selected from O and N, with the proviso that Het¹ is not piperidinyl, pyrrolidinyl and azetidinyl;
Het² is a 6 to 12-membered saturated or partially unsaturated multicyclic heterocycle containing 1 or 2 heteroatoms selected from O and N, with the proviso that Het² is not a bridged piperidinyl, pyrrolidinyl or azetidinyl ring;
Het³ is (i) a 6-membered aromatic heterocycle containing 1-3 N atoms or (ii) a 5-membered aromatic heterocycle containing either (a) 1-4 N atoms or (b) 1 O or S atom and 0-3 N atoms;
Het⁴ is (i) a 10-membered bicyclic aromatic heterocycle containing 1-4 N atoms or (ii) a 9-membered bicyclic aromatic heterocycle containing either (a) 1-4 N atoms or (b) 1 O or S atom and 0-3 N atoms;
Het⁵ is a 3 to 8-membered saturated or partially unsaturated monocyclic heterocycle, containing 1 or 2 heteroatoms selected from O and N;
Het⁶ is a 6 to 12-membered saturated or partially unsaturated multicyclic heterocycle containing 1 or 2 heteroatoms selected from O and N;
Het⁷ is (i) a 6-membered aromatic heterocycle containing 1-3 N atoms or (ii) a 5-membered aromatic heterocycle containing either (a) 1-4 N atoms or (b) 1 O or S atom and 0-3 N atoms;
Het⁸ is (i) a 10-membered bicyclic aromatic heterocycle containing 1-4 N atoms or (ii) a 9-membered bicyclic aromatic heterocycle containing either (a) 1-4 N atoms or (b) 1 O or S atom and 0-3 N atoms;
R^{c} is in each instance independently selected from C₁-C₆ alkyl, C₃-C₈ cycloalkyl, C₆-C₁₂ bicycloalkyl, Aryl², Het⁹, Het¹⁰, Het¹¹ and Het¹², said C₁-C₆ alkyl, C₃-C₈ cycloalkyl, C₆-C₁₂ bicycloalkyl, Aryl², Het⁹, Het¹⁰, Het¹¹ and Het¹² each being optionally substituted by 1-3 substituents selected from R^{e} and one or more halo atoms;
R^{d} is in each instance independently selected from H, C₁-C₆ alkyl, C₃-C₈ cycloalkyl, C₆-C₁₂ bicycloalkyl, Aryl², Het⁹, Het¹⁰, Het¹¹ and Het¹², said C₁-C₆ alkyl, C₃-C₈ cycloalkyl, C₆-C₁₂ bicycloalkyl, Aryl², Het⁹, Het¹⁰, Het¹¹ and Het¹² each being optionally substituted by 1-3 substituents selected from R^{e} and one or more halo atoms;
Aryl² is phenyl or naphthyl;
Het⁹ is a 3 to 8-membered saturated or partially unsaturated monocyclic heterocycle, containing 1 or 2 heteroatoms selected from O and N;
Het¹⁰ is a 6 to 12-membered saturated or partially unsaturated multicyclic heterocycle containing 1 or 2 heteroatoms selected from O and N;
Het¹¹ is (i) a 6-membered aromatic heterocycle containing 1-3 N atoms or (ii) a 5-membered aromatic heterocycle containing either (a) 1-4 N atoms or (b) 1 O or S atom and 0-3 N atoms;
Het¹² is (i) a 10-membered bicyclic aromatic heterocycle containing 1-4 N atoms or (ii) a 9-membered bicyclic aromatic heterocycle containing either (a) 1-4 N atoms or (b) 1 O or S atom and 0-3 N atoms; and
R^{e} is —OR^{x}, —S(O)ₙR^{x}, —COR^{x}, —NR^{x}R^{x}, —OCOR^{x}, —COOR^{x}, —NR^{x}COR^{x}, —CONR^{x}R^{x} —NR^{x}SO₂R^{x}, —SO₂NR^{x}R^{x}, —NR^{x}SO₂NR^{x}NR^{x}, —NR^{x}COOR^{x}, —NR^{x}CONR^{x}R^{x}, —OCONR^{x}R^{x}, —OCOOR^{x}, —CONR^{x}SO₂R^{x}, oxo or —CN.

The disease or condition mediated at least in part by prostaglandin D₂ produced by H-PGDS is preferably an allergic or respiratory condition such as allergic rhinitis, nasal congestion, rhinorrhea, perennial rhinitis, nasal inflammation, asthma of all types, chronic obstructive pulmonary disease (COPD), chronic or acute bronchoconstriction, chronic bronchitis, small airways obstruction, emphysema, chronic eosinophilic pneumonia, adult respiratory distress syndrome, exacerbation of airways hyper-reactivity consequent to other drug therapy, airways disease that is associated with pulmonary hypertension, acute lung injury, bronchiectasis, sinusitis, allergic conjunctivitis or atopic dermatitis, particularly asthma or chronic obstructive pulmonary disease.

Types of asthma include atopic asthma, non-atopic asthma, allergic asthma, atopic bronchial IgE-mediated asthma, bronchial asthma, essential asthma, true asthma, intrinsic asthma caused by pathophysiologic disturbances, extrinsic asthma caused by environmental factors, essential asthma of unknown or inapparent cause, bronchitic asthma, emphysematous asthma, exercise-induced asthma, allergen induced asthma, cold air induced asthma, occupational asthma, infective asthma caused by bacterial, fungal, protozoal, or viral infection, non-allergic asthma, incipient asthma, wheezy infant syndrome and bronchiolytis.

Included in the use of the compounds of formula (I) for the treatment of asthma, is palliative treatment for the symptoms and conditions of asthma such as wheezing, coughing, shortness of breath, tightness in the chest, shallow or fast breathing, nasal flaring (nostril size increases with breathing), retractions (neck area and between or below the ribs moves inward with breathing), cyanosis (gray or bluish tint to skin, beginning around the mouth), runny or stuffy nose, and headache.

The present invention also provides any of the uses, methods or compositions as defined above wherein the compound of formula (I), or pharmaceutically acceptable salt or solvate thereof, is used in combination with another pharmacologically active compound, particularly one of the compounds listed in Table 1 below. Specific combinations useful according to the present invention include combinations comprising a compound of formula (I), or a pharmaceutically acceptable salt or solvate thereof, and (i) a glucocorticosteroid or DAGR (dissociated agonist of the corticoid receptor); (ii) a β₂ agonist, an example of which is a long-acting β₂ agonist; (iii) a muscarinic M3 receptor antagonist or an anticholinergic agent; (iv) a histamine receptor antagonist, which may be an H1 or an H3 antagonist; (v) a 5-lypoxygenase inhibitor; (vi) a thromboxane inhibitor; or (vii) an LTD₄ inhibitor. Generally, the compounds of the combination will be administered together as a formulation in association with one or more pharmaceutically acceptable excipients.

TABLE I

(a) 5-lipoxygenase activating protein (FLAP) antagonists;
(b) Leukotriene antagonists (LTRAs) including antagonists
of LTB₄, LTC₄, LTD₄, and LTE₄;
(c) Histamine receptor antagonists including H1 and H3
antagonists;
(d) α₁- and α₂-adrenoceptor agonist vasoconstrictor
sympathomimetic agents for decongestant use;
(e) muscarinic M3 receptor antagonists or anticholinergic
agents;
(f) PDE inhibitors, e.g. PDE3, PDE4 and PDE5 inhibitors,
such as theophylline;
(g) Sodium cromoglycate;
(h) COX inhibitors both non-selective and selective COX-1
or COX-2 inhibitors (such as NSAIDs);
(i) glucocorticosteroids or DAGR (dissociated agonists of
the corticoid receptor);
(j) Monoclonal antibodies active against endogenous inflam-
matory entities;
(k) β2 agonists, including long-acting β2 agonists;
(l) Integrin antagonists;
(m) Adhesion molecule inhibitors including VLA-4 antagonists;
(n) Kinin-B₁ - and B₂ -receptor antagonists;
(o) Immunosuppressive agents, including inhibitors of the
IgE pathway, and cyclosporin;
(p) Inhibitors of matrix metalloproteases (MMPs), such as.,
MMP9, and MMP12;
(q) Tachykinin NK₁, NK₂ and NK₃ receptor antagonists;
(r) Protease inhibitors, such as elastase inhibitors,
chymase and cathepsin G;
(s) Adenosine A2a receptor agonists and A2b antagonists;
(t) Inhibitors of urokinase;
(u) Compounds that act on dopamine receptors, such as D2
agonists;
(v) Modulators of the NFκB pathway, such as IKK
inhibitors;
(w) modulators of cytokine signaling pathways such as syk
kinase, JAK kinase inhibitors, p38 kinase, SPHK-1 kinase,
Rho kinase, EGF-R or MK-2;
(x) Agents that can be classed as mucolytics or anti-tussive,
and mucokinetics;
(y) Antibiotics;
(z) Antivirals;
(aa) Vaccines;
(bb) Chemokines;
(cc) Epithelial sodium channel (ENaC) blockers or Epithelial
sodium channel (ENaC) inhibitors;
(dd) P2Y2 Agonists and other Nucleotide receptor agonists;
(ee) Inhibitors of thromboxane;
(ff) Niacin;
(gg) Inhibitors of 5-lypoxygenase (5-LO); and
(hh) Adhesion factors including VLAM, ICAM, and ELAM.

Besides being useful for human treatment, compounds of formula (I) are also useful for veterinary treatment of companion animals, exotic animals and farm animals.

When used in the present application, the following abbreviations have the meanings set out below:

APCI (in relation to mass spectrometry) is atmospheric pressure chemical ionization;
BOC or Boc is tert-butyloxycarbonyl;
BOP is (benzotriazol-1-yloxy)tris(dimethylamino)phosphonium hexafluorophosphate;
CDI is 1,1-carbonyldiimidazole;
CH₂Cl₂ is dichloromethane;
CO₂Et is ethyl carboxylate;
DCC is N,N′-dicyclohexylcarbodiimide;
DCM is dichloromethane;
CDCl₃ is deuterochloroform;
DEA is diethylamine;
DIEA is diisopropylethylamine;

### DIPEA is N,N-diisopropylethylamine;

### DMA is N,N-dimethylacetamide;

DMAP is 4-dimethylaminopyridine
DMF is dimethylformamide;
DMSO is dimethyl sulphoxide;
DMSO-d₆ is fully deuterated dimethyl sulphoxide;
EDC/EDAC is N-(3-Dimethylaminopropyl)-N′-ethylcarbodiimide hydrochloride;
ES (in relation to mass spectrometry) is electrospray;
Et is ethyl;
EtOAc is ethyl acetate;
GCMS is gas chromatography mass spectrometry;
h is hour(s);
HATU is N,N,N′,N′-tetramethyl-O-(7-azabenzotriazol-1-yl)uronium hexafluorophosphate;
HBTU is N,N,N′N-tetramethyl-O-(1H-benzotriazol-1-yl)uronium hexafluorophosphate;
¹H NMR or ¹H NMR is proton nuclear magnetic resonance;
HOAt is 1-hydroxy-7-azabenzotriazole;
HOBt is 1-hydroxybenzotriazole;
HPLC is high performance liquid chromatography;
HRMS is high resolution mass spectrometry;
IPA is isopropyl alcohol;
iPr is isopropyl;
LCMS is liquid chromatography mass spectrometry;
LRMS is low resolution mass spectrometry;
Me is methyl;
MeCN is acetonitrile;
MeOH is methanol;
MeOD-d₄ is fully deuterated methanol;
MgSO₄ is magnesium sulphate;
min is minute(s);
NH₄Cl is ammonium chloride;
NH₄OH is a solution of ammonia in water;
MS is mass spectroscopy;
NMM is 4-methylmorpholine;

### NMP is N-methylpyrrolidinone;

RT is retention time;
TBTU is O-(benzotriazol-1-yl)-N,N,N′,N′-tetramethyluronium tetrafluoroborate;
TEA is triethylamine;
TFA is trifluoroacetic acid; and
THF is tetrahydrofuran.

Unless otherwise defined herein, scientific and technical terms used in connection with the present invention shall have the meanings that are commonly understood by those of ordinary skill in the art.

The phrase “therapeutically effective” is intended to qualify the amount of compound or pharmaceutical composition, or the combined amount of active ingredients in the case of combination therapy. This amount or combined amount will achieve the goal of treating the relevant condition.

The term “treatment,” as used herein to describe the present invention and unless otherwise qualified, means administration of the compound, pharmaceutical composition or combination to effect preventative, palliative, supportive, restorative or curative treatment. The term treatment encompasses any objective or subjective improvement in a subject with respect to a relevant condition or disease.

The term “preventive treatment,” as used herein to describe the present invention, means that the compound, pharmaceutical composition or combination is administered to a subject to inhibit or stop the relevant condition from occurring in a subject, particularly in a subject or member of a population that is significantly predisposed to the relevant condition.

The term “palliative treatment,” as used herein to describe the present invention, means that the compound, pharmaceutical composition or combination is administered to a subject to remedy signs and/or symptoms of a condition, without necessarily modifying the progression of, or underlying etiology of, the relevant condition.

The term “supportive treatment,” as used herein to describe the present invention, means that the compound, pharmaceutical composition or combination is administered to a subject as a part of a regimen of therapy, but that such therapy is not limited to administration of the compound, pharmaceutical composition or combination. Unless otherwise expressly stated, supportive treatment may embrace preventive, palliative, restorative or curative treatment, particularly when the compounds or pharmaceutical compositions are combined with another component of supportive therapy.

The term “restorative treatment,” as used herein to describe the present invention, means that the compound, pharmaceutical composition or combination is administered to a subject to modify the underlying progression or etiology of a condition. Non-limiting examples include an increase in forced expiratory volume in one second (FEV 1) for lung disorders, decreased rate of a decline in lung function over time, inhibition of progressive nerve destruction, reduction of biomarkers associated and correlated with diseases or disorders, a reduction in relapses, improvement in quality of life, reduced time spent in hospital during an acute exacerbation event and the like.

The term “curative treatment,” as used herein to describe the present invention, means that compound, pharmaceutical composition or combination is administered to a subject for the purpose of bringing the disease or disorder into complete remission, or that the disease or disorder is undetectable after such treatment.

The term “alkyl”, alone or in combination, means an acyclic, saturated hydrocarbon group of the formula CₙH₂ₙ₊₁ which may be linear or branched. Examples of such groups include methyl, ethyl, n-propyl, isopropyl, n-butyl, isobutyl, sec-butyl, tert-butyl, pentyl, iso-amyl and hexyl. Unless otherwise specified, an alkyl group comprises from 1 to 6 carbon atoms.

The term “alkylene” means a bivalent acyclic, saturated hydrocarbon group of the formula CₙH₂ₙ which may be linear or branched. Example of such groups include —CH₂—, —CH(CH₃)—, —CH₂CH₂—, —CH(CH₃)CH₂—, —CH(CH₃)CH(CH₃)— and —CH₂CH₂CH₂—. Unless otherwise specified, an alkyl group comprises from 1 to 6 carbon atoms.

The carbon atom content of alkyl and various other hydrocarbon-containing moieties is indicated by a prefix designating a lower and upper number of carbon atoms in the moiety, that is, the prefix Cᵢ-Cⱼ indicates a moiety of the integer “i” to the integer “j” carbon atoms, inclusive. Thus, for example, C₁-C₆ alkyl refers to alkyl of one to six carbon atoms, inclusive.

The term “hydroxy,” as used herein, means an OH radical.

Het¹, Het⁵ and Het⁹ are saturated or partially saturated (i.e. non aromatic) heterocycles and may be attached via a ring nitrogen atom or a ring carbon atom. Equally, when substituted, the substituent may be located on a ring nitrogen atom or a ring carbon atom. Specific examples include oxiranyl, aziridinyl, oxetanyl, azetidinyl, tetrahydrofuranyl, pyrrolidinyl, tetrahydropyranyl, piperidinyl, 1,4-dioxanyl, morpholinyl, piperazinyl, azepanyl, oxepanyl, oxazepanyl and diazepinyl.

Het², Het⁶ and Het¹⁰ are saturated or partially saturated heterocycles and may be attached via a ring nitrogen atom or a ring carbon atom. Equally, when substituted, the substituent may be located on a ring nitrogen atom or a ring carbon atom. Het², Het⁶ and Het¹⁰ are multicyclic heterocyclic groups, containing two or more rings. Such rings may be joined so as to create a bridged, fused or spirofused ring system, as illustrated with two six-membered rings below (heteroatoms not shown):

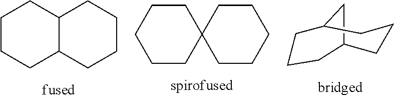

Het², Het⁶ and Het¹⁰ may be fully saturated or partially unsaturated, i.e. they may have one or more degrees of unsaturation but may not be fully aromatic. In the case of a fused ring system, one of the rings may be aromatic but not both of them. An Example of Het² is tropanyl (azabicyclo[3.2.1]octanyl).

Het³, Het⁷ and Het¹¹ are aromatic heterocycles and may be attached via a ring carbon atom or a ring nitrogen atom with an appropriate valency. Equally, when substituted, the substituent may be located on a ring carbon atom or a ring nitrogen atom with an appropriate valency. Specific examples include thienyl, furanyl, pyrrolyl, pyrazolyl, imidazoyl, oxazolyl, isoxazolyl, thiazolyl, isothiazolyl, triazolyl, oxadiazolyl, thiadiazolyl, tetrazolyl, pyridyl, pyridazinyl, pyrimidinyl and pyrazinyl.

Het⁴, Het⁸ and Het¹² are aromatic heterocycles and may be attached via a ring carbon atom or a ring nitrogen atom with an appropriate valency. Equally, when substituted, the substituent may be located on a ring carbon atom or a ring nitrogen atom with an appropriate valency. Het⁴ and Het⁸ are aromatic and are therefore necessarily fused bicycles. Specific examples include benzofuranyl, benzothienyl, indolyl, benzimidazolyl, indazolyl, benzotriazolyl, pyrrolo[2,3-b]pyridyl, pyrrolo[2,3-c]pyridyl, pyrrolo[3,2-c]pyridyl, pyrrolo[3,2-b]pyridyl, imidazo[4,5-b]pyridyl, imidazo[4,5-c]pyridyl, pyrazolo[4,3-d]pyridyl, pyrazolo[4,3-c]pyridyl, pyrazolo[3,4-c]pyridyl, pyrazolo[3,4-b]pyridyl, isoindolyl, indazolyl, purinyl, indolizinyl, imidazo[1,2-a]pyridyl, imidazo[1,5-a]pyridyl, pyrazolo[1,5-a]pyridyl, pyrrolo[1,2-b]pyridazinyl, imidazo[1,2-c]pyrimidinyl, quinolinyl, isoquinolinyl, cinnolinyl, quinazolinyl, quinoxalinyl, phthalazinyl, 1,6-naphthyridinyl, 1,7-naphthyridinyl, 1,8-naphthyridinyl, 1,5-naphthyridinyl, 2,6-naphthyridinyl, 2,7-naphthyridinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-d]pyrazinyl, pyrido[3,4-b]pyrazinyl, pyrimido[5,4-d]pyrimidinyl, pyrazino[2,3-b]pyrazinyl and pyrimido[4,5-d]pyrimidine.

The term “cycloalkyl” means a means a monocyclic, saturated hydrocarbon group of the formula CₙH₂ₙ₋₁. Examples include cyclopropyl, cyclobutyl, cyclopentyl, cyclohexyl, and cycloheptyl. Unless otherwise specified, a cycloalkyl group comprises from 3 to 8 carbon atoms.

The term bicycloalkyl means a bicyclic, saturated hydrocarbon group of the formula CₙH₂ₙ₋₃ in which the two rings are joined in a fused, spiro-fused or bridged manner (see above). The following groups are illustrative of C₅-C₁₂ bicycloalkyl (note that as drawn, these groups have an extra hydrogen atom where the linking bond would be):

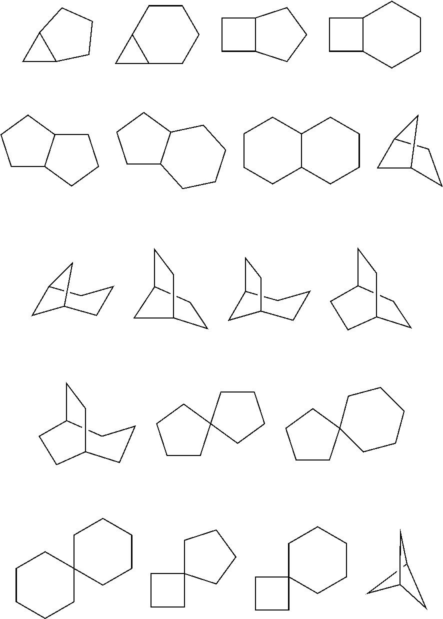

In the definition of R⁷, the C₃-C₈ cycloalkyl ring may be fused to a phenyl ring or a 5- or 6-membered aromatic heterocylic ring. In the case of such fusion, the R⁷ group may be attached to the amide nitrogen through the cycloalkyl ring or through the fused ring but is preferably attached through the cycloalkyl ring. Equally, in the case where the R⁷ group is substituted, such substitution may occur on the cycloalkyl ring, the fused ring or both. The 5- or 6-membered aromatic heterocyclic ring is preferably (i) a 6-membered aromatic heterocycle containing 1-3 N atoms or (ii) a 5-membered aromatic heterocycle containing either (a) 1-4 N atoms or (b) 1 O or S atom and 0-3 N atoms. Specific examples of preferred 5- or 6-membered aromatic heterocyclic rings are given above in relation to Het³/Het⁷. Where the C₃-C₈ cycloalkyl ring of R⁷ is fused, it is particularly preferred that it is fused to a phenyl, imidazolyl, pyridyl or pyrazolyl ring.

The term “oxo” means a doubly bonded oxygen.

The term “alkoxy” means a radical comprising an alkyl radical that is bonded to an oxygen atom, such as a methoxy radical. Examples of such radicals include methoxy, ethoxy, propoxy, isopropoxy, butoxy and tert-butoxy.

As used herein, the terms “co-administration”, “co-administered” and “in combination with”, referring to a combination of a compound of formula (I) and one or more other therapeutic agents, is intended to mean, and does refer to and include the following:
simultaneous administration of such a combination of a compound of formula (I) and a further therapeutic agent to a patient in need of treatment, when such components are formulated together into a single dosage form which releases said components at substantially the same time to said patient,
substantially simultaneous administration of such a combination of a compound of formula (I) and a further therapeutic agent to a patient in need of treatment, when such components are formulated apart from each other into separate dosage forms which are taken at substantially the same time by said patient, whereupon said components are released at substantially the same time to said patient, and
sequential administration of such a combination of a compound of formula (I) and a further therapeutic agent to a patient in need of treatment, when such components are formulated apart from each other into separate dosage forms which are taken at consecutive times by said patient with a significant time interval between each administration, whereupon said components are released at substantially different times to said patient; and
sequential administration of such a combination of a compound of formula (I) and a further therapeutic agent to a patient in need of treatment, when such components are formulated together into a single dosage form which releases said components in a controlled manner.

The term ‘excipient’ is used herein to describe any ingredient other than a compound of formula (I). The choice of excipient will to a large extent depend on factors such as the particular mode of administration, the effect of the excipient on solubility and stability, and the nature of the dosage form. The term “excipient” encompasses diluent, carrier or adjuvant.

Pharmaceutically acceptable salts of the compounds of formula (I) include the acid addition and base salts thereof.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, naphatlene-1,5-disulfonic acid and xinofoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002).

Pharmaceutically acceptable salts of compounds of formula (I) may be prepared by one or more of three methods:
(i) by reacting the compound of formula (I) with the desired acid or base;
(ii) by removing an acid- or base-labile protecting group from a suitable precursor of the compound of formula (I) or by ring-opening a suitable cyclic precursor, for example, a lactone or lactam, using the desired acid or base; or
(iii) by converting one salt of the compound of formula (I) to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of formula (I) may also exist in unsolvated and solvated forms. The term ‘solvate’ is used herein to describe a molecular complex comprising the compound of formula (I), or a pharmaceutically acceptable salt thereof, and one or more pharmaceutically acceptable solvent molecules, for example, ethanol. The term ‘hydrate’ is employed when said solvent is water.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together—see Chem Commun, 17, 1889-1896, by O. Almarsson and M. J. Zaworotko (2004). For a general review of multi-component complexes, see J Pharm Sci, 64 (8), 1269-1288, by Haleblian (August 1975).

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (glass transition'). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (melting point').

The compounds of formula (I) may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

Hereinafter all references to compounds of formula (I) (also referred to as compounds of the invention) include references to salts, solvates, multi-component complexes and liquid crystals thereof and to solvates, multi-component complexes and liquid crystals of salts thereof.

Also included within the scope of the invention are all polymorphs and crystal habits of compounds of formula (I), prodrugs and isomers thereof (including optical, geometric and tautomeric isomers) as hereinafter defined and isotopically-labeled forms thereof.

As indicated, so-called ‘prodrugs’ of the compounds of formula (I) are also within the scope of the invention. Thus certain derivatives of a compound of formula (I) which may have little or no pharmacological activity themselves can, when administered into or onto the body, be converted into a compound of formula (I) having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association).

Prodrugs in accordance with the invention can, for example, be produced by replacing appropriate functionalities present in the compounds of formula (I) with certain moieties known to those skilled in the art as ‘pro-moieties’ as described, for example, in Design of Prodrugs by H. Bundgaard (Elsevier, 1985).

Some examples of prodrugs in accordance with the invention include:
(i) where the compound of formula (I) contains a carboxylic acid functionality (—COOH), an ester thereof, for example, a compound wherein the hydrogen of the carboxylic acid functionality of the compound of formula (I) is replaced by (C₁-C₈)alkyl;
(ii) where the compound of formula (I) contains an alcohol functionality (—OH), an ether thereof, for example, a compound wherein the hydrogen of the alcohol functionality of the compound of formula (I) is replaced by (C₁-C₆)alkanoyloxymethyl; and
(iii) where the compound of formula (I) contains a primary or secondary amino functionality (—NH₂ or —NHR where R≠H), an amide thereof, for example, a compound wherein, as the case may be, one or both hydrogens of the amino functionality of the compound of formula (I) is/are replaced by (C₁-C₁₀)alkanoyl.

Further examples of replacement groups in accordance with the foregoing examples and examples of other prodrug types may be found in the aforementioned references.

Moreover, certain compounds of formula (I) may themselves act as prodrugs of other compounds of formula (I).

Compounds of formula (I) containing one or more asymmetric carbon atoms can exist as two or more stereoisomers. Where a compound of formula (I) contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds of formula (I) containing, for example, an imino, keto, or oxime group, or so-called valence tautomerism in compounds which contain an aromatic moiety. It follows that a single compound may exhibit more than one type of isomerism.

Included within the scope of the present invention are all stereoisomers, geometric isomers and tautomeric forms of the compounds of formula (I), including compounds exhibiting more than one type of isomerism, and mixtures of one or more thereof. Also included are acid addition or base salts wherein the counterion is optically active, for example, d-lactate or l-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, an alcohol, or, in the case where the compound of formula (I) contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person. Chiral compounds of formula (I) (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture. Chiral chromatography using sub- and supercritical fluids may be employed. Methods for chiral chromatography useful in some embodiments of the present invention are known in the art (see, for example, Smith, Roger M., Loughborough University, Loughborough, UK; Chromatographic Science Series (1998), 75 (Supercritical Fluid Chromatography with Packed Columns), pp. 223-249 and references cited therein). In some relevant examples herein, columns were obtained from Chiral Technologies, Inc, West Chester, Pa., USA, a subsidiary of Daicel® Chemical Industries, Ltd., Tokyo, Japan.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer. While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

The present invention includes all pharmaceutically acceptable isotopically-labelled compounds of formula (I) wherein one or more atoms are replaced by atoms having the same atomic number, but an atomic mass or mass number different from the atomic mass or mass number which predominates in nature. Isotopically-labelled compounds of formula (I) can generally be prepared by conventional techniques known to those skilled in the art or by processes analogous to those described in the accompanying Examples and Preparations using an appropriate isotopically-labelled reagent in place of the non-labelled reagent previously employed.

Also included within the scope of the invention are metabolites of compounds of formula (I), that is, compounds formed in vivo upon administration of the drug. Some examples of metabolites in accordance with the invention include
(i) where the compound of formula (I) contains a methyl group, an hydroxymethyl derivative thereof (—CH₃->—CH₂OH):
(ii) where the compound of formula (I) contains an alkoxy group, an hydroxy derivative thereof (—OR->—OH);
(iii) where the compound of formula (I) contains a tertiary amino group, a secondary amino derivative thereof (—NR¹R²->—NHR¹ or —NHR²);
(iv) where the compound of formula (I) contains a secondary amino group, a primary derivative thereof (—NHR¹->—NH₂);
(v) where the compound of formula (I) contains a phenyl moiety, a phenol derivative thereof (-Ph->-PhOH); and
(vi) where the compound of formula (I) contains an amide group, a carboxylic acid derivative thereof (—CONH₂->COOH).

For administration to human patients, the total daily dose of a compound of formula (I) is typically in the range of 0.01 mg to 500 mg depending, of course, on the mode of administration. In another embodiment of the present invention, the total daily dose of a compound of formula (I) is typically in the range of 0.1 mg to 300 mg. In yet another embodiment of the present invention, the total daily dose of a compound of formula (I) is typically in the range of 1 mg to 30 mg. The total daily dose may be administered in single or divided doses and may, at the physician's discretion, fall outside of the typical range given herein. These dosages are based on an average human subject having a weight of about 65 kg to 70 kg. The physician will readily be able to determine doses for subjects whose weight falls outside this range, such as infants and the elderly.

In the case of dry powder inhalers and aerosols, the dosage unit is determined by means of a prefilled capsule, blister or pocket or by a system that utilises a gravimetrically fed dosing chamber. Units in accordance with the invention are typically arranged to administer a metered dose or “puff” containing from 1 to 5000 μg of drug. The overall daily dose will typically be in the range 1 μg to 20 mg which may be administered in a single dose or, more usually, as divided doses throughout the day.

A compound of formula (I) can be administered per se, or in the form of a pharmaceutical composition, which, as active constituent contains an efficacious dose of at least one compound of the invention, in addition to customary pharmaceutically innocuous excipients and/or additives.

Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

Compounds of formula (I) may be administered orally. Oral administration may involve swallowing, so that the compound enters the gastrointestinal tract, or buccal or sublingual administration may be employed by which the compound enters the blood stream directly from the mouth. Formulations suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations. Oral administration, particularly in the form of a tablet or capsule, is preferred for compounds of formula (I).

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

Compounds of formula (I) may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %. In one embodiment of the present invention, the disintegrant will comprise from 5 weight % to 20 weight % of the dosage form. Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate. Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet. Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %. In one embodiment of the present invention, lubricants comprise from 0.5 weight % to 3 weight % of the tablet. Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated. Formulations of tablets are discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of formula (I), a film-forming polymer, a binder, a solvent, a humectant, a plasticiser, a stabiliser or emulsifier, a viscosity-modifying agent and a solvent. Some components of the formulation may perform more than one function. The film-forming polymer may be selected from natural polysaccharides, proteins, or synthetic hydrocolloids and is typically present in the range 0.01 to 99 weight %, more typically in the range 30 to 80 weight %. Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents. Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release includes delayed, sustained, pulsed, controlled, targeted and programmed release. Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

Compounds of formula (I) may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Compounds of the invention may also be administered topically to the skin or mucosa, that is, dermally or transdermally.

The compounds of formula (I) can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler, as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, for example, chitosan or cyclodextrin.

The pressurised container, pump, spray, atomizer, or nebuliser contains a solution or suspension of the compound of formula (I) comprising, for example, ethanol, aqueous ethanol, or a suitable alternative agent for dispersing, solubilising, or extending release of the compound, a propellant as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for intranasal administration. Formulations for intranasal administration may be formulated to be immediate and/or modified release using, for example, PGLA. Modified release includes delayed, sustained, pulsed, controlled, targeted and programmed release.

Compounds of formula (I) may also be administered directly to the eye or ear, typically in the form of drops of a micronised suspension or solution in isotonic, pH-adjusted, sterile saline.

Compounds of formula (I) may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration. Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in international patent publications WO-A-91/11172, WO-A-94/02518 and WO-A-98/55148.

Inasmuch as it may desirable to administer a combination of active compounds, for example, for the purpose of treating a particular disease or condition, it is within the scope of the present invention that two or more pharmaceutical compositions, at least one of which contains a compound of formula (I), may conveniently be combined in the form of a kit suitable for coadministration of the compositions. Thus, a kit of the invention comprises two or more separate pharmaceutical compositions, at least one of which contains a compound of formula (I), and means for separately retaining said compositions, such as a container, divided bottle, or divided foil packet. An example of such a kit is the familiar blister pack used for the packaging of tablets, capsules and the like. Such a kit is particularly suitable for administering different dosage forms, for example, oral and parenteral, for administering separate compositions at different dosage intervals, or for titrating the separate compositions against one another. To assist compliance, the kit typically comprises directions for administration and may be provided with a so-called memory aid.

All the compound of formula (I) can be made by the specific and general experimental procedures described below in combination with the common general knowledge of one skilled in the art (see, for example, Comprehensive Organic Chemistry, Ed. Barton and Ollis, Elsevier; Comprehensive Organic Transformations: A Guide to Functional Group Preparations, Larock, John Wiley and Sons).

The compounds of formula (I), being amides, are conveniently prepared by coupling an amine of formula (III) and an acid of formula (II) in accordance with Scheme 1.

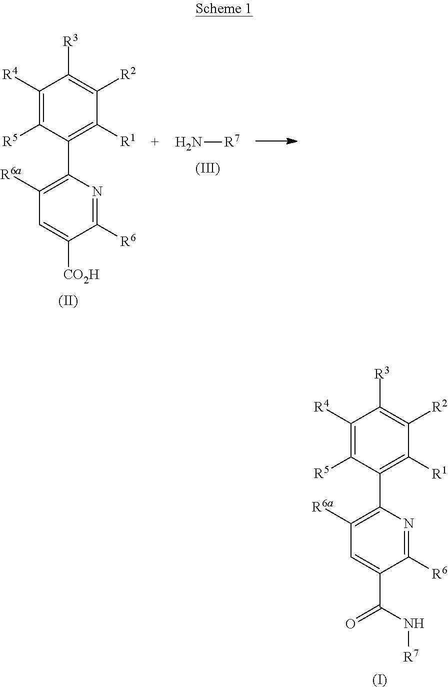

Those skilled in the art will appreciate that there are many known ways of preparing amides. For example, see Montalbetti, C. A. G. N and Falque, V., Amide bond formation and peptide coupling, Tetrahedron, 2005, 61(46), pp. 10827-10852 and references cited therein. The examples provided herein are thus not intended to be exhaustive, but merely illustrative.

The following general methods i, ii and iii have been used.
(i) To the carboxylic acid (0.15 mmol) and 1-hydroxybenzotriazole (0.3 mmol) in DMF (1.0 mL) was added 0.3.mmol of PS-Carbodiimide resin (Argonaut, 1.3 mmol/g). The mixture was shaken for 10 min and then the amine (0.1 mmol) in DMF (1 mL) was added. The mixture was allowed to agitate overnight at room temperature and subsequently treated with 0.60 mmole of PS-trisamine (Argonaut, 3.8 mmol/g). The reaction mixture was filtered, concentrated in vacuo and purified by reverse phase chromatography.
(ii) To the carboxylic acid (0.15 mmol) and HBTU (0.175 mmol) in DMF (1.0 mL) was added 0.45 mmol triethylamine. The mixture was stirred for 30 minutes and then the amine (0.2 mmol) in DMF (1.0 mL) was added. The mixture was allowed to stir overnight at room temperature and subsequently partitioned between water and a suitable organic solvent. The organic phase was separated, concentrated in vacuo and purified by either by reverse phase chromatography, normal phase chromatography or crystallisation.
(iii) To the carboxylic acid (0.15 mmol) in DMF was added N,N-carbonyldiimidazole (0.18 mmol) in DMF (1.0 mL). The mixture was stirred for 30 min and then the amine (0.18 mmol) in DMF (1.0 mL) was added. The mixture was allowed to stir overnight at room temperature and subsequently partitioned between water and a suitable organic solvent. The organic layer was separated, concentrated in vacuo and purified by reverse phase chromatography, normal phase chromatography or crystallisation.

Where it is stated that compounds were prepared in the manner described for an earlier Example, the skilled person will appreciate that reaction times, number of equivalents of reagents and reaction temperatures may be modified for each specific reaction, and that it may nevertheless be necessary or desirable to employ different work-up or purification conditions.

Those skilled in the art will appreciate that there are many known ways of preparing aryl pyridines of formula (II). Such methods are disclosed in patent textbooks and laboratory handbooks which constitute the common general knowledge of the skilled person, including the textbooks referenced above and references cited therein. Typically, an aryl (or heteroaryl) halide (Cl, Br, I) or trifluoromethanesulphonate is stirred with an organometallic species such as a stannane, organomagnesium derivative or a boronate ester or boronic acid in the presence of a catalyst, usually a palladium derivative between 0° C. and 120° C. in solvents including tetrahydrofuran, toluene, DMF and water for 1 to 24 hours. For example, an aryl (or heteroaryl) bromide may be heated to 100° C. in a mixture of water/toluene with a base such as sodium carbonate or sodium hydroxide, a palladium catalyst such as tetrakis(triphenylphosphine)palladium (0), a phase transfer catalyst such as tetra-n-butyl ammonium bromide and an aryl (or heteroaryl) boronic acid or ester. As a second example, an aryl (or heteroaryl) boronic ester an aryl (or heteroaryl) halide (Cl, Br, I) or aryl (or heteroaryl) trifluoromethanesulphonate and a fluoride source such as KF or CsF in a non-aqueous reaction medium such as 1,4-dioxane may be employed. It may be necessary to protect the acid functionality in the compound of formula (II) during such a coupling reaction—suitable protecting groups and their use are well known to the skilled person (see, e.g., ‘Protective Groups in Organic Synthesis’ by Theorora Greene and Peter Wuts (third edition, 1999, John Wiley and Sons).

Amines of formula (III) are in many cases commercially available and may otherwise be prepared by standard methodology well known the skilled person—see, for example, ‘Comprehensive Organic Transformations’ by Richard Larock (1999, VCH Publishers Inc.).

The following tabulated compounds have been prepared using the methodology described above. Data relating to purification and characterization are provided in the tables and relevant HPLC and LCMS methods are described in detail below the tables, along with more specific details relating to the preparation and characterization of selected compounds. Examples 1-573 are defined with reference to formula (Ib) in which R¹, R², R³ and R⁵ are each H unless a different meaning for one or more of them is specified.

(Ib)

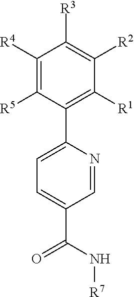

| | | | | Purification and |
|---|---|---|---|---|
| Ex | R⁷ | R¹⁻⁵ | Name | Characterisation |
| 1 | 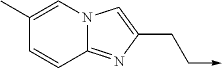 | R² = F | 6-(3-Fluorophenyl)-N-[2-(6- methylimidazo[1,2-a]pyridin- 2-yl)ethyl]nicotinamide | LCMS Method (C) RT 1.49 min m/z Obs [M + 1] 375.0 calc [M + 1] 374.15428 |
| 2 | 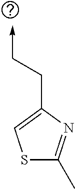 | R² = F | 6-(3-Fluorophenyl)-N-[2-(2- methyl-1,3-thiazol-4- yl)ethyl]nicotinamide | LCMS Method (C) RT 1.64 min m/z Obs [M + 1] 342.1 calc [M + 1] 341.09981 |
| 3 | 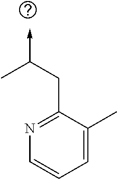 | R² = F | 6-(3-Fluorophenyl)-N-[1- methyl-2-(3-methylpyridin-2- yl)ethyl]nicotinamide | LCMS Method (C) RT 1.41 min m/z Obs [M + 1] 342.1 calc [M + 1] 349.15903 |
| 4 | 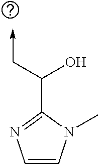 | R² = F | 6-(3-Fluorophenyl)-N-[2- hydroxy-2-(1-methyl-1H- imidazol-2- yl)ethyl]nicotinamide | LCMS Method (C) RT 1.41 min m/z Obs [M + 1] 350.1 calc [M + 1] 349.15903 |
| 5 | 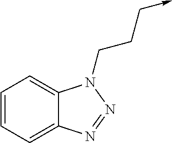 | R² = F | N-[3-(1H-Benzotriazol-1- yl)propyl]-6-(3- fluorophenyl)nicotinamide | LCMS Method (C) RT 2.01 min m/z Obs [M + 1] 376.1 calc [M + 1] 375.14953 |
| 6 | 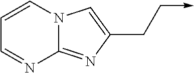 | R² = F | 6-(3-Fluorophenyl)-N-(2- imidazo[1,2-a]pyrimidin-2- ylethyl)nicotinamide | LCMS Method (C) RT 1.33 min m/z Obs [M + 1] 362.5 calc [M + 1] 361.13388 |
| 7 | 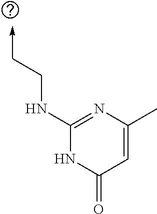 | R² = F | 6-(3-Fluorophenyl)-N-{2-[(4- methyl-6-oxo-1,6- dihydropyrimidin-2- yl)amino]ethyl}nicotinamide | LCMS Method (C) RT 1.38 min m/z Obs [M + 1] 368.3 calc [M + 1] 367.14445 |
| 8 | 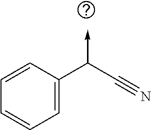 | R² = F | N-[Cyano(phenyl)methyl]-6- (3-fluorophenyl)nicotinamide | LCMS Method (C) RT 2.44 min m/z Obs [M + 1] 332.3 calc [M + 1] 331.11208 |
| 9 | 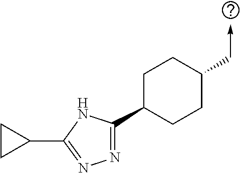 | R² = F | N-{[trans-4-(5-Cyclopropyl- 4H-1,2,4-triazol-3- yl)cyclohexyl]methyl}-6-(3- fluorophenyl)nicotinamide | LCMS Method (C) RT 1.59 min m/z Obs [M + 1] 420.4 calc [M + 1] 419.21213 |
| 10 | 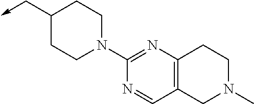 | R² = F | 6-(3-Fluorophenyl)-N-{[1-(6- methyl-5,6,7,8- tetrahydropyrido[4,3- d]pyrimidin-2-yl)piperidin-4- yl]methyl}nicotinamide | LCMS Method (C) RT 1.51 min m/z Obs [M + 1] 461.3 calc [M + 1] 460.23868 |
| 11 | 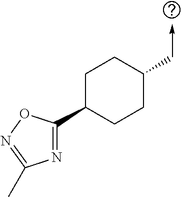 | R² = F | 6-(3-Fluorophenyl)-N-{[trans- 4-(3-methyl-1,2,4-oxadiazol- 5-yl)cyclohexyl]methyl} nicotinamide | LCMS Method (C) RT 2.21 min m/z Obs [M + 1] 395.1 calc [M + 1] 394.1805 |
| 12 | 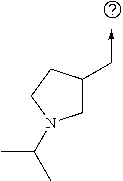 | R² = F | 6-(3-Fluorophenyl)-N-[(1- isopropylpyrrolidin-3- yl)methyl]nicotinamide | LCMS Method (C) RT 1.38 min m/z Obs [M + 1] 342.1 calc [M + 1] 341.19033 |
| 13 | 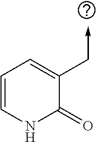 | R² = F | 6-(3-Fluorophenyl)-N-[(2- oxo-1,2-dihydropyridin-3- yl)methyl]nicotinamide | LCMS Method (C) RT 1.52 min m/z Obs [M + 1] 324.1 calc [M + 1] 323.107 |
| 14 | 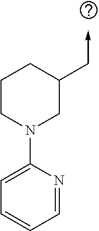 | R² = F | 6-(3-Fluorophenyl)-N-[(1- pyridin-2-ylpiperidin-3- yl)methyl]nicotinamide | LCMS Method (C) RT 1.54 min m/z Obs [M + 1] 391.4 calc [M + 1] 390.18558 |
| 15 | 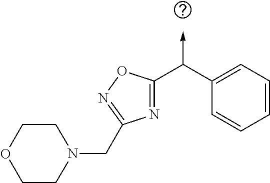 | R² = F | 6-(3-Fluorophenyl)-N-{[3- (morpholin-4-ylmethyl)-1,2,4- oxadiazol-5-yl](phenyl) methyl}nicotinamide | LCMS Method (C) RT 1.78 min m/z Obs [M + 1] 375.0 calc [M + 1] 473.18631 |
| 16 | 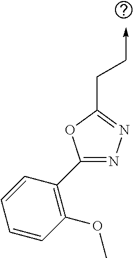 | R² = F | 6-(3-Fluorophenyl)-N-{2-[5- (2-methoxyphenyl)-1,3,4- oxadiazol-2- yl]ethyl}nicotinamide | LCMS Method (C) RT 2.04 min m/z Obs [M + 1] 419.5 calc [M + 1] 418.14411 |
| 17 | 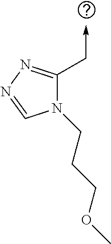 | R² = F | 6-(3-Fluorophenyl)-N-{[4-(3- methoxypropyl)-4H-1,2,4- triazol-3- yl]methyl}nicotinamide | LCMS Method (C) RT 1.51 min m/z Obs [M + 1] 370.0 calc [M + 1] 369.1601 |
| 18 | 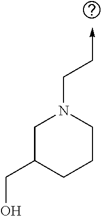 | R² = F | 6-(3-Fluorophenyl)-N-{2-[3- (hydroxymethyl)piperidin-1- yl]ethyl}nicotinamide | LCMS Method (C) RT 1.28 min m/z Obs [M + 1] 358.1 calc [M + 1] 357.18525 |
| 19 | 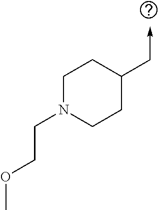 | R² = F | 6-(3-Fluorophenyl)-N-{[1-(2- methoxyethyl)piperidin-4- yl]methyl}nicotinamide | LCMS Method (C) RT 1.36 min m/z Obs [M + 1] 372.4 calc [M + 1] 371.20089 |
| 20 | 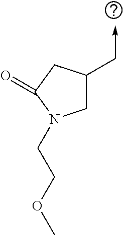 | R² = F | 6-(3-Fluorophenyl)-N-{[1-(2- methoxyethyl)-5- oxopyrrolidin-3- yl]methyl}nicotinamide | LCMS Method (C) RT 1.58 min m/z Obs [M + 1] 372.3 calc [M + 1] 31.16451 |
| 21 | 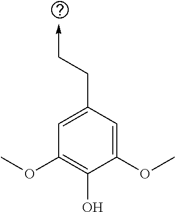 | R² = F | 6-(3-Fluorophenyl)-N-[2-(4- hydroxy-3,5- dimethoxyphenyl)ethyl] nicotinamide | LCMS Method (C) RT 1.95 min m/z Obs [M + 1] 397.0 calc [M + 1] 396.14853 |
| 22 | 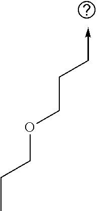 | R² = F | 6-(3-Fluorophenyl)-N-(3- propoxypropyl)nicotinamide | LCMS Method (C) RT 2.21 min m/z Obs [M + 1] 317.0 calc [M + 1] 316.1587 |
| 23 | 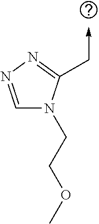 | R² = F | 6-(3-Fluorophenyl)-N-{[4-(2- methoxyethyl)-4H-1,2,4- triazol-3- yl]methyl}nicotinamide | LCMS Method (C) RT 1.49 min m/z Obs [M + 1] 356.0 calc [M + 1] 355.14445 |
| 24 | 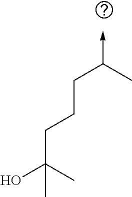 | R² = F | 6-(3-Fluorophenyl)-N-(5- hydroxy-1,5- dimethylhexyl)nicotinamide | LCMS Method (C) RT 1.94 min m/z Obs [M + 1] 345.3 calc [M + 1] 344.19 |
| 25 | 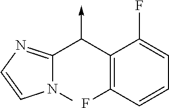 | R² = F | N-[(2,6-Difluorophenyl)(1- methyl-1H-imidazol-2- yl)methyl]-6-(3- fluorophenyl)nicotinamide | LCMS Method (C) RT 1.65 min m/z Obs [M + 1] 423.0 calc [M + 1] 422.13544 |
| 26 | 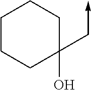 | R² = F | 6-(3-Fluorophenyl)-N-[(1- hydroxycyclohexyl)methyl] nicotinamide | LCMS Method (C) RT 1.98 min m/z Obs [M + 1] 329.0 calc [M + 1] 328.1587 |
| 27 | 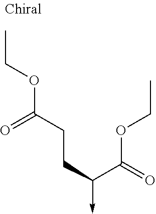 | R² = F | Diethyl N-{[6-(3- fluorophenyl)pyridin-3- yl]carbonyl}-L-glutamate | LCMS Method (C) RT 2.33 min m/z Obs [M + 1] 403.4 calc [M + 1] 402.15909 |
| 28 | 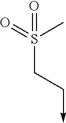 | R² = F | 6-(3-Fluorophenyl)-N-[2- (methylsulfonyl)ethyl] nicotinamide | LCMS Method (C) RT 1.58 min m/z Obs [M + 1] 323.0 calc [M + 1] 322.07874 |
| 29 | 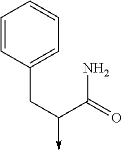 | R² = F | Nalpha-{[6-(3- fluorophenyl)pyridin-3- yl]carbonyl} phenylalaninamide | LCMS Method (C) RT 1.96 min m/z Obs [M + 1] 364.4 calc [M + 1] 363.1383 |
| 30 | 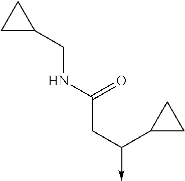 | R² = F | N-{1-Cyclopropyl-3- [(cyclopropylmethyl)amino]- 3-oxopropyl}-6-(3- fluorophenyl)nicotinamide | LCMS Method (C) RT 1.99 min m/z Obs [M + 1] 382.4 calc [M + 1] 381.18525 |
| 31 | 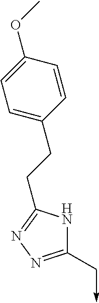 | R² = F | 6-(3-Fluorophenyl)-N-({5-[2- (4-methoxyphenyl)ethyl]-4H- 1,2,4-triazol-3- yl}methyl)nicotinamide | LCMS Method (C) RT 1.85 min m/z Obs [M + 1] 423.3 calc [M + 1] 431.17574 |
| 32 | 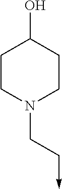 | R² = F | 6-(3-Fluorophenyl)-N-[2-(4- hydroxypiperidin-1- yl)ethyl]nicotinamide | LCMS Method (C) RT 1.22 min m/z Obs [M + 1] 344.3 calc [M + 1] 343.1696 |
| 33 | 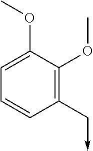 | R² = F | N-(2,3-Dimethoxybenzyl)-6- (3-fluorophenyl)nicotinamide | LCMS Method (C) RT 2.30 min m/z Obs [M + 1] 367.4 calc [M + 1] 366.13796 |
| 34 | 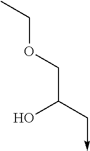 | R² = F | N-(3-Ethoxy-2- hydroxypropyl)-6-(3- fluorophenyl)nicotinamide | LCMS Method (C) RT 1.61 min m/z Obs [M + 1] 319.0 calc [M + 1] 318.13796 |
| 35 | 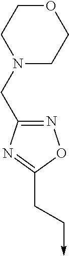 | R² = F | 6-(3-Fluorophenyl)-N-{2-[3- (morpholin-4-ylmethyl)-1,2,4- oxadiazol-5- yl]ethyl}nicotinamide | LCMS Method (C) RT 1.37 min m/z Obs [M + 1] 412.1 calc [M + 1] 411.17066 |
| 36 | 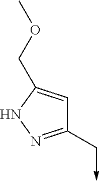 | R² = F | 6-(3-Fluorophenyl)-N-{[5- (methoxymethyl)-1H-pyrazol- 3-yl]methyl}nicotinamide | LCMS Method (C) RT 1.63 min m/z Obs [M + 1] 341.4 calc [M + 1] 340.13355 |
| 37 | 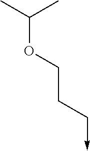 | R² = F | 6-(3-Fluorophenyl)-N-(3- isopropoxypropyl) nicotinamide | LCMS Method (C) RT 2.13 min m/z Obs [M + 1] 317.1 calc [M + 1] 316.1587 |
| 38 | 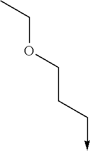 | R² = F | N-(3-Ethoxypropyl)-6-(3- fluorophenyl)nicotinamide | LCMS Method (C) RT 1.96 min m/z Obs [M + 1] 303.3 calc [M + 1] 302.14305 |
| 39 | 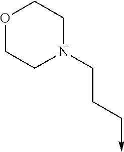 | R² = F | 6-(3-Fluorophenyl)-N-(3- morpholin-4- ylpropyl)nicotinamide | LCMS Method (C) RT 1.29 min m/z Obs [M + 1] 344.3 calc [M + 1] 343.1696 |
| 40 | 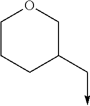 | R² = F | 6-(3-Fluorophenyl)-N- (tetrahydro-2H-pyran-3- ylmethyl)nicotinamide | LCMS Method (C) RT 1.87 min m/z Obs [M + 1] 315.1 calc [M + 1] 314.14305 |
| 41 | 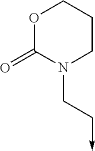 | R² = F | 6-(3-Fluorophenyl)-N-[2-(2- oxo-1,3-oxazinan-3- yl)ethyl]nicotinamide | LCMS Method (C) RT 1.55 min m/z Obs [M + 1] 344.1 calc [M + 1] 343.13321 |
| 42 | 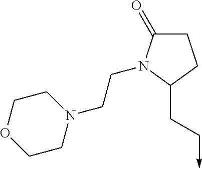 | R² = F | 6-(3-Fluorophenyl)-N-{2-[1- (2-morpholin-4-ylethyl)-5- oxopyrrolidin-2- yl]ethyl}nicotinamide | LCMS Methoc (C) RT 1.35 min m/z Obs [M + 1] 441.5 calc [M + 1] 440.22236 |
| 43 | 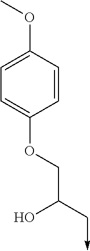 | R² = F | 6-(3-Fluorophenyl)-N-[2- hydroxy-3-(4- methoxyphenoxy)propyl] nicotinamide | LCMS Method (C) RT 2.09 min m/z Obs [M + 1] 397.3 calc [M + 1] 396.14853 |
| 44 | 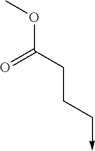 | R² = F | Methyl 4-({[6-(3- fluorophenyl)pyridin-3- yl]carbonyl}amino)butanoate | LCMS Method (C) RT 1.88 min m/z Obs [M + 1] 317.3 calc [M + 1] 316.12231 |
| 45 | 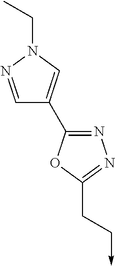 | R² = F | N-{2-[5-(1-Ethyl-1H-pyrazol- 4-yl)-1,3,4-oxadiazol-2- yl]ethyl}-6-(3- fluorophenyl)nicotinamide | LCMS Method (C) RT 1.78 min m/z Obs [M + 1] 407.1 calc [M + 1] 406.15534 |
| 46 | 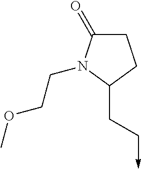 | R² = F | 6-(3-Fluorophenyl)-N-{2-[1- (2-methoxyethyl)-5- oxopyrrolidin-2- yl]ethyl}nicotinamide | LCMS Method (C) RT 1.66 min m/z Obs [M + 1] 386.3 calc [M + 1] 385.18016 |
| 47 | 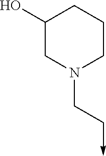 | R² = F | 6-(3-Fluorophenyl)-N-[2-(3- hydroxypiperidin-1- yl)ethyl]nicotinamide | LCMS Method (C) RT 1.27 min m/z Obs [M + 1] 344.1 calc [M + 1] 343.1696 |
| 48 | 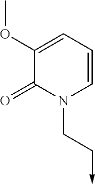 | R² = F | 6-(3-Fluorophenyl)-N-[2-(3- methoxy-2-oxopyridin-1(2H)- yl)ethyl]nicotinamide | LCMS Method (C) RT 1.65 min m/z Obs [M + 1] 368.3 calc [M + 1] 367.13321 |
| 49 | 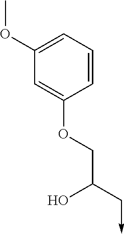 | R² = F | 6-(3-Fluorophenyl)-N-[2- hydroxy-3-(3- methoxyphenoxy)propyl] nicotinamide | LCMS Method (C) RT 2.13 min m/z Obs [M + 1] 397.1 calc [M + 1] 396.14853 |
| 50 | 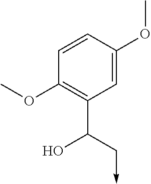 | R² = F | N-[2-(2,5-Dimethoxyphenyl)- 2-hydroxyethyl]-6-(3- fluorophenyl)nicotinamide | LCMS Method (C) RT 2.06 min m/z Obs [M + 1] 397.0 calc [M + 1] 396.14853 |
| 51 | 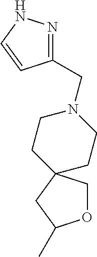 | R² = F | 6-(3-Fluorophenyl)-N-{[8- (1H-pyrazol-3-ylmethyl)-2- oxa-8-azaspiro[4.5]dec-3- yl]methyl}nicotinamide | LCMS Method (C) RT 1.36 min m/z Obs [M + 1] 450.3 calc [M + 1] 449.22269 |
| 52 | 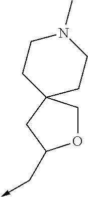 | R² = F | 6-(3-Fluorophenyl)-N-[(8- methyl-2-oxa-8- azaspiro[4.5]dec-3- yl)methyl]nicotinamide | LCMS Method (C) RT 1.32 min m/z Obs [M + 1] 384.4 calc [M + 1] 383.20089 |
| 53 | 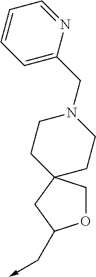 | R² = F | 6-(3-Fluorophenyl)-N-{[8- (pyridin-2-ylmethyl)-2-oxa-8- azaspiro[4.5]dec-3- yl]methyl}nicotinamide | LCMS Method (C) RT 1.44 min m/z Obs [M + 1] 461.3 calc [M + 1] 460.22744 |
| 54 | 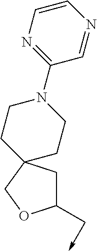 | R² = F | 6-(3-Fluorophenyl)-N-[(8- pyrazin-2-yl-2-oxa-8- azaspiro[4.5]dec-3- yl)methyl]nicotinamide | LCMS Method (C) RT 1.89 min m/z Obs [M + 1] 448.3 calc [M + 1] 447.20704 |
| 55 | 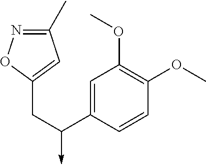 | R² = F | N-[1-(3,4-Dimethoxyphenyl)- 2-(3-methylisoxazol-5- yl)ethyl]-6-(3- fluorophenyl)nicotinamide | LCMS Method (C) RT 2.25 min m/z Obs [M + 1] 462.3 calc [M + 1] 461.17508 |
| 56 | 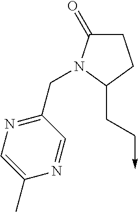 | R² = F | 6-(3-Fluorophenyl)-N-(2-{1- [(5-methylpyrazin-2- yl)methyl]-5-oxopyrrolidin-2- yl}ethyl)nicotinamide | LCMS Method (C) RT 1.64 min m/z Obs [M + 1] 434.3 calc [M + 1] 433.19139 |
| 57 | 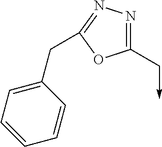 | R² = F | N-[(5-Benzyl-1,3,4- oxadiazol-2-yl)methyl]-6-(3- fluorophenyl)nicotinamide | LCMS Method (C) RT 2.14 min m/z Obs [M + 1] 389.5 calc [M + 1] 388.13355 |
| 58 | 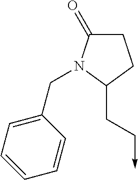 | R² = F | N-[2-(1-Benzyl-5- oxopyrrolidin-2-yl)ethyl]-6- (3-fluorophenyl)nicotinamide | LCMS Method (C) RT 2.05 min m/z Obs [M + 1] 418.5 calc [M + 1] 417.18525 |
| 59 | 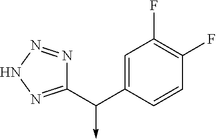 | R² = F | N-[(3,4-Difluorophenyl)(2H- tetrazol-5-yl)methyl]-6-(3- fluorophenyl)nicotinamide | LCMS Method (C) RT 2.18 min m/z Obs [M + 1] 411.4 calc [M + 1] 410.11029 |
| 60 | 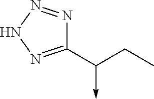 | R² = F | 6-(3-Fluorophenyl)-N-[1-(2H- tetrazol-5- yl)propyl]nicotinamide | LCMS Method (C) RT 1.72 min m/z Obs [M + 1] 327.3 calc [M + 1] 326.12913 |
| 61 | 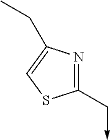 | R² = F | N-[(4-Ethyl-1,3-thiazol-2- yl)methyl]-6-(3- fluorophenyl)nicotinamide | LCMS Method (C) RT 2.09 min m/z Obs [M + 1] 342.1 calc [M + 1] 341.09981 |
| 62 | 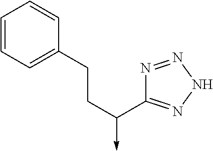 | R² = F | 6-(3-Fluorophenyl)-N-[3- phenyl-1-(2H-tetrazol-5- yl)propyl]nicotinamide | LCMS Method (C) RT 2.24 min m/z Obs [M + 1] 403.3 calc [M + 1] 402.16043 |
| 63 | 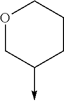 | R² = F | 6-(3-Fluorophenyl)-N- (tetrahydro-2H-pyran-3- yl)nicotinamide | LCMS Method (C) RT 1.79 min m/z Obs [M + 1] 300.9 calc [M + 1] 300.1274; |
| 64 | 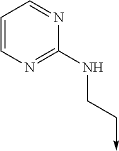 | R² = F | 6-(3-Fluorophenyl)-N-[2- (pyrimidin-2- ylamino)ethyl]nicotinamide | LCMS Method (C) RT 1.41 min m/z Obs [M + 1] 338.5 calc [M + 1] 337.13388 |
| 65 | 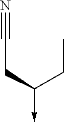 | R² = F | N-[(1R)-1- (Cyanomethyl)propyl]-6-(3- fluorophenyl)nicotinamide | LCMS Method (C) RT 1.97 min m/z Obs [M + 1] 298.1 calc [M + 1] 297.12773 |
| 66 | 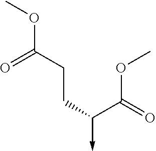 | R² = F | Dimethyl N-{[6-(3- fluorophenyl)pyridin-3- yl]carbonyl}-D-glutamate | LCMS Method (C) RT 1.99 min m/z Obs [M + 1] 375.0 calc [M + 1] 374.12779 |
| 67 | 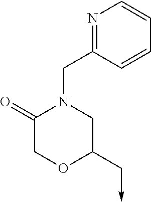 | R² = F | 6-(3-Fluorophenyl)-N-{[5- oxo-4-(pyridin-2- ylmethyl)morpholin-2- yl]methyl}nicotinamide | LCMS Method (C) RT 1.35 min m/z Obs [M + 1] 421.3 calc [M + 1] 420.15976 |
| 68 | 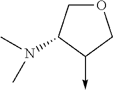 | R² = F | N-[(3S,4S)-4- (Dimethylamino)tetrahydro- furan-3-yl]-6-(3- fluorophenyl)nicotinamide | LCMS method (C) RT 1.28 min m/z Obs [M + 1] 330.4 calc [M + 1] 329.15395 |
| 69 | 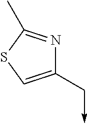 | R² = F | 6-(3-Fluorophenyl)-N-[(2- methyl-1,3-thiazol-4- yl)methyl]nicotinamide | LCMS Method (C) RT 1.77 min m/z Obs [M + 1] 328.3 calc [M + 1] 327.08416 |
| 70 | 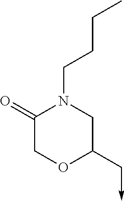 | R² = F | N-[(4-Butyl-5-oxomorpholin- 2-yl)methyl]-6-(3- fluorophenyl)nicotinamide | LCMS Method (C) RT 1.93 min m/z Obs [M + 1] 386.3 calc [M + 1] 385.18016 |
| 71 | 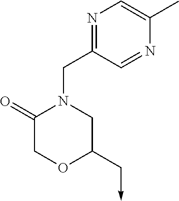 | R² = F | 6-(3-Fluorophenyl)-N-({4-[(5- methylpyrazin-2-yl)methyl]-5- oxomorpholin-2- yl}methyl)nicotinamide | LCMS Method (C) RT 1.59 min m/z Obs [M + 1] 436.0 calc [M + 1] 435.17066 |
| 72 | 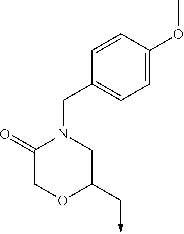 | R² = F | 6-(3-Fluorophenyl)-N-{[4-(4- methoxybenzoyl)-5- oxomorpholin-2- yl]methyl}nicotinamide | LCMS Method (C) RT 2.04 min m/z Obs [M + 1] 450.3 calc [M + 1] 449.17508 |
| 73 | 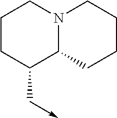 | R² = F | 6-(3-Fluorophenyl)-N- [(1S,9aR)-octahydro-2H- quinolizin-1- ylmethyl]nicotinamide | LCMS Method (C) RT 1.41 min m/z Obs [M + 1] 368.3 calc [M + 1] 367.20598 |
| 74 | 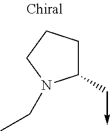 | R² = F | N-{[(2R)-1-Ethylpyrrolidin-2- yl]methyl}-6-(3-fluorophenyl) nicotinamide | LCMS Method (C) RT 1.35 min m/z Obs [M + 1] 328.3 calc [M + 1] 327.17468 |
| 75 | 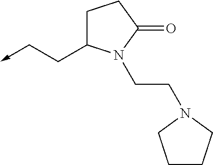 | R² = F | 6-(3-Fluorophenyl)-N-{2-[5- oxo-1-(2-pyrrolidin-1- ylethyl)pyrrolidin-2- yl]ethyl}nicotinamide | LCMS Method (C) RT 1.35 min m/z Obs [M + 1] 425.5 calc [M + 1] 425.23 |
| 76 | 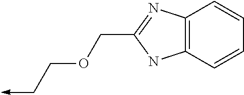 | R² = F | N-[2-(1H-Benzimidazol-2- ylmethoxy)ethyl]-6-(3- fluorophenyl)nicotinamide | LCMS Method (C) RT 1.49 min m/z Obs [M + 1] 391.4 calc [M + 1] 391.15 |
| 77 | 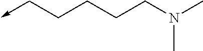 | R² = F | N-[5-(Dimethylamino)pentyl]- 6-(3-fluorophenyl)nicotinamide | LCMS Method (C) RT 1.32 min m/z Obs [M + 1] 330.4 calc [M + 1] 330.20 |
| 78 | 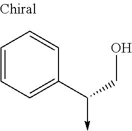 | R² = F | 6-(3-Fluorophenyl)-N-[(1R)- 2-hydroxy-1-phenylethyl] nicotinamide | LCMS Method (C) RT 1.93 min m/z Obs [M + 1] 337.5 calc [M + 1] 337.14 |
| 79 | 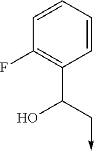 | R² = F | 6-(3-Fluorophenyl)-N-[2-(2- fluorophenyl)-2- hydroxyethyl]nicotinamide | LCMS Method (C) RT 2.02 min m/z Obs [M + 1] 355.1 calc [M + 1] 355.13 |
| 80 | 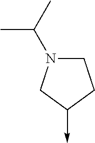 | R² = F | 6-(3-Fluorophenyl)-N-(1- isopropylpyrrolidin-3- yl)nicotinamide | LCMS Method (C) RT 1.33 min m/z Obs [M + 1] 328.1 calc [M + 1] 328.18 |
| 81 | 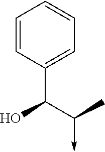 | R² = F | 6-(3-Fluorophenyl)-N- [(1R,2S)-2-hydroxy-1- methyl-2- phenethyl]nicotinamide | LCMS Method (C) RT 2.06 min m/z Obs [M + 1] 351.3 calc [M + 1] 351.15 |
| 82 | 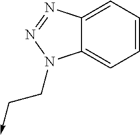 | R² = F | N-[2-(1H-Benzotriazol-1- yl)ethyl]-6-(3- fluorophenyl)nicotinamide | LCMS Method (C) RT 1.93 min m/z Obs [M + 1] 362.4 calc [M + 1] 362.14 |
| 83 | 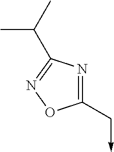 | R² = F | 6-(3-Fluorophenyl)-N-[(3- isopropyl-1,2,4-oxadiazol-5- yl)methyl]nicotinamide | LCMS Method (C) RT 2.11 min m/z Obs [M + 1] 341.3 calc [M + 1] 341.14 |
| 84 | 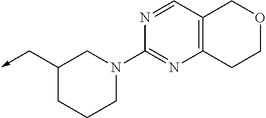 | R² = F | N-{[1-(7,8-Dihydro-5H- pyrano[4,3-d]pyrimidin-2- yl)piperidin-3-yl]methyl}-6- (3-fluorophenyl)nicotinamide | LCMS Method (C) RT 1.80 min m/z Obs [M + 1] 448.3 calc [M + 1] 448.22 |
| 85 | 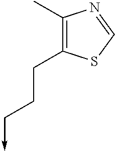 | R² = F | 6-(3-Fluorophenyl)-N-[3-(4- methyl-1,3-thiazol-5- yl)propyl]nicotinamide | LCMS Method (C) RT 1.62 min m/z Obs [M + 1] 356.1 calc [M + 1] 356.12 |
| 86 | 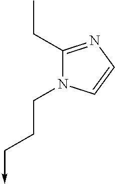 | R² = F | N-[3-(2-Ethyl-1H-imidazol-1- yl)propyl]-6-(3- fluorophenyl)nicotinamide | LCMS Method (C) RT 1.37 min m/z Obs [M + 1] 353.3 calc [M + 1] 353.18 |
| 87 | 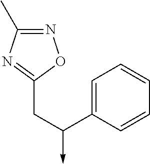 | R² = F | 6-(3-Fluorophenyl)-N-[2-(3- methyl-1,2,4-oxadiazol-5-yl)- 1-phenylethyl]nicotinamide | LCMS Method (C) RT 2.31 min m/z Obs [M + 1] 403.3 calc [M + 1] 403.16 |
| 88 | 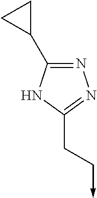 | R² = F | N-[2-(5-Cyclopropyl-4H- 1,2,4-triazol-3-yl)ethyl]-6-(3- fluorophenyl)nicotinamide | LCMS Method (C) RT 1.40 min m/z Obs [M + 1] 352.3 calc [M + 1] 352.16 |
| 89 | 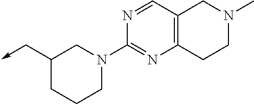 | R² = F | 6-(3-Fluorophenyl)-N-{[1-(6- methyl-5,6,7,8- tetrahydropyrido[4,3- d]pyrimidin-2-yl)piperidin-3- yl]methyl}nicotinamide | LCMS Method (C) RT 1.51 min m/z Obs [M + 1] 461.4 calc [M + 1] 461.25 |
| 90 | 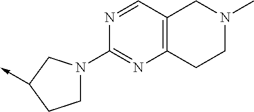 | R² = F | 6-(3-Fluorophenyl)-N-[1-(6- methyl-5,6,7,8- tetrahydropyrido[4,3- d]pyrimidin-2-yl)pyrrolidin-3- yl]nicotinamide | LCMS Method (C) RT 1.36 min m/z Obs [M + 1] 43.4 calc [M + 1] 433.21 |
| 91 | 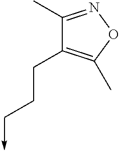 | R² = F | N-[3-(3,5-Dimethylisoxazol- 4-yl)propyl]-6-(3- fluorophenyl)nicotinamide | LCMS Method (C) RT 2.07 min m/z Obs [M + 1] 354.1 calc [M + 1] 354.16 |
| 92 | 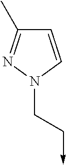 | R² = F | 6-(3-Fluorophenyl)-N-[2-(3- methyl-1H-pyrazol-1- yl)ethyl]nicotinamide | LCMS Method (C) RT 1.75 min m/z Obs [M + 1] 325.4 calc [M + 1] 325.15 |
| 93 | 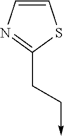 | R² = F | 6-(3-Fluorophenyl)-N-[2-(1,3- thiazol-2- yl)ethyl]nicotinamide | LCMS Method (C) RT 1.71 min m/z Obs [M + 1] 328.0 calc [M + 1] 328.09 |
| 94 | 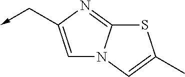 | R² = F | 6-(3-Fluorophenyl)-N-[(2- methylimidazo[2,1- b][1,3]thiazol-6- yl)methyl]nicotinamide | LCMS Method (C) RT 1.43 min m/z Obs [M + 1] 367.1 calc [M + 1] 367.10 |
| 95 | 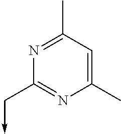 | R² = F | N-[(4,6-Dimethylpyrimidin-2- yl)methyl]-6-(3- fluorophenyl)nicotinamide | LCMS Method (C) RT 1.68 min m/z Obs [M + 1] 337.5 calc [M + 1] 337.15 |
| 96 | 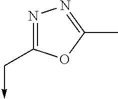 | R² = F | 6-(3-Fluorophenyl)-N-[(5- methyl-1,3,4-oxadiazol-2- yl)methyl]nicotinamide | LCMS Method (C) RT 1.56 min m/z Obs [M + 1] 313.0 calc [M + 1] 313.11 |
| 97 | 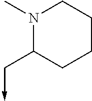 | R² = F | 6-(3-Fluorophenyl)-N-[(1- methylpiperidin-2- yl)methyl]nicotinamide | LCMS Method (C) RT 1.31 min m/z Obs [M + 1] 328.3 calc [M + 1] 328.18 |
| 98 | 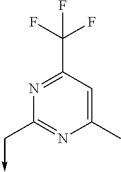 | R² = F | 6-(3-Fluorophenyl)-N-{[4- methyl-6- (trifluoromethyl)pyrimidin-2- yl]methyl}nicotinamide | LCMS Method (C) RT 2.24 min m/z Obs [M + 1] 391.4 calc [M + 1] 391.12 |
| 99 | 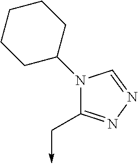 | R² = F | N-[(4-Cyclohexyl-4H-1,2,4- triazol-3-yl)methyl]-6-(3- fluorophenyl)nicotinamide | LCMS Method (C) RT 1.82 min m/z Obs [M + 1] 380.3 calc [M + 1] 380.19 |
| 100 | 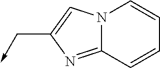 | R² = F | 6-(3-Fluorophenyl)-N- (imidazo[1,2-a]pyridin-2- ylmethyl)nicotinamide | LCMS Method (C) RT 1.35 min m/z Obs [M + 1] 347.1 calc [M + 1] 347.14 |
| 101 | 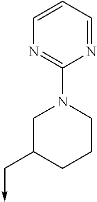 | R² = F | 6-(3-Fluorophenyl)-N-[(1- pyrimidin-2-ylpiperidin-3- yl)methyl]nicotinamide | LCMS Method (C) RT 1.76 min m/z Obs [M + 1] 392.4 calc [M + 1] 392.20 |
| 102 | 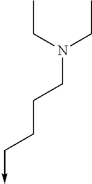 | R² = F | N-[4-(Diethylamino)butyl]-6- (3-fluorophenyl)nicotinamide | LCMS Method (C) RT 1.35 min m/z Obs [M + 1] 344.1 calc [M + 1] 344.21 |
| 103 | 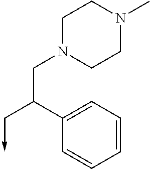 | R² = F | 6-(3-Fluorophenyl)-N-[3-(4- methylpiperazin-1-yl)-2- phenylpropyl]nicotinamide | LCMS Method (C) RT 1.42 min m/z Obs [M + 1] 433.4 calc [M + 1] 433.24 |
| 104 | 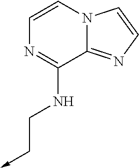 | R² = F | 6-(3-Fluorophenyl)-N-[2- (imidazo[1,2-a]pyrazin-8- ylamino)ethyl]nicotinamide | LCMS Method (C) RT 1.36 min m/z Obs [M + 1] 377.3 calc [M + 1] 377.15 |
| 105 | 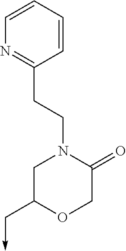 | R² = F | 6-(3-Fluorophenyl)-N-{[5- oxo-4-(2-pyridin-2- ylethyl)morpholin-2- yl]methyl}nicotinamide | LCMS Method (C) RT 1.31 min m/z Obs [M + 1] 435.3 calc [M + 1] 435.18 |
| 106 | 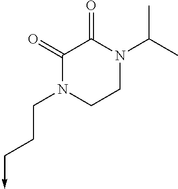 | R² = F | 6-(3-Fluorophenyl)-N-[3-(4- isopropyl-2,3-dioxopiperazin- 1-yl)propyl]nicotinamide | LCMS Method (C) RT 1.63 min m/z Obs [M + 1] 416.1 calc [M + 1] 413.1 |
| 107 | 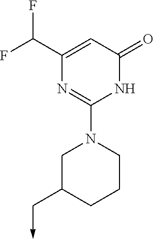 | R² = F | N-({1-[4-(Difluoromethyl)-6- oxo-1,6-dihydropyrimidin-2- yl]piperidin-3-yl}methyl)-6-(3- fluorophenyl)nicotinamide | LCMS Method (C) RT 2.04 min m/z Obs [M + 1] 458.1 calc [M + 1] 458.18 |
| 108 | 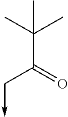 | R² = F | N-(3,3-Dimethyl-2-oxobutyl)- 6-(3- fluorophenyl)nicotinamide | LCMS Method (C) RT 2.17 min m/z Obs [M + 1] 315.3 calc [M + 1] 315.15 |
| 109 | 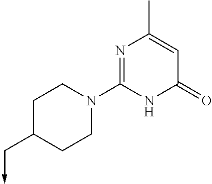 | R² = F | 6-(3-Fluorophenyl)-N-{[1-(4- methyl-6-oxo-1,6- dihdyropyrimidin-2- yl)piperidin-4- yl]methyl}nicotinamide | LCMS Method (C) RT 1.42 min m/z Obs [M + 1] 422.3 calc [M + 1] 422.20 |
| 110 | 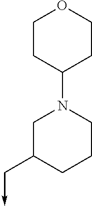 | R² = F | 6-(3-Fluorophenyl)-N-{[1- (tetrahydro-2H-pyran-4- yl)piperidin-3- yl]methyl}nicotinamide | LCMS Method (C) RT 1.35 min m/z Obs [M + 1] 398.0 calc [M + 1] 398.23 |
| 111 | 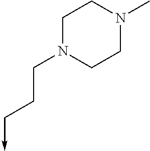 | R² = F | 6-(3-Fluorophenyl)-N-[3-(4- methylpiperazin-1- yl)propyl]nicotinamide | LCMS Method (C) RT 1.10 min m/z Obs [M + 1] 357.3 calc [M + 1] 357.21 |
| 112 | 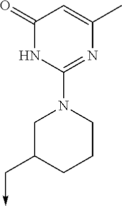 | R² = F | 6-(3-Fluorophenyl)-N-{[1-(4- methyl-6-oxo-1,6- dihydropyrimidin-2- yl)piperidin-3- yl]methyl}nicotinamide | LCMS Method (C) RT 1.48 min m/z Obs [M + 1] 422.3 calc [M + 1] 422.20 |
| 113 | 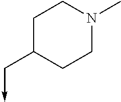 | R² = F | 6-(3-Fluorophenyl)-N-[(1- methylpiperidin-4- yl)methyl]nicotinamide | LCMS Method (C) RT 1.26 min m/z Obs [M + 1] 328.1 calc [M + 1] 328.18 |
| 114 | 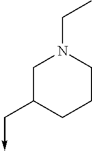 | R² = F | N-[(1-Ethylpiperidin-3- yl)methyl]-6-(3- fluorophenyl)nicotinamide | LCMS Method (C) RT 1.34 min m/z Obs [M + 1] 342.1 calc [M + 1] 342.20 |
| 115 | 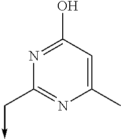 | R² = F | 6-(3-Fluorophenyl)-N-[(4- hydroxy-6-methylpyrimidin-2- yl)methyl]nicotinamide | LCMS Method (C) RT 1.43 min m/z Obs [M + 1] 339.4 calc [M + 1] 339.13 |
| 116 | 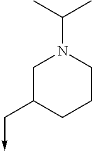 | R² = F | 6-(3-Fluorophenyl)-N-[(1- isopropylpiperidin-3- yl)methyl]nicotinamide | LCMS Method (C) RT 1.39 min m/z Obs [M + 1] 356.3 calc [M + 1] 356.21 |
| 117 | 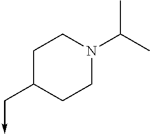 | R² = F | 6-(3-Fluorophenyl)-N-[(1- isopropylpiperidin-4- yl)methyl]nicotinamide | LCMS Method (C) RT 1.37 min m/z Obs [M + 1] 356.3 calc [M + 1] 356.21 |
| 118 | 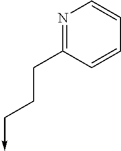 | R² = F | 6-(3-Fluorophenyl)-N-(3- pyridin-2- ylpropyl)nicotinamide | LCMS Method (C) RT 1.31 min m/z Obs [M + 1] 336.5 calc [M + 1] 336.15 |
| 119 | 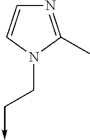 | R² = F | 6-(3-Fluorophenyl)-N-[2-(2- methyl-1H-imidazol-1- yl)ethyl]nicotinamide | LCMS Method (C) RT 1.26 min m/z Obs [M + 1] 325.4 calc [M + 1] 325.15 |
| 120 | 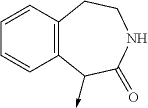 | R² = F | 6-(3-Fluorophenyl)-N-(2-oxo- 2,3,4,5-tetrahydro-1H-3- benzazepin-1- yl)nicotinamide | LCMS Method (C) RT 2.07 min m/z Obs [M + 1] 376.0 calc [M + 1] 376.15 |
| 121 | 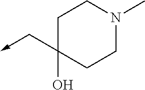 | R² = F | 6-(3-Fluorophenyl)-N-[(4- hydroxy-1-methylpiperidin-4- yl)methyl]nicotinamide | LCMS Method (C) RT 1.20 min m/z Obs [M + 1] 344.3 calc [M + 1] 344.18 |
| 122 | 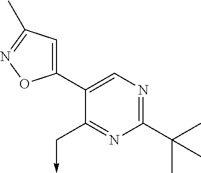 | R² = F | N-{[2-tert-Butyl-5-(3- methylisoxazol-5- yl)pyrimidin-4-yl]methyl}-6- (3-fluorophenyl)nicotinamide | LCMS Method (C) RT 2.74 min m/z Obs [M + 1] 446.5 calc [M + 1] 446.20 |
| 123 | 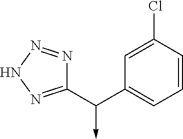 | R² = F | N-[(3-Chlorophenyl)(2H- tetrazol-5-yl)methyl]-6-(3- fluorophenyl)nicotinamide | LCMS Method (C) RT 2.23 min m/z Obs [M + 1] 409.0 calc [M + 1] 409.10 |
| 124 | 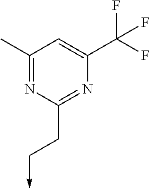 | R² = F | 6-(3-Fluorophenyl)-N-{2-[4- methyl-6- (trifluoromethyl)pyrimidin-2- yl]ethyl}nicotinamide | LCMS Method (C) RT 2.21 min m/z Obs [M + 1] 405.4 calc [M + 1] 405.13 |
| 125 | 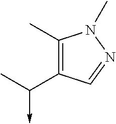 | R² = F | N-[1-(1,5-Dimethyl-1H- pyrazol-4-yl)ethyl]-6-(3- fluorophenyl)nicotinamide | LCMS Method (C) RT 1.75 min m/z Obs [M + 1] 339.4 calc [M + 1] 339.16 |
| 126 | 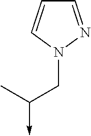 | R² = F | 6-(3-Fluorophenyl)-N-[1- methyl-2-(1H-pyrazol-1- yl)ethyl]nicotinamide | LCMS Method (C) RT 1.82 min m/z Obs [M + 1] 325.4 calc [M + 1] 325.15 |
| 127 | 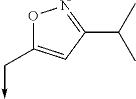 | R² = F | 6-(3-Fluorophenyl)-N-[(3- isopropylisoxazol-5- yl)methyl]nicotinamide | LCMS Method (C) RT 2.23 min m/z Obs [M + 1] 340.3 calc [M + 1] 340.15 |
| 128 | 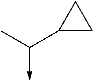 | R² = F | N-(1-Cyclopropylethyl)-6-(3- fluorophenyl)nicotinamide | LCMS Method (C) RT 2.19 min m/z Obs [M + 1] 285.4 calc [M + 1] 285.14 |
| 129 | 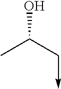 | R² = F | 6-(3-Fluorophenyl)-N-[(2S)- 2-hydroxypropyl]nicotinamide | LCMS Method (C) RT 1.45 min m/z Obs [M + 1] 275.0 calc [M + 1] 275.12 |
| 130 | 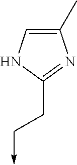 | R² = F | 6-(3-Fluorophenyl)-N-[2-(4- methyl-1H-imidazol-2- yl)ethyl]nicotinamide | LCMS Method (C) RT 1.31 min m/z Obs [M + 1] 325.4 calc [M + 1] 325.15 |
| 131 | 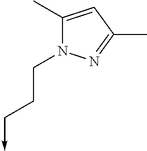 | R² = F | N-[3-(3,5-Dimethyl-1H- pyrazol-1-yl)propyl]-6-(3- fluorophenyl)nicotinamide | LCMS Method (C) RT 1.66 min m/z Obs [M + 1] 353.3 calc [M + 1] 353.18 |
| 132 | 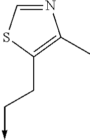 | R² = F | 6-(3-Fluorophenyl)-N-[2-(4- methyl-1,3-thiazol-5- yl)ethyl]nicotinamide | LCMS Method (C) RT 1.58 min m/z Obs [M + 1] 342.1 calc [M + 1] 342.11 |
| 133 | 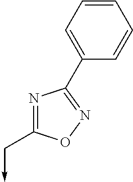 | R² = F | 6-(3-Fluorophenyl)-N-[(3- phenyl-1,2,4-oxadiazol-5- yl)methyl]nicotinamide | LCMS Method (C) RT 2.43 min m/z Obs [M + 1] 375.0 calc [M + 1] 375.13 |
| 134 | 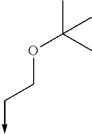 | R² = F | N-(2-tert-Butoxyethyl)-6-(3- fluorophenyl)nicotinamide | LCMS Method (C) RT 2.17 min m/z Obs [M + 1] 317.3 calc [M + 1] 317.17 |
| 135 | 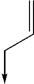 | R² = F | N-Allyl-6-(3- fluorophenyl)nicotinamide | LCMS Method (C) RT 1.89 min m/z Obs [M + 1] 257.0 calc [M + 1] 257.11 |
| 136 | 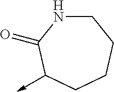 | R² = F | 6-(3-Fluorophenyl)-N-(2- oxoazepan-3-yl)nicotinamide | LCMS Method (C) RT 1.68 min m/z Obs [M + 1] 328.1 calc [M + 1] 328.15 |
| 137 | 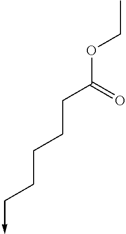 | R² = F | Ethyl 6-({[6-(3- fluorophenyl)pyridin-3- yl]carbonyl}amino)hexanoate | LCMS Method (C) RT 2.29 min m/z Obs [M + 1] 359.1 calc [M + 1] 359.18 |
| 138 | 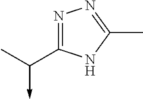 | R² = F | 6-(3-Fluorophenyl)-N-[1-(5- methyl-4H-1,2,4-triazol-3- yl)ethyl]nicotinamide | LCMS Method (C) RT 1.37 min m/z Obs [M + 1] 326.1 calc [M + 1] 326.14 |
| 139 | 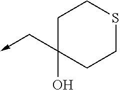 | R² = F | 6-(3-Fluorophenyl)-N-[(4- hydroxytetrahydro-2H- thiopyran-4- yl)methyl]nicotinamide | LCMS Method (C) RT 1.81 min m/z Obs [M + 1] 347.1 calc [M + 1] 347.12 |
| 140 | 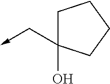 | R² = F | 6-(3-Fluorophenyl)-N-[(1- hydroxycyclopentyl)methyl] nicotinamide | LCMS Method (C) RT 1.80 min m/z Obs [M + 1] 315.1 calc [M + 1] 315.15 |
| 141 | 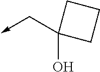 | R² = F | 6-(3-Fuorophenyl)-N-[(1- hydroxycyclobutyl)methyl] nicotinamide | LCMS Method (C) RT 1.69 min m/z Obs [M + 1] 301.1 calc [M + 1] 301.14 |
| 142 | 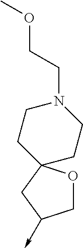 | R² = F | 6-(3-Fluorophenyl)-N-[8-(2- methoxyethyl)-1-oxa-8- azaspiro[4.5]dec-3- yl]nicotinamide | LCMS Method (C) RT 1.40 min m/z Obs [M + 1] 414.5 calc [M + 1] 414.22 |
| 143 | 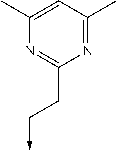 | R² = F | N-[2-(4,6-Dimethylpyrimidin- 2-yl)ethyl]-6-(3- fluorophenyl)nicotinamide | LCMS Method (C) RT 1.49 min m/z Obs [M + 1] 351.3 calc [M + 1] 351.16 |
| 144 | 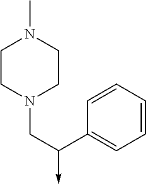 | R² = F | 6-(3-Fluorophenyl)-N-[2-(4- methylpiperazin-1-yl)-1- phenylethyl]nicotinamide | LCMS Method (C) RT 1.58 min m/z Obs [M + 1] 419.5 calc [M + 1] 419.23 |
| 145 | 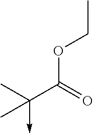 | R² = F | Ethyl N-{[6-(3- fluorophenyl)pyridin-3- yl]carbonyl}-2- methylalaninate | LCMS Method (C) RT 2.16 min m/z Obs [M + 1] 331.3 calc [M + 1] 331.15 |
| 146 | 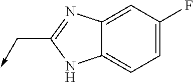 | R² = F | N-[(5-Fluoro-1H- benzimidazol-2-yl)methyl]-6- (3-fluorophenyl)nicotinamide | LCMS Method (C) RT 1.54 min m/z Obs [M + 1] 365.5 calc [M + 1] 365.12 |
| 147 | 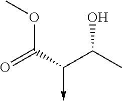 | R² = F | Methyl N-{[6-(3- fluorophenyl)pyridin-3- yl]carbonyl}-L-threoninate | LCMS Method (C) RT 1.68 min m/z Obs [M + 1] 333.3 calc [M + 1] 333.13 |
| 148 | 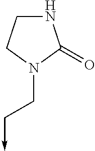 | R² = F | 6-(3-Fluorophenyl)-N-[2-(2- oxoimidazolidin-1- yl)ethyl]nicotinamide | LCMS Method (C) RT 1.42 m/z Obs [M + 1] 329.1 calc [M + 1] 329.14 |
| 149 | 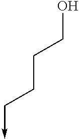 | R² = F | 6-(3-Fluorophenyl)-N-(4- hydroxybutyl)nicotinamide | LCMS Method (C) RT 1.51 min m/z Obs [M + 1] 289.0 calc [M + 1] 289.14 |
| 150 | 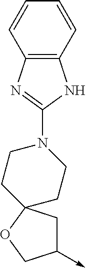 | R² = F | N-[8-(1H-Benzimidazol-2-yl)- 1-oxa-8-azaspiro[4.5]dec-3- yl]-6-(3- fluorophenyl)nicotinamide | LCMS Method (C) RT 1.61 min m/z Obs [M + 1] 472.6 calc [M + 1] 472.54 |

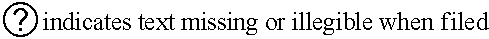

| | | | | Purification and |
|---|---|---|---|---|
| Ex | R₇ | R¹⁻⁵ | Name | Characterisation |
| 151 | 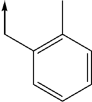 | | N-(2-Methylbenzyl)-6- phenylnicotinamide | LCMS Method (E) RT 4.86 min m/z Obs [M + 1] 303.1 calc [M + 1] 302.38 |
| 152 | 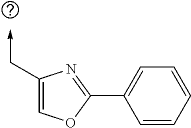 | R² = F | 6-(3-Fluorophenyl)-N-[(2- phenyl-1,3-oxazol-4- yl)methyl]nicotinamide | LCMS Method (E) RT 4.91 min m/z Obs [M + 1] 373.1 calc [M + 1] 373.3 |
| 153 | 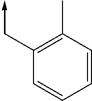 | R² = F | 6-(3-Fluorophenyl)-N-(2- methylbenzyl)nicotinamide | LCMS Method (E) RT 5.19 min m/z Obs [M + 1] 320.1 calc [M + 1] 320.3 |
| 154 | 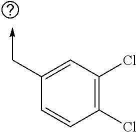 | R² = F | N-(3,4-Dichlorobenzyl)-6-(3- fluorophenyl)nicotinamide | LCMS Method (E) RT 5.40 min m/z Obs [M + 1] 375.0 calc [M + 1] 375.2 |
| 155 | 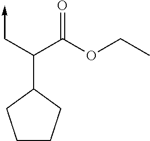 | R² = F | Ethyl 2-cyclopentyl-3-({[6-(3- fluorophenyl)pyridin-3- yl]carbonyl}amino)propanoate | LCMS Method (E) RT 5.37 min m/z Obs [M + 1] 385.2 calc [M + 1] 385.2 |
| 156 | 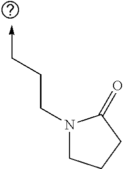 | R² = F | 6-(3-Fluorophenyl)-N-[3-(2- oxopyrrolidin-1- yl)propyl]nicotinamide | LCMS Method (E) RT 3.87 min m/z Obs [M + 1] 341.1 calc [M + 1] 341.4 |
| 157 | 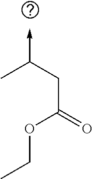 | R² = F | Ethyl 3-({[6-(3- fluorophenyl)pyridin-3- yl]carbonyl}amino)butanoate | LCMS Method (E) RT 4.45 min m/z Obs [M + 1] 331.1 calc [M + 1] 331.1 |
| 158 | 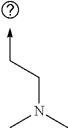 | R² = F | N-[2-(Dimethylamino)ethyl]- 6-(3- fluorophenyl)nicotinamide | LCMS Method (E) RT 2.81 min m/z Obs [M + 1] 287.1 calc [M + 1] 287.3 |
| 159 | 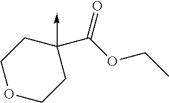 | R² = F | Ethyl 4-({[6-(3- fluorophenyl)pyridin-3- yl]carbonyl}amino)tetrahydro- 2H-pyran-4-carboxylate | LCMS Method (E) RT 4.34 min m/z Obs [M + 1] 372.2 calc [M + 1] 372.4 |
| 160 | 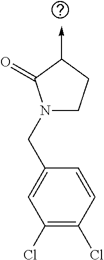 | R² = F | N-[1-(3,4-Dichlorobenzyl)-2- oxopyrrolidin-3-yl]-6-(3- fluorophenyl)nicotinamide | LCMS Method (E) RT 5.14 min m/z Obs [M + 1] 458.1 calc [M + 1] 458.3 |
| 161 | 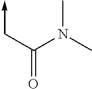 | R² = F | N-[2-(dimethylamino)-2- oxoethyl]-6-(3- fluorophenyl)nicotinamide | LCMS Method (E) RT 3.89 min m/z Obs [M + 1] 301.1 calc [M + 1] 301.3 |
| 162 | 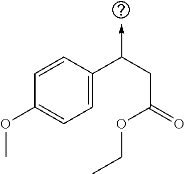 | R² = F | Ethyl 3-({[6-(3- fluorophenyl)pyridin-3- yl]carbonyl}amino)-3-(4- methoxyphenyl)propanoate | LCMS Method (E) RT 5.22 min m/z Obs [M + 1] 422.2 calc [M + 1] 422.5 |
| 163 | 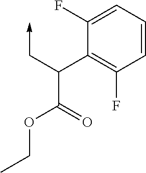 | R² = F | Ethyl 2-(2,6-difluorophenyl)- 3-({[6-(3- fluorophenyl)pyridin-3- yl]carbonyl}amino)propanoate | LCMS Method (E) RT 5.17 min m/z Obs [M + 1] 428.1 calc [M + 1] 428.4 |
| 164 | 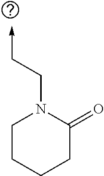 | R² = F | 6-(3-Fluorophenyl)-N-[2-(2- oxopiperidin-1- yl)ethyl]nicotinamide | LCMS Method (E) RT 4.07 min m/z Obs [M + 1] 341.2 calc [M + 1] 341.4 |
| 165 | 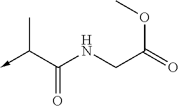 | R² = F | Methyl N-{[6-(3- fluorophenyl)pyridin-3- yl]carbonyl}-L-alanylglycinate | LCMS Method (E) RT 3.86 min m/z Obs [M + 1] 359.1 calc [M + 1] 359.4 |
| 166 | 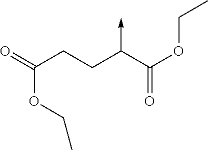 | R² = F | Diethyl N-{[6-(3- fluorophenyl)pyridin-3- yl]carbonyl}-D-glutamate | LCMS Method (E) RT 4.91 min m/z Obs [M + 1] 402.2 calc [M + 1] 402.4 |
| 167 | 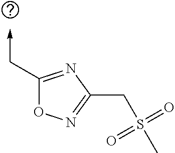 | R² = F | 6-(3-Fluorophenyl)-N-({3- [(methylsulfonyl)methyl]- 1,2,4-oxadiazol-5- yl}methyl)nicotinamide | LCMS Method (E) RT 4.21 min m/z Obs [M + 1] 390.1 calc [M + 1] 390.4 |
| 168 | 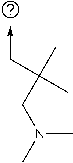 | R² = F | N-[3-(Dimethylamino)-2,2- dimethylpropyl]-6-(3- fluorophenyl)nicotinamide | LCMS Method (E) RT 2.88 min m/z Obs [M + 1] 330.2 calc [M + 1] 330.2 |
| 169 | 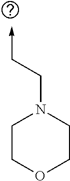 | R² = F | 6-(3-Fluorophenyl)-N-(2- morpholin-4- ylethyl)nicotinamide | LCMS Method (E) RT 2.84 min m/z Obs [M + 1] 330.2 calc [M + 1] 330.7 |
| 170 | 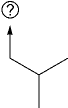 | R² = F | 6-(3-Fluorophenyl)-N- isobutylnicotinamide | LCMS Method (E) RT 4.61 min m/z Obs [M + 1] 273.1 calc [M + 1] 273.1 |
| 171 | 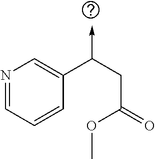 | R² = F | Methyl 3-({[6-(3- fluorophenyl)pyridin-3- yl]carbonyl}amino)-3-pyridin- 3-ylpropanoate | LCMS Method (E) RT 3.76 min m/z Obs [M + 1] 380.1 calc [M + 1] 380.1 |
| 172 | 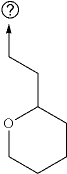 | R² = F | 6-(3-Ffluorophenyl)-N-[2- (tetrahydro-2H-pyran-2- yl)ethyl]nicotinamide | LCMS Method (E) RT 4.64 min m/z Obs [M + 1] 329.2 calc [M + 1] 329.2 |
| 173 | 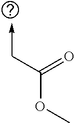 | R² = F | Methyl N-{[6-(3- fluorophenyl)pyridin-3- yl]carbonyl}glycinate | LCMS Method (E) RT 3.93 min m/z Obs [M + 1] 289.1 calc [M + 1] 289.1 |
| 174 | 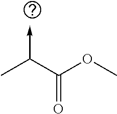 | R² = F | Methyl N-{[6-(3- fluorophenyl)pyridin-3- yl]carbonyl}alaninate | LCMS Method (E) RT 4.20 min m/z Obs [M + 1] 303.1 calc [M + 1] 303.1 |
| 174 | 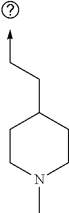 | R² = F | 6-(3-Fluorophenyl)-N-[2-(1- methylpiperidin-4- yl)ethyl]nicotinamide | LCMS Method (E) RT 2.88 min m/z Obs [M + 1] 342.2 calc [M + 1] 342.2 |
| 176 | 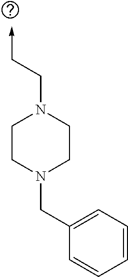 | R² = F | N-[2-(4-Benzylpiperazin-1- yl)ethyl]-6-(3- fluorophenyl)nicotinamide | LCMS Method (E) RT 3.20 min m/z Obs [M + 1] 419.2 calc [M + 1] 419.2 |
| 177 | 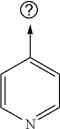 | R² = F | 6-(3-Fluorophenyl)-N-pyridin- 4-ylnicotinamide | LCMS Method (E) RT 3.17 min m/z Obs [M + 1] 294.1 calc [M + 1] 294.1 |
| 178 | 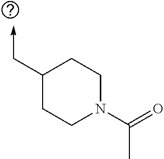 | R² = F | N-[(1-Acetylpiperidin-4- yl)methyl]-6-(3- fluorophenyl)nicotinamide | LCMS Method (E) RT 3.90 min m/z Obs [M + 1] 356.2 calc [M + 1] 356.2 |
| 179 | 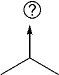 | R² = F | 6-(3-Fluorophenyl)-N- isopropylnicotinamide | LCMS Method (E) RT 4.49 min m/z Obs [M + 1] 259.1 calc [M + 1] 259.1 |
| 180 | 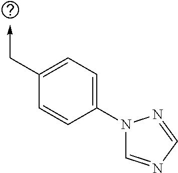 | R² = F | 6-(3-Fluorophenyl)-N-[4-(1H- 1,2,4-triazol-1- yl)benzyl]nicotinamide | LCMS Method (E) RT 4.18 min m/z Obs [M + 1] 374.1 calc [M + 1] 374.1 |
| 181 | 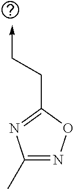 | R² = F | 6-(3-Fluorophenyl)-N-[2-(3- methyl-1,2,4-oxadiazol-5- yl)ethyl]nicotinamide | LCMS Method (E) RT 4.13 min m/z Obs [M + 1] 327.1 calc [M + 1] 327.1 |
| 182 | 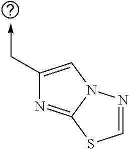 | R² = F | 6-(3-Fluorophenyl)-N- (imidazo[2,1- b][1,3,4]thiadiazol-6- ylmethyl)nicotinamide | LCMS Method (E) RT 3.90 min m/z Obs [M + 1] 354.1 calc [M + 1] 354.1 |
| 183 | 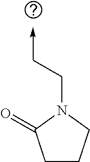 | R² = F | 6-(3-Fluorophenyl)-N-[2-(2- oxopyrrolidin-1- yl)ethyl]nicotinamide | LCMS Method (E) RT 3.91 min m/z Obs [M + 1] 328.1 calc [M + 1] 328.1 |
| 184 | 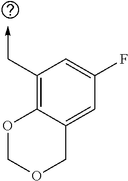 | R² = F | N-[(6-Fluoro-4H-1,3- benzodioxin-8-yl)methyl]-6- (3-fluorophenyl)nicotinamide | LCMS Method (E) RT 4.82 min m/z Obs [M + 1] 383.1 calc [M + 1] 383.1 |
| 185 | 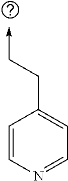 | R² = F | 6-(3-Fluorophenyl)-N-(2- pyridin-4- ylethyl)nicotinamide | LCMS Method (E) RT 3.11 min m/z Obs [M + 1] 322.1 calc [M + 1] 322.1 |
| 186 | 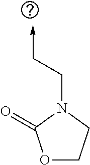 | R² = F | 6-(3-Fluorophenyl)-N-[2-(2- oxo-1,3-oxazolidin-3- yl)ethyl]nicotinamide | LCMS Method (E) RT 3.76 min m/z Obs [M + 1] 330.1 calc [M + 1] 330.1 |
| 187 | 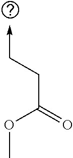 | R² = F | Methyl N-{[6-(3- fluorophenyl)pyridin-3- yl]carbonyl}-beta-alaninate | LCMS Method (E) RT 4.11 min m/z Obs [M + 1] 303.1 calc [M + 1] 303.1 |
| 188 | 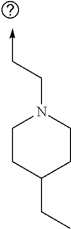 | R² = F | N-[2-(4-Ethylpiperidin-1- yl)ethyl]-6-(3- fluorophenyl)nicotinamide | LCMS Method (E) RT 3.14 min m/z Obs [M + 1] 356.2 calc [M + 1] 356.2 |
| 189 | 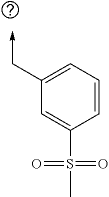 | R² = F | 6-(3-Fluorophenyl)-N-[3- (methylsulfonyl)benzyl] nicotinamide | LCMS Method (E) RT 4.32 min m/z Obs [M + 1] 385.1 calc [M + 1] 385.1 |
| 190 | 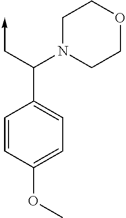 | R² = F | 6-(3-Fluorophenyl)-N-[2-(4- methoxyphenyl)-2- morpholin-4- ylethyl]nicotinamide | LCMS Method (E) RT 3.63 min m/z Obs [M + 1] 436.2 calc [M + 1] 436.2 |
| 191 | 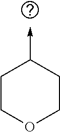 | R² = F | 6-(3-Ffluorophenyl)-N- (tetrahydro-2H-pyran-4- yl)nicotinamide | LCMS Method (E) RT 4.03 min m/z Obs [M + 1] 301.1 calc [M + 1] 301.1 |
| 192 | 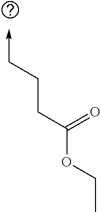 | R² = F | Ethyl 4-({[6-(3- fluorophenyl)pyridin-3- yl]carbonyl}amino)butanoate | LCMS Method (E) RT 4.61 min m/z Obs [M + 1] 331.1 calc [M + 1] 331.1 |
| 193 | 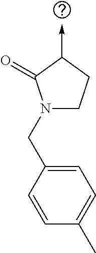 | R² = F | 6-(3-Fluorophenyl)-N-[1-(4- methylbenzyl)-2- oxopyrrolidin-3- yl]nicotinamide | LCMS Method (E) RT 4.78 min m/z Obs [M + 1] 404.2 calc [M + 1] 404.2 |
| 194 | 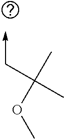 | R² = F | 6-(3-Fluorophenyl)-N-(2- methoxy-2- methylpropyl)nicotinamide | LCMS Method (E) RT 4.27 min m/z Obs [M + 1] 303.2 calc [M + 1] 303.2 |
| 195 | 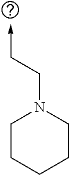 | R² = F | 6-(3-Fluorophenyl)-N-(2- piperidin-1- ylethyl)nicotinamide | LCMS Method (E) 3.01 min m/z Obs [M + 1] 328.2 calc [M + 1] 328.2 |
| 196 | 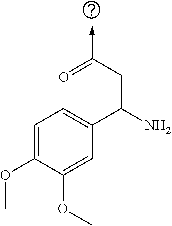 | R² = F | N-[3-Amino-3-(3,4- dimethoxyphenyl)propanoyl]- 6-(3- fluorophenyl)nicotinamide | LCMS Method (E) RT 3.92 min m/z Obs [M + 1] 424.2 calc [M + 1] 424.2 |
| 197 | 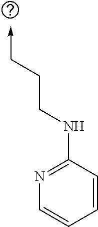 | R² = F | 6-(3-Fluorophenyl)-N-[3- (pyridin-2- ylamino)propyl]nicotinamide | LCMS Method (E) RT 3.08 min m/z Obs [M + 1] 351.2 calc [M + 1] 351.2 |
| 198 | 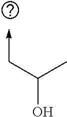 | R² = F | 6-(3-Fluorophenyl)-N-(2- hydroxypropyl)nicotinamide | LCMS Method (E) RT 3.86 min m/z Obs [M + 1] 275.1 calc [M + 1] 275.1 |
| 199 | 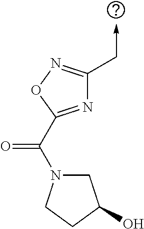 | R² = F | 6-(3-Fluorophenyl)-N-[(5- {[(3S)-3-hydroxypyrrolidin-1- yl]carbonyl}-1,2,4-oxadiazol- 3-yl)methyl]nicotinamide | LCMS Method (E) RT 4.12 min m/z Obs [M + 1] 412.1 calc [M + 1] 412.1 |
| 200 | 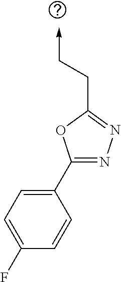 | R² = F | 6-(3-Fluorophenyl)-N-{2-[5- (4-fluorophenyl)-1,3,4- oxadiazol-2- yl]ethyl}nicotinamide | LCMS Method (E) RT 4.66 min m/z Obs [M + 1] 407.1 calc [M + 1] 407.1 |
| 201 | 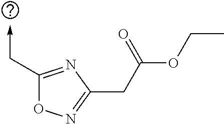 | R² = F | Ethyl {5-[({[6-(3- fluorophenyl)pyridin-3- yl]carbonyl}amino)methyl]- 1,2,4-oxadiazol-3-yl}acetate | LCMS Method (E) RT 4.51 min m/z Obs [M + 1] 385.1 calc [M + 1] 385.1 |
| 202 | 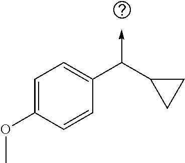 | R² = F | N-[Cyclopropyl(4- methoxyphenyl)methyl]-6-(3- fluorophenyl)nicotinamide | LCMS Method (E) RT 5.30 min m/z Obs [M + 1] 377.2 calc [M + 1] 377.2 |
| 203 | 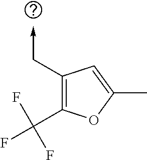 | R² = F | 6-(3-Fluorophenyl)-N-{[5- methyl-2-(trifluoromethyl)-3- furyl]methyl}nicotinamide | LCMS Method (E) RT 5.38 min m/z Obs [M + 1] 379.1 calc [M + 1] 379.1 |
| 204 | 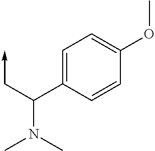 | R² = F | N-[2-(Dimethylamino)-2-(4- methoxyphenyl)ethyl]-6-(3- fluorophenyl)nicotinamide | LCMS Method (E) RT 3.57 min m/z Obs [M + 1] 394.2 calc [M + 1] 394.2 |
| 205 | 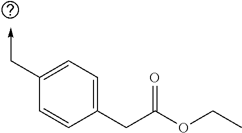 | R² = F | Ethyl {4-[({[6-(3- fluorophenyl)pyridin-3- yl]carbonyl}amino)methyl] phenyl}acetate | LCMS Method (E) RT 5.03 min m/z Obs [M + 1] 393.2 calc [M + 1] 393.2 |
| 206 | 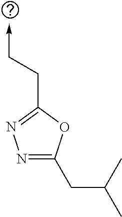 | R² = F | 6-(3-Fluorophenyl)-N-[2-(5- isobutyl-1,3,4-oxadiazol-2- yl)ethyl]nicotinamide | LCMS Method (E) RT 4.43 min m/z Obs [M + 1] 369.2 calc [M + 1] 369.2 |
| 207 | 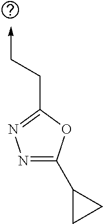 | R² = F | N-[2-(5-Cyclopropyl-1,3,4- oxadiazol-2-yl)ethyl]-6-(3- fluorophenyl)nicotinamide | LCMS Method (E) RT 4.02 min m/z Obs [M + 1] 353.1 calc [M + 1] 353.1 |
| 208 | 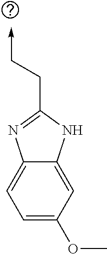 | R² = F | 6-(3-Fluorophenyl)-N-[2-(6- methoxy-1H-benzimidazol-2- yl)ethyl]nicotinamide | LCMS Method (E) RT 3.32 min m/z Obs [M + 1] 391.6 calc [M + 1] 391.6 |
| 209 | 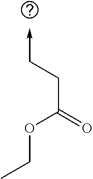 | R² = F | Ethyl N-{[6-(3- fluorophenyl)pyridin-3- yl]carbonyl}-beta-alaninate | LCMS Method (E) RT 4.28 min m/z Obs [M + 1] 317.1 calc [M + 1] 317.1 |
| 210 | 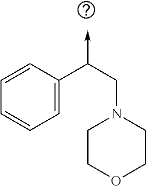 | R² = F | 6-(3-Fluorophenyl)-N-(2- morpholin-4-yl-1- phenylethyl)nicotinamide | LCMS Method (E) RT 3.58 min m/z Obs [M + 1] 406.2 calc [M + 1] 406.2 |
| 211 | 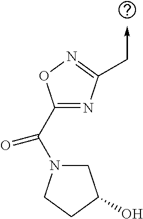 | R² = F | 6-(3-Fluorophenyl)-N-[(5- {[(3R)-3-hydroxypyrrolidin-1- yl]carbonyl}-1,2,4-oxadiazol- 3-yl)methyl]nicotinamide | LCMS Method (E) RT 3.83 min m/z Obs [M + 1] 412.1 calc [M + 1] 412.1 |
| 212 | 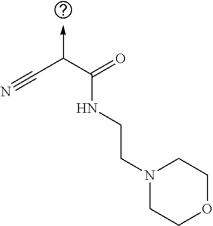 | R² = F | N-{1-Cyano-2-[(2-morpholin- 4-ylethyl)amino]-2-oxoethyl}- 6-(3-fluorophenyl)nicotinamide | LCMS Method (E) RT 3.05 min m/z Obs [M + 1] 412.2 calc [M + 1] 412.2 |
| 213 | 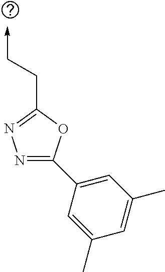 | R² = F | N-{2-[5-(3,5- Dimethylphenyl)-1,3,4- oxadiazol-2-yl]ethyl}-6-(3- fluorophenyl)nicotinamide | LCMS Method (E) RT 5.05 min m/z Obs [M + 1] 417.2 calc [M + 1] 417.2 |
| 214 | 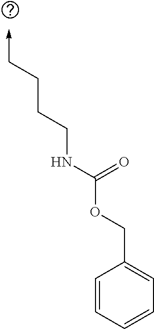 | R² = F | Benzyl [4-({[6-(3- fluorophenyl)pyridin-3- yl]carbonyl}amino)butyl] carbamate | LCMS Method (E) RT 4.84 min m/z Obs [M + 1] 422.2 calc [M + 1] 422.2 |
| 215 | 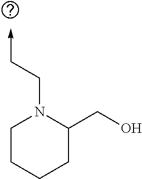 | R² = F | 6-(3-Fluorophenyl)-N-{2-[2- (hydroxymethyl)piperidin-1- yl]ethyl}nicotinamide | LCMS Method (E) RT 2.84 min m/z Obs [M + 1] 358.2 calc [M + 1] 358.2 |
| 216 | 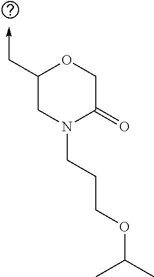 | R² = F | 6-(3-Fluorophenyl)-N-{[4-(3- isopropoxypropyl)-5- oxomorpholin-2- yl]methyl}nicotinamide | LCMS Method (E) RT 4.25 min m/z Obs [M + 1] 430.2 calc [M + 1] 430.2 |
| 217 | 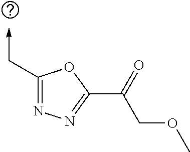 | R² = F | Ethyl 5-[({[6-(3- fluorophenyl)pyridin-3- yl]carbonyl}amino)methyl]- 1,3,4-oxadiazole-2- carboxylate | LCMS Method (E) RT 4.06 min m/z Obs [M + 1] 371.1 calc [M + 1] 371.1 |
| 218 | 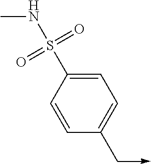 | R² = CH₃ | N-{4- [(Methylamino)sulfonyl] benzyl}-6-(3- methylphenyl)nicotinamide | LCMS Method (E) RT 4.46 min m/z Obs [M + 1] 396.1 calc [M + 1] 396.1 |
| 219 | 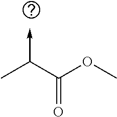 | R² = CH₃ | Methyl N-{[6-(3- methylphenyl)pyridin-3- yl]carbonyl}alaninate | LCMS Method (E) RT 4.32 min m/z Obs [M + 1] 299.1 calc [M + 1] 299.1 |
| 220 | 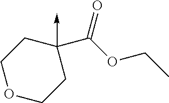 | R² = CH₃ | Ethyl 4-({[6-(3- methylphenyl)pyridin-3- yl]carbonyl}amino)tetrahydro- 2H-pyran-4-carboxylate | LCMS Method (E) RT 4.42 min m/z Obs [M + 1] 369.2 calc [M + 1] 369.2 |
| 221 | 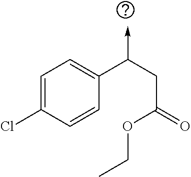 | R² = CH₃ | Ethyl 3-(4-chlorophenyl)-3- ({[6-(3-methylphenyl)pyridin- 3-yl]carbonyl}amino)propanoate | LCMS Method (E) RT 5.51 min m/z Obs [M + 1] 423.1 calc [M + 1] 423.1 |
| 222 | 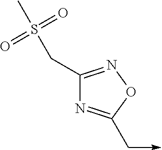 | R² = CH₃ | 6-(3-Methylphenyl)-N-({3- [(methylsulfonyl)methyl]- 1,2,4-oxadiazol-5- yl}methyl)nicotinamide | LCMS Method (E) RT 4.05 min m/z Obs [M + 1] 387.1 calc [M + 1] 387.1 |
| 223 | 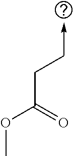 | R² = CH₃ | Methyl N-{[6-(3- methylphenyl)pyridin-3- yl]carbonyl}-beta-alaninate | LCMS Method (E) RT 4.14 min m/z Obs [M + 1] 299.1 calc [M + 1] 299.1 |
| 224 | 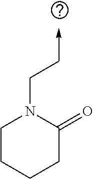 | R² = CH₃ | 6-(3-Methylphenyl)-N-[2-(2- oxopiperidin-1- yl)ethyl]nicotinamide | LCMS Method (E) RT 4.03 min m/z Obs [M + 1] 338.2 calc [M + 1] 338.2 |
| 225 | 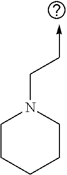 | R² = CH₃ | 6-(3-Methylphenyl)-N-(2- piperidin-1- ylethyl)nicotinamide | LCMS Method (E) RT 3.05 min m/z Obs [M + 1] 324.2 calc [M + 1] 324.2 |
| 226 | 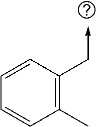 | R² = CH₃ | N-(2-Methylbenzyl)-6-(3- methylphenyl)nicotinamide | LCMS Method (E) RT 5.13 min m/z Obs [M + 1] 317.2 calc [M + 1] 317.2 |
| 227 | 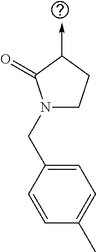 | R² = CH₃ | N-[1-(4-Methylbenzyl)-2- oxopyrrolidin-3-yl]-6-(3- methylphenyl)nicotinamide | LCMS Method (E) RT 4.82 min m/z Obs [M + 1] 400.2 calc [M + 1] 400.2 |
| 228 | 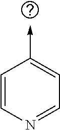 | R² = CH₃ | 6-(3-Methylphenyl)-N- pyridin-4-ylnicotinamide | LCMS Method (E) RT 3.31 min m/z Obs [M + 1] 290.1 calc [M + 1] 290.1 |
| 229 | 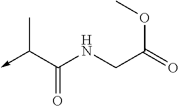 | R² = CH₃ | Methyl N-{[6-(3- methylphenyl)pyridin-3- yl]carbonyl}-L-alanylglycinate | LCMS Method (E) RT 3.95 min m/z Obs [M + 1] 356.2 calc [M + 1] 356.2 |
| 230 | 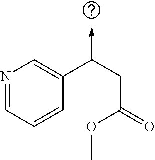 | R² = CH₃ | Methyl 3-({[6-(3- methylphenyl)pyridin-3- yl]carbonyl}amino)-3-pyridin- 3-ylpropanoate | LCMS Method (E) RT 3.80 min m/z Obs [M + 1] 376.2 calc [M + 1] 376.2 |
| 231 | 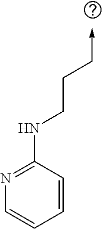 | R² = CH₃ | 6-(3-Methylphenyl)-N-[3- (pyridin-2- ylamino)propyl]nicotinamide | LCMS Method (E) RT 3.17 min m/z Obs [M + 1] 347.2 calc [M + 1] 347.2 |
| 232 | 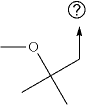 | R² = CH₃ | N-(2-Methoxy-2- methylpropyl)-6-(3- methylphenyl)nicotinamide | LCMS Method (E) RT 4.38 min m/z Obs [M + 1] 299.2 calc [M + 1] 299.2 |
| 233 | 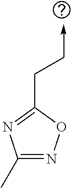 | R² = CH₃ | N-[2-(3-Methyl-1,2,4- oxadiazol-5-yl)ethyl]-6-(3- methylphenyl)nicotinamide | LCMS Method (E) RT 4.12 min m/z Obs [M + 1] 323.2 calc [M + 1] 323.2 |
| 234 | 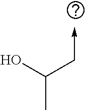 | R² = CH₃ | N-(2-Hydroxypropyl)-6-(3- methylphenyl)nicotinamide | LCMS Method (E) RT 3.81 min m/z Obs [M + 1] 271.1 calc [M + 1] 271.1 |
| 235 | 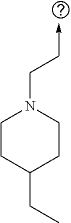 | R² = CH₃ | N-[2-(4-Ethylpiperidin-1- yl)ethyl]-6-(3- methylphenyl)nicotinamide | LCMS Method (E) RT 3.30 min m/z Obs [M + 1] 352.2 calc [M + 1] 352.2 |
| 236 | 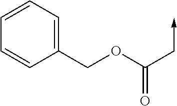 | R² = CH₃ | Benzyl N-{[6-(3- methylphenyl)pyridin-3- yl]carbonyl}glycinate | LCMS Method (E) RT 5.03 min m/z Obs [M + 1] 361.2 calc [M + 1] 361.2 |
| 237 | 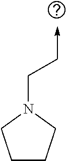 | R² = CH₃ | 6-(3-Methylphenyl)-N-(2- pyrrolidin-1- ylethyl)nicotinamide | LCMS Method (E) RT 3.00 min m/z Obs [M + 1] 310.2 calc [M + 1] 310.2 |
| 238 | 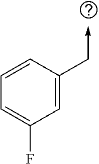 | R² = CH₃ | N-(3-Fluorobenzyl)-6-(3- methylphenyl)nicotinamide | LCMS Method (E) RT 3.99 min m/z Obs [M + 1] 327.1 calc [M + 1] 327.1 |
| 239 | 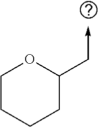 | R² = CH₃ | 6-(3-Methylphenyl)-N- (tetrahydro-2H-pyran-2- ylmethyl)nicotinamide | LCMS Method (E) RT 4.53 min m/z Obs [M + 1] 311.2 calc [M + 1] 311.2 |
| 240 | 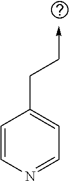 | R² = CH₃ | 6-(3-Methylphenyl)-N-(2- pyridin-4- ylethyl)nicotinamide | LCMS Method (E) RT 3.18 min m/z Obs [M + 1] 318.2 calc [M + 1] 318.2 |
| 241 | 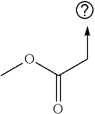 | R² = CH₃ | Methyl N-{[6-(3- methylphenyl)pyridin-3- yl]carbonyl}glycinate | LCMS Method (E) RT 4.10 min m/z Obs [M + 1] 285.1 calc [M + 1] 285.1 |
| 242 | 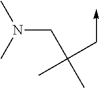 | R² = CH₃ | N-[3-(Dimethylamino)-2,2- dimethylpropyl]-6-(3- methylphenyl)nicotinamide | LCMS Method (E) RT 3.10 min m/z Obs [M + 1] 326.2 calc [M + 1] 326.2 |
| 243 | 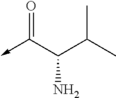 | R² = CH₃ | 6-(3-Methylphenyl)-N-L- valylnicotinamide | LCMS Method (E) RT 4.06 min m/z Obs 312.2 [M + 1] calc [M + 1] 312.2 |
| 244 | 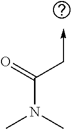 | R² = CH₃ | N-[2-(Dimethylamino)-2- oxoethyl]-6-(3- methylphenyl)nicotinamide | LCMS Method (E) RT 3.8 min m/z Obs [M = 1] 298.2 calc [M + 1] 298.2 |
| 245 | 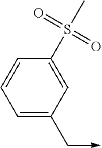 | R² = CH₃ | 6-(3-Methylphenyl)-N-[3- (methylsulfonyl)benzyl] nicotinamide | LCMS Method (E) RT min m/z Obs [M + 1] 381.1 calc [M + 1] 381.1 |
| 246 | 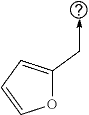 | R² = CH₃ | N-(2-Furylmethyl)-6-(3- methylphenyl)nicotinamide | m/z Obs [M + 1] 292.1 calc [M + 1] 293.1 |
| 247 | 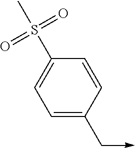 | R² = CH₃ | 6-(3-Methylphenyl)-N-[4- (methylsulfonyl)benzyl] nicotinamide | LCMS Method (E) RT 4.34 min m/z Obs [M + 1] 381.1 calc [M + 1] 381.1 |
| 248 | 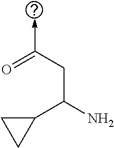 | R² = CH₃ | N-(3-Amino-3- cyclopropylpropanoyl)-6-(3- methylphenyl)nicotinamide | LCMS Method (E) RT 3.92 min m/z Obs [M = 1] 324.2 calc [M + 1] 324.2 |
| 249 | 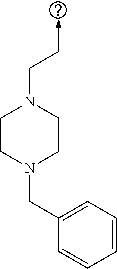 | R² = CH₃ | N-[2-(4-Benzylpiperazin-1- yl)ethyl]-6-(3- methylphenyl)nicotinamide | LCMS Method (E) RT 3.33 min m/z Obs [M + 1] 415.2 calc [M + 1] 415.2 |
| 250 | 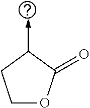 | R² = CH₃ | 6-(3-Methylphenyl)-N-(2- oxotetrahydrofuran-3- yl)nicotinamide | LCMS Method (E) RT 3.95 min m/z Obs [M + 1] 297.1 calc [M + 1] 297.1 |
| 251 | 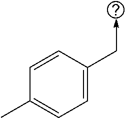 | R² = CH₃ | N-(4-Methylbenzyl)-6-(3- methylphenyl)nicotinamide | LCMS Method (E) RT 5.16 min m/z Obs [M + 1] 317.2 calc [M + 1] 317.2 |
| 252 | 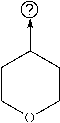 | R² = CH₃ | 6-(3-Methylphenyl)-N- (tetrahydro-2H-pyran-4- yl)nicotinamide | LCMS Method (E) RT 4.10 min m/z Obs [M + 1] 297.2 calc [M + 1] 297.2 |
| 253 | 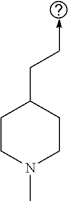 | R² = CH₃ | 6-(3-Methylphenyl)-N-[2-(1- methylpiperidin-4- yl)ethyl]nicotinamide | LCMS Method (E) RT 3.10 min m/z Obs [M + 1] 338.2 calc [M + 1] 338.2 |
| 254 | 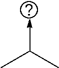 | R² = CH₃ | N-Isopropyl-6-(3- methylphenyl)nicotinamide | ¹H NMR (400 MHz CDCl₃) ppm 0.96-1.32 (m, 6 H), 2.32-2.44 (m, 3 H), 3.96-4.23 (m, 1 H), 7.22-7.50 (m, 2 H), 7.82-8.11 (m, 3 H), 8.16- 8.31 (m, 1 H), 8.31-8.46 (m, 1 H), 8.94-9.15 (m, 1 H). |
| 255 | 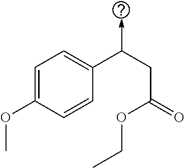 | R² = CH₃ | Ethyl 3-(4-methoxyphenyl)-3- ({[6-(3-methylphenyl)pyridin-3- yl]carbonyl}amino)propanoate | LCMS Method (E) RT 5.09 min m/z Obs [M + 1] 419.2 calc [M + 1] 419.2 |
| 256 | 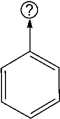 | R³ = F | 6-(4-Fluorophenyl)-N- phenylnicotinamide | LCMS Method (E) RT 4.84 min m/z Obs [M + 1] 293.1 calc [M + 1] 293.1 |
| 257 | 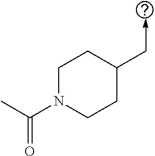 | R³ = F | N-[(1-Acetylpiperidin-4- yl)methyl]-6-(4- fluorophenyl)nicotinamide | LCMS Method (E) RT 3.88 min m/z Obs [M + 1] 356.2 calc [M + 1] 356.2 |
| 258 | 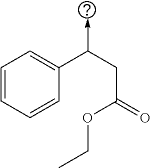 | R³ = F | Ethyl 3-({[6-(4- fluorophenyl)pyridin-3- yl]carbonyl}amino)-3- phenylpropanoate | LCMS Method (E) RT 4.97 min m/z Obs [M + 1] 393.2 calc [M + 1] 393.2 |
| 259 | 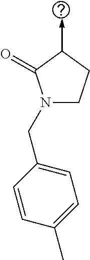 | R³ = F | 6-(4-Fluorophenyl)-N-[1-(4- methylbenzyl)-2- oxopyrrolidin-3- yl]nicotinamide | LCMS Method (E) RT 4.72 min m/z Obs [M + 1] 404.2 calc [M + 1] 404.2 |
| 260 | 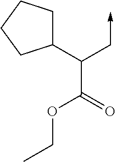 | R³ = F | Ethyl 2-cyclopentyl-3-({[6-(4- fluorophenyl)pyridin-3- yl]carbonyl}amino)propanoate | LCMS Method (E) RT 5.29 min m/z Obs [M + 1] 385.2 calc [M + 1] 385.2 |
| 261 | 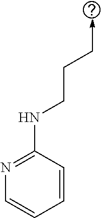 | R³ = F | 6-(4-Fluorophenyl)-N-[3- (pyridin-2- ylamino)propyl]nicotinamide | LCMS Method (E) RT 3.03 min m/z Obs [M + 1] 351.2 calc [M + 1] 351.2 |
| 262 | 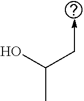 | R³ = F | 6-(4-Fluorophenyl)-N-(2- hydroxypropyl)nicotinamide | LCMS Method (E) RT 3.67 min m/z Obs [M + 1] 275.1 calc [M + 1] 275.1 |
| 263 | 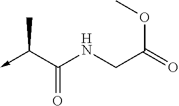 | R³ = F | Methyl N-{[6-(4- fluorophenyl)pyridin-3- yl]carbonyl}-L-alanylglycinate | LCMS Method (E) RT 3.80 min m/z Obs [M + 1] 360.1 calc [M + 1] 360.1 |
| 264 | 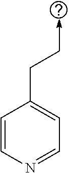 | R³ = F | 6-(4-Fluorophenyl)-N-(2- pyridin-4- ylethyl)nicotinamide | LCMS Method (E) RT 3.08 min m/z Obs [M + 1] 322.1 calc [M + 1] 322.1 |
| 265 | 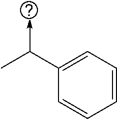 | R³ = F | 6-(4-Fluorophenyl)-N-(1- phenylethyl)nicotinamide | LCMS Method (E) RT 4.84 min m/z Obs [M + 1] 321.1 calc [M + 1] 321.1 |
| 266 | 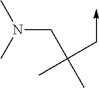 | R³ = F | N-[3-(Dimethylamino)-2,2- dimethylpropyl]-6-(4- fluorophenyl)nicotinamide | LCMS Method (E) RT 2.90 min m/z Obs [M + 1] 330.2 calc [M + 1] 330.2 |
| 267 | 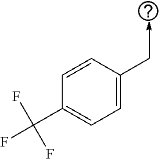 | R³ = F | 6-(4-Fluorophenyl)-N-[4- (trifluoromethyl)benzyl] nicotinamide | LCMS Method (E) RT 5.30 min m/z Obs [M + 1] 375.1 calc [M + 1] 375.1 |
| 268 | 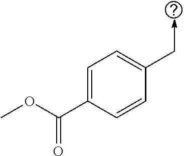 | R³ = F | Methyl 4-[({[6-(4- fluorophenyl)pyridin-3- yl]carbonyl}amino)methyl benzoate | LCMS Method (E) RT 4.75 min m/z Obs [M + 1] 365.1 calc [M + 1] 365.1 |
| 267 | 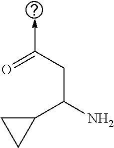 | R³ = F | N-(3-Amino-3- cyclopropylpropanoyl)- 6-(4-fluorophenyl)nicotinamide | LCMS Method (E) RT 3.82 min m/z Obs [M + 1] 328.2 calc [M + 1] 328.2 |
| 270 | 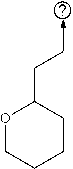 | R³ = F | 6-(4-Fluorophenyl)-N-[2- (tetrahydro-2H-pyran-2- yl)ethyl]nicotinamide | LCMS Method (E) RT 4.49 min m/z Obs [M + 1] 329.2 calc [M + 1] 329.2 |
| 271 | 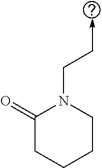 | R² = OCH₃ | 6-(3-Methoxyphenyl)-N-[2- (2-oxopiperidin-1- yl)ethyl]nicotinamide | LCMS Method (E) RT 3.84 min m/z Obs [M + 1] 354.2 calc 354.2 [M + 1] |
| 272 | 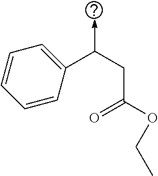 | R² = OCH₃ | Ethyl 3-({[6-(3- methoxyphenyl)pyridin-3- yl]carbonyl}amino)-3- phenylpropanoate | LCMS Method (E) RT 4.97 min m/z Obs [M + 1] 405.2 calc 405.2 [M + 1] |
| 273 | 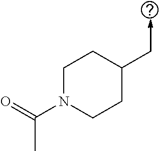 | R² = OCH₃ | N-[(1-Acetylpiperidin-4- yl)methyl]-6-(3- methoxyphenyl)nicotinamide | ¹H NMR (400 MHz, DMSO-d₆) ppm 1.13-1.16 (m, 3 H), 1.72-1.82 (m, 3 H), 1.93 (s, 3 H), 2.90-3.21 (m, 3 H), 3.80 (s, 3 H), 4.38 (m, 2 H), 7.05 (m, 1 H) 7.41 (m, 1 H) 7.67 (m, 1 H), 8.07 (m, 1 H), 8.26 (m, 1 H), 8.67 (m, 1 H.), 9.07 (m, 1 H). |
| 274 | 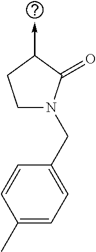 | R² = OCH₃ | 6-(3-Methoxyphenyl)-N-[1- (4-methylbenzyl)-2- oxopyrrolidin-3- yl]nicotinamide | LCMS Method (E) RT 4.64 min m/z Obs [M + 1] 416.2 calc 416.2 [M + 1] |
| 275 | 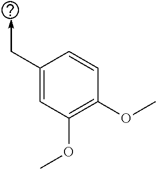 | R² = OCH₃ | N-(3,4-Dimethoxybenzyl)-6- (3-methoxyphenyl)nicotinamide | LCMS Method (E) RT 4.34 min m/z Obs [M + 1] 379.2 calc 379.2 [M + 1] |
| 276 | 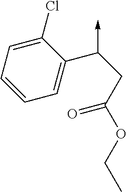 | R² = OCH₃ | Ethyl 3-(2-chlorophenyl)-3- ({[6-(3-methoxyphenyl) pyridin-3-yl]carbonyl}amino) propanoate | LCMS Method (E) 5.22 RT min m/z Obs [M + 1] 439.2 calc 439.2 [M + 1] |
| 277 | 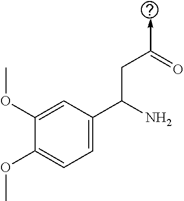 | R² = OCH₃ | N-[3-Amino-3-(3,4- dimethoxyphenyl)propanoyl]- 6-(3-methoxyphenyl) nicotinamide | LCMS Method (E) RT 3.91 min m/z Obs [M + 1] 436.2 calc 436.2 [M + 1] |
| 278 | 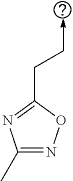 | R² = OCH₃ | 6-(3-Methoxyphenyl)-N-[2- (3-methyl-1,2,4-oxadiazol-5- yl)ethyl]nicotinamide | LCMS Method (E) RT 3.92 min m/z Obs [M + 1] 339.1 calc 339.1 [M + 1] |
| 279 | 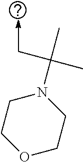 | R² = OCH₃ | 6-(3-Methoxyphenyl)-N-(2- methyl-2-morpholin-4- ylpropyl)nicotinamide | LCMS Method (E) RT 2.87 min m/z Obs [M + 1] 370 calc 370 [M + 1] |
| 280 | 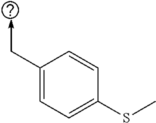 | R² = OCH₃ | 6-(3-Methoxyphenyl)-N-[4- (methylthio)benzyl]nicotinamide | LCMS Method (E) RT 4.89 min m/z Obs [M + 1] 365.1 calc 365.1 [M + 1] |
| 281 | 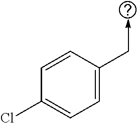 | R² = OCH₃ | N-(4-Chlorobenzyl)-6-(3- methoxyphenyl)nicotinamide | ¹H NMR (400 MHz, DMSO-d₆) ppm 3.85 (s, 3 H), 4.50 (s, 2 H), 7.05 (m, 1 H), 7.38 (m, 4 H), 7.70 (m, 2 H) 8.09 (m, 1 H) 8.30 (m, 1 H), 9.12 (m, 1 H), 9.24 (m, 1 H). |
| 282 | 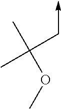 | R² = OCH₃ | N-(2-Methoxy-2- methylpropyl)-6-(3- methoxyphenyl)nicotinamide | LCMS Method (E) RT 4.18 min m/z Obs [M + 1] 315.2 calc 315.2 [M + 1] |
| 283 | 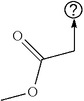 | R² = OCH₃ | Methyl N-{[6-(3- methoxyphenyl)pyridin-3- yl]carbonyl}glycinate | LCMS Method (E) RT 3.84 min m/z Obs [M + 1] 301.1 calc 301.1 [M + 1] |
| 284 | 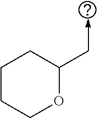 | R² = OCH₃ | 6-(3-Methoxyphenyl)-N- (tetrahydro-2H-pyran-2- ylmethyl)nicotinamide | ¹H NMR (400 MHz, DMSO-d₆) ppm 1.15-1.78 (m, 6 H), 2.54 (m, 2 H), 3.15 (m, 1 H), 3.50 (m, 2 H), 3.80 (s, 3 H), 7.05 (m, 1 H), 7.42 (m, 1 H), 7.69 (m, 2 H), 8.07 (m, 1 H), 8.27 (m, 1 H), 8.68 (m, 1 H), 9.07 (m, 1 H). |
| 285 | 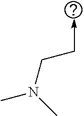 | R² = OCH₃ | N-[2-(Dimethylamino)ethyl]- 6-(3-methoxyphenyl) nicotinamide | LCMS Method (E) RT 2.81 min m/z Obs [M + 1] 300.2 calc 300.2 [M + 1] |
| 286 | 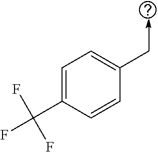 | R² = OCH₃ | 6-(3-Methoxyphenyl)-N-[4- (trifluoromethyl)benzyl] nicotinamide | LCMS Method (E) RT 5.10 min m/z Obs [M + 1] 387.1 calc 387.1 [M + 1] |
| 287 | 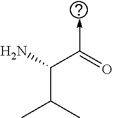 | R² = OCH₃ | 6-(3-Methoxyphenyl)-N-L- valylnicotinamide | LCMS Method (E) RT 3.82 min m/z Obs [M + 1] 328.2 calc 328.2 [M + 1] |
| 288 | 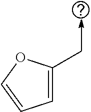 | R² = OCH₃ | N-(2-Furylmethyl)-6-(3- methoxyphenyl)nicotinamide | LCMS Method (E) RT 4.30 min m/z Obs [M + 1] 309.1 calc 309.1 [M + 1] |
| 289 | 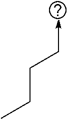 | R² = OCH₃ | N-Butyl-6-(3- methoxyphenyl)nicotinamide | LCMS Method (E) RT 4.46 min m/z Obs [M + 1] 285.2 calc 285.2 [M + 1] |
| 290 | 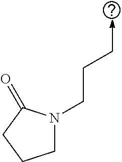 | R² = OCH₃ | 6-(3-Methoxyphenyl)-N-[3- (2-oxopyrrolidin-1- yl)propyl]nicotinamide | LCMS Method (E) RT 3.80 min m/z Obs [M + 1] 354.2 calc 354.2 [M + 1] |
| 291 | 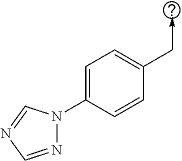 | R² = OCH₃ | 6-(3-Methoxyphenyl)-N-[4- (1H-1,2,4-triazol-1- yl)benzyl]nicotinamide | LCMS Method (E) 4.15 RT min m/z Obs [M + 1] 386.2 calc 386.2 [M + 1] |
| 292 | 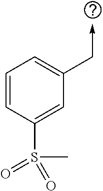 | R² = OCH₃ | 6-(3-Methoxyphenyl)-N-[3- (methylsulfonyl)benzyl] nicotinamide | LCMS Method (E) RT 4.15 min m/z Obs [M + 1] 397.1 calc 397.1 [M + 1] |
| 293 | 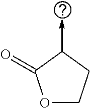 | R² = OCH₃ | 6-(3-Methoxyphenyl)-N-(2- oxotetrahydrofuran-3- yl)nicotinamide | LCMS Method (E) RT 3.81 min m/z Obs [M + 1] 313.1 calc 313.1 [M + 1] |
| 294 | 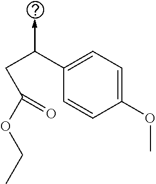 | R² = OCH₃ | Ethyl 3-(4-methoxyphenyl)-3- ({[6-(3-methoxyphenyl) pryidin-3-yl]carbonyl}amino) propanoate | LCMS Method (E) 4.87 RT min m/z Obs [M + 1] 435.2 calc 435.2 [M + 1] |
| 295 | 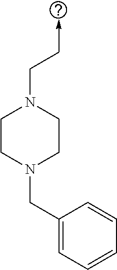 | R² = OCH₃ | N-[2-(4-Benzylpiperazin-1- yl)ethyl]-6-(3- methoxyphenyl)nicotinamide | LCMS Method (E) 3.24 RT min m/z Obs [M + 1] 431.2 calc 431.2 [M + 1] |
| 296 | 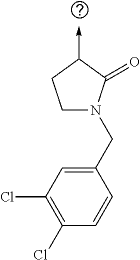 | R² = OCH₃ | N-[1-(3,4-Dichlorobenzyl)-2- oxopyrrolidin-3-yl]-6-(3- methoxyphenyl)nicotinamide | LCMS Method (E) 4.90 RT min m/z Obs [M + 1] 470.1 calc 470.1 [M + 1] |
| 297 | 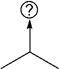 | R² = OCH₃ | N-Isopropyl-6-(3- methoxyphenyl)nicotinamide | LCMS Method (E) 4.17 RT min m/z Obs [M + 1] 271.1 calc 271.1 [M + 1] |
| 298 | 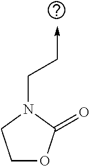 | R² = OCH₃ | 6-(3-Methoxyphenyl)-N-[2- (2-oxo-1,3-oxazolidin-3- yl)ethyl]nicotinamide | LCMS Method (E) RT 3.68 min m/z Obs [M + 1] 342.1 calc 342.1 [M + 1] |
| 299 | 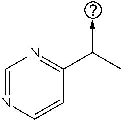 | R² = OCH₃ | 6-(3-Methoxyphenyl)-N-(1- pyrimidin-4- ylethyl)nicotinamide | LCMS Method (E) RT 3.86 min m/z Obs [M + 1] 335.2 calc 335.2 [M + 1] |
| 300 | 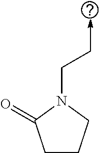 | R² = OCH₃ | 6-(3-methoxyphenyl)-N-[2- (2-oxopyrrolidin-1- yl)ethyl]nicotinamide | LCMS Method (E) RT 3.71 min m/z Obs [M + 1] 340.2 calc 340.2 [M + 1] |
| 301 | 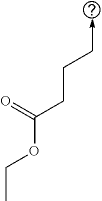 | R² = OCH₃ | Ethyl 4-({[6-(3- methoxyphenyl)pyridin-3- yl]carbonyl}amino)butanoate | LCMS Method (E) RT 4.32 min m/z Obs [M + 1] 343.2 calc 343.2 [M + 1] |
| 302 | 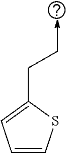 | R² = OCH₃ | 6-(3-Methoxyphenyl)-N-[2- (2-thienyl)ethyl]nicotinamide | LCMS Method (E) RT 4.66 min m/z Obs [M + 1] 339.1 calc 339.1 [M + 1] |
| 303 | 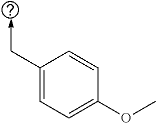 | R² = OCH₃ | N-(4-Methoxybenzyl)-6-(3- methoxyphenyl)nicotinamide | LCMS Method (E) RT 4.66 min m/z Obs [M + 1] 349.2 calc 349.2 [M + 1] |
| 304 | 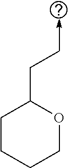 | R² = OCH₃ | 6-(3-Methoxyphenyl)-N-[2- (tetrahydro-2H-pyran-2- yl)ethyl]nicotinamide | LCMS Method (E) RT 4.47 min m/z Obs [M + 1] 341.2 calc 341.2 [M + 1] |
| 305 | 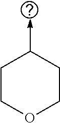 | R² = OCH₃ | 6-(3-Methoxyphenyl)-N- (tetrahydro-2H-pyran-4- yl)nicotinamide | LCMS Method (E) RT 3.92 min m/z Obs [M + 1] 313.2 calc 313.2 [M + 1] |
| 306 | 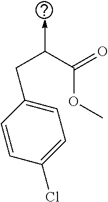 | R² = OCH₃ | Methyl 4-chloro-N-{[6-(3- methoxyphenyl)pyridin-3- yl]carbonyl}phenylalaninate | LCMS Method (E) RT 5.14 min m/z Obs [M + 1] 425.1 calc 425.1 [M + 1] |
| 307 | 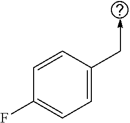 | R² = OCH₃ | N-(4-Fluorophenyl)-6-(3- methoxyphenyl)nicotinamide | LCMS Method (E) RT 4.71 min m/z Obs [M + 1] 337.1 calc 337.1 [M + 1] |
| 308 | 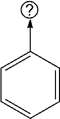 | R² = OCH₃ | 6-(3-Methoxyphenyl)-N- phenylnicotinamide | LCMS Method (E) RT 4.77 min m/z Obs [M + 1] 305.1 calc 305.1 [M + 1] |
| 309 | 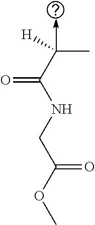 | R² = OCH₃ | Methyl N-{[6-(3- methoxyphenyl)pyridin-3- yl]carbonyl}-L-alanylglycinate | LCMS Method (E) RT 3.83 min m/z Obs [M + 1] 372.2 calc 372.2 [M + 1] |
| 310 | 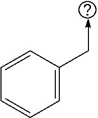 | R² = OCH₃ | N-Benzyl-6-(3- methylphenyl)nicotinamide | LCMS Method (E) RT 4.93 min m/z Obs [M + 1] 303.1 calc 303.1 [M + 1] |
| 311 | 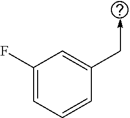 | R² = OCH₃ | N-(3-Fluorobenzyl)-6-(3- methoxyphenyl)nicotinamide | LCMS Method (E) RT 4.70 min m/z Obs [M + 1] 337.1 calc 337.1 [M + 1] |
| 312 | 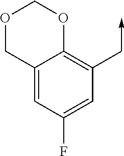 | R² = CH₃ | N-[(6-Fluoro-4H-1,3- benzodioxin-8-yl)methyl]-6- (3-methylphenyl)nicotinamide | LCMS Method (E) RT 5.00 min m/z Obs [M + 1] 379.1 calc 379.1 [M + 1] |
| 313 | 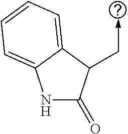 | R² = OCH₃ | 6-(3-Methoxyphenyl)-N-[(2- oxo-2,3-dihydro-1H-indol-3- yl)methyl]nicotinamide | LCMS Method (E) RT 4.20 min m/z Obs [M + 1] 374.1 calc 374.1 [M + 1] |
| 314 | 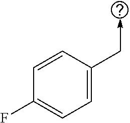 | R² = CH₃ | N-(4-Fluorobenzyl)-6-(3- methylphenyl)nicotinamide | LCMS Method (E) RT 4.98 min m/z Obs [M + 1] 321.1 calc 321.1 [M + 1] |
| 315 | 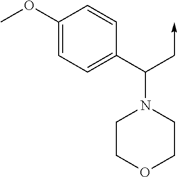 | R² = CH₃ | N-[2-(4-Methoxyphenyl)-2- morpholin-4-ylethyl]-6-(3- methylphenyl)nicotinamide | LCMS Method (E) RT 3.50 min m/z Obs [M + 1] 432.2 calc 432.2 [M + 1] |
| 316 | 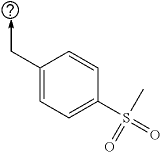 | R² = OCH₃ | 6-(3-Methoxyphenyl)-N-[4- (methylsulfonyl)benzyl] nicotinamide | LCMS Method (E) RT 4.21 min m/z Obs [M + 1] 397.1 calc 397.1 [M + 1] |
| 317 | 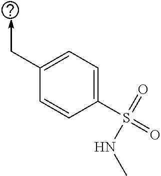 | R² = OCH₃ | 6-(3-Methoxyphenyl)-N-{4- [(methylamino)sulfonyl] benzyl}nicotinamide | LCMS Method (E) RT 4.28 min m/z Obs [M + 1] 412.1 calc 412.1 [M + 1] |
| 318 | 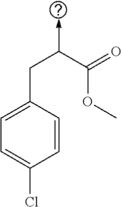 | R² = OCH₃ | Methyl 4-chloro-N-{[6-(3- yl]carbonyl}phenylalaninate | LCMS Method (E) RT 5.47 min m/z Obs [M + 1] 409.1 calc 409.1 [M + 1] |
| 319 | 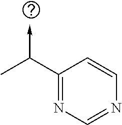 | R² = CH₃ | 6-(3-Methylphenyl)-N-(1- pyrimidin-4- ylethyl)nicotinamide | LCMS Method (E) RT 4.03 min m/z Obs [M + 1] 319.2 calc 319.2 [M + 1] |
| 320 | 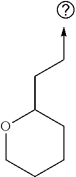 | R² = CH₃ | 6-(3-Methylphenyl)-N-[2- (tetrahydro-2H-pyran-2- yl)ethyl]nicotinamide | LCMS Method (E) RT 4.68 min m/z Obs [M + 1] 325.2 calc 325.2 [M + 1] |
| 321 | 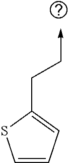 | R² = CH₃ | 6-(3-Methylphenyl)-N-[2-(2- thienyl)ethyl]nicotinamide | LCMS Method (E) RT 4.96 min m/z Obs [M + 1] 323.1 calc 323.1 [M + 1] |
| 322 | 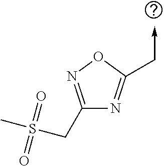 | R³ = F | 6-(4-Fluorophenyl)-N-({3- [(methylsulfonyl)methyl]- 1,2,4-oxadiazol-5- yl}methyl)nicotinamide | LCMS Method (E) RT 4.01 min m/z Obs [M + 1] 391.1 calc 391.1 [M + 1] |
| 323 | 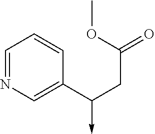 | R³ = F | Methyl 3-({[6-(4- fluorophenyl)pyridin-3- yl]carbonyl}amino)-3- pyridin-3-ylpropanoate | LCMS Method (E) RT 3.67 min m/z Obs [M + 1] 380.1 Calc [M + 1] 380.14 |
| 324 | 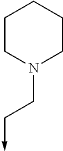 | R³ = F | 6-(4-Fluorophenyl)-N-(2- piperidin-1- ylethyl)nicotinamide | LCMS Method (E) RT 2.86 min m/z Obs [M + 1] 328.18 Calc [M + 1] 328.2 |
| 325 | 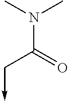 | R³ = F | N-[2-(Dimethylamino)-2- oxoethyl]-6-(4- fluorophenyl)nicotinamide | LCMS Method (E) 3.62 min m/z Obs [M + 1] 302.1 Calc [M + 1] 302.13 |
| 326 | 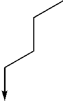 | R³ = F | N-Butyl-6-(4- fluorophenyl)nicotinamide | LCMS Method (E) RT 4.65 min m/z Obs [M] 272.1 Calc [M + 1] 273.14 |
| 327 | 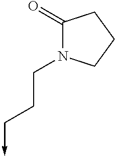 | R³ = F | 6-(4-Fluorophenyl)-N-[3-(2- oxopyrrolidin-1- yl)propyl]nicotinamide | LCMS Method (E) RT 3.86 min m/z Obs [M + 1] 342.2 Calc [M + 1] 342.16 |
| 328 | 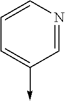 | R³ = F | 6-(4-Fluorophenyl)-N- pyridin-3-ylnicotinamide | LCMS Method (E) RT 3.89 min m/z Obs [M + 1] 294.1 Calc [M + 1] 294.10 |
| 329 | 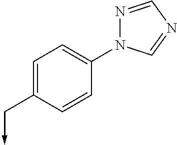 | R³ = F | 6-(4-Fluorophenyl)-N-[4- (1H-1,2,4-triazol-1- yl)benzyl]nicotinamide | LCMS Method (E) RT 4.23 min m/z Obs [M + 1] 374.1 Calc [M + 1] 374.14 |
| 330 | 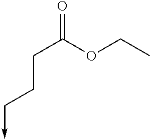 | R³ = F | Ethyl 4-({[6-(4- fluorophenyl)pyridin-3- yl]carbonyl}amino)butanoate | LCMS Method (E) RT 4.32 min m/z Obs [M + 1] 331.1 Calc [M + 1] 331.15 |
| 331 | 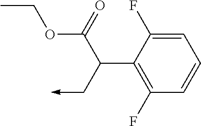 | R² = OCH₃ | Ethyl 2-(2,6-difluorophenyl)- 3-({[6-(3-methoxyphenyl) pyridin-3-yl]carbonyl}amino) propanoate | LCMS Method (E) RT 4.97 min m/z Obs [M + 1] 441.2 Calc [M + 1] 441.16 |
| 332 | 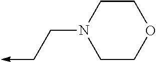 | R² = OCH₃ | 6-(3-Methoxyphenyl)-N-(2- morpholin-4- ylethyl)nicotinamide | LCMS Method (E) RT 2.81 min m/z Obs [M + 1] 342.2 Calc [M + 1] 342.18 |
| 333 | 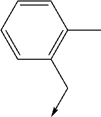 | R² = OCH₃ | 6-(3-Methoxyphenyl)-N-(2- methylbenzyl)nicotinamide | LCMS Method (E) RT 4.84 min m/z Obs [M + 1] 333.2 Calc [M + 1] 333.16 |
| 334 | 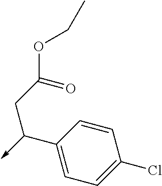 | R² = OCH₃ | Ethyl 3-(4-chlorophenyl)-3- ({[6-(3-methoxyphenyl)pyridin- 3-yl]cabonyl}amino)propanoate | LCMS Method (E) RT 5.22 min m/z Obs [M + 1] 439.1 Calc [M + 1] 439.14 |
| 335 | 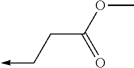 | R² = OCH₃ | Methyl N-{[6-(3- methoxyphenyl)pyridin-3- yl]carbonyl}-beta-alaninate | LCMS Method (E) RT 3.99 min m/z Obs [M + 1] 315.1 Calc [M + 1] 315.13 |
| 336 | 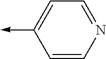 | R² = OCH₃ | 6-(3-Methoxyphenyl)-N- pyridin-4-ylnicotinamide | LCMS Method (E) RT 3.13 min m/z Obs [M] 305.1 Calc [M + 1] 306.12 |
| 337 | 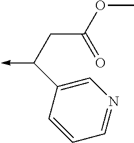 | R² = OCH₃ | Methyl 3-({[6-(3- methoxyphenyl)pyridin-3- yl]carbonyl}amino)-3- pyridin-3-ylpropanoate | LCMS Method (E) RT 3.66 min m/z Obs [M + 1] 392.2 Calc [M + 1] 392.16 |
| 338 | 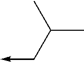 | R² = OCH₃ | N-isobutyl-6-(3- methoxyphenyl)nicotinamide | LCMS Method (E) RT 4.40 min m/z Obs [M + 1] 285.2 Calc [M + 1] 285.16 |
| 339 | 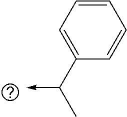 | R² = OCH₃ | 6-(3-Methoxyphenyl)-N-(1- phenylethyl)nicotinamide | LCMS Method (E) RT 4.78 min m/z Obs [M + 1] 333.2 Calc [M + 1] 333.16 |
| 340 | 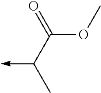 | R² = OCH₃ | Methyl N-{[6-(3- methoxyphenyl)pyridin-3- yl]carbonyl}alaninate | LCMS Method (E) RT 4.11 min m/z Obs [M + 1] 315.1 Calc [M + 1] 315.13 |
| 341 | 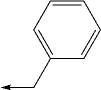 | R² = OCH₃ | N-Benzyl-6-(3- methoxyphenyl)nicotinamide | LCMS Method (E) RT 4.70 min m/z Obs [M + 1] 318.1 Calc [M + 1] 319.14 |
| 342 | 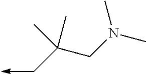 | R² = OCH₃ | N-[3-(Dimethylamino)-2,2- dimethylpropyl]-6-(3- methoxyphenyl)nicotinamide | LCMS Method (E) RT 2.97 min m/z Obs [M + 1] 342.2 Calc [M + 1] 342.22 |
| 343 | 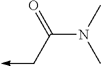 | R² = OCH₃ | N-[2-(Dimethylamino)-2- oxoethyl]-6-(3- methoxyphenyl)nicotinamide | LCMS Method (E) RT 3.59 min m/z Obs [M + 1] 314.1 Calc [M + 1] 314.15 |
| 344 | 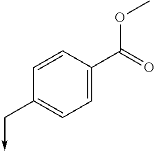 | R² = OCH₃ | Methyl 4-[({[6-(3- methoxyphenyl)pyridin-3- yl]carbonyl}amino)methyl] benzoate | LCMS Method (E) RT 4.68 min m/z Obs [M + 1] 377.1 Calc [M + 1] 377.15 |
| 345 | 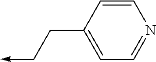 | R² = OCH₃ | 6-(3-Methoxyphenyl)-N-(2- pyridin-4- ylethyl)nicotinamide | LCMS Method (E) RT 3.10 min m/z Obs [M + 1] 334.2 Calc [M + 1] 334.16 |
| 346 | 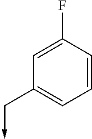 | | N-(3-Fluorobenzyl)-6- phenylnicotinamide | LCMS Method (E) RT 4.75 min m/z Obs [M + 1] 307.1 Calc [M + 1] 307.12 |
| 347 | 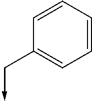 | | N-Benzyl-6- phenylnicotinamide | LCMS Method (E) RT 4.54 min m/z Obs [M + 1] 289.1 Calc [M + 1] 289.13 |
| 348 | 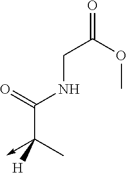 | | Methyl N-[(6-phenylpyridin- 3-yl)carbonyl]-L- alanylglycinate | LCMS Method (E) RT 3.60 min m/z Obs [M + 1] 342.1 Calc [M + 1] 342.14 |
| 349 | 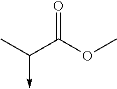 | | Methyl N-[(6-phenylpyridin- 3-yl)carbonyl]alaninate | LCMS Method (E) RT 3.91 min m/z Obs [M + 1] 285.1 Calc [M + 1] 285.12 |
| 350 | 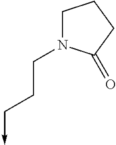 | | N-[3-(2-Oxopyrrolidin-1- yl)propyl]-6- phenylnicotinamide | LCMS Method (E) RT 3.72 min m/z Obs [M + 1] 324.2 Calc [M + 1] 324.17 |
| 351 | 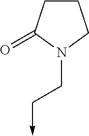 | | N-[2-(2-Oxopyrrolidin-1- yl)ethyl]-6- phenylnicotinamide | LCMS Method (E) RT 3.53 min m/z Obs [M + 1] 310.2 Calc [M + 1] 310.16 |
| 352 | 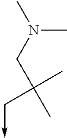 | | N-[3-(Dimethylamino)-2,2- dimethylpropyl]-6- phenylnicotinamide | LCMS Method (E) RT 2.78 min m/z Obs [M + 1] 312.2 Calc [M + 1] 312.21 |
| 353 | 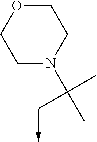 | | N-(2-Methyl-2-morpholin-4- ylpropyl)-6- phenylnicotinamide | LCMS Method (E) RT 2.75 min m/z Obs [M + 1] 340.2 Calc [M + 1] 340.20 |
| 354 | 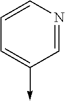 | | 6-Phenyl-N-pyridin-3- ylnicotinamide | LCMS Method (E) RT 3.68 min m/z Obs [M + 1] 276.1 Calc [M + 1] 276.11 |
| 355 | 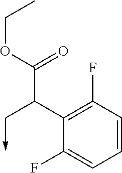 | | Ethyl 2-(2,6-difluorophenyl)- 3-{[(6-phenylpyridin-3- yl)carbonyl]amino}propanoate | LCMS Method (E) RT 4.93 min m/z Obs [M + 1] 411.2 Calc [M + 1] 411.15 |
| 356 | 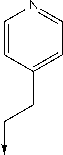 | | 6-Phenyl-N-(2-pyridin-4- ylethyl)nicotinamide | LCMS Method (E) RT 2.88 min m/z Obs [M + 1] 304.1 Calc [M + 1] 304.14 |
| 357 | 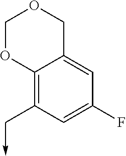 | | N-[(6-Fluoro-4H-1,3- benzodioxin-8-yl)methyl]-6- phenylnicotinamide | LCMS Method (E) RT 4.72 min m/z Obs [M + 1] 365.1 Calc [M + 1] 365.13 |
| 358 | 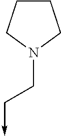 | | 6-Phenyl-N-(2-pyrrolidin-1- ylethyl)nicotinamide | LCMS Method (E) RT 2.86 min m/z Obs [M + 1] 296.2 Calc [M + 1] 296.18 |
| 359 | 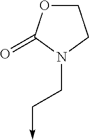 | | N-[2-(2-Oxo-1,3-oxazolidin- 3-yl)ethyl]-6- phenylnicotinamide | LCMS Method (E) RT 3.51 min m/z Obs [M + 1] 312.2 Calc [M + 1] 312.13 |
| 360 | 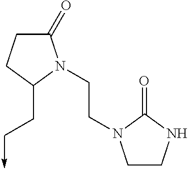 | | N-(2-{5-Oxo-1-[2-(2- oxoimidazolidin-1- yl)ethyl]pyrrolidin-2-yl}ethyl)- 6-phenylnicotinamide | LCMS Method (E) RT 3.53 min m/z Obs [M + 1] 422.2 Calc [M + 1] 422.2 |
| 361 | 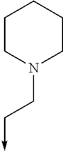 | | 6-Phenyl-N-(2-piperidin-1- ylethyl)nicotinamide | LCMS Method (E) RT 2.91 min m/z Obs [M + 1] 310.2 Calc [M + 1] 310.19 |
| 362 | 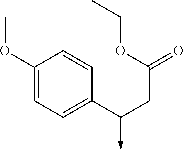 | | Ethyl 3-(4-methoxyphenyl)- 3-{[(6-phenylpyridin-3- yl)carbonyl]amino}propanoate | LCMS Method (E) RT 4.81 min m/z Obs [M + 1] 405.2 Calc [M + 1] 405.18 |
| 363 | 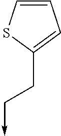 | | 6-Phenyl-N-[2-(2- thienyl)ethyl]nicotinamide | LCMS Method (E) RT 4.59 min m/z Obs [M + 1] 309.1 Calc [M + 1] 309.11 |
| 364 | 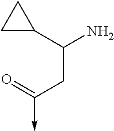 | | N-(3-Amino-3- cyclopropylpropanoyl)-6- phenylnicotinamide | LCMS Method (E) RT 3.59 min m/z Obs [M + 1] 310.2 Calc [M + 1] 310.16 |
| 365 | 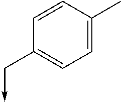 | | N-(4-Methylbenzyl)-6- phenylnicotinamide | LCMS Method (E) RT 4.80 min m/z Obs [M] 302.1 Calc [M + 1] 303.13 |
| 366 | 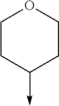 | | 6-Phenyl-N-(tetrahydro-2H- pyran-4-yl)nicotinamide | LCMS Method (E) RT 3.71 min m/z Obs [M + 1] 283.1 Calc [M + 1] 283.14 |
| 367 | 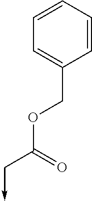 | | Benzyl N-[(6-phenylpyridin- 3-yl)carbonyl]glycinate | LCMS Method (E) RT 4.73 min m/z Obs [M + 1] 347.1 Calc [M + 1] 347.14 |
| 368 | 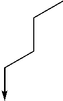 | | N-Butyl-6- phenylnicotinamide | LCMS Method (E) RT 4.43 min m/z Obs [M + 1] 255.2 Calc [M + 1] 255.15 |
| 369 | 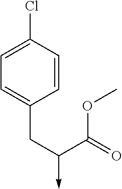 | | Methyl 4-chloro-N-[(6- phenylpyridin-3- yl)carbonyl]phenylalaninate | LCMS Method (E) RT 5.20 min m/z Obs [M + 1] 395.1 Calc [M + 1] 395.12 |
| 370 | 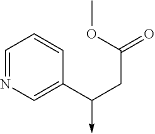 | | Methyl 3-{[(6-phenylpyridin- 3-yl)carbonyl]amino}-3- pyridin-3-ylpropanoate | LCMS Method (E) 3.59 min m/z Obs [M + 1] 362.1 Calc [M + 1] 362.15 |
| 371 | 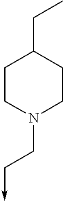 | | N-[2-(4-Ethylpiperidin-1- yl)ethyl]-6- phenylnicotinamide | LCMS Method (E) RT 3.22 min m/z Obs [M + 1] 338.2 Calc [M + 1] 338.22 |
| 372 | 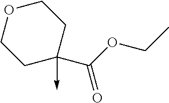 | | Ethyl 4-{[(6-phenylpyridin-3- yl)carbonyl]amino}tetrahydro- 2H-pyran-4-carboxylate | LCMS Method (E) RT 4.12 min m/z Obs [M + 1] 355.2 Calc [M + 1] 355.17 |
| 373 | 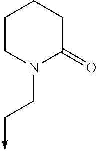 | | N-[2-(2-Oxopiperidin-1- yl)ethyl]-6- phenylnicotinamide | LCMS Method (E) RT 3.74 min m/z Obs [M + 1] 324.2 Calc [M + 1] 324.17 |
| 374 | 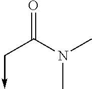 | | N-[2-(Dimethylamino)-2- oxoethyl]-6- phenylnicotinamide | LCMS Method (E) RT 3.52 min m/z Obs [M + 1] 284.1 Calc [M + 1] 284.14 |
| 375 | 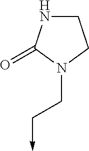 | | N-[2-(2-Oxoimidazolidin-1- yl)ethyl]-6- phenylnicotinamide | LCMS Method (E) RT 3.49 min m/z Obs [M + 1] 311.2 Calc [M + 1] 311.15 |
| 376 | 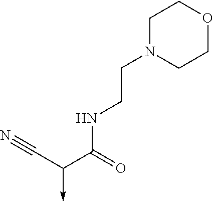 | | N-{1-Cyano-2-[(2- morpholin-4-ylethyl)amino]- 2-oxoethyl}-6- phenylnicotinamide | LCMS Method (E) RT 2.95 min m/z Obs [M + 1] 394.2 Calc [M + 1] 394.19 |
| 377 | 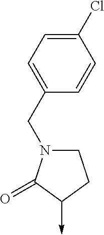 | | N-[1-(4-Chlorobenzyl)-2- oxopyrrolidin-3-yl]-6- phenylnicotinamide | LCMS Method (E) RT 4.59 min m/z Obs [M + 1] 406.1 Calc [M + 1] 406.13 |
| 378 | 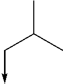 | | N-Isobutyl-6- phenylnicotinamide | LCMS Method (E) RT 4.43 min m/z Obs [M + 1] 255.1 Calc [M + 1] 255.15 |
| 379 | 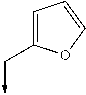 | | N-(2-Furylmethyl)-6- phenylnicotinamide | LCMS Method (E) RT 4.33 min m/z Obs [M + 1] 279.1 Calc [M + 1] 279.11 |
| 380 | 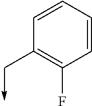 | | N-(2-Fluorobenzyl)-6- phenylnicotinamide | LCMS Method (E) RT 4.61 min m/z Obs [M + 1] 307.1 Calc [M + 1] 307.12 |
| 381 | 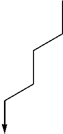 | | N-Pentyl-6- phenylnicotinamide | LCMS Method (E) RT 4.81 min m/z Obs [M + 1] 269.2 Calc [M + 1] 269.17 |
| 382 | 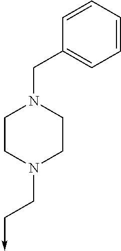 | | N-[2-(4-Benzylpiperazin-1- yl)ethyl]-6- phenylnicotinamide | LCMS Method (E) RT 3.13 min m/z Obs [M + 1] 401.2 Calc [M + 1] 401.23 |
| 383 | 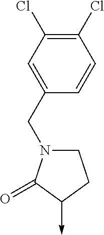 | | N-[1-(3,4-Dichlorobenzyl)-2- oxopyrrolidin-3-yl]-6- phenylnicotinamide | LCMS Method (E) RT 4.84 min m/z Obs [M + 1] 440.1 Calc [M + 1] 440.09 |
| 384 | 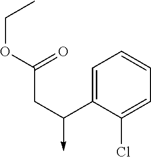 | | ethyl 3-(2-Chlorophenyl)-3- {[(6-phenylpyridin-3- yl)carbonyl]amino}propanoate | LCMS Method (E) RT 5.10 min m/z Obs [M + 1] 409.1 Calc [M + 1] 409.13 |
| 385 | 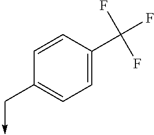 | | 6-Phenyl-N-[4- (trifluoromethyl)benzyl] nicotinamide | LCMS Method (E) RT 3.56 min m/z Obs [M + 1] 357.1 Calc [M + 1] 357.12 |
| 386 | 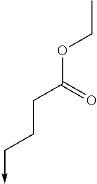 | | Ethyl 4-{[(6-phenylpyridin-3- yl)carbonyl]amino}butanoate | LCMS Method (E) RT 4.17 min m/z Obs [M + 1] 313.2 Calc [M + 1] 313.16 |
| 387 | 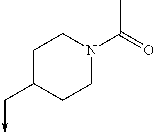 | | N-[(1-Acetylpiperidin-4- yl)methyl]-6- phenylnicotinamide | LCMS Method (E) RT 3.71 min m/z Obs [M + 1] 338.2 Calc [M + 1] 338.19 |
| 388 | 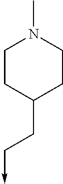 | | N-[2-(1-Methylpiperidin-4- yl)ethyl]-6- phenylnicotinamide | LCMS Method (E) RT 2.93 min m/z Obs [M + 1] 323.2 Calc [M + 1] 324.21 |
| 389 | 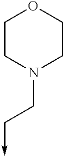 | | N-(2-Morpholin-4-ylethyl)-6- phenylnicotinamide | LCMS Method (E) RT 2.77 min m/z Obs [M + 1] 312.2 Calc [M + 1] 312.17 |
| 390 | 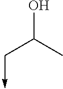 | | N-(2-Hydroxypropyl)-6- phenylnicotinamide | LCMS Method (E) RT 3.48 min m/z Obs [M + 1] 257.1 Calc [M + 1] 257.13 |
| 391 | 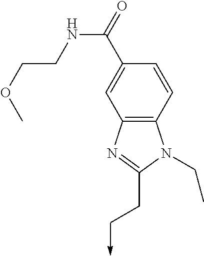 | | 1-Ethyl-N-(2-methoxyethyl)- 2-(2-{[(6-phenylpyridin-3- yl)carbonyl]amino}ethyl)- 1H-benzimidazole-5- carboxamide | LCMS Method (E) RT 3.58 min m/z Obs [M + 1] 472.2 Calc [M + 1] 472.73 |
| 392 | 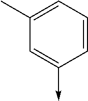 | | N-(3-Methylphenyl)-6- phenylnicotinamide | LCMS Method (E) RT 4.95 min m/z Obs [M + 1] 289.1 Calc [M + 1] 289.13 |
| 393 | 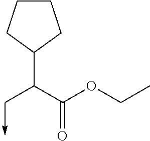 | | Ethyl 2-cyclopentyl-3-{[(6- phenylpyridin-3- yl)carbonyl]amino}propanoate | LCMS Method (E) RT 5.16 min m/z Obs [M + 1] 367.2 Calc [M + 1] 367.20 |
| 394 | 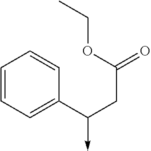 | | Ethyl 3-phenyl-3-{[(6- phenylpyridin-3- yl)carbonyl]amino}propanoate | LCMS Method (E) RT 4.84 min m/z Obs [M + 1] 375.2 Calc [M + 1] 375.17 |
| 395 | 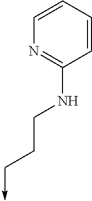 | | 6-Phenyl-N-[3-(pyridin-2- ylamino)propyl]nicotinamide | LCMS Method (E) RT 2.89 min m/z Obs [M + 1] 333.1 Calc [M + 1] 333.17 |
| 396 | 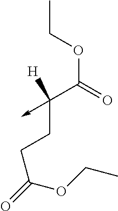 | | Diethyl N-[(6-phenylpyridin- 3-yl)carbonyl]-L-glutamate | LCMS Method (E) RT 4.61 min m/z Obs [M + 1] 385.2 Calc [M + 1] 385.18 |
| 397 | 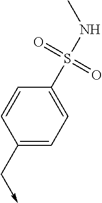 | | N-{4- [(Methylamino)sulfonyl] benzyl}-6-phenylnicotinamide | LCMS Method (E) RT 4.10 min m/z Obs [M + 1] 382.1 Calc [M + 1] 382.12 |
| 398 | 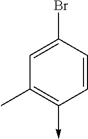 | | N-(4-Bromo-2- methylphenyl)-6- phenylnicotinamide | LCMS Method (E) RT 5.15 min m/z Obs [M + 1] 367.0 Calc [M + 1] 367.04 |
| 399 | 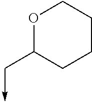 | | 6-Phenyl-N-(tetrahydro-2H- pyran-2-ylmethyl) nicotinamide | LCMS Method (E) RT 4.15 min m/z Obs [M + 1] 297.2 Calc [M + 1] 297.16 |
| 400 | 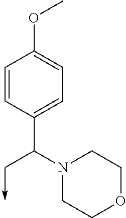 | | N-[2-(4-Methoxyphenyl)-2- morpholin-4-ylethyl]-6- phenylnicotinamide | LCMS Method (E) RT 3.37 min m/z Obs [M + 1] 418.2 Calc [M + 1] 418.21 |
| 401 | 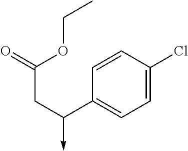 | | Ethyl 3-(4-chlorophenyl)-3- {[(6-phenylpyridin-3- yl)carbonyl]amino}propanoate | LCMS Method (E) RT 5.26 min m/z Obs [M + 1] 409.1 Calc [M + 1] 409.13 |
| 402 | 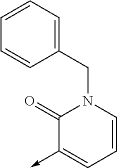 | | N-(1-Benzyl-2-oxo-1,2- dihydropyridin-3-yl)-6- phenylnicotinamide | LCMS Method (E) RT 5.19 min m/z Obs [M + 1] 382.2 Calc [M + 1] 382.16 |
| 403 | 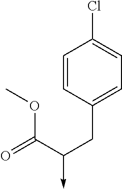 | R² = F | Methyl 4-chloro-N-{[6-(3- fluorophenyl)pyridin-3- yl]carbonyl}phenylalaninate | LCMS Method (E) RT 5.32 min m/z Obs [M + 1] 413.1 Calc [M + 1] 413.10 |
| 404 | 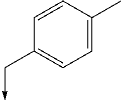 | R² = F | 6-(3-Fluorophenyl)-N-(4- methylbenzyl)nicotinamide | LCMS Method (E) RT 5.11 min m/z Obs [M + 1] 321.1 Calc [M + 1] 321.14 |
| 405 | 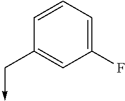 | R² = F | N-(3-Fluorobenzyl)-6-(3- fluorophenyl)nicotinamide | LCMS Method (E) RT 5.06 min m/z Obs [M + 1] 325.1 Calc [M + 1] 325.11 |
| 406 | 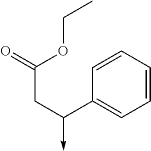 | R² = F | Ethyl 3-({[6-(3- fluorophenyl)pyridin-3- yl]carbonyl}amino)-3- phenylpropanoate | LCMS Method (E) RT 5.09 min m/z Obs [M + 1] 393.2 Calc [M + 1] 393.16 |
| 407 | 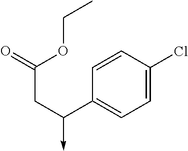 | R² = F | Ethyl 3-(4-chlorophenyl)-3- ({[6-(3-fluorophenyl)pyridin- 3-yl]carbonyl}amino)propanoate | LCMS Method (E) RT 5.46 min m/z Obs [M + 1] 427.1 Calc [M + 1] 427.12 |
| 408 | 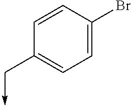 | R² = F | N-(4-Bromobenzyl)-6-(3- fluorophenyl)nicotinamide | LCMS Method (E) RT 5.39 min m/z Obs [M + 1] 385.0 Calc [M + 1] 385.03 |
| 409 | 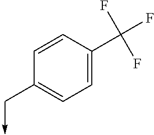 | R² = F | 6-(3-Fluorophenyl)-N-[4- (trifluoromethyl)benzyl] nicotinamide | LCMS Method (E) RT 5.38 min m/z Obs [M + 1] 375.1 Calc [M + 1] 375.11 |
| 410 | 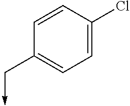 | R² = F | N-(4-Chlorobenzyl)-6-(3- fluorophenyl)nicotinamide | LCMS Method (E) RT 5.12 min m/z Obs [M + 1] 341.1 Calc [M + 1] 341.09 |
| 411 | 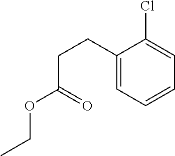 | R² = F | Ethyl 3-(2-chlorophenyl)-3- ({[6-(3-fluorophenyl)pyridin-3- yl]carbonyl}amino)propanoate | LCMS Method (E) RT 5.38 min m/z Obs [M + 1] 427.1 calc [M + 1] 427.1 |
| 412 | 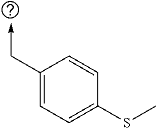 | R² = F | 6-(3-Fluorophenyl)-N-[4- (methylthio)benzyl] nicotinamide | LCMS Method (E) RT 5.14 min m/z Obs [M + 1] 353.1 calc [M + 1] 353.1 |
| 413 | 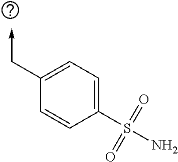 | R² = F | N-[4- (Aminosulfonyl)benzyl]-6- (3-fluorophenyl)nicotinamide | LCMS Method (E) RT 4.07 min m/z Obs [M + 1] 386.1 calc [M + 1] 386.1 |
| 414 | 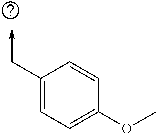 | R² = F | 6-(3-Fluorophenyl)-N-(4- methoxybenzyl)nicotinamide | LCMS Method (E) RT 4.82 min m/z Obs [M + 1] 337.1 calc [M + 1] 337.1 |
| 415 | 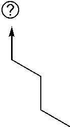 | R² = F | N-Butyl -6-(3- fluorophenyl)nicotinamide | LCMS Method (E) RT 4.71 min m/z Obs [M + 1] 273.1 calc [M + 1] 273.1 |
| 416 | 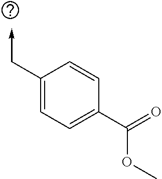 | R² = F | Methyl 4-[({[6-(3- fluorophenyl)pyridin-3- yl]carbonyl}amino)methyl] benzoate | LCMS Method (E) RT 4.72 min m/z Obs [M + 1] 365.1 calc [M + 1] 365.1 |
| 417 | 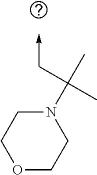 | R² = F | 6-(3-Fluorophenyl)-N-(2- methyl-2-morpholin-4- ylpropyl)nicotinamide | LCMS Method (E) RT 2.84 min m/z Obs [M + 1] 358.2 calc [M + 1] 358.2 |
| 418 | 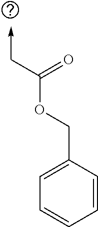 | R² = F | Benzyl N-{[6-(3- fluorophenyl)pyridin-3- yl]carbonyl}glycinate | LCMS Method (E) RT 4.94 min m/z Obs [M + 1] 365.1 calc [M + 1] 365.1 |
| 419 | 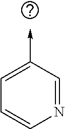 | R² = F | 6-(3-Fluorophenyl)-N- pyridin-3-ylnicotinamide | LCMS Method (E) RT 4.03 min m/z Obs [M + 1] 294.1 calc [M + 1] 294.1 |
| 420 | 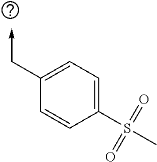 | R² = F | 6-(3-Fluorophenyl)-N-[4- (methylsulfonyl)benzyl] nicotinamide | LCMS Method (E) RT 4.47 min m/z Obs [M + 1] 385.1 calc [M + 1] 385.1 |
| 421 | 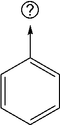 | R² = F | 6-(3-Fluorophenyl)-N- phenylnicotinamide | LCMS Method (E) RT 4.94 min m/z Obs [M + 1] 293.1 calc [M + 1] 293.1 |
| 422 | 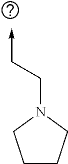 | R² = F | 6-(3-Fluorophenyl)-N-(2- pyrrolidin-1- ylethyl)nicotinamide | LCMS Method (E) RT 2.79 min m/z Obs [M + 1] 314.2 calc [M + 1] 314.2 |
| 423 | 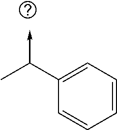 | R² = F | 6-(3-Fluorophenyl)-N-(1- phenylethyl)nicotinamide | LCMS Method (E) RT 5.13 min m/z Obs [M + 1] 321.1 calc [M + 1] 321.1 |
| 424 | 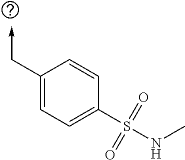 | R² = F | 6-(3-Fluorophenyl)-N-{4- [(methylamino)sulfonyl] benzyl}nicotinamide | LCMS Method (E) RT 4.34 min m/z Obs [M + 1] 400.1 calc [M + 1] 400.1 |
| 425 | 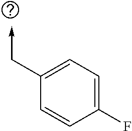 | R² = F | N-(4-Fluorophenyl)-6-(3- fluorophenyl)nicotinamide | LCMS Method (E) RT 4.85 min m/z Obs [M + 1] 325.1 calc [M + 1] 325.1 |
| 426 | 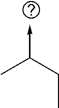 | R² = F | N-sec-Butyl-6-(3- fluorophenyl)nicotinamide | LCMS Method (E) RT 4.55 min m/z Obs [M + 1] 273.1 calc [M + 1] 273.14 |
| 427 | 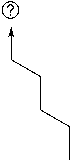 | R² = F | 6-(3-Fluorophenyl)-N- pentylnicotinamide | LCMS Method (E) RT 5.18 min m/z Obs [M + 1] 287.2 calc [M + 1] 287.2 |
| 428 | 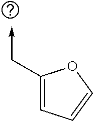 | R² = F | 6-(3-Fluorophenyl)-N-(2- furylmethyl)nicotinamide | LCMS Method (E) RT 4.68 min m/z Obs [M + 1] 297.1 calc [M + 1] 297.1 |
| 429 | 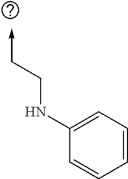 | R² = F | N-(2-Anilinoethyl)-6-(3- fluorophenyl)nicotinamide | LCMS Method (E) RT 4.91 min m/z Obs [M + 1] 336.2 calc [M + 1] 336.2 |
| 430 | 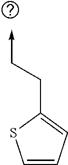 | R² = F | 6-(3-Fluorophenyl)-N-[2-(2- thienyl)ethyl]nicotinamide | LCMS Method (E) RT 5.03 min m/z Obs [M + 1] 327.1 calc [M + 1] 327.1 |
| 431 | 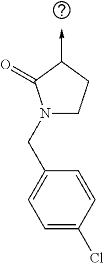 | R² = F | N-[1-(4-Chlorobenzyl)-2- oxopyrrolidin-3-yl]-6-(3- fluorophenyl)nicotinamide | LCMS Method (E) RT 4.78 min m/z Obs [M + 1] 424.1 calc [M + 1] 424.1 |
| 432 | 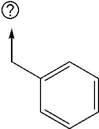 | R² = F | N-Benzyl-6-(3- fluorophenyl)nicotinamide | LCMS Method (E) RT 4.77 min m/z Obs [M + 1] 307.1 calc [M + 1] 307.1 |
| 433 | 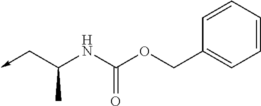 | R² = F | Benzyl [(1S)-2-({[6-(3- fluorophenyl)pyridin-3- yl]carbonyl}amino)-1- methylethyl]carbamate | LCMS Method (E) RT 4.88 min m/z Obs [M + 1] 408.2 calc [M + 1] 408.2 |
| 434 | 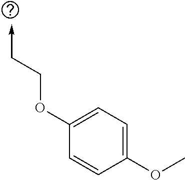 | R² = F | 6-(3-Fluorophenyl)-N-[2-(4- methoxyphenoxy)ethyl] nicotinamide | LCMS Method (E) RT 4.93 min m/z Obs [M + 1] 367.2 calc [M + 1] 367.2 |
| 435 | 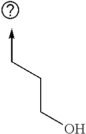 | R² = F | 6-(3-Fluorophenyl)-N-(3- hydroxypropyl)nicotinamide | LCMS Method (E) RT 3.10 min m/z Obs [M + 1] 257.1 calc [M + 1] 257.1 |
| 436 | 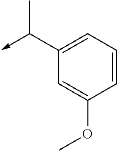 | R² = F | 6-(3-Fluorophenyl)-N- [(1S)-1-(3- methoxyphenyl)ethyl] nicotinamide | LCMS Method (E) RT 5.08 min m/z Obs [M + 1] 351.2 calc [M + 1] 351.2 |
| 437 | 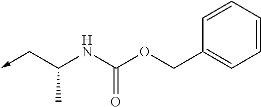 | R² = F | Benzyl [(1R)-2-({[6-(3- fluorophenyl)pyridin-3- yl]carbonyl}amino)-1- methylethyl]carbamate | LCMS Method (E) RT 4.98 min m/z Obs [M + 1] 408.2 calc [M + 1] 408.2 |
| 438 | 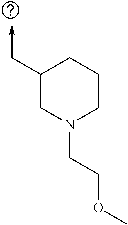 | R² = F | 6-(3-Fluorophenyl)-N-{[1- (2-methoxyethyl)piperidin- 3-yl]methyl}nicotinamide | LCMS Method (E) RT 3.16 min m/z Obs [M + 1] 354.2 calc [M + 1] 354.2 |
| 439 | 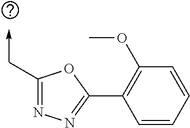 | R² = F | 6-(3-Fluorophenyl)-N-{[5- (2-methoxyphenyl)-1,3,4- oxadiazol-2- yl]methyl}nicotinamide | LCMS Method (E) RT 4.48 min m/z Obs [M + 1] 405.1 calc [M + 1] 405.1 |
| 440 | 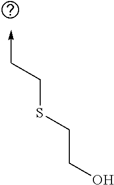 | R² = F | 6-(3-Fluorophenyl)-N-{2- [(2-hydroxyethyl)thio]ethyl} nicotinamide | LCMS Method (E) RT 3.90 min m/z Obs [M + 1] 321.1 calc [M + 1] 321.1 |
| 441 | 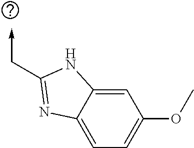 | R² = F | 6-(3-Fluorophenyl)-N-[(6- methoxy-1H-benzimidazol- 2-yl)methyl]nicotinamide | LCMS Method (E) RT 3.56 min m/z Obs [M + 1] 377.1 calc [M + 1] 377.1 |
| 442 | 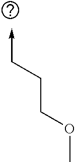 | R² = F | 6-(3-Fluorophenyl)-N-(3- methoxypropyl)nicotinamide | LCMS Method (E) RT 4.07 min m/z Obs [M + 1] 289.1 calc [M + 1] 289.1 |
| 443 | 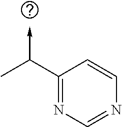 | R² = F | 6-(3-Fluorophenyl)-N-(1- pyrimidin-4- ylethyl)nicotinamide | LCMS Method (E) RT 3.94 min m/z Obs [M + 1] 323.1 calc [M + 1] 323.1 |
| 444 | 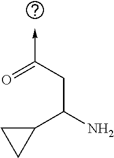 | R² = F | N-(3-Amino-3- cyclopropylpropanoyl)-6- (3-fluorophenyl) nicotinamide | LCMS Method (E) RT 3.79 min m/z Obs [M + 1] 328.1 calc [M + 1] 328.1 |
| 445 | 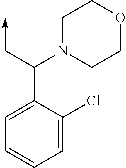 | R² = F | N-[2-(2-Chlorophenyl)-2- morpholin-4-ylethyl]-6-(3- fluorophenyl)nicotinamide | LCMS Method (E) RT 4.14 min m/z Obs [M + 1] 440.2 calc [M + 1] 440.2 |
| 446 | 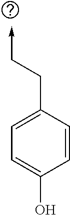 | R² = F | 6-(3-Fluorophenyl)-N-[2-(4- hydroxyphenyl)ethyl] nicotinamide | LCMS Method (E) RT 4.27 min m/z Obs [M + 1] 337.1 calc [M + 1] 337.1 |
| 447 | 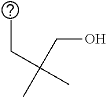 | R² = F | 6-(3-Fluorophenyl)-N-(3- hydroxy-2,2- dimethylpropyl) nicotinamide | LCMS Method (E) RT 4.16 min m/z Obs [M + 1] 303.2 calc [M + 1] 303.2 |
| 448 | 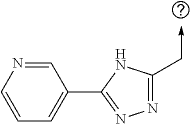 | R² = CH₃ | 6-(3-Methylphenyl)-N-[(5- pyridin-3-yl-4H-1,2,4- triazol-3- yl)methyl]nicotinamide | LCMS Method (E) RT 3.79 min m/z Obs [M + 1] 371.2 calc [M + 1] 371.2 |
| 449 | 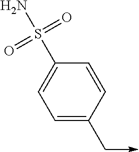 | R² = CH₃ | N-[4- (Aminosulfonyl)benzyl]-6- (3-methylphenyl)nicotinamide | LCMS Method (E) RT 4.23 min m/z Obs [M + 1] 382.1 calc [M + 1] 382.1 |
| 450 | 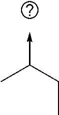 | R² = CH₃ | N-sec-Butyl-6-(3- methylphenyl)nicotinamide | LCMS Method (E) RT 4.61 min m/z Obs [M + 1] 269.2 calc [M + 1] 269.2 |
| 451 | 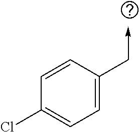 | R² = CH₃ | N-(4-Chlorobenzyl)-6-(3- methylphenyl)nicotinamide | LCMS Method (E) RT 5.15 min m/z Obs [M + 1] 337.1 calc [M + 1] 337.1 |
| 452 | 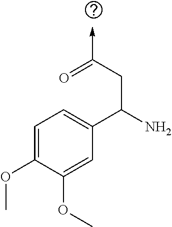 | R² = CH₃ | N-[3-Amino-3-(3,4- dimethoxyphenyl)propanoyl]- 6-(3-methylphenyl) nicotinamide | LCMS Method (E) RT 4.01 min m/z Obs [M + 1] 420.2 calc [M + 1] 420.2 |
| 453 | 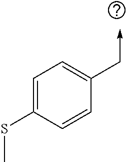 | R² = CH₃ | 6-(3-Methylphenyl)-N-[4- (methylthio)benzyl]nicotinamide | LCMS Method (E) RT 5.10 min m/z Obs [M + 1] 349.1 calc [M + 1] 349.1 |
| 454 | 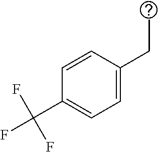 | R² = CH₃ | 6-(3-Methylphenyl)-N-[4- (trifluoromethyl)benzyl] nicotinamide | LCMS Method (E) RT 5.41 min m/z Obs [M + 1] 371.1 calc [M + 1] 371.1 |
| 455 | 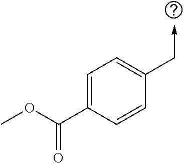 | R² = CH₃ | Methyl 4-[({[6-(3- methylphenyl)pyridin-3- yl]carbonyl}amino)methyl] benzoate | LCMS Method (E) RT 4.86 min m/z Obs [M + 1] 361.2 calc [M + 1] 361.2 |
| 456 | 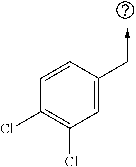 | R² = CH₃ | N-(3,4-Dichlorobenzyl)-6- (3-methylphenyl)nicotinamide | LCMS Method (E) RT 5.45 min m/z Obs [M + 1] 371.1 calc [M + 1] 371.1 |
| 457 | 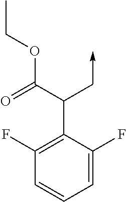 | R³ = F | Ethyl 2-(2,6- difluorophenyl)-3-({[6-(4- fluorophenyl)pyridin-3- yl]carbonyl}amino) propanoate | LCMS Method (E) RT 5.03 min m/z Obs [M + 1] 429.1 calc [M + 1] 429.1 |
| 458 | 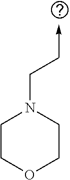 | R³ = F | 6-(4-Fluorophenyl)-N-(2- morpholin-2- ylethyl)nicotinamide | LCMS Method (E) RT 2.82 min m/z Obs [M + 1] 330.2 calc [M + 1] 330.2 |
| 459 | 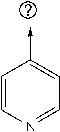 | R³ = F | 6-(4-Fluorophenyl)-N- pyridin-4-ylnicotinamide | LCMS Method (E) RT 3.21 min m/z Obs [M + 1] 294.1 calc [M + 1] 294.1 |
| 460 | 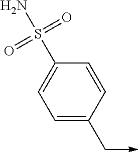 | R³ = F | N-[4- (Aminosulfonyl)benzyl]- 6-(4-fluorophenyl) nicotinamide | LCMS Method (E) RT 4.09 min m/z Obs [M + 1] 386.1 calc [M + 1] 386.1 |
| 461 | 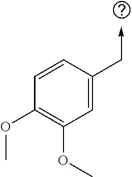 | R³ = F | N-(3,4-Dimethoxybenzyl)- 6-(4-fluorophenyl) nicotinamide | LCMS Method (E) RT 4.49 min m/z Obs [M + 1] 367.1 calc [M + 1] 367.1 |
| 462 | 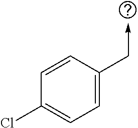 | R³ = F | N-(4-Chlorobenzyl)-6-(4- fluorophenyl)nicotinamide | LCMS Method (E) RT 5.04 min m/z Obs [M + 1] 341.1 calc [M + 1] 341.1 |
| 463 | 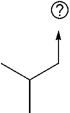 | R³ = F | 6-(4-Fluorophenyl)-N- isobutylnicotinamide | LCMS Method (E) RT 4.50 min m/z Obs [M + 1] 273.1 calc [M + 1] 273.1 |
| 464 | 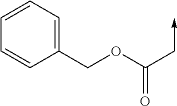 | R³ = F | Benzyl N-{[6-(4- fluorophenyl)pyridin-3- yl]carbonyl}glycinate | LCMS Method (E) RT 4.78 min m/z Obs [M + 1] 365.1 calc [M + 1] 365.1 |
| 465 | 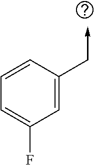 | R³ = F | N-(3-Fluorobenzyl)-6-(4- fluorophenyl)nicotinamide | LCMS Method (E) RT 4.86 min m/z Obs [M + 1] 325.1 calc [M + 1] 325.1 |
| 466 | 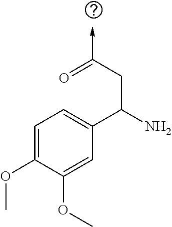 | R³ = F | N-[3-Amino-3-(3,4- dimethoxyphenyl)propanoyl]- 6-(4-fluorophenyl)nicotinamide | LCMS Method (E) RT 3.95 min m/z Obs [M + 1] 424.2 calc [M + 1] 424.2 |
| 467 | 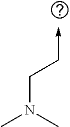 | R³ = F | N-[2- (Dimethylamino)ethyl]-6- (4-fluorophenyl)nicotinamide | LCMS Method (E) RT 2.72 min m/z Obs [M + 1] 288.2 calc [M + 1] 288.2 |
| 468 | 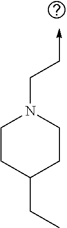 | R³ = F | N-[2-(4-Ethylpiperidin-1- yl)ethyl]-6-(4- fluorophenyl)nicotinamide | LCMS Method (E) RT 3.28 min m/z Obs [M + 1] 356.2 calc [M + 1] 356.2 |
| 469 | 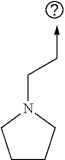 | R³ = F | 6-(4-Fluorophenyl)-N-(2- pyrrolidin-1- ylethyl)nicotinamide | LCMS Method (E) RT 2.80 min m/z Obs [M + 1] 314.2 calc [M + 1] 314.2 |
| 470 | 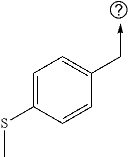 | R³ = F | 6-(4-Fluorophenyl)-N-[4- (methylthio)benzyl]nicotinamide | LCMS Method (E) RT 5.04 min m/z Obs [M + 1] 353.1 calc [M + 1] 353.1 |
| 471 | 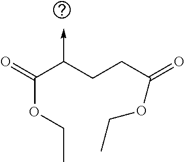 | R³ = F | Diethyl N-{[6-(4- fluorophenyl)pyridin-3- yl]carbonyl}-L-glutamate | LCMS Method (E) RT 4.76 min m/z Obs [M + 1] 403.2 calc [M + 1] 403.2 |
| 472 | 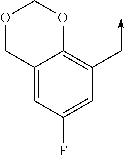 | R³ = F | N-[(6-Fluoro-4H-1,3- benzodioxin-8-yl)methyl]-6- (4-fluorophenyl)nicotinamide | LCMS Method (E) RT 4.74 min m/z Obs [M + 1] 383.1 calc [M + 1] 383.1 |
| 473 | 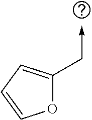 | R³ = F | 6-(4-Fluorophenyl)-N-(2- furylmethyl)nicotinamide | LCMS Method (E) RT 4.39 min m/z Obs [M + 1] 297.1 calc [M + 1] 297.1 |
| 474 | 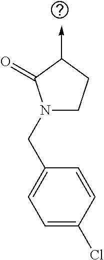 | R³ = F | N-[1-(4-Chlorobenzyl)-2- oxopyrrolidin-3-yl]-6-(4- fluorophenyl)nicotinamide | LCMS Method (E) RT 4.80 min m/z Obs [M + 1] 424.1 calc [M + 1] 424.1 |
| 475 | 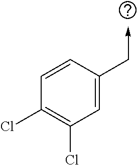 | R³ = F | N-(3,4-Dichlorobenzyl)-6- (4-fluorophenyl)nicotinamide | LCMS Method (E) RT 5.34 min m/z Obs [M + 1] 375.0 calc [M + 1] 375.0 |
| 476 | 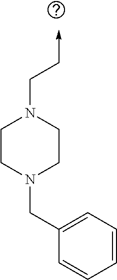 | R³ = F | N-[2-(4-Benzylpiperazin-1- yl)ethyl]-6-(4- fluorophenyl)nicotinamide | LCMS Method (E) RT 3.26 min m/z Obs [M + 1] 419.2 calc [M + 1] 419.2 |
| 477 | 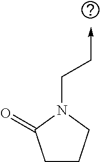 | R³ = F | 6-(4-Fluorophenyl)-N-[2-(2- oxopyrrolidin-1- yl)ethyl]nicotinamide | LCMS Method (E) RT 3.74 min m/z Obs [M + 1] 328.1 calc [M + 1] 328.1 |
| 478 | 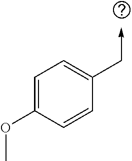 | R³ = F | 6-(4-Fluorophenyl)-N-(4- methoxybenzyl)nicotinamide | LCMS Method (E) RT 4.64 min m/z Obs [M + 1] 337.1 calc [M + 1] 337.1 |
| 479 | 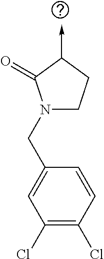 | R³ = F | N-[1-(3,4-Dichlorobenzyl)- 2-oxopyrrolidin-3-yl]-6-(4- fluorophenyl)nicotinamide | LCMS Method (E) RT 5.06 min m/z Obs [M + 1] 458.1 calc [M + 1] 458.1 |
| 480 | 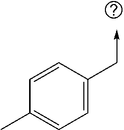 | R³ = F | 6-(4-Fluorophenyl)-N-(4- methylbenzyl)nicotinamide | LCMS Method (E) RT 5.01 min m/z Obs [M + 1] 321.1 calc [M + 1] 321.1 |
| 481 | 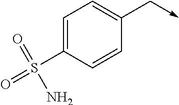 | R² = OCH₃ | N-[4-(Aminosulfonyl)benzyl]- 6-(3-methoxyphenyl) nicotinamide | LCMS Method (E) RT 3.95 min m/z Obs [M + 1] 398.1 calc [M + 1] 398.1 |
| 482 | 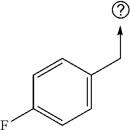 | R³ = F | N-(4-Fluorobenzyl)-6-(4- fluorophenyl)nicotinamide | LCMS Method (E) RT 4.84 min m/z Obs [M + 1] 325.1 calc [M + 1] 325.1 |
| 483 | 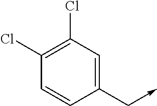 | R² = OCH₃ | N-(3,4-Dichlorobenzyl)-6- (3-methoxyphenyl) nicotinamide | LCMS Method (E) RT 5.40 min m/z Obs [M + 1] 387.1 calc [M + 1] 387.1 ¹H NMR (400 MHz DMSO-d6) ppm 3.80-3.91 (m, 3 H) 4.46-4.57 (m, 2 H) 7.02-7.09 (m, 1 H) 7.31-7.38 (m, 1 H) 7.40-7.48 (m, 1 H) 7.56-7.64 (m, 2 H) 7.67-7.76 (m, 2 H) 8.06-8.14 (m, 1 H) 8.27-8.34 (m, 1 H) 9.09-9.15 (m, 1 H) 9.21-9.30 (m, 1 H) |
| 484 | 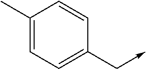 | R² = OCH₃ | 6-(3-Methoxyphenyl)-N-(4- methylbenzyl)nicotinamide | LCMS Method (E) RT 4.88 min m/z Obs [M + 1] 333.2 calc [M + 1] 333.2 |
| 485 | 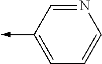 | R² = OCH₃ | 6-(3-Methoxyphenyl)-N- pyridin-3-ylnicotinamide | LCMS Method (E) RT 3.73 min m/z Obs [M + 1] 306.1 calc [M + 1] 306.1 |
| 486 | 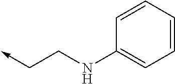 | R² = OCH₃ | N-(2-Anilinoethyl)-6-(3- methoxyphenyl)nicotinamide | LCMS Method (E) RT 4.61 min m/z Obs [M + 1] 348.2 calc [M + 1] 348.2 |
| 487 | 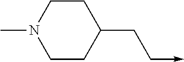 | R² = OCH₃ | 6-(3-Methoxyphenyl)-N-[2- (1-methylpiperidin-4- yl)ethyl]nicotinamide | LCMS Method (E) RT 2.99 min m/z Obs [M + 1] 354.2 calc [M + 1] 354.2 |
| 488 | 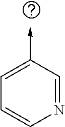 | R² = CH₃ | 6-(3-Methylphenyl)-N- pyridin-3-ylnicotinamide | LCMS Method (E) RT 3.95 min m/z Obs [M + 1] 290.1 calc [M + 1] 290.1 |
| 489 | 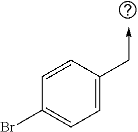 | R² = CH₃ | N-(4-Bromobenzyl)-6-(3- methylphenyl)nicotinamide | LCMS Method (E) RT 5.28 min m/z Obs [M + 1] 381.1 calc [M + 1] 381.1 |
| 490 | 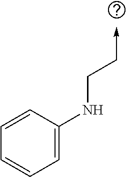 | R² = CH₃ | N-(2-Anilinoethyl)-6-(3- methylphenyl)nicotinamide | LCMS Method (E) RT 4.84 min m/z Obs [M + 1] 332.2 calc [M + 1] 332.2 |
| 491 | 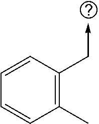 | R³ = F | 6-(4-Fluorophenyl)-N-(2- methylbenzyl)nicotinamide | LCMS Method (E) RT 4.93 min m/z Obs [M + 1] 321.1 calc [M + 1] 321.1 |
| 492 | 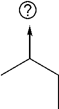 | R³ = F | N-sec-Butyl-6-(4- fluorophenyl)nicotinamide | LCMS Method (E) RT 4.46 min m/z Obs [M + 1] 273.1 calc [M + 1] 273.1 |
| 493 | 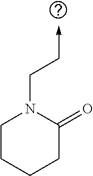 | R³ = F | 6-(4-Fluorophenyl)-N-[2-(2- oxopiperidin-1- yl)ethyl]nicotinamide | LCMS Method (E) RT 3.84 min m/z Obs [M + 1] 342.2 calc [M + 1] 342.2 |
| 494 | 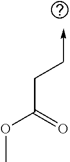 | R³ = F | Methyl N-{[6-(4- fluorophenyl)pyridin-3- yl]carbonyl}-beta-alaninate | LCMS Method (E) RT 3.99 min m/z Obs [M + 1] 303.1 calc [M + 1] 303.1 |
| 495 | 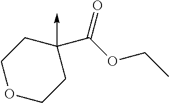 | R³ = F | Ethyl 4-({[6-(4- fluorophenyl)pyridin-3- yl]carbonyl}amino)tetrahydro- 2H-pyran-4-carboxylate | LCMS Method (E) RT 4.28 min m/z Obs [M + 1] 373.2 calc [M + 1] 373.2 |
| 496 | 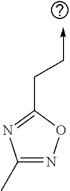 | R³ = F | 6-(4-Fluorophenyl)-N-[2-(3- methyl-1,2,4-oxadiazol-5- yl)ethyl]nicotinamide | LCMS Method (E) RT 4.06 min m/z Obs [M + 1] 327.1 calc [M + 1] 327.1 |
| 497 | 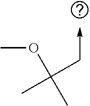 | R³ = F | 6-(4-Fluorophenyl)-N-(2- methoxy-2- methylpropyl)nicotinamide | LCMS Method (E) RT 4.19 min m/z Obs [M + 1] 303.2 calc [M + 1] 303.2 |
| 498 | 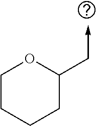 | R³ = F | 6-(4-Fluorophenyl)-N- (tetrahydro-2H-pyran-2- ylmethyl)nicotinamide | LCMS Method (E) RT 4.31 min m/z Obs [M + 1] 315.2 calc [M + 1] 315.2 |
| 499 | 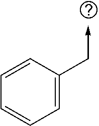 | R³ = F | N-Benzyl-6-(4- fluorophenyl)nicotinamide | LCMS Method (E) RT 4.74 min m/z Obs [M + 1] 307.1 calc [M + 1] 307.1 |
| 500 | 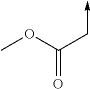 | R³ = F | Methyl N-{[6-(4- fluorophenyl)pyridin-3- yl]carbonyl}glycinate | LCMS Method (E) RT 3.97 min m/z Obs [M + 1] 289.1 calc [M + 1] 289.1 |

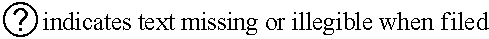

| Ex | R′ | R¹⁻⁵ | Name | Preparation and Characterisation |
|---|---|---|---|---|
| 501 | 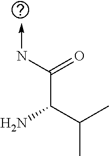 | R³ = F | 6-(4-Fluorophenyl)-N-L- valylnicotinamide | LCMS Method (E) RT 3.89 min m/z Obs [M + 1] 316.1 calc [M + 1] 316.1 |
| 502 | 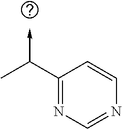 | R³ = F | 6-(4-Fluorophenyl)-N-(1- pyrimidin-4-ylethyl) nicotinamide | LCMS Method (E) RT 3.84 min m/z Obs [M + 1] 323.1 calc [M + 1] 323.1 |
| 503 | 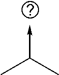 | R³ = F | 6-(4-Fluorophenyl)-N- isopropylnicotinamide | LCMS Method (E) RT 4.18 min m/z Obs [M + 1] 259.1 calc [M + 1] 259.1 |
| 504 | 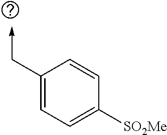 | R³ = F | 6-(4-Fluorophenyl)-N-[4- (methylsulfonyl)benzyl] nicotinamide | LCMS Method (E) RT 4.26 min m/z Obs [M + 1] 385.1 calc [M + 1] 385.1 |
| 505 | 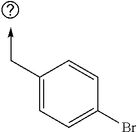 | R³ = F | N-(4-Bromobenzyl)-6-(4- fluorophenyl)nicotinamide | LCMS Method (E) RT 5.20 min m/z Obs [M + 1] 385.0 calc [M + 1] 385.0 |
| 506 | 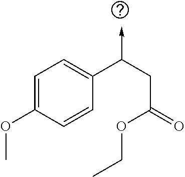 | R³ = F | Ethyl 3-({[6-(4- fluorophenyl)pyridin-3- yl]carbonyl}amino)-3-(4- methoxyphenyl)propanoate | LCMS Method (E) RT 4.92 min m/z Obs [M + 1] 423.2 calc [M + 1] 423.2 |
| 507 | 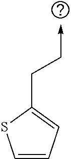 | R³ = F | 6-(4-Fluorophenyl)-N-[2-(2- thienyl)ethyl]nicotinamide | LCMS Method (E) RT 4.81 min m/z Obs [M + 1] 327.1 calc [M + 1] 327.1 |
| 508 | 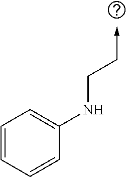 | R³ = F | N-(2-Anilinoethyl)-6-(4- fluorophenyl)nicotinamide | PF-03961414 LCMS Method (E) RT 4.66 min m/z Obs [M + 1] 336.2 calc [M + 1] 336.2 |
| 509 | 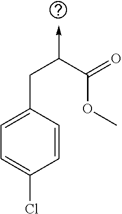 | R³ = F | Methyl 4-chloro-N-{[6-(4- fluorophenyl)pyridin-3- yl]carbonyl}phenylalaninate | LCMS Method (E) RT 5.22 min m/z Obs [M + 1] 413.1 calc [M + 1] 413.1 |
| 510 | 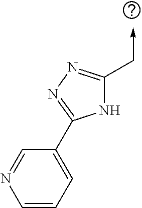 | R² = CH₃ | 6-(3-Methoxyphenyl)-N-[(5- pyridin-3-yl-4H-1,2,4- triazol-3- yl)methyl]nicotinamide | LCMS Method (E) RT 3.55 min m/z Obs [M + 1] 387.2 calc [M + 1] 387.2 |
| 511 | 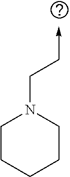 | R ² = OCH₃ | 6-(3-methoxyphenyl)-N-(2- piperidin-1- ylethyl)nicotinamide | LCMS Method (E) RT 2.95 min m/z Obs [M + 1] 340.2 calc [M + 1] 340.2 |
| 512 | 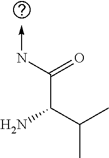 | | 6-Phenyl-N-L- valylnicotinamide | LCMS Method (E) RT 3.79 min m/z Obs [M + 1] 298.2 calc [M + 1] 298.2 |
| 513 | 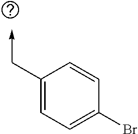 | | N-(4-Bromobenzyl)-6- phenylnicotinamide | LCMS Method (E) RT 5.04 min m/z Obs [M + 1] 367.0 calc [M + 1] 367.0 |
| 514 | 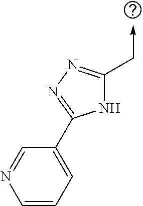 | | 6-Phenyl-N-[(5-pyridin-3-yl- 4H-1,2,4-triazol-3- yl)methyl]nicotinamide | LCMS Method (E) RT 3.33 min m/z Obs [M + 1] 357.1 calc [M + 1] 357.1 |
| 515 | 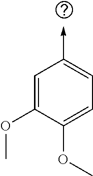 | | N-(3,4-Dimethoxyphenyl)- 6-phenylnicotinamide | LCMS Method (E) RT 4.41 min m/z Obs [M + 1] 335.1 calc [M + 1] 335.1 |
| 516 | 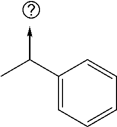 | | 6-Phenyl-N-(1- phenylethyl)nicotinamide | LCMS Method (E) RT 4.72 min m/z Obs [M + 1] 303.1 calc [M + 1] 303.1 |
| 517 | 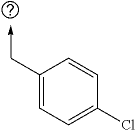 | | N-(4-Chlorobenzyl)-6- phenylnicotinamide | LCMS Method (E) RT 4.97 min m/z Obs [M + 1] 323.1 calc [M + 1] 323.1 |
| 518 | 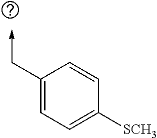 | | N-[4-(Methylthio)benzyl]-6- phenylnicotinamide | LCMS Method (E) RT 4.84 min m/z Obs [M + 1] 335.1 calc [M + 1] 335.1 |
| 519 | 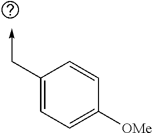 | | N-(4-Methoxybenzyl)-6- phenylnicotinamide | LCMS Method (E) RT4.63 min m/z Obs [M + 1] 319.1 calc [M + 1] 319.1 |
| 520 | 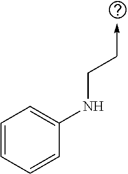 | | N-(2-Anilinoethyl)-6- phenylnicotinamide | LCMS Method (E) RT 4.57 min m/z Obs [M + 1] 318.2 calc [M + 1] 318.2 |
| 521 | 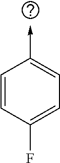 | | N-(4-Fluorophenyl)-6- phenylnicotinamide | LCMS Method (E) RT 4.78 min m/z Obs [M + 1] 293.1 calc [M + 1] 293.1 |
| 522 | 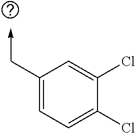 | All = H | N-(3,4-Dichlorobenzyl)-6- phenylnicotinamide | LCMS Method (E) RT 5.20 min m/z Obs [M + 1] 357.1 calc [M + 1] 357.1 |
| 523 | 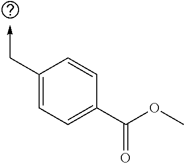 | | Methyl 4-({[(6- phenylpyridin-3- yl)carbonyl]amino}methyl) benzoate | LCMS Method (E) RT 4.61 min m/z Obs [M + 1] 347.1 calc [M + 1] 347.1 |
| 524 | 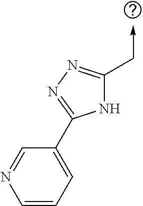 | R² = F | 6-(3-Fluorophenyl)-N-[(5- pyridin-3-yl-4H-1,2,4- triazol-3- yl)methyl]nicotinamide | LCMS Method (E) RT 3.65 min m/z Obs [M + 1] 375.1 calc [M + 1] 375.1 |
| 525 | 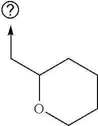 | R² = F | 6-(3-Fluorophenyl)-N- (tetrahydro-2H-pyran-2- ylmethyl)nicotinamide | LCMS Method (E) RT 4.52 min m/z Obs [M + 1] 315.1 calc [M + 1] 315.1 |
| 526 | 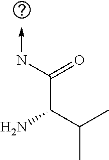 | R² = F | 6-(3-Fluorophenyl)-N-L- valylnicotinamide | LCMS Method (E) RT 3.96 min m/z Obs [M + 1] 316.1 calc [M + 1] 316.1 |
| 527 | 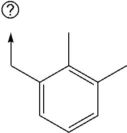 | | N-(2,3-Dimethylphenyl)-6- phenylnicotinamide | LCMS Method (E) RT 4.84 min m/z Obs [M + 1] 303.1 calc [M + 1] 303.1 |
| 528 | 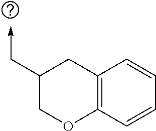 | R² = F | N-(3,4-Dihydro-2H- chromen-3-ylmethyl)-6-(3- fluorophenyl)nicotinamide | LCMS Method (E) RT 5.11 min m/z Obs [M + 1] 363.2 calc [M + 1] 363.2 |

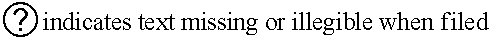

| Ex | R7 | R¹⁻⁵ | Name | Purification and Characterisation |
|---|---|---|---|---|
| 529 | 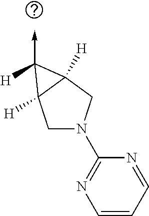 | R² = F | 6-(3-Fluorophenyl)-N- [(1R,5S,6s)-3-pyrimidin-2- yl-3-azabicyclo[3.1.0]hex- 6-yl]nicotinamide | 1H NMR (400 MHz, METHANOL-d₄) ppm 2.02-2.07 (m, 2 H), 2.62-2.65 (m, 1 H), 3.59-3.65 (m, 2 H), 4.00- 4.05 (m, 2 H), 6.62-6.64 (m, 1 H), 7.18-7.25 (m, 1 H), 7.49-7.56 (m, 1 H), 7.80-7.90 (m, 2 H), 7.97-8.00 (m, 1 H), 8.25-8.29 (m, 1 H), 8.30- 8.34 (m, 2 H), 9.03-9.06 (m, 1 H). LCMS 376 [M + 1] |
| 530 | 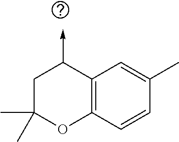 | R² = F | 6-(3-Fluorophenyl)-N- (2,2,6-trimethyl-3,4- dihydro-2H-chromen-4- yl)nicotinamide | Purified by HPLC Method (E) LCMS Method (F) RT 5.18 min, (ES) m/z 390.1743 [M] calc 390.456 [M] |
| 531 | 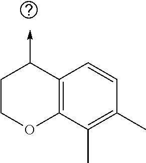 | R² = F | N-(7,8-Dimethyl-3,4- dihydro-2H-chromen-4-yl)- 6-(3-fluorophenyl) nicotinamide | Purified by HPLC Method (E) LCMS Method (F) RT 5.16 min, (ES) m/z 376.1587 [M] calc 376.429 [M] |
| 532 | 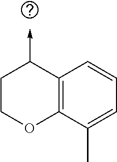 | R² = F | 6-(3-Fluorophenyl)-N-(8- methyl-3,4-dihydro-2H- chromen-4-yl)nicotinamide | Purified by HPLC Method (E) LCMS Method (F) RT 4.95 min, (ES) m/z 362.1431 [M] calc 360.402 [M] |
| 533 | 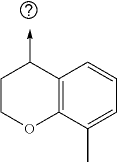 | R² = F R⁴ = F | 6-(3,5-Difluorophenyl)-N- (8-methyl-3,4-dihydro-2H- chromen-4-yl)nicotinamide | Purified by HPLC Method (E) LCMS Method (F) RT 5.11 min, (ES) m/z 380.1336 [M] calc 380.392 [M] |
| 534 | 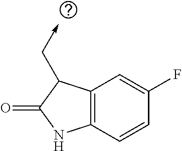 | R² = F | N-[(5-Fluoro-2-oxo-2,3- dihydro-1H-indol-3- yl)methyl]-6-(3- fluorophenyl)nicotinamide | Purified by HPLC Method (E) LCMS Method (F) RT 4.43 min, (ES) m/z 379.11 [M] calc 379.365 [M] |
| 535 | 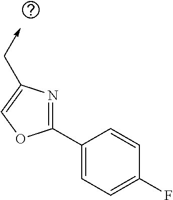 | R² = F | 6-(3-Fluorophenyl)-N-{[2- (4-fluorophenyl)-1,3- oxazol-4- yl]methyl}nicotinamide | Purified by HPLC Method (E) LCMS Method (F) RT 5.03 min, (ES) m/z 391.1132 [M] calc 391.1375 [M] |
| 536 | 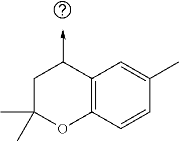 | R² = F R⁴ = F | 6-(3,5-Difluorophenyl)-N- (2,2,6-trimethyl-3,4- dihydro-2H-chromen-4- yl)nicotinamide | Purified by HPLC Method (E) LCMS Method (F) RT 5.45 min, (ES) m/z 408.1649 [M] calc 408.466 [M] |
| 537 | 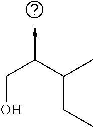 | | 6-(3-Fluorophenyl)-N-[1- (hydroxymethyl)-2- methylbutyl]nicotinamide | Purified by HPLC Method (E) LCMS Method (F) RT 3.66 min, (ES) m/z 316.1587 [M] calc 316.374 [M] |
| 538 | 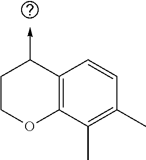 | R² = F R⁴ = F | 6-(3,5-Difluorophenyl)-N- (7,8-dimethyl-3,4-dihydro- 2H-chromen-4- yl)nicotinamide | Purified by HPLC Method (E) LCMS Method (F) RT 5.40 min, (ES) m/z 394.1493 [M] calc 394.419 [M] |
| 539 | 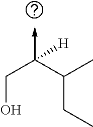 | | 6-(3-Fluorophenyl)-N- [(1S)-1-(hydroxymethyl)- 2,2-dimethylpropyl] nicotinamide | Purified by HPLC Method (E) LCMS Method (F) RT 3.92 min, (ES) m/z 316.1587 [M] calc 316.374 [M] |
| 540 | 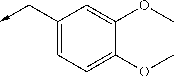 | | N-(3,4-Dimethoxybenzyl)- 6-phenylnicotinamide | ¹H NMR (DMSO-d₆, 400 MHz) δ 3.70-3.71 (m, 6 H) 4.41-4.43 (m, 2 H) 6.83-6.88 (m, 2 H) 6.94 (s, 1 H) 7.43- 7.51 (m, 3 H) 8.05-8.07 (m, 1 H) 8.27-8.29 (m, 1 H) 9.10 (s, 1 H) 9.13- 9.15 (m, 1 H) |
| 541 | 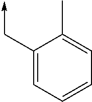 | R² = F R⁴ = F | 6-(3,5-Difluorophenyl)-N- (2-methylbenzyl) nicotinamide | ¹H NMR (400 MHz, DMSO-d6) d ppm 1.54 (s, 3 H) 1.66-1.75 (m, 1 H) 3.70 (d, J = 5.5 Hz, 2 H) 6.33- 6.41 (m, 2 H) 6.47 (br s, 1 H) 6.51- 6.60 (m, 1 H), 7.08 (d, J = 7.1 Hz, 2 H) 7.40 (d, J = 8.2 Hz, 1 H), 7.56 (,dd, J = 8.2, 1.8 Hz 1 H) 8.34 (br. S, 2 H) |
| 542 | 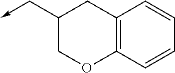 | R² = F R⁴ = F | 6-(3,5-Difluorophenyl)-N- (3,4-dihydro-2H-chromen- 3-ylmethyl)nicotinamide | ¹H NMR (400 MHz, DMSO-d6) δ ppm 1.52 (br s, 1 H) 1.70 (br s, 2 H), 1.72-1.85 (m, 2 H) 2.02-2.13 (m, 1 H) 3.03-3.15 (m, 1 H) 3.44 (d, J = 12.8 Hz, 1 H), 5.95 (d, J = 7.7 Hz, 1 H), 5.97-6.07 (m, 1 H), 6.20-6.32 (m, 2 H), 7.09 (d, J = 7.3 Hz, 2 H), 7.40 (d, J = 8.2 Hz, 1 H) 7.54 (d, J = 8.2 Hz, 1 H), 8.06 (br. S, 1 H), 8.32 (s, 1 H) |
| 543 | 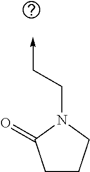 | R² = F R⁴ = OCH₃ | 6-(3-Fluoro-5- methoxyphenyl)-N-[3-(2- oxopyrrolidin-1- yl)propyl]nicotinamide | ¹H NMR (400 MHz, DMSO-d6) δ ppm 0.99 (t, J = 7.0 Hz, 3 H) 1.09- 1.24 (m, 3 H) 1.47 (t, J = 8.1 Hz, 3 H) 1.74 (br. s, 1 H) 2.61 (t, J = 7.0 Hz 4 H) 6.19 (d, J = 10.6 Hz, 1 H) 6.75- 6.85 (m, 2 H), 7.39 (d, J = 8.4 Hz, 1 H) 7.51 (dd, J = 8.3, 2.1 Hz, 1 H) 7.90 (br. s, 1 H) 8.32 (s, 1 H) |

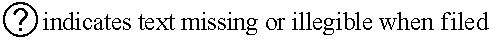

| Ex | R8 | R¹⁻⁵ | Name | Purification and characterisation |
|---|---|---|---|---|
| 544 | 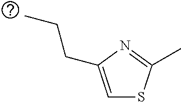 | R² = F | 5-Chloro-6-(3- fluorophenyl)-N-[2-(2- methyl-1,3-thiazol-4- yl)ethyl]nicotinamide | ¹H NMR (400 MHz, DMSO-d₆) ppm 2.56-2.63 (s, 3H), 2.87-2.96 (m, 2H), 3.52-3.61 (m, 2H), 7.14 (s, 1H), 7.29-7.35 (m, 1H), 7.47-7.55 (m, 3H) 8.35 (s, 1H), 8.82-8.87 (m, 1H), 8.96 (s, 1H). |
| 545 | 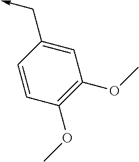 | R² = F | 5-Chloro-N-(3,4- dimethoxybenzyl)-6-(3- fluorophenyl)nicotinamide | ¹H NMR (400 MHz, DMSO-d₆) ppm 3.71-3.76 (m, 6H), 4.43-4.49 (m, 2H), 6.84-7.02 (m, 3H), 7.31-7.39 (m, 1H), 7.52-7.59 (m, 3H), 8.46 (s, 1H), 9.07 (s, 1H) 9.22-9.30 (m, 1H). |
| 546 | 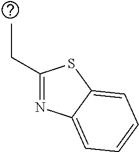 | R² = F | N-(1,3-Benzothiazol-2- ylmethyl)-5-chloro-6-(3- fluorophenyl)nicotinamide | ¹H NMR (400 MHz, DMSO-d₆) ppm 4.87-4.95 (m, 2H), 7.29-7.43 (m, 2H), 7.46-7.57 (m, 4H), 7.91-7.97 (m, 1H), 8.01-8.06 (m, 1H), 8.47 (s, 1H), 9.08 (s, 1H), 9.78-9.85 (m, 1H). LCMS (ES+) 398 (M + 1) |
| 547 | 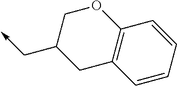 | R² = F | 5-Chloro-N-(3,4-dihydro- 2H-chromen-3-ylmethyl)-6- (3- fluorophenyl)nicotinamide | ¹H NMR (400 MHz, DMSO-d₆) ppm 2.23-2.33 (m, 1H), 2.52-2.61 (m, 1H), 2.80-2.89 (m, 1H), 3.29-3.36 (m, 2H), 3.82-3.91 (m, 1H), 4.17-4.24 (m, 1H), 6.77-6.81 (m, 2H), 6.99-7.08 (m, 2H), 7.29-7.37 (m, 1H), 7.48-7.56 (m, 3H), 8.41 (s, 1H), 8.95-8.93 (m, 1H), 9.01 (s, 1H). LCMS (ES+) 397 (M + 1) |
| 548 | 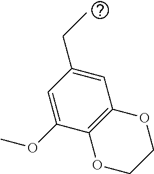 | R² = F | 5-Chloro-6-(3- fluorophenyl)-N-[(8- methoxy-2,3-dihydro-1,4- benzodioxin-6- yl)methyl]nicotinamide | ¹H NMR (400 MHz, DMSO-d₆) ppm 3.74 (s, 3H), 4.19 (s, 4H), 4.35-4.43 (m, 2H), 6.48 (s, 1H), 6.58 (s, 1H), 7.32-7.40 (m, 1H), 7.51-7.60 (m, 3H), 8.46 (s, 1H), 9.06 (s, 1H) 9.19-9.27 (m, 1H). LCMS (ES+) 429 (M + 1) |
| 549 | 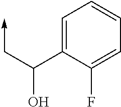 | R² = F | 5-Chloro-6-(3- fluorophenyl)-N-[2-(2- fluorophenyl)-2- hydroxyethyl]nicotinamide | ¹H NMR (DMSO-d₆, 400 MHz) δ 3.44-3.52 (m, 2H) 5.06-5.10 (m, 1H) 5.62-5.53 (m, 1H) 7.09-7.14 (m, 1H) 7.20-7.22 (m, 1H) 7.27-7.32 (m, 2H) 7.53-7.56 (m, 3H) 8.36-8.37 (m, 1H) 8.88-8.90 (m, 1H) 8.97 (s, 1H) |
| 550 | 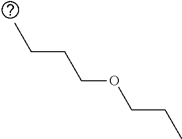 | R² = F | 5-Chloro-6-(3- fluorophenyl)-N-(3- propoxypropyl)nicotinamide | ¹H NMR (400 MHz, DMSO-d₆) ppm 0.84-0.92 (m, 3H), 1.48-1.54 (m, 2H), 1.75-1.84 (m, 2H), 3.26-3.49 (m, 6H), 7.31-7.40 (m, 1H), 7.51-7.59 (m, 3H), 8.72-8.81 (m, 1H), 9.02 (s, 1H). |
| 551 | 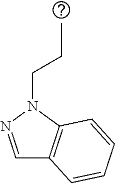 | R² = F | 5-Chloro-6-(3- fluorophenyl)-N-[3-(1H- indazol-1- yl)propyl]nicotinamide | Purified by HPLC Method (E) LCMS Method (F) RT 4.57 min, (ES) m/z 408.12 [M] calc 408.87 [M] |
| 552 | 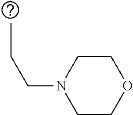 | R² = F | 5-Chloro-6-(3- fluorophenyl)-N-(2- morpholin-4- ylethyl)nicotinamide | 1H NMR (400 MHz, DMSO-d₆) ppm 2.37-2.45 (m, 5H), 3.37-3.45 (m, 2H), 3.52-3.60 (m, 5H), 7.28-7.37 (m, 1H), 7.49-7.57 (m, 3H), 8.38 (s, 1H) 8.71-8.75 (m, 1H), 8.99 (s, 1H). |
| 553 | 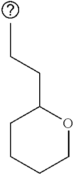 | R² = F | 5-Chloro-6-(3- fluorophenyl)-N-[2- (tetrahydro-2H-pyran-2- yl)ethyl]nicotinamide | ¹H NMR (400 MHz, DMSO-d₆) ppm 1.09-1.21 (m, 1H), 1.35-1.46 (m, 4H) 1.51-1.77 (m, 5H) 3.28-3.40 (m, 2H), 3.79-3.87 (m, 1H), 7.28-7.35 (m, 1H), 7.46-7.57 (m, 3H), 8.37 (s, 1H) 8.68-8.73 (m, 1H), 8.97 (s, 1H). LCMS (ES+) 363 (M + 1) |
| 554 | 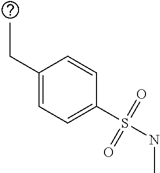 | R² = F | 5-Chloro-6-(3- fluorophenyl)-N-{4- [(methylamino)sulfonyl] benzyl}nicotinamide | ¹H NMR (400 MHz, DMSO-d₆) ppm 2.33-2.41 (m, 3H), 4.54-4.62 (m, 2H), 7.29-7.39 (m, 2H), 7.48-7.57 (m, 5H), 7.68-7.75 (m, 2H), 8.45 (s, 1H), 9.05 (s, 1H), 9.37-9.44 (m, 1H). |
| 555 | 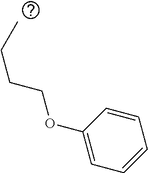 | R² = F | N-[2-(Benzyloxy)ethyl]-5- chloro-6-(3- fluorophenyl)nicotinamide | 1H NMR (400 MHz, DMSO-d₆) ppm 3.45-3.52 (m, 2H), 3.53-3.61 (m, 2H), 4.49 (s, 2H), 7.20-7.36 (m, 6H), 7.48-7.55 (m, 3H), 8.37 (s, 1H) 8.82-8.91 (m, 1H), 8.99 (s, 1H) LCMS (ES+) 363 (M + 1) |

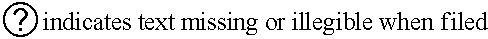

| Ex | R⁷ | R¹⁻⁵ | Name | Characterisation Data |
|---|---|---|---|---|
| 556 | 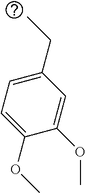 | R² = F R⁴ = OCH₃ | N-(3,4-Dimethoxybenzyl)- 6-(3-fluoro-5- methoxyphenyl)nicotinamide | Purified by HPLC Method (E) LCMS Method (F) RT 4.68 min, (ES) m/z 396.15 [M] calc 396.416 [M] |
| 557 | 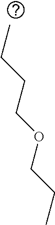 | R² = F R⁴ = OCH₃ | 6-(3-Fluoro-5- methoxypheny)-N-(3- propoxypropyl)nicotinamide | ¹H NMR (400 MHz, DMSO-d6) δ ppm 0.09 (t, J = 7.3 Hz , 3H) 0.58-0.80 (m, 2H) 1.01 (t, J = 6.7 Hz 3 H) 1.72 ( br. s, 1H) 2.52-2.61 (m, 4 H) 2.66 (t, J = 6.2 Hz, 3H) 6.16 (d, J = 10.8 Hz, 1H) 6.70-6.82 (m, 2H) (7.35 d, J = 8.2 Hz, 1H) 8.29 (s, 1H) 7.41-7.52 (m, 1H) 7.86 (br. s, 1H) |
| 558 | 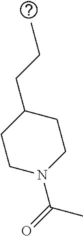 | R² = F R⁴ = OCH₃ | N-[(1-Acetylpiperidin-4- yl)methyl]-6-(3-fluoro-5- methoxyphenyl)nicotinamide | Purified by HPLC Method (E) LCMS Method (F) RT 4.05 min, (ES) m/z 385.18 [M] calc 385.44 [M] |
| 559 | 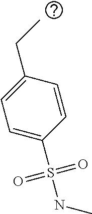 | R² = F R⁴ = OCH₃ | 6-(3-Fluoro-5- methoxyphenyl)-N-{4- [(methylamino)sulfonyl] benzyl}nicotinamide | ¹H NMR (400 MHz, DMSO-d6) δ ppm 1.63 (d, J = 4.9 Hz, 2 H,) 1.72 (br. s, 2 H) 3.09 (s, 3 H) 3.83 (d, J = 5.5 Hz, 2 H) 6.18 (d, J = 13.0 Hz, 1 H) 6.59 (d, J = 5.1 Hz, 1 H) 6.74-6.83 (m 3 H) 6.97 (d, J = 8.1 Hz 1 H) 7.39 (d, J = 8.2 Hz, 1 H) 7.56 (d, J = 10.2 Hz, 1 H) 8.37 (s, 1 H) 8.56 (br. s1 H) |
| 560 | 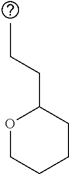 | R² = F R⁴ = OCH₃ | 6-(3-Fluoro-5- methoxyphenyl)-N-[2- (tetrahydro-2H-pyran-2- yl)ethyl]nicotinamide | ¹H NMR (400 MHz, DMSO-d6) δ ppm 0.43 (t, J = 11.3 Hz, 1H) 0.68 (br. s, 4 H) 0.81 (d, J = 12.8 Hz, 1H) 0.88 (q, J = 7.1 Hz, 3 H) 0.98 (br. s, 1 H) 1.72 (br. s, 1H) 2.59 (br. s, 5 H) 6.17 (d, J = 10.6 Hz, 1H) 6.71-6.82 (m, 2 H) 7.35 (d, J = 8.4 Hz, 1H) 7.45-7.53 (m ,1H) 7.84 (br. s, 1H) 8.28 (s, 1H) |
| 561 | 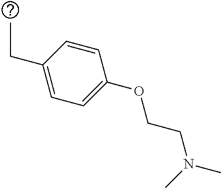 | R² = F | 5-Chloro-N-{4-[2- (dimethylamino)ethoxy] benzyl}-6-(3- fluorophenyl)nicotinamide | ¹H NMR (400 MHz, DMSO-d₆) ppm 2.17 (s, 6H), 2.52-2.60 (m, 2H), 3.96-4.02 (m, 2H), 4.38-4.48 (m, 2H), 6.83-6.91 (m, 2H), 7.20-7.37 (m, 3H), 7.48-7.55 (m, 3H), 8.42 (s, 1H), 9.02 (s, 1H), 9.19-9.28 (m, 1H) |
| 562 | 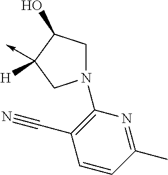 | R² = F | N-[(3S,4S)-1-(3-Cyano-6- methylpyridin-2-yl)-4- hydroxypyrrolidin-3-yl]-6- (3- fluorophenyl)nicotinamide | ¹H NMR (400 MHz, DMSO-d₆) ppm 2.33 (s, 3H), 3.58-3.65 (m, 1H), 3.70-3.78 (m, 1H), 3.91-4.05 (m, 2H), 4.23-4.39 (m, 2H), 5.41-5.48 (m, 1H), 6.54-6.61 (m, 1H), 7.24-7.32 (m, 1H), 7.49-7.57 (m, 1H), 7.75-7.81 (m, 1H), 7.88-8.02 (m, 2H), 8.06-8.13 (m, 1H), 8.24-8.30 (m, 1H), 8.64-8.72 (m, 1H), 9.05 (s, 1H). LCMS 418 [M + 1] |
| 563 | 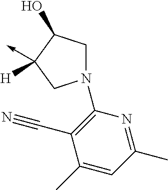 | R² = F | N-[(3S,4S)-1-(3-Cyano- 4,6-dimethylpyridin-2-yl)-4- hydroxypyrrolidin-3-yl]-6- (3- fluorophenyl)nicotinamide | ¹H NMR (400 MHz, DMSO-d₆) ppm 2.22-2.36 (m, 6H), 3.58-3.65 (m, 1H), 3.70-3.78 (m, 1H), 3.91-4.08 (m, 2H), 4.23-4.37 (m, 2H), 5.39-5.46 (m, 1H), 6.54 (s, 1H), 7.24-7.32 (m, 1H), 7.49-7.57 (m, 1H), 7.88-8.02 (m, 2H), 8.06-8.13 (m, 1H), 8.24-8.31 (m, 1H), 8.64-8.72 (m, 1H), 9.05 (s, 1H). LCMS 432 [M + 1] |
| 564 | 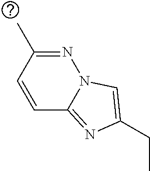 | R² = F | N-[(3S,4S)-1-(2- ethylimidazo[1,2- b]pyridazin-6-yl)-4- hydroxypyrrolidin-3-yl]-6- (3- fluorophenyl)nicotinamide | ¹H NMR (400 MHz, DMSO-d₆) ppm 1.15-1.25 (m, 3H), 2.56-2.65 (m, 2H), 3.30-3.38 (m, 1H), 3.44-3.52 (m, 1H), 3.67-73 (m, 1H), 3.75-3.83 (m, 1H), 4.23-4.44 (m, 2H), 5.40-5.48 (m, 1H), 6.67-6.77 (m, 1H) 7.24-7.33 (m, 1H), 7.49-7.57 (m, 1H), 7.59-7.69 (m, 2H), 7.87-8.00 (m, 2H), 8.06-8.13 (m, 1H) 8.24-8.31 (m, 1H), 8.66-8.75 (m, 1H), 9.06 (s, 1H). LCMS 447 [M + 1] |
| 565 | 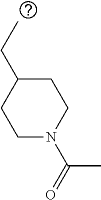 | R² = F R⁵ = OH | N-[(1-Acetylpiperidin-4- yl)methyl]-6-(5-fluoro-2- hydroxyphenyl)nicotinamide | Purified by HPLC Method (B) LCMS Method (A) RT 2.85 min (100%) 372.45 m/z [M +H] |
| 566 | 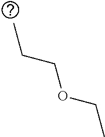 | R² = F R⁵ = OH | N-(2-Ethoxyethyl)-6-(5- fluoro-2- hydroxyphenyl)nicotinamide | Purified by HPLC Method (B) LCMS Method (A) RT 3.04 min (100%) 305.48 m/z [M + H] |
| 567 | 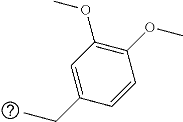 | R² = F R⁴ = F | 6-(3,5-Difluorophenyl)-N- (3,4- dimethoxybenzyl)nicotinamide | ¹H NMR (400 MHz, DMSO-d6) ppm, 3.73 (s, 3H) 3.75 (s, 3H), 4.45 (d, J = 5.5 Hz, 2H), 6.86-6.94 (m, 2H), 6.98 (s, 1H), 7.32-7.40 (m, 1H), 7.88 (d, J = 7.3 Hz, 2H), 8.20 (d, J = 8.1 Hz, 1H), 8.35 (dd, J = 8.4, 1.8 Hz, 1H), 9.16 (d, J = 16.1 Hz, 2H). LCMS 385 [M + 1] |
| 568 | 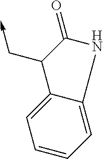 | R² = F R⁴ = F | 6-(3,5-Difluorophenyl)-N- [(2-oxo-2,3-dihydro-1H- indol-3- yl)methyl]nicotinamide | ¹H NMR (400 MHz, DMSO-d6) ppm 2.71-2.79 (m, 2H), 5.35-5.46 (m, 1H), 6.92-7.04 (m, 2H), 7.20-7.33 (m, 2H), 7.37 (t, J = 8.6 Hz, 1H), 7.89 (d, J = 8.8 Hz, 2H), 8.20 (d, J = 8.1 Hz, 1H), 8.36 (d, J = 2.2 Hz, 2H), 9.10-9.21 (m, 2H), 10.24 (s, 1H). LCMS 380 [M + 1 |
| 569 | 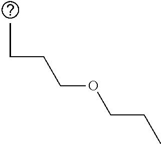 | R² = F R⁴ = F | 6-(3,5-Difluorophenyl)-N- (3- propoxypropyl)nicotinamide | ¹H NMR (400 MHz, DMSO-d6) ppm 0.87 (t, J = 7.5 Hz, 2H), 1.44-1.58 (m, 2H), 1.72-1.87 (m, 3H), 3.31-3.40 (m, 4H), 3.44 (t, J = 6.2 Hz, 2H), 7.28-7.41 (m, 1H), 7.88 (d, J = 7.0 Hz, 2H), 8.19 (d, J = 8.4 Hz, 1H), 8.30 (dd, J = 8.4, 2.2 Hz, 1H), 8.67 (br. s. 1H), 9.08 (s, 1H). LCMS 335 [M + 1] |
| 570 | 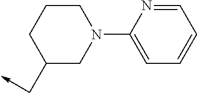 | R² = F R⁴ = F | 6-(3,5-Difluorophenyl)-N- [(1-pyridin-2-ylpiperidin-3- yl)methyl]nicotinamide | ¹H NMR (400 MHz, DMSO-d6) ppm 1.32-1.46 (m, 1H) 1.52-1.58 (m, 1H), 1.75-2.02 (m, 3H), 2.95-3.27 (m, 4H), 3.94-4.17 (m, 2H), 6.76-6.91 (m, 1H), 7.20-7.43 (m, 2H), 7.89 (d, J = 7.0 Hz, 3H), 7.97-8.04 (m, 1H), 8.21 (d, J = 8.1 Hz, 1H), 8.28-8.36 (m, 1H), 8.70-8.82 (m, 1H), 9.12 (s, 1H). LCMS 409 [M + 1] |
| 571 | 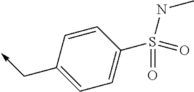 | R² = F R⁴ = F | 6-(3,5-Difluorophenyl)-N- {4- [(methylamino)sulfonyl] benzyl}nicotinamide | ¹H NMR (400 MHz, DMSO-d6) ppm 2.41 (d, J = 5.1 Hz, 3H), 4.61 (d, J = 5.5 Hz, 2H), 7.33-7.43 (m, 2H), 7.57 (d, J = 8.4 Hz, 2H), 7.75 (d, J = 8.1 Hz, 2H), 7.85-7.95 (m, 2H), 8.22 (d, J = 8.1 Hz, 1H), 8.37 (dd, J = 8.2, 2.0 Hz, 1H), 9.16 (s, 1H), 9.32-9.41 (m, 1H,). LCMS 418 [M + 1] |
| 572 | 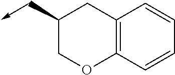 | R² = F | N-[(3R)-3,4-Dihydro-2H- chromen-3-ylmethyl)]-6-(3- fluorophenyl)nicotinamide | Enantiomer Peak 1, see experimental |
| 573 | 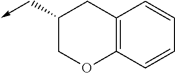 | R² = F | N-[(3S)-3,4-Dihydro-2H- chromen-3-ylmethyl)]-6-(3- fluorophenyl)nicotinamide | Enantiomer Peak 2, see experimental |

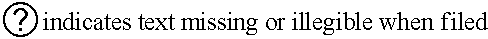

Examples 574-583 are defined by reference to formula (Ic)

(Ic)

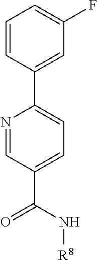

| Ex | R⁸ | Name | Purification and characterisation |
|---|---|---|---|
| 574 | 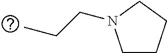 | 6-(3- Fluorophenyl)-N- (2-pyrrolidin-1- ylethyl) nicotinamide | LRMS obs 314 [M+ H] calc 314.38 ¹H NMR (CDCl3, 400 MHz) δ δ2.066-2.101 (m, 4H), 3.292-3.338 (m, 6H), 3.865-3.877 (m, 2H), 7.116-7.162 (m, 1H), 7.424-7.478 (m, 1H), 7.776-7.819 (m, 3H), 8.308-8.335 (m, 1H), 8.500-8.600 (m, 1H), 9.239-9.243 (m, 1H), 9.500-9.600 (m, 1H) |
| 575 | 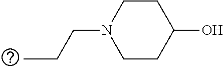 | 6-(3- Fluorophenyl)-N- [2-(4- hydroxypiperidin- 1-yl)ethyl] nicotinamide | LRMS: obs 344 [M + H] calc 344.40 ¹H NMR (CDCl₃, 400 MHz) δ 1.569-1.656 (m, 4H + H2O), 1.931-1.956 (m, 2H), 2.176-2.279 (m, 2H), 2.614-2.643 (m, 2H), 2.813-2.842 (m, 2H), 3.549-3.590 (m, 2H), 3.770 (br s, 1H), 6.900-7.000 (m, 1h) 7.137-7.183 (m, 1H), 7.443-7.498 (m, 1H), 7.772-7.828 (m, 3H), 8.207-8.233 (m, 1H), 9.035-9.040 (m, 1H) |
| 576 | —CH₂CH₂CH₃ | 6-(3- | LRMS Obs 259 [M + H] calc 258.30 [M+ H] ¹H |
| | | Fluorophenyl)-N- | NMR (CDCl₃, 400 MHz) δ 1.002-1.039 (m, |
| | | propylnicotinamide | 3H), 1.643-1.735 (m, 3H), 3.452-3.503 (m, 2H), |
| | | | 6.195 (br s, 1H), 7.132-7.179 (m, 1H), 7.264-7.492 |
| | | | (m, 1H), 7.765-7.797 (m, 3H), 8.184-8.210 |
| | | | (m, 1H), 8.019-9.023 (m, 1H) |
| 577 | —CH₂CH₂OH | 6-(3- | LRMS Obs 261 [M + H] calc 260.27 ¹H NMR |
| | | Fluorophenyl)-N- | (CDCl₃, 400 MHz) δ 3.676-3.715 (m, 2H), |
| | | (2- | 3.893-3.905 (m, 2H), 6.764 (br s, 1H), 7.136-7.182 |
| | | hydroxyethyl) | (m, 1H), 7.436-7.491 (m, 1H), 7.759-7.810 |
| | | nicotinamide | (m, 3H), 8.197-8.224 (m, 1H), 9.063-9.068 |
| | | | (m, 1H) |
| 578 | —CH₂CH₂NHCH₃ | 6-(3- | LRMS Obs 274 [M + H] calc 274.31 [M + H] ¹H |
| | | Fluorophenyl)-N- | NMR (DMSO-D6, 400 MHz) δ 2.578-2.604 (m, |
| | | [2- | 3H), 3.097-3.140 (m, 2H), 3.606-3.648 (m, 2H), |
| | | (methylamino)ethyl] | 7.323-7.371 (m, 1H), 7.558-7.614 (m, 1H), |
| | | nicotinamide | 7.981-8.051 (m, 2H), 8.189-8.209 (m, 1H), |
| | | hydrochloride salt | 8.422-8.449 (m, 1H), 8.950-9.050 (m, 2H), |
| | | | 9.179-9.196 (m, 2H). |
| 579 | 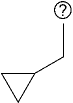 | N- (Cyclopropylmethyl)- 6-(3- fluorophenyl) nicotinamide | LRMS Obs 271 [M + H] calc 271.31 [M + H] ¹H NMR (CDCl₃, 400 MHz) δ 0.296-0.334 (m, 2H), 0.579-0.624 (m, 2H), 1.065-1.134 (m, 1H), 3.352-3.383 (m, 2H), 6.276 (br s, 1H) 7.134-7.180 (m, 1H), 7.438-7.493 (m, 1H), 7.771-7.816 (m, 3H), 8.196-8.222 (m, 1H), 9.048-9.052 (m, 1H). |
| 580 | —CH₂CH₃ | N-Ethyl-6-(3- | LRMS [M + H] 245, calc [M + H] 244.27 ¹H NMR |
| | | fluorophenyl) | (CDCl₃, 400 MHz) δ 1.252-1.321 (m, 3H), |
| | | nicotinamide | 3.524-3.592 (m, 2H), 6.145-6.155 (m, 1H), |
| | | | 7.132-7.178 (m, 1H), 7.436-7.491 (m, 1H), |
| | | | 7.766-7.812 (m, 3H), 8.183-8.210 (m, 1H), |
| | | | 9.017-9.021 (m, 1H) |
| 581 | —CH₂CH₂CO₂H | N-{[6-(3- | LRMS Obs [M + H] 289 calc 289.3 [M + H] ¹H |
| | | Fluorophenyl)pyridin- | NMR (CDCl₃, 400 MHz) δ 2.654-2.684 (m, |
| | | 3-yl]carbonyl}-beta- | 2H), 3.746-3.775 (m, 2H), 7.129-7.175 (m, 1H), |
| | | alanine | 7.345-7.493 (m, 2H), 7.767-7.813 (m, 3H), |
| | | | 8.198-8.224 (m, 1H), 9.080-9.085 (m, 1H) |
| 582 | 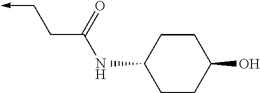 | 6-(3- Fuorophenyl)-N- {3-[(trans-4- hydroxycyclohexyl) amino]-3- oxopropyl} nicotinamide | LRMS Obs 386 [M + H] calc 386.44 [M + H] ¹H NMR (CDCl₃, 400 MHz) δ 1.125-1.185 (m, 4H), 1.178-1.795 (m, 4H), 2.330-2.366 (m, 2H), 3.450-3.499 (m, 3H), 4.524-4.535 (m, 1H), 7.308-7.355 (m, 1H), 7.546-7.601 (m, 1H), 7.768-7.787 (m, 1H), 7.957-8.030 (m, 1H), 8.143-8.164 (m, 1H), 8.255-8.282 (m, 1H), 8.774-8.801 (m, 1H), 9.061-9.065 (m, 1H). |
| 583 | 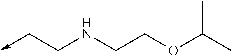 | 6-(3- Fluorophenyl)-N- {2-[(2- isopropoxyethyl) amino] ethyl}nicotinamide | LRMS Obs 346 [M + H], calc 345.42 [M + H] ¹H NMR (DMSO-D₆, 400 MHz) δ 1.050-1.075 (m, 6H) 2.660-2.719 (m, 4H) 3.354-3.426 (m, 4H) 3.517-3.532 (m, 1H) 7.330-7.400 (m, 1H), 7.560-7.585 (m, 1H), 7.962-8.035 (m, 2H), 8.148-8.169 (m, 1H), 8.283-8.309 (m, 1H) 8.710-8.720 (m, 1H) 9.089-9.093 (m, 1H) |

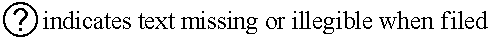

[symbol]

Examples 584-591 are defined by reference to Formula (Id):

(Id)

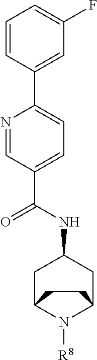

| Ex | R⁸ | Name | Characterisation Data |
|---|---|---|---|
| 584 | H | N-[(3-endo)-8- | (ES+) 326 [M + 1] ¹H NMR (400 MHz |
| | | Azabicyclo[3.2.1]oct-3- | MeOD-d₄) δ ppm 1H NMR (400 MHz, |
| | | yl]-6-(3- | METHANOL-d₄) ppm 1.94-2.03 (m, 4H), |
| | | fluorophenyl)nicotinamide | 2.12-2.20 (m, 4H), 3.54-3.61 (m, 2H), |
| | | | 4.11-4.17 (m, 1H), 7.17-7.24 (m, 1H), |
| | | | 7.49-7.55 (m, 1H), 7.80-7.91 (m, 2H), |
| | | | 7.98- 8.02 (m, 1H), 8.19-8.24 (m, 1H), |
| | | | 8.96-9.00 (m, 1H) |
| 585 | —CH₂CH₂CH₃ | 6-(3-Fluorophenyl)-N-[(3- | Purified by HPLC method (A) |
| | | endo)-8-propyl-8- | LCMS method (basic conditions) RT 3.2 |
| | | azabicyclo[3.2.1]oct-3- | min (100% area) ES m/z 368 [M + 1] |
| | | yl]nicotinamide | |
| 586 | —CO₂C(CH₃)₃ | tert-Butyl (3-endo)-3-({[6- | LCMS (ES+) 426 [M + 1] ¹H NMR (400 |
| | | (3-fluorophenyl)pyridin-3- | MHz MeOD-d₄) δ ppm 1.49 (s, 9H), 1.93 |
| | | yl]carbonyl}amino)-8- | (m, 2H), 2.03-213 (m, 4H), 2.18-3.01 |
| | | azabicyclo[3.2.1]octane- | (m, 2H), 4.10-4.18 (m, 1H), 4.20-4.26 (m, |
| | | 8-carboxylate | 2H), 7.17-7.26 (m, 1H), 7.48-7.56 (m, |
| | | | 1H), 7.80-7.91 (m, 2H), 7.97-8.02 (m, |
| | | | 1H), 8.20-8.26 (m, 1H), 8.98-9.02 (m, |
| | | | 1H). |

Examples 587-591 are defined by reference to formula (Ie):

(Ie)

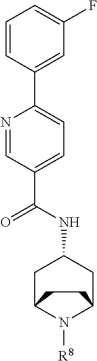

| Ex | R⁸ | Name | Characterisation Data |
|---|---|---|---|
| 587 | H | N-[(3-exo)-8- | LCMS (ES+) 326 [M + 1] ¹H NMR (400 MHz MeOD-d₄) |
| | | Azabicyclo[3.2.1] | δ ppm 1.58-1.69 (m, 2H), 1.84-1.97 (m, 6H), 3.55-3.62 |
| | | oct-3-yl]-6-(3- | (m, 2H), 4.32-4.41 (m, 1H), 7.18-724 (m, 1H), |
| | | fluorophenyl) | 748-7.56 (m, 1H), 7.80-7.89 (m, 2H), 7.95-8.00 (m, |
| | | nicotinamide | 1H), 8.22-8.28 (m, 1H), 9.00-9.04 (m, 1H) |
| 588 | —CO₂C(CH₃)₃ | tert-Butyl (3-exo)- | LCMS (ES+) 426 [M + 1] ¹H NMR (400 MHz MeOD-d₄) |
| | | 3-({[6-(3- | δ ppm 1.49 (s, 9H), 1.69-1.82 (m, 2H) 1.84-1.98 (m, |
| | | fluorophenyl)pyridin- | 4H), 2.00-2.08 (m, 2H) 4.22-4.31 (m, 2H), 4.49-4.61 |
| | | 3- | (m, 1H), 7.18-7.24 (m, 1H), 7.48-7.56 (m, 1H), 7.80-7.89 |
| | | yl]carbonyl}amino)- | (m, 2H) 7.95-8.00 (m, 1H), 8.23-8.26 (m, 1H), |
| | | 8- | 9.01-9.04 (m, 1H). |
| | | azabicyclo[3.2.1] | |
| | | octane-8- | |
| | | carboxylate | |
| 589 | —CH₂CH₂CH₃ | 6-(3- | LCMS (ES+) 368 [M + 1] 1H NMR (400 MHz, MeOD-d₄) |
| | | Fluorophenyl)-N- | δ ppm 0.91-1.00 (m, 3H), 1.51-1.63 (m, 2H), 1.72-1.89 |
| | | [(3-exo)-8-propyl- | (m, 6H), 2.03-2.13 (m, 2H), 2.43-2.53 (m, 2H), 3.37-3.45 |
| | | 8- | (m, 2H), 4.31-4.42 (m, 1H), 7.17-7.25 (m, 1H), |
| | | azabicyclo[3.2.1] | 7.48-7.56 (m, 1H), 7.80-7.90 (m, 2H), 7.96-8.01 (m, |
| | | oct-3- | 1H), 8.23-8.29 (m, 1H), 9.01-9.06 (m, 1H). |
| | | yl]nicotinamide | |
| 590 | —COCH₃ | N-[(3-exo)-8- | LCMS (ES+) 368 [M + 1] ¹H NMR (400 MHz, MeOD-d₄) |
| | | Acetyl-8- | δ ppm 1.66-1.80 (m, 2H), 1.89-2.04 (m, 4H), 2.06-2.18 |
| | | azabicyclo[3.2.1]o | (m, 5H), 4.32-4.39 (m, 1H), 4.57-4.69 (m, 2H), 7.18-7.24 |
| | | 5ct-3-yl]-6-(3- | (m, 1H), 7.48-7.56 (m, 1H), 7.80-7.90 (m, 2H), |
| | | fluorophenyl) | 7.96-8.00 (m, 1H), 8.23-8.28 (m, 1H), 9.01-9.05 (m, |
| | | nicotinamide | 1H). |
| 591 | —SO₂CH(CH₃)₂ | 6-(3- | Purified by by HPLC method (B) |
| | | Fluorophenyl)-N- | LCMS method (basic conditions) RT 3.12 min (100% |
| | | [(3-exo)-8- | area) ES m/z 432 [M + 1] |
| | | (isopropylsulfonyl)- | |
| | | 8- | |
| | | azabicyclo[3.2.1] | |
| | | oct-3- | |
| | | yl]nicotinamide | |

Details of purification methods referenced in the tables above along with further details concerning the preparation and characterization of selected tabulated Examples are provided in the following section.

### Method A

| HPLC | LCMS Method A | | HPLC Method A | |
|---|---|---|---|---|
| conditions | (analytical) | | (preparative) | |
| Column | Sunfire C18 | | Sunfire Prep C18 | |
| | 5 μm 4.6 × 50 mm | | 5 μm 19 × 100 mm | |
| Temperature | Ambient | | Ambient | |
| Detection | UV 225 nm - ELSD - MS | | ELSD-MS | |
| System/Data file | CTC-MUX1 | | Fractionlynx 1 | |
| Injection volume | 5 μL | | 1000 μL | |
| Flow rate | 1.5 mL/min | | 18 mL/min | |
| Mobile phase | A: H₂O + 0.1% formic | | A: H₂O + 0.1% formic | |
| | acid | | acid | |
| | B: MeCN + 0.1% formic | | B: MeCN + 0.1% formic | |
| | acid | | acid | |
| | Time | | Time | |
| Gradient | (min) | % B | (min) | % B |
| | 0 | 5 | 0-1.0 | 5 |
| | 0-3.0 | 5-95 | 1.0-7.0 | 5-98 |
| | 3.0-4.0 | 95 | 7.0-9.0 | 98 |
| | 4.0-4.1 | 95-5 | 9.0-9.10 | 98-5 |
| | 4.1-5.0 | 5 | 9.10-10 | 5 |

### Method B

| HPLC | LCMS Method B | | HPLC Method B | |
|---|---|---|---|---|
| conditions | (analytical) | | (preparative) | |
| Column | XTerra C18 | | Sunfire Prep C18 | |
| | 5 μm 4.6 × 50 mm | | 5 μm 19 × 50 mm | |
| Temperature | Ambient | | Ambient | |
| Detection | UV 225 nm - ELSD - MS | | ELSD-MS | |
| System/Data file | CTC - MUX1 | | Fractionlynx 1 | |
| Injection volume | 5 μL | | 1000 μL | |
| Flow rate | 1.5 mL/min | | 18 mL/min | |
| Mobile phase | A: H₂O + 0.1% ammonia | | A: H₂O + 0.1% DEA | |
| | B: MeCN + 0.1% | | B: MeCN + 0.1% | |
| | ammonia | | ammonia | |
| | Time | | Time | |
| Gradient | (min) | % B | (min) | % B |
| | 0 | 5 | 0-1.0 | 5 |
| | 0-3.0 | 5-95 | 1.0-7.0 | 5-98 |
| | 3.0-4.0 | 95 | 7.0-9.0 | 98 |
| | 4.0-4.1 | 95-5 | 9.0-9.10 | 98-5 |
| | 4.1-5.0 | 5 | 9.10-10 | 5 |

### LCMS Method C

### Analytical

| HPLC | | | |
|---|---|---|---|
| conditions | LCMS | | |
| Column | Analytical S&P Advantage Armor C18 | | |
| | 5 μm 4.6 × 50 mm | | |
| Temperature | Ambient | | |
| Detection | UV 220-400 nm - ELSD - MS | | |
| Injection | 12 μL | | |
| volume | | | |
| Flow rate | 4.0 mL/min | | |
| Mobile | A: H₂O + 0.5% trifluoroacetic acid | | |
| phase | B: MeCN | | |
| Gradient | Time (min) | % A | % B |
| | 0 | 95 | 5 |
| | 0.50 | 95 | 5 |
| | 3.60 | 5 | 95 |
| | 3.95 | 95 | 5 |
| | 4.00 | 95 | 5 |

### HPLC Method D

### Preparative

| | |
|---|---|
| HPLC | |
| conditions | Preparative |

| Column | Phenomenex Luna C18(2) 5 μm 21.2 × 50 mm | | |
|---|---|---|---|
| Temperature | Ambient | | |
| Detection | ELSD | | |
| Injection | 2000 μL | | |
| volume | | | |
| Flow rate | 45.0 mL/min | | |
| Mobile | A: H₂O + 0.5% trifluoroacetic acid | | |
| phase | B: MeCN +0.5% trifluoroacetic acid | | |
| Gradient | Time (min) | % A | % B |
| | 0 | 90 | 10 |
| | 0.10 | 90 | 10 |
| | 2.30 | 30 | 70 |
| | 2.70 | 5 | 95 |
| | 3.70 | 5 | 95 |
| | 3.90 | 90 | 10 |
| | 4.00 | 90 | 10 |

### HPLC Method E

### Preparative

Purification was achieved using a Waters Sunfire C18 Column 20×50 mm×5 μm eluting with a water/acetonitrile/0.1% formic acid gradient, typically from 85% water to 5% water over 8 minutes. The flow rate was 30 ml/min and the trigger was by mass spectrometry.

### LCMS Method F

### Analytical

Analysis was conducted using a Sunfire C18 Column, 2.1×50 mm×5 μm. Gradient elution was carried out with water/acetonitrile/0.1% formic acid, gradient 95-5% water over 8 minutes, 1 min hold at the end of the run, flow rate 1 mL/min, purity assessment by UV (215 nM).

### EXAMPLE 1

### 6-(3-Fluorophenyl)-N-[2-(6-methylimidazo[1,2-a]pyridin-2-yl)ethyl]nicotinamide

6-(3-Fluorophenyl)nicotinic acid (50 mmol), HATU (50 mmol) and triethylamine (50 mmol) were dissolved into DM. 2-(6-Methyl-imidazo[1,2-a]pyridine-2-yl)ethylamine (50 mmol) was added and the solution was agitated at room temperature for 16 hours. The solvent was evaporated and the residue was purified by HPLC to give the title compound. Methods C (analytical) and D (preparative) were used.

Examples 2-150 were similarly prepared.

### EXAMPLE 151

### N-(2-Methylbenzyl)-6-phenylnicotinamide

6-Phenylnicotinic acid (30 mg, 0.15 mmol), HOBT (46 mg, 0.3 mmol) and 2-methylbenzylamine (18 mg, 0.15 mmol) were added to a suspension of polymer suspended carbodiimide (0.2 mmol) in DMF (1 mL). The reaction was stirred at room temperature for 18 hours. The solvent was removed under reduced pressure and the residue was purified by reverse phase HPLC chromatography using Method E. The products were analysed using Method F. This gave the title compound.

Examples 152-528 were similarly prepared.

### EXAMPLE 529

### 6-(3-Fluorophenyl)-N-[(1R,5S,6s)-3-pyrimidin-2-yl-3-azabicyclo[3.1.0]hex-6-yl]nicotinamide

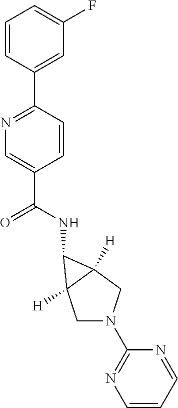

This Example was prepared using CDI as the coupling agent as described in the general methods section above using 6-(3-fluorophenyl)nicotinic acid (100 mg, 0.46 mmol) and (1S,5R,6S)-3-pyrimidin-2-yl-3-aza-bicyclo[3.1.0]hex-6-ylamine (81 mg, 0.46 mmol). The product was purified by flash chromatography over silica gel eluting ethyl acetate/heptane (1:3).

### EXAMPLE 534

### N-[(5-Fluoro-2-oxo-2,3-dihydro-1H-indol-3-yl)methyl]-6-(3-fluorophenyl)nicotinamide

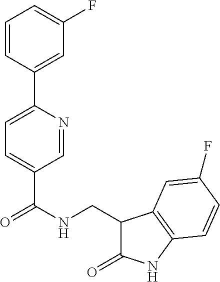

6-(3-Fluorophenyl)nicotinic acid (109 mg, 0.5 mmol), 3-aminomethyl-5-fluoro-1,3-dihydroindol-2-one (108 mg, 0.5 mmol), TBTU (193 mg, 0.60 mmol) and triethylamine (152 mg, 1.5 mmol) were stirred together in dichloromethane (3 mL) overnight. Dichloromethane (4 mL) and water (5 mL) were added and the precipitated solid was filtered and washed with water and diethyl ether to give 100 mg of the product.

### EXAMPLE 535

### 6-(3-Fluorophenyl)-N-{[2-(4-fluorophenyl)-1,3-oxazol-4-yl]methyl}nicotinamide

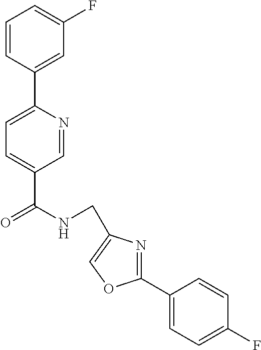

6-(3-Fluorophenyl)nicotinic acid (109 mg, 0.5 mmol), 1-[2-(4-fluorophenyl)-1,3-oxazol-4-yl]methanamine (96.1 mg, 0.5 mmol), TBTU (193 mg, 0.60 mmol) and triethylamine (152 mg, 1.5 mmol) were stirred together in dichloromethane (3 mL) overnight. Dichloromethane (4 mL) and water (5 mL) were added and the precipitated solid was filtered and washed with water and diethyl ether to give 100 mg of the product.

### EXAMPLE 542

### 6-(3,5-Difluorophenyl)-N-(3,4-dihydro-2H-chromen-3-ylmethyl)nicotinamide

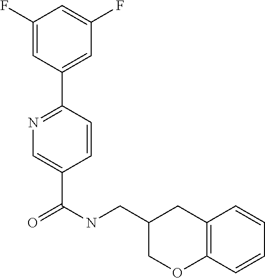

6-(3,5-Difluorophenyl)nicotinic acid (49.0 mg, 0.217 mmol), 1-(3,4-dihydro-2H-chromen-3-yl)methanamine (43.3 mg, 0.217 mmol), HATU (98.5 mg, 0.259 mmol) and diisopropylamine (214 mg, 1.66 mmol) were mixed in acetonitrile (2 mL) and shaken over night. The reaction was concentrated and purified by reverse phase HPLC Method (E).

### EXAMPLE 562

### trans-N-1-(3-Cyano-6-methylpyridin-2-yl)-4-hydroxypyrrolidin-3-yl]-6-(3-fluorophenyl)nicotinamide

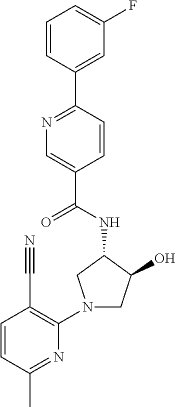

To a vial was added trans-6-(3-fluorophenyl)-N-[4-hydroxypyrrolidin-3-yl]nicotinamide (40 mg, 0.12 mmol), 2-chloro-6-methyl-nicotinonitrile (27.2 mg, 0.18 mmol), n-butanol, water and triethylamine (0.3 mL of each). The reaction mixture was heated to 90° C. overnight and then cooled to room temperature and evaporated. The residue was purified by HPLC Method (E) to give the desired product, trans-N-1-(3-cyano-6-methylpyridin-2-yl)-4-hydroxypyrrolidin-3-yl]-6-(3-fluorophenyl)nicotinamide (40 mg, 81%).

### EXAMPLE 563

### trans-N-1-(3-Cyano-4,6-dimethylpyridin-2-yl)-4-hydroxypyrrolidin-3-yl]-6-(3-fluorophenyl)nicotinamide

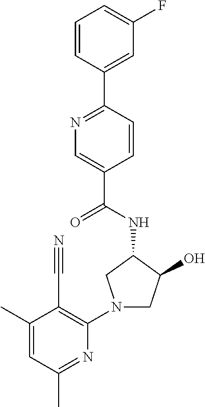

This Example was prepared in a similar manner to Example 562 using trans-6-(3-fluorophenyl)-N-[4-hydroxypyrrolidin-3-yl]nicotinamide (40 mg, 0.12 mmol), and 2-chloro-4,6-dimethyl-nicotinonitrile (29.0 mg, 0.18 mmol). The product was purified by HPLC Method (E).

### EXAMPLE 564

### trans-1-(2-Ethylimidazo[1,2-b]pyridazin-6-yl)-4-hydroxypyrrolidin-3-yl]-6-(3-fluorophenyl)nicotinamide

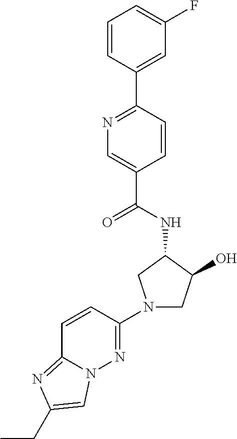

This Example was prepared in a similar manner to Example 562 using trans-6-(3-fluorophenyl)-N-[4-hydroxypyrrolidin-3-yl]nicotinamide (40 mg, 0.12 mmol), and 6-chloro-2-ethyl-imidazo[1,2-b]pyridazine (29.6 mg, 0.18 mmol). The product was purified by HPLC Method (E).

### EXAMPLE 567

### 6-(3,5-Difluorophenyl)-N-(3,4-dimethoxybenzyl)nicotinamide

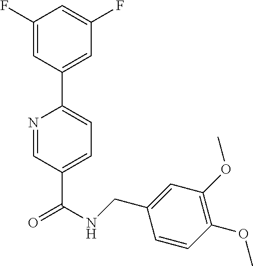

This Example was prepared using PS-carbodiimide as described in the general methods above from 6-(3,5-difluorophenyl)nicotinic acid (54 mg, 0.23 mmol) and 3,4-dimethoxy-benzylamine (38.0 mg, 0.23 mmol). The product was purified by HPLC Method (E).

### EXAMPLE 568

### 6-(3,5-Difluorophenyl)-N-[(2-oxo-2,3-dihydro-1H-indol-3-yl)methyl]nicotinamide

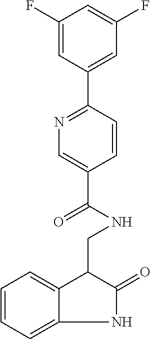

This Example was prepared using HATU, as in Example 542, with 6-(3,5-difluorophenyl)nicotinic acid (54 mg, 0.23 mmol) and 3-aminomethyl-1,3-dihydro-indol-2-one (44.0 mg, 0.23 mmol) as the starting materials. The product was purified by HPLC Method (E).

### EXAMPLE 569

### 6-(3,5-Difluorophenyl)-N-(3-propoxypropyl)nicotinamide

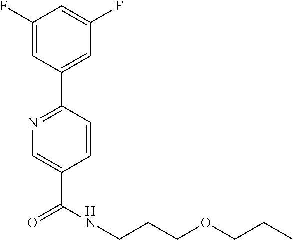

This Example was prepared with PS-carbodiimide as described in the general methods using 6-(3,5-difluorophenyl)nicotinic acid (54 mg, 0.23 mmol) and 3-propoxy-propylamine (27.0 mg, 0.23 mmol). The product was purified by HPLC Method (E).

### EXAMPLE 570

### 6-(3,5-Difluorophenyl)-N-[(1-pyridin-2-ylpiperidin-3-yl)methyl]nicotinamide

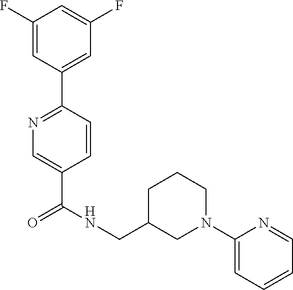

This Example was prepared using HATU, as in Example 542, with 6-(3,5-difluorophenyl)nicotinic acid (54 mg, 0.23 mmol) and 3,4,5,6-tetrahydro-2H-[1,2]bipyridinyl-3-yl)-methylamine (68.0 mg, 0.23 mmol) as the starting materials. The product was purified by HPLC Method (E).

### EXAMPLE 571

### 6-(3,5-Difluorophenyl)-N-{4-[(methylamino)sulfonyl]benzyl}nicotinamide

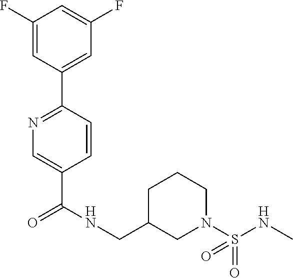

This Example was prepared using PS-carbodiimide as described in the general methods section with 6-(3,5-difluorophenyl)nicotinic acid (54 mg, 0.23 mmol) and 4-aminomethyl-N-methyl-benzenesulfonamide (71.0 mg, 0.36 mmol) as the starting materials. The residue was purified by flash chromatography over silica gel eluting dichloromethane/methanol/ammonia (95:5:0.5) to give 6-(3,5-difluorophenyl)-N-{4 [(methylamino)sulfonyl]benzyl}nicotinamide.

### EXAMPLES 572 AND 573

### N-[(3R)-3,4-Dihydro-2H-chromen-3-ylmethyl)]-6-(3-fluorophenyl)nicotinamide and N-[(3S)-3,4-dihydro-2H-chromen-3-ylmethyl)]-6-(3-fluorophenyl)nicotinamide

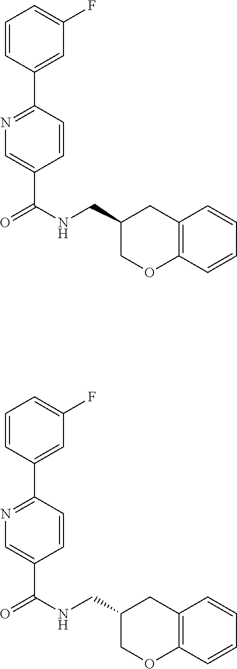

The racemate of the title compounds was prepared analogously to Example 542 and was then purified using an AD-H column, 30×250 mm, flow rate 70 mL./min, sample dissolved at 2 mg/mL in isopropanol, eluant 50% EtOH/CO₂ isocratic. The two peaks were analysed on a Chiral Technologies AD-H column, eluant 50% EtOH/CO₂.

Peak 1, retention time 2.2 min gave a negative CD-spectrum at 280 nM.

Peak 2, retention time 2.5 min gave a positive CD-spectrum at 280 nM.

### EXAMPLE 578

### 6-(3-Fluorophenyl)-N-(2-(methylamino)ethyl)nicotinamide hydrochloride

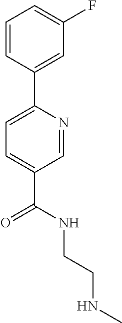

tert-Butyl 2-(6-(3-fluorophenyl)nicotinamido)ethyl(methyl)carbamate (0.24 g, 0.643 mmol) was dissolved in 1,4-dioxane (2 mL) and 4M HCl in dioxane was added (2 mL). The reaction mixture was stirred for 18 hours. The resulting solids were removed by filtration, washed with Et₂O (10 mL) and air dried. The product was obtained in 93% yield (0.185 g, 0.597 mmol).

### EXAMPLE 579

### N-(Cyclopropylmethyl)-6-(3-fluorophenyl)nicotinamide

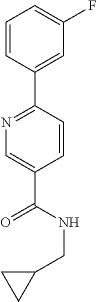

6-(3-Fluorophenyl)nicotinic acid (0.15 g, 0.691 mmol) was dissolved in 3 mL of DCM. To this stirred solution were added 1-ethyl-3-(3-dimethylaminopropyl)carbodiimide (0.146 g, 0.760 mmol) and 1-hydroxy-7-azabenzotriazole (0.094 g, 0.691 mmol), followed by aminomethylcyclopropane (0.049 g, 0.691 mmol). After 18 hours stirring at room temperature, water (3 mL) was added and the phases were separated. The organic phase was evaporated in vacuo, and the product was purified by flash column chromatography using a DCM to DCM/MeOH 85/15 gradient, followed by flash column chromatography using a DCM to DCM/MeOH 10/90 gradient. The title compound was obtained after lyophilisation (0.051 g, 0.189 mmol, 27% yield).

Examples 574-577 and 580-582 were similarly prepared.

### EXAMPLE 583

### 6-(3-Fluorophenyl)-N-(2-(2-isopropoxyethylamino)ethyl)nicotinamide

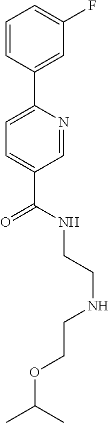

A suspension of benzyl 2-(6-(3-fluorophenyl)nicotinamido)ethyl(2-isopropoxyethyl) carbamate (67 mg, 0.140 mmol) and 10% Palladium on activated charcoal (14.87 mg, 0.140 mmol) in ethanol (3 mL) was stirred at room temperature under hydrogen for 18 hours. The reaction mixture was filtered and the filtrate was concentrated in vacuo, yielding 45 mg of a pale yellow, sticky solid. This material was purified by flash chromatography (EtOAc containing 1-2% 7 M NH₃ in MeOH) yielding 29.9 mg of a pale yellow solid (0.082 mmol, 59% yield)

### EXAMPLE 584

### N-[(3-endo)-8-azabicyclo[3.2.1]oct-3-yl]-6-(3-fluorophenyl)nicotinamide

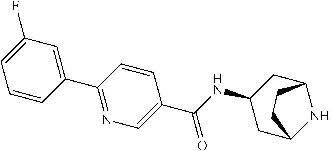

tert-Butyl (3-endo)-3-({[6-(3-fluorophenyl)pyridin-3-yl]carbonyl}amino)-8-azabicyclo[3.2.1]octane-8-carboxylate (220 mg, 0.517 mmol) was dissolved in a solution of HCl in anhydrous methanol (1N, 30 mL) and stirred at 50° C. for 3 hours. The mixture was concentrated and the residue was purified on an Isolute SCX-2® ion exchange resin to give N-[(3-endo)-8-azabicyclo[3.2.1]oct-3-yl]-6-(3-fluorophenyl)nicotinamide (140 mg).

### EXAMPLE 585

### 6-(3-Fluorophenyl)-N-[(3-endo)-8-propyl-8-azabicyclo[3.2.1]oct-3-yl]nicotinamide

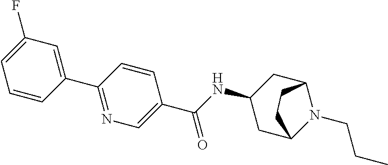

To a solution of N-[(3-endo)-8-azabicyclo[3.2.1]oct-3-yl]-6-(3-fluorophenyl)nicotinamide (145 mg, 0.446 mmol) in isopropyl alcohol (15 mL) was added 1-iodopropane (146 mg, 0.862 mmol) and potassium carbonate (198 mg, 1.44 mmol), and the mixture was heated to 75° C. for 16 hours. The solvent was evaporated and the residue was partitioned between ethyl acetate (20 mL) and water (5 mL). The organic layer was separated, dried over anhydrous MgSO₄, filtered and evaporated to give an off white solid.

### EXAMPLE 586

### tert-Butyl (3-endo)-3-({[6-(3-fluorophenyl)pyridin-3-yl]carbonyl}amino)-8-azabicyclo[3.2.1]octane-8-carboxylate

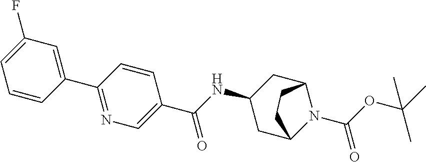

This Example was prepared as outlined in general methods from 6-(3-fluorophenyl)nicotinic acid (480 mg, 2.21 mmol) and (1S,3R,5R)-3-amino-8-aza-bicyclo[3.2.1]octane-8-carboxylic acid tert-butyl ester (500 mg, 2.21 mmol) to give tert-butyl (3-endo)-3-({[6-(3-fluorophenyl)pyridin-3-yl]carbonyl}amino)-8-azabicyclo[3.2.1]octane-8-carboxylate as a white solid (270 mg).

### EXAMPLE 587

### N-[(3-exo)-8-azabicyclo[3.2.1]oct-3-yl]-6-(3-fluorophenyl)nicotinamide

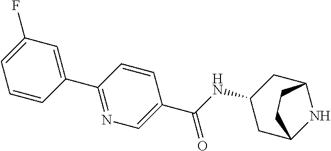

tert-Butyl (3-exo)-3-({[6-(3-fluorophenyl)pyridin-3-yl]carbonyl}amino)-8-azabicyclo[3.2.1]octane-8-carboxylate (550 mg, 1.29 mmol) was dissolved in a solution of HCl in anhydrous methanol (1N, 50 mL) and the reaction mixture was stirred at 50° C. for 3 hours. The mixture was concentrated and the residue was purified on an Isolute SCX-2® ion exchange resin to give N-[(3-exo)-8-azabicyclo[3.2.1]oct-3-yl]-6-(3-fluorophenyl)nicotinamide (330 mg).

### EXAMPLE 588

### tert-Butyl (3-exo)-3-({[6-(3-fluorophenyl)pyridin-3-yl]carbonyl}amino)-8-azabicyclo[3.2.1]octane-8-carboxylate.

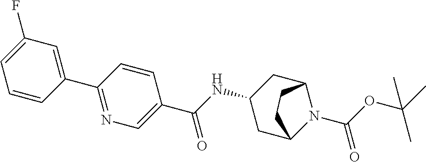

This Example was prepared as outlined in the general methods section from 6-(3-fluorophenyl)nicotinic acid (480 mg, 2.21 mmol) and (1S,3S,5R)-3-amino-8-azabicyclo[3.2.1]octane-8-carboxylic acid tert-butyl ester (500 mg, 2.21 mmol) to give tert-butyl (3-exo)-3-({[6-(3-fluorophenyl)pyridin-3-yl]carbonyl}amino)-8-azabicyclo[3.2.1]octane-8-carboxylate as a white solid (760 mg).

### EXAMPLE 589

### 6-(3-Fluorophenyl)-N-[(3-exo)-8-propyl-8-azabicyclo[3.2.1]oct-3-yl]nicotinamide

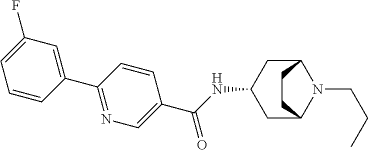

This Example was prepared in a similar manner to Example 585 using N-[(3-exo)-8-azabicyclo[3.2.1]oct-3-yl]-6-(3-fluorophenyl)nicotinamide (100 mg, 0.307 mmol) and 1-iodopropane (120 mg, 0.705 mmol) to give 6-(3-fluorophenyl)-N-[(3-exo)-8-propyl-8-azabicyclo[3.2.1]oct-3-yl]nicotinamide.

### EXAMPLE 590

### N-[(3-exo)-8-Acetyl-8-azabicyclo[3.2.1]oct-3-yl]-6-(3-fluorophenyl)nicotinamide

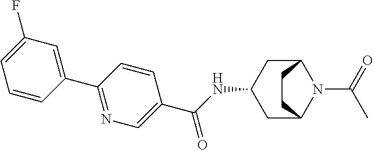

To a solution of N-[(3-exo)-8-azabicyclo[3.2.1]oct-3-yl]-6-(3-fluorophenyl)nicotinamide (100 mg, 0.307 mmol) in dichloromethane (5 mL) was added triethylamine (0.086 mL, 0.614 mmol) and acetyl chloride (0.024 mL, 0.338 mmol) and the reaction mixture was stirred at room temperature for 2 hours. The reaction was diluted with dichloromethane (5 mL) and washed with water (5 mL). The organic layer was separated, dried over anhydrous MgSO₄, filtered and evaporated. The residue was purified by flash chromatography over silica gel eluting with dichloromethane/methanol/ammonia (95:5:0.5) to give N-[(3-exo)-8-acetyl-8-azabicyclo[3.2.1]oct-3-yl]-6-(3-fluorophenyl)nicotinamide as a white solid (100 mg).

### EXAMPLE 591

### 6-(3-Fluorophenyl)-N-[(3-exo)-8-(isopropylsulfonyl)-8-azabicyclo[3.2.1]oct-3-yl]nicotinamide

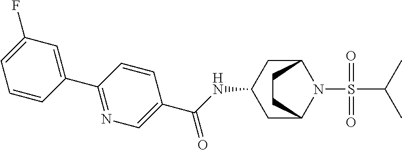

This Example was prepared from N-[(3-exo)-8-azabicyclo[3.2.1]oct-3-yl]-6-(3-fluorophenyl)nicotinamide (113 mg, 0.347 mmol) and isopropylsulfonyl chloride (0.086 mL, 0.764 mmol) and the product was purified by HPLC.

Further Examples 592 and 293 may be prepared as follows.

### EXAMPLE 592

### tert-Butyl 2-(6-(3-fluorophenyl)nicotinamido)ethyl(methyl)carbamate

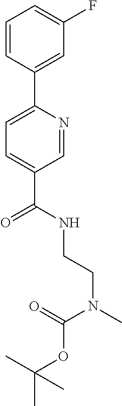

tert-Butyl 2-(6-(3-fluorophenyl)nicotinamido)ethyl(methyl)carbamate was prepared analogously to N-(cyclopropylmethyl)-6-(3-fluorophenyl)nicotinamide in 70% yield. LRMS: observed 374 [M+H], calculated 374.31 [M+H].

### EXAMPLE 593

### Benzyl 2-(6-(3-fluorophenyl)nicotinamido)ethyl(2-isopropoxyethyl)carbamate

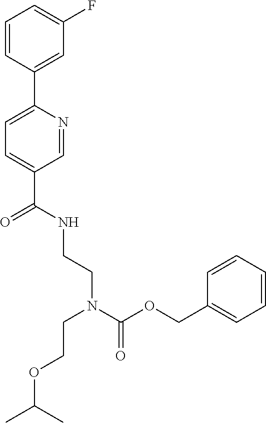

EDCI (267 mg, 1.391 mmol) and 1-hydroxy-7-azabenzotriazole (151 mg, 1.113 mmol) were added to a solution of benzyl 2-aminoethyl(2-isopropoxyethyl) carbamate (260 mg, 0.927 mmol) and 6-(3-fluorophenyl)nicotinic acid (302 mg, 1.391 mmol) in N,N-dimethylformamide (20 mL) at room temperature and stirred overnight at room temperature. The majority of the DMF was removed in vacuo. Water (10 mL) and 1 M NaOH (2 mL) were added to the crude product and this mixture extracted twice with 10 mL EtOAc. The combined organic layers were washed with brine, dried over Na₂SO₄ and concentrated in vacuo yielding 410 mg pale yellow oil. The crude product was purified by flash chromatography (heptane/EtOAc 70:30) to give 75 mg colourless oil. LRMS: observed 480 [M+H], calculated 480.56 [M+H].

### EXAMPLE 594

### (3-exo)-3-({[6-(3-Fluorophenyl)-pyridin-3-yl]carbonyl}amino)-N-methyl-8-azabicyclo[3.2.1]octane-8-carboxamide

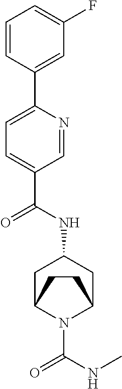

A solution of N-((1R,3s,5S)-8-azabicylo[3.2.1]octan-3-yl)-6-(3-fluorophenyl)nicotinamide (Example 582, 125 mg, 0.384 mmol) and diisopropylethylamine (0.074 mL) in anhydrous tetrahydrofuran (2 mL) was added dropwise to a stirred, ice-cold solution of triphosgene (57 mg, 0.192 mmol) in anhydrous tetrahydrofuran (2 mL) and after the addition was complete the reaction mixture was stirred at room temperature for 1 hour. A solution of 2.0 M methylamine in tetrahydrofuran (0.96 mL, 1.921 mmol) was then added and the reaction mixture was stirred over night at room temperature. The reaction mixture was diluted with methanol (5 mL), silica (60-200 μm, approximately 1 g) was added and the solvent was removed in vacuo. The absorbed material was purified on flash silica eluting with a dichloromethane/methanol eluant in a gradient from 100:0 to 98:2 by volume to give the title compound as an oil which solidified. This crude product was dissolved in dichloromethane (2 mL) and triturated by the slow addition of diethyl ether (25 mL). The suspension which formed was stirred for 5 min and then the solid was filtered off, washed with diethyl ether (25 mL) and dried to give a beige powder, 79 mg.

LRMS (m/z): obs 383 [M+1]; calc 383.2 [M+1].

¹HNMR (DMSO-d₆): 1.63-1.80 (m, 6H), 1.81-2.05 (m, 2H), 2.55-2.70 (m, 3H), 4.20 (bs, 2H), 4.35-4.51 (m, 1H), 6.40-6.51 (m, 1H), 7.30-7.40 (m, 1H), 7.50-7.60 (m, 1H), 7.79-8.12 (m, 1H), 8.10-8.20 (m, 1H), 8.25-8.35 (m, 1H), 8.45-8.55 (m, 1H), 9.05-9.10 (m, 1H).

### EXAMPLE 595

### (3-exo)-3-({[6-(3-Fluorophenyl)-pyridin-3-yl]carbonyl}amino)-N,N-dimethyl-8-azabicyclo[3.2.1]octane-8-carboxamide

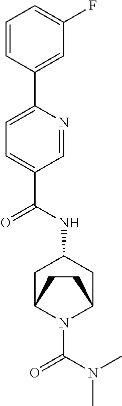

The title compound was prepared in a similar way to Example 594 but using a solution of 2M dimethylamine in tetrahydrofuran (0.96 mL, 1.921 mmol) instead of methylamine. The title compound was isolated by chromatography on flash silica eluting with a dichloromethane:methanol eluant in a gradient from 100:0 to 96:4 by volume. The title compound was isolated as an oil which solidified. This crude product was dissolved in dichloromethane (2 mL) and triturated by the slow addition of diethyl ether (25 mL). The suspension which formed was stirred for 5 minutes and then the solid was filtered off, washed with diethyl ether (25 mL) and dried to give a white powder, 84 mg.

LRMS (m/z): obs 397 [M+1]; calc 397.46 [M+1].

¹HNMR (DMSO-d₆): 1.65-1.89 (m, 8H), 2.82 (s, 6H), 4.00-4.09 (bs, 2H), 4.34-4.44 (m, 1H), 7.34-7.44 (m, 1H), 7.52-7.59 (m, 1H), 7.90-8.05 (m, 1H), 8.10-8.19 (m, 1H), 8.25-8.30 (m, 1H), 8.50-8.60 (m, 1H), 9.05-9.10 (m, 1H).

### EXAMPLE 596

### 6-(3-Fluorophenyl)-N-(3-exo)-8-[(4-hydroxypiperidin-1-ylcarbonyl]-8-azabicyclo[3.2.1]octyl-3-yl}nicotinamide

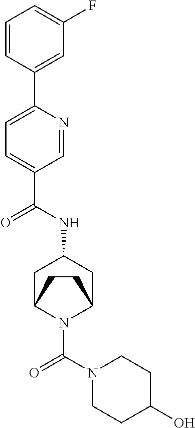

The title compound was prepared in a similar way to Example 594 but using a solution of 4-hydroxypiperidine (194 mg, 1.921 mmol) in tetrahydrofuran (1 mL) instead of methylamine. The title compound was isolated by chromatography on flash silica eluting with a dichloromethane:methanol eluant in a gradient from 100:0 to 90:10 by volume. The title compound was isolated as an oil which solidified. This crude product was dissolved in dichloromethane (2 mL) and triturated by the slow addition of diethyl ether (25 mL). The suspension which formed was stirred for 5 minutes and then the solid was filtered off, washed with diethyl ether (25 mL) and dried to give a pale yellow powder, 102 mg.

LRMS (m/z): obs 453 [M+1]; calc 453.52 [M+1].

¹HNMR (DMSO-d₆): 1.20-1.35 (m, 2H), 1.65-1.90 (m, 10), 2.89-3.01 (m, 2H), 3.50-3.69 (m, 3H), 3.95-4.02 (bs, 2H), 4.25-4.42 (m, 1H), 4.70-4.78 (m, 1H), 7.29-7.36 (m, 1H), 7.50-7.60 (m, 1H), 7.91-8.01 (m, 1H), 8.10-8.20 (m, 1H), 8.20-8.30 (m, 1H), 8.46-8.56 (m, 1H), 9.05-9.10 (m, 1H).

### EXAMPLE 597

### (3-exo)-3-({[6-(3-Fluorophenyl)-pyridin-3-yl]carbonyl}amino)-N-(2-hydroxyethyl)-8-azabicyclo[3.2.1]octane-8-carboxamide

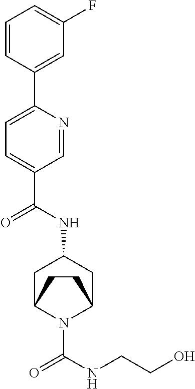

The title compound was prepared in a similar way to Example 594 but using a solution of 2-aminoethanol (117 mg, 1.921 mmol) in tetrahydrofuran (1 mL) instead of methylamine. The title compound was isolated by chromatography on flash silica eluting with a dichloromethane:methanol eluant in a gradient from 100:0 to 90:10 by volume. The title compound was isolated as an oil which solidified. This crude product was dissolved in dichloromethane (2 mL) and triturated by the slow addition of diethyl ether (25 mL). The resulting suspension was stirred for 5 minutes and then the solid was filtered off, washed with diethyl ether (25 mL) and dried to give a white powder, 87 mg.

LRMS (m/z): obs 413 [M+1]; calc 413.46 [M+1].

¹HNMR (DMSO-d₆): 1.60-1.75 (m, 6H), 1.85-1.95 (m, 2H), 3.05-3.15 (m, 2H), 3.35-3.46 (m, 2H), 4.18-4.25 (bs, 2H), 4.35-4.42 (m, 1H), 4.62-4.70 (m, 1H), 6.40-6.50 (m, 1H), 7.28-7.35 (m, 1H), 7.50-7.60 (m, 1H), 7.92-8.00 (m, 1H), 8.10-8.17 (m, 1H), 8.22-8.28 (m, 1H), 8.45-8.52 (m, 1H), 9.05-9.10 (m, 1H).

The following section describes the synthesis of intermediates which were used in the preparation of the foregoing examples.

### Preparation 1

### 6-(3-Fluorophenyl)nicotinic acid

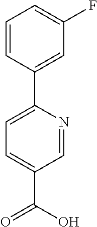

3-Fluorophenylboronic acid (39.5 g, 0.282 mol), a solution of K₂CO₃ (150 g) in water (700 mL), [Bu₄N]Br (3.5 g, 0.0107 mol), and Pd(PPh₃)₄ (12.4 g, 0.0107 mol) were added to a solution of 6-chloronicotinic acid (37.0 g, 0.235 mol) in toluene. The reaction mixture was stirred under reflux for 20 hours. After cooling, the reaction mixture was filtered and acidified with 2 M HCl to pH 3. The precipitate which formed was separated by filtration and dried to give 6-(3-fluorophenyl)nicotinic acid (49.9 g). ¹H NMR (400 MHz, DMSO-d₆) δ ppm 7.29 (td, J=8.46, 2.42 Hz, 1H) 7.50-7.56 (m, 1H) 7.93 (dd, J=10.47, 2.15 Hz, 1H) 7.97 (d, J=7.79 Hz, 1H) 8.11 (d, J=8.06 Hz, 1H) 8.30 (dd, J=8.32, 2.15 Hz, 1H) 9.11 (d, J=1.88 Hz, 1H), 13.48 (bs, 1H).

### Preparation 2

### 5-Chloro-6-(3-fluorophenyl)nicotinic acid

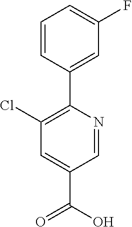

To a round bottom flask was added 5,6-dichloronicotinic acid (500 mg, 2.60 mmol), 3-fluorophenylboronic acid (364 mg, 2.60 mmol), DMF (25 mL), 2M Cs₂CO₃ (6 mL) and Pd(Ph₃)₄ (30.1 mg, 0.026 mmol). The reaction mixture was heated to 90° C. for 3 hours and then allowed to cool to room temperature. The mixture was diluted with ethyl acetate/water and the layers were separated. The organic layer was washed with brine, dried (MgSO₄) and evaporated to give a solid, which was purified by chromatography (silica, DCM/MeOH) to give the desired product, 5-chloro-6-(3-fluorophenyl)nicotinic acid (623 mg, 95%). LRMS observed 252 [M+H], calc 252.02 [M+H]

### Preparation 3

### 6-(3,5-Difluoro-phenyl)-nicotinic acid

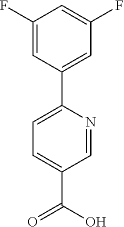

Step A: Preparation of tert-butyl 6-bromonicotinate To a round bottom flask containing 2-bromo-5-pyridinecarboxylic acid (10.0 g, 49 mmol) in DCM (500 mL) were added oxalyl bromide (7.4 mL) and 5 drops of DMF. After some gas evolution, the reaction mixture was stirred at reflux for approximately 6 hours, then cooled to room temperature and heptane (100 mL) was added, followed by concentration of the mixture. The mixture was then suspended in THF (400 mL) and cooled to 0° C. t-BuOK (5.8 g, 52 mmol) was added and the reaction was allowed to warm to room temperature and stirred for 2 hours. The mixture was poured into EtOAc, washed with 1 N NaOH, water and brine, dried over MgSO₄, filtered and concentrated. The residue was purified by silica gel chromatography on a Biotage™ 40S (Heptane EtOAc 0-80%, 3 L) to afford the title compound 4.2 g (36%) as a white solid. ¹H NMR (400 MHz, DMSO-d₆) δ ppm 8.78-8.86 (1H, m), 8.14 (1H, dd, J=8.4, 2.4 Hz), 7.81 (1H, d, J=8.4 Hz), 1.56 (9H, s).

Step B: Preparation of tert-butyl 6-(3,5-difluorophenyl)nicotinate To a round-bottom flask was added 3,5-difluoro phenylboronic acid (1.84 g, 11.6 mmol), palladium tetrakis(triphenylphosphine) (89.5 mg, 0.08 mmol) and tert-butyl 6-bromonicotinate (2.0 g, 7.75 mmol) and the mixture was evacuated 3 times with N₂. The solids were dissolved in DMF (50 mL), followed by addition of 2M cesium carbonate (11 mL). The resulting mixture was heated to ˜90° C. until no starting bromide material was apparent by HPLC. The mixture was cooled to room temperature and then poured into a separating funnel, followed by addition of EtOAc and water (1×200 mL). The layers were separated and the organic extract was washed with brine (1×200 mL), dried over MgSO₄, filtered and concentrated to afford an orange oil. The crude mixture was purified by silica gel column chromatography on Biotage™ (silica, 2-10% EtOAc in Heptane, 2.5 L) to afford the title compound 2.1 g (93%) as a white solid. ¹H NMR (400 MHz, DMSO-d₆) δ ppm 9.10-9.14 (1H, m), 8.29-8.35 (1H, m), 8.20-8.25 (1H, m), 7.90 (2H, dd, J=9.0, 1.5 Hz), 7.42 (1H, s), 1.59 (9H, s).

Step C: Preparation of 6-(3,5-difluoro-phenyl)-nicotinic acid To tert-butyl 6-(3,5-difluorophenyl)nicotinate in DCM (80 mL) was added trifluoroacetic acid (20 mL). After stirring at room temperature overnight, toluene was added (100 mL) and the solvent was removed to give the crude product as a white powder. The solid was re-crystallized from MeOH to afford the title compound 1.269 g (74%) as a white solid. ¹H NMR (400 MHz, DMSO-d₆) δ ppm 9.16 (1H, d, J=1.7 Hz), 8.37 (1H, dd, J=8.2, 2.0 Hz), 8.23 (1H, d, J=8.2 Hz), 7.86-7.95 (2H, m), 7.36-7.47 (1H, m).

### Preparation 4

### 6-(5-Fluoro-2-hydroxyphenyl)nicotinic acid

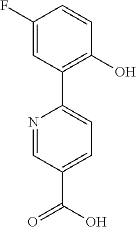

Step A: Methyl 6-(5-fluoro-2-hydroxyphenyl)nicotinate To a degassed mixture of 1,4-dioxane (12 mL) and water (3 mL) was added (5-fluoro-2-hydroxyphenyl)boronic acid (0.781 g, 5.0 mmol), methyl 6-chloronicotinate (0.86 g, 5.0 mmol), potassium carbonate (2.08 g, 15.0 mmol) and tetrakis(triphenylphosphine)palladium(0) (0.29 g, 0.05 mmol) and the resulting mixture was stirred at 80° C. for 2 hours. After this time additional tetrakis(triphenylphosphine)palladium(0) (0.29 g, 0.05 mmol) was added and heating was continued at 80° C. for a further 3 hours. The mixture was then stirred at room temperature overnight. The solvent was evaporated in vacuo and the residue was suspended in ethyl acetate (50 mL). The suspension was filtered through a plug of Arbocel™ and the filtrate was concentrated in vacuo. The resulting residue was dissolved in ethyl acetate (100 mL) and washed with saturated aqueous sodium carbonate (3×100 mL). The aqueous washings were combined and extracted with ethyl acetate (3×50 mL). The ethyl acetate layers were combined, dried with anhydrous MgSO₄ and concentrated in vacuo to afford a solid which was re-crystallised from dichloromethane/heptane to afford the title compound as a yellow solid (0.71 g) (57%). ¹H NMR (400 MHz, CDCl₃) δ ppm 9.14 (1H, s), 8.46-8.40 (1H, m), 7.91-7.86 (1H, m), 7.53-7.46 (1H, m), 7.11-7.03 (1H, m), 7.02-6.96 (1H, m), 3.99 (3H, s). LRMS: AP m/z 248 [M+H]⁺.

Step B: 6-(5-Fluoro-2-hydroxyphenyl)nicotinic acid Methyl 6-(5-fluoro-2-hydroxyphenyl)nicotinate (1.47 g, 6.0 mmol) was dissolved in MeOH (35 mL) and cooled to 0° C. Lithium hydroxide (0.71 g, 30.0 mmol) was then added and the mixture was stirred at 0° C. for 0.5 hours. The mixture was then allowed to warm to room temperature. Additional lithium hydroxide (0.43 g, 18.0 mmol) was added and the reaction mixture was allowed to stir at room temperature for 72 hours. The mixture was then concentrated in vacuo and the resulting yellow solid was dissolved in water (150 mL). The solution was acidified to pH 1 by addition of 1N aqueous HCl and the resulting precipitate was filtered and washed with 0.5M aqueous HCl to afford the title compound as a yellow powder (1.15 g) (72%). ¹H NMR (400 MHz, DMSO-d6) δ ppm 9.11 (1H, s), 8.42-8.28 (2H, m) 7.94-7.84 (1H, m), 7.26-7.15 (1H, m), 7.02-6.92 (1 H, m). LRMS: ES m/z 234 [M+H]⁺.

### Preparation 5

### trans-tert-Butyl 3-({[6-(3-fluorophenyl)pyridin-3-yl]carbonyl}amino)-4-hydroxypyrrolidine-1-carboxylate

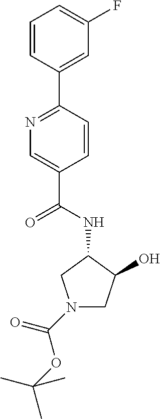

To a solution of 6-(3-fluorophenyl)nicotinic acid (391 mg, 1.8 mmol) in DMF (10 mL) at 0° C. was added HATU (753 mg, 1.98 mmol) and DIPEA (0.47 mL, 2.07 mmol). After 15 min, trans-tert-butyl 3-amino-4-hydroxypyrrolidine-1-carboxylate was added and the reaction mixture was stirred at room temperature for 5 hours. The solvent was removed in vacuo and the residue was diluted with ethyl acetate and water. The layers were separated and the organic layer was washed with brine, dried (MgSO₄) and evaporated to give an oil. Purification by chromatography (silica, 65% ethyl acetate:hexane) gave the desired product, trans-tert-butyl-3-({[6-(3-fluorophenyl)pyridin-3-yl]carbonyl}amino)-4-hydroxypyrrolidine-1-carboxylate (420 mg, 58%). LC/MS (M+H)=401.9 observed, 402.18 calc.

### Preparation 6

### trans-6-(3-Fluorophenyl)-N-[4-hydroxypyrrolidin-3-yl]nicotinamide

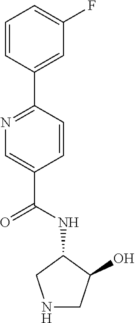

To a solution of trans-tert-butyl 3-({[6-(3-fluorophenyl)pyridin-3-yl]carbonyl}amino)-4-hydroxypyrrolidine-1-carboxylate (500 mg, 1.24 mmol) in dioxane was added a solution of 4N HCl in dioxane (10 mL). The reaction was stirred at room temperature for ˜4 hours and then diluted with ether to give a white solid, which was filtered and collected to give the desired product as the hydrochloride salt, trans-6-(3-fluorophenyl)-N-[4-hydroxypyrrolidin-3-yl]nicotinamide (390 mg, 92%). LC/MS (M+H)=301.9 observed, 302.13 calc.

### Preparation 8

### tert-Butyl 2-(2-isopropoxyethylamino)ethylcarbamate

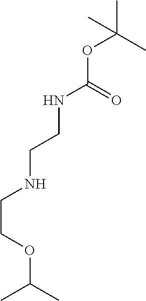

A solution of tert-butyl 2-bromoethylcarbamate (900 mg, 4.02 mmol) in 5 ml N,N-dimethylformamide was added dropwise to a suspension of 2-isopropoxyethanamine (829 mg, 8.03 mmol) and KI (133 mg, 0.803 mmol) in 5 ml N,N-dimethylformamide at room temperature and under an inert atmosphere. The reaction mixture was and stirred for 72 hours at 45° C. Water (20 mL) was added and the reaction mixture was extracted twice with Et₂O (20 mL). The combined organic layers were washed with 20 mL 0.5 M HCl and brine. The combined acidic aqueous layers were neutralized with saturated Na₂CO₃ and extracted with 20 mL Et₂O. The resulting organic phase was washed with brine, dried over Na₂SO₄ and concentrated in vacuo, yielding 400 mg of a colourless oil (1.624 mmol, 40% yield).

¹H NMR (CDCl₃, 400 MHz) δ ppm 1.152-1.167 (m, 6H) 1.447 (s, 9H) 3.343-3.602 (m, 7H) 4.132-4.145 (m, 2H) 4.795-4.885 (br m, 1H) 5.100-5.150 (br m, 1H)

### Preparation 9

### Benzyl 2-tert-butoxycarbonylaminoethyl(2-isopropoxyethyl) carbamate

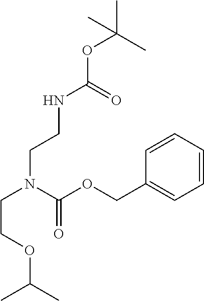

Benzyl chloroformate (305 mg, 1.786 mmol) was added dropwise to a stirred solution of tert-butyl 2-(2-isopropoxyethylamino)ethylcarbamate (400 mg, 1.624 mmol) and triethylamine (0.272 ml, 1.948 mmol) in dichloromethane (10 mL). The reaction mixture was stirred for 18 hours after which TLC (Heptane/EtOAc 1:1+1% NH3 in MeOH) showed complete conversion to a new compound. The reaction mixture was diluted with EtOAc (30 mL) and washed with water (30 mL) and brine (30 mL), dried over Na₂SO₄ and concentrated in vacuo, yielding 460 mg of a colourless oil (1.209 mmol, 75% yield).

¹H NMR (CDCl₃, 400 MHz) δ ppm 1.122-1.200 (m, 6H) 1.428 (s, 9H) 3.316-3.613 (m, 9H) 5.134-5.143 (m, 2H) 5.350-5.400 (m, 1H) 7.322-7.366 (m, 5H).

### Preparation 10

### Benzyl 2-aminoethyl(2-isopropoxyethyl)carbamate

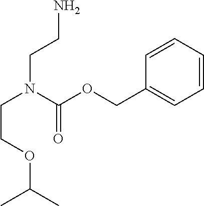

A solution of benzyl 2-tert-butoxycarbonylaminoethyl(2-isopropoxyethyl) carbamate (460 mg, 1.209 mmol) in trifluoroacetic acid (20 mL, 260 mmol) was stirred at temperature for 2 hours and subsequently concentrated in vacuo yielding 460 mg of an oil (1.641 mmol, 136% yield, still contains residual trifluoroacetic acid). The product was used without further purification.

LRMS: observed 281 [M+H], calculated 281.37 [M+H].

### Preparation 12

### 1-(4-Chlorobenzyl)-3-aminopyrrolidin-2-one

### Step 1. Preparation of 2,4-dibromo-butyryl chloride

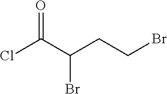

A mixture of compound γ-butyrolactone (200 g, 2.32 mol) and PBr₃ (4 mL) was heated at 100° C., and Br₂ (100 mL) was added slowly below the surface of the reaction mixture while keeping the reaction temperature at 110˜115° C. DMF (0.2 mL) was added at 50° C., and then SOCl₂ (200 mL) was added dropwise at 90° C. Stirring was continued for a further 3 hours. The mixture was distilled and the fraction boiling at 42˜44° C. (5 mmHg) was collected to yield 323 g, (52.6%) of 2,4-dibromo-butyryl chloride as a yellow liquid. 1H NMR (400 MHz CDCl3) δ ppm 2.49-2.73 (m, 2H), 3.60 (m, 2H), 4.83 (m, 1H).

### Step 2. Preparation of N-(4-chlorobenzyl)-2,4-dibromobutanamide

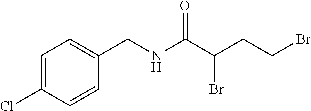

To a stirred solution of 4-chlorobenzylamine (250 g, 1.77 mol) and Et₃N (232 g, 2.29 mol) in anhydrous dichloromethane (3 L) was added, dropwise, 2,4-dibromo-butyryl chloride (552 g, 2.13 mol) at 0° C. Two hours later, TLC (EtOAc/Petroleum ether=1:1) showed that the material was consumed completely. The mixture was washed with water (1 L×2), and the organic layer was separated, dried over Na₂SO₄ and evaporated to give 508 g (78%) of N-(4-chlorobenzyl)-2,4-dibromobutanamide as a brown syrup, which was used for the following step without further purification.

¹H NMR (400 MHz CDCl₃) δ ppm 2.40-2.80 (m, 2H), 3.58 (m, 2H), 4.38-4.61 (m, 3H), 7.20-7.40 (m, 4H).

### Step 3. Preparation of 1-(4-chlorobenzyl)-3-bromopyrrolidin-2-one

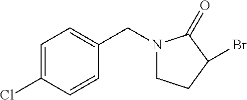

To a stirred suspension of NaH (84 g, 2.1 mol) in absolute THF (4 L) was added dropwise a solution of N-(4-chlorobenzyl)-2,4-dibromobutanamide (505 g, 1.38 mol) in absolute THF (1500 mL) at 0° C. After the addition, the reaction mixture was allowed to warm to room temperature and stirred overnight. TLC (EtOAc/Petroleum ether=1:5) showed that the material was consumed completely. The reaction mixture was filtered and the filtrate was concentrated in vacuo to give crude 1-(4-chlorobenzyl)-3-bromopyrrolidin-2-one (260 g, 66%) as a black liquid, which was used for the following step without further purification.

### Step 4. Preparation of 1-(4-chlorobenzyl)-3-aminopyrrolidin-2-one

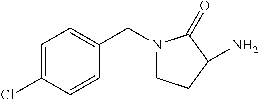

Ammonia (1250 mL) was added to a solution of 1-(4-chlorobenzyl)-3-bromopyrrolidin-2-one (260 g, 0.94 mol) in acetonitrile (2 L). The mixture was stirred at room temperature overnight. TLC (MeOH/CH₂Cl₂=1:15) showed that the material was consumed completely and the mixture was evaporated in vacuo. The crude product (180 g, 92%) was purified by column chromatography (CH₂Cl₂) to give crude 1-(4-chlorobenzyl)-3-aminopyrrolidin-2-one (108 g, 55%) as a brown liquid. The amino group of this crude compound was protected as the tert-butyl carbamate derivative and was purified using column chromatography. This pure material was deprotected with 4 M HCl in MeOH to afford the corresponding salt, which was then basified to obtain 1-(4-chlorobenzyl)-3-aminopyrrolidin-2-one (50 g, 25.6%) as a brown oil.

LRMS: observed 225 [M+H], calc 225.69 [M+H].

### Preparation 13

### 3-Amino-1-(4-methyl-benzyl)-pyrrolidin-2-one

### Step 1. Preparation of 2-tert-butoxycarbonylamino-4-methyl sulfanyl-butyric acid

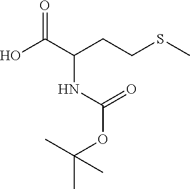

To a suspension of methionine (161 g, 1.081 mol) in dioxane (2.5 L) and water (2.5 L), an aqueous solution of NaOH (78 g, 1.95 mol) in water (500 mL) was added. Then, di-tert-butyl dicarbonate (306 g, 1.4 mol) was added to the reaction mixture dropwise at 0° C. The reaction mixture was stirred for 12 hours at room temperature. The dioxane was evaporated off and the residue was diluted with ethyl acetate (1×1 L). The organic phase was separated, dried over anhydrous Na₂SO₄ and evaporated in vacuo. The crude product was purified by column chromatography on silica gel (100-200 mesh) eluting with 10% EtOAc in hexane to give the compound as a colourless liquid (215 g, 80%).

### Step 2: Preparation of [1-(4-methyl-benzylcarbamoyl)-3-methylsulfanyl-propyl]-carbamic acid tert-butyl ester

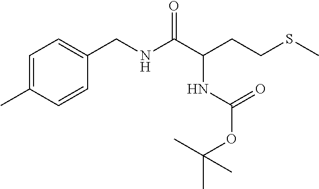

To a stirred solution of 2-tert-butoxycarbonylamino-4-methyl sulfanyl-butyric acid (212 g, 0.851 mol) in dry DCM (4 L), under nitrogen atmosphere, cooled to 0° C. (ice-bath), were added anhydrous HOBT (150 g, 1.11 mol), EDCI (213 g, 1.11 mol), N,N di-isopropyl ethyl amine (220 g, 1.702 mol) and 4-methyl benzyl amine (108 g, 0.894 mol). The reaction mixture was stirred for 18 hours at room temperature. The reaction was quenched with ice cold 1N HCl (aq) (1×250 ml). The organic phase was separated, washed with saturated sodium bicarbonate solution and brine and dried over sodium sulphate. The crude product was crystallized with CH₂Cl₂:ether (2:8) to yield the product as white solid (180 g, 60%).

### Step 3: Preparation of [1-(4-methyl-benzyl)-2-oxo-pyrrolidin-3-yl]-carbamic acid tert-butyl ester

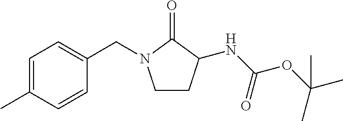

[1-(4-Methyl-benzylcarbamoyl)-3-methylsulfanyl-propyl]-carbamic acid tert-butyl ester (175 g, 0.497 mol) was dissolved in iodomethane (690 g, 4.94 mol) and the solution was stirred under a nitrogen atmosphere for 48 hours. The iodomethane was removed by distillation under reduced pressure to give the sulfonium salt as a yellow solid (213 g, 0.433 mol, 88%). This was stirred in dry THF (4 L), under nitrogen, at 0° C. (ice-bath) and lithium bis(trimethylsilyl)amide (1.0M in THF, 431 mL, 0.431 mol) was added dropwise. The reaction mixture was stirred at this temperature for 3 hours. Then the reaction mixture was quenched with saturated aqueous ammonium chloride (200 mL) and most of the THF was removed under reduced pressure. The residual solvent was partitioned between aqueous NaHCO₃ and CH₂Cl₂. The aqueous layer was further extracted with CH₂Cl₂. The combined organic phases were dried over sodium sulphate, filtered and concentrated in vacuo. The crude product was crystallized from CH₂Cl₂:Ether (2:8) to yield the product as white solid (92 g, 60%).

### Step 4: Preparation of 3-Amino-1-(4-methyl-benzyl)-pyrrolidin-2-one hydrochloride salt

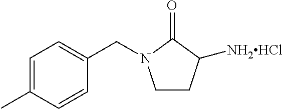

Dry HCl gas was passed over a solution of [1-(4-methyl-benzyl)-2-oxo-pyrrolidin-3-yl]-carbamic acid tert-butyl ester (90 g, 0.296 mol) in dry DCM (1.5 L) at 0° C. (ice-bath) for 1 hour. The solution was concentrated in vacuo to yield the desired compound as the hydrochloride salt (57 g, 80%). MS: observed 205.4 [M+H], calculated 205.3 [M+H].

### Preparation 14

### [1-(6-Methyl-5,6,7,8-tetrahydropyrido[4,3-d]pyrimidin-2-yl)piperidin-3-yl]methylamine trihydrochloride

### Step 1. Preparation of 3-[(dimethylamino)methylene]-1-methylpiperidin-4-one

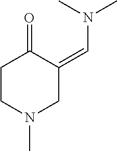

1-Methylpiperidin-4-one (48 g, 0.425 mol) and N,N-dimethylformamide dimethyl acetal (61 g, 0.513 mol) in o-xylol (350 mL) and K₂CO₃ (27 g) were heated at (140-150° C.) with continual removal of the volatile fraction (mainly methanol) with boiling point 64-65° C. until the boiling point of the volatile fraction began to increase (˜2.5 h). The reaction was mixture was then cooled to RT, filtered and evaporated to give the title compound as a red oil (50.4 g).

### Step 2. Preparation of tert-butyl [(1-benzylpiperidin-3-yl)methyl]carbamate

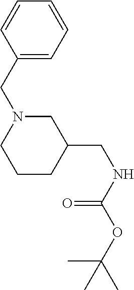

A solution of [(1-benzylpiperidin-3-yl)methyl]amine (377.3 g, 1.85 mol), di-tert-butyl dicarbonate (403.2 g, 1.85 mol) and triethylamine (257.3 ml, 1.85 mol) in acetonitrile (400 mL) was stirred for 12 hours at room temperature. The mixture was then evaporated and the residue was stirred with hexane (500 mL). The precipitate which formed was filtered, washed with hexane, and dried to give the title compound (528.4 g).

### Step 3. Preparation of tert-butyl (piperidin-3-ylmethyl)carbamate

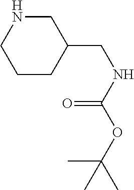

tert-Butyl [(1-benzylpiperidin-3-yl)methyl]carbamate (251 g) was hydrogenated (80 psi) in methanol (1 L) in the presence of 5% Pd/C (50 g) for 10 hours. The mixture was filtered through celite, the filtrate was evaporated and the residue was stirred with hexane. The precipitate which formed was filtered, washed with hexane, and dried to give the title compound (156.5 g).

### Step 4. Preparation of tert-butyl ({1-[amino(imino)methyl]piperidin-3-yl}methyl)carbamate

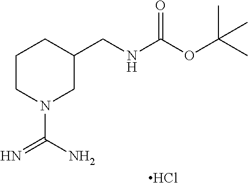

A solution of tert-butyl (piperidin-3-ylmethyl)carbamate (324.0 g, 1.5 mol), 1H-pyrazole-1-carboximidamide hydrochloride (221.8 g, 1.5 mol) and diisopropylethylamine (263.2 mL, 1.5 mol) in DMF (700 mL) was stirred for 48 h at room temperature. Then the mixture was evaporated until dry, the residue was stirred with ether and the formed precipitate filtered, washed with ether and dried to give the title compound (435.9 g).

### Step 5. Preparation of tert-butyl {[1-(6-methyl-5,6,7,8-tetrahydropyrido[4,3-d]pyrimidin-2-yl)piperidin-3-yl]methyl}carbamate

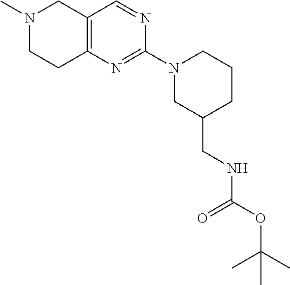

A suspension of tert-butyl ({1-[amino(imino)methyl]piperidin-3-yl}methyl)carbamate (50 g, 0.17 mol), 3-[(dimethylamino)methylene]-1-methylpiperidin-4-one (29 g. 0.17 mol), and sodium methoxide (13.5 g, 0.25 mol) in absolute ethanol (500 mL) was refluxed for 8 hours. The reaction mixture was evaporated and the residue was stirred with water. The precipitate which formed was filtered, washed with water and ether, and dried to give the title compound (46.5 g).

### Step 6. Preparation of [1-(6-Methyl-5,6,7,8-tetrahydropyrido[4,3-d]pyrimidin-2-yl)piperidin-3-yl]methylamine trihydrochloride

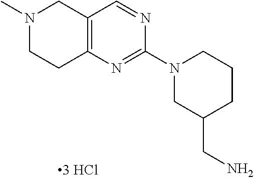

tert-Butyl ({1-[amino(imino)methyl]piperidin-3-yl}methyl)carbamate (46.5 g, 0.177 mol) was added to a solution of methanol (50 mL) and 4 N HCl solution in dioxane (250 mL). The mixture was stirred at room temperature for 12 hours and evaporated and the residue was purified by chromatography to give the title compound (23.1 g).

¹H NMR (DMSO-d₆, 400 MHz) δ ppm 1.20-1.44 (m, 2H), 1.68-1.82 (m, 3H), 2.65-2.89 (m, 6H), 2.96-3.20 (m, 1H), 3.21-3.40 (m, 1H), 3.31-3.46 (m, 1H), 3.55-3.68 (m, 1H), 4.05-4.12 (m, 1H), 4.22-4.35 (m, 1H), 4.37-4.45 (m, 1H), 4.51-4.59 (m, 1H), 8.15 (b, 2H), 8.23 (s, 1H). LCMS gave [M+H]⁺=371.

### Preparation 15

### 2-[5-(2-Methoxy-phenyl)-[1,3,4]oxadiazol-2-yl]-ethylamine

### Step 1. Preparation of N-Boc-β-Alanine-methyl ester

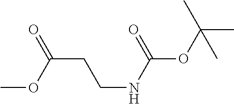

To a solution of β-Alanine methyl ester hydrochloride (710 g, 5.07 mol) in methanol (2000 mL) was added freshly distilled triethylamine (750 mL, 545 g, 5.4 mol) with vigorous stirring. The reaction mixture was cooled in an ice bath during the addition of triethylamine. Di-tert-butyl dicarbonate was then added to the mixture in portions (50 g at a time, 1110 g, 5.1 mol total) and the reaction was stirred for 12 hours. The mixture was concentrated to half its volume under reduced pressure, and triethylammonium hydrochloride was filtered from solution, washing with chloroform (500 mL). The filtrate was diluted with chloroform (2000 mL), and the mixture was washed with water (2500 mL), and then with 10% w/w aqueous citric acid (2500 mL). The organic layer was evaporated in vacuo to give N-Boc-β-Alanine-methyl ester as a transparent colourless oil (1030 g). The product was used in the next stage without further purification.

### Step 2. Preparation of N-Boc-β-Alanine hydrazide

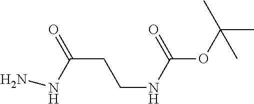

To N-Boc-β-Alanine-methyl ester (1030 g) in isopropanol (1500 mL) was added hydrazine hydrate (1000 mL, 1032 g, 20 mol) and the mixture was refluxed with a reflux condenser for 16 hours. The reaction mixture was evaporated to dryness and redissolved in chloroform (2000 mL). The solution was then washed with water (2000 mL), dried over sodium sulfate, and evaporated to dryness. The product was crystallized from diethyl ether (2000 mL), filtered, and dried under vacuum to give N-Boc β-Alanine hydrazide (771 g).

### Step 3. Preparation of {2-[5-(2-methoxy-phenyl)-[1,3,4]oxadiazol-2-yl]-ethyl}-carbamic acid tert-butyl ester

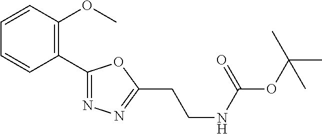

A mixture of 2-methoxybenzoic acid (34.65 g, 0.228 mol), triphenylphosphine (179.2 g, 0.684 mol) and triethylamine (73.73 g, 0.73 mol) in anhydrous acetonitrile (900 mL) was stirred under an argon atmosphere for 10-15 minutes and cooled to 0° C. Anhydrous carbon tetrachloride (139.1 mL) was added, and the mixture was stirred for another 15 minutes at this temperature. N-Boc-β-Alanine hydrazide (46.28 g, 0.228 mol) was added as one portion and the mixture was stirred for 15 minutes with the temperature maintained at <5° C. The ice bath was removed, and the mixture was stirred at room temperature for 3 hours. The precipitate which formed was filtered and washed with acetonitrile (1000 mL). Solvent was removed in vacuo, and the residue re-dissolved in ethyl acetate (100 mL). The mixture was stirred with slight heating for 15 minutes. The residue was filtered off and washed with ethyl acetate. The filtrate was concentrated under reduced pressure and purified by column chromatography eluting with ethyl acetate to give the title compound as a light-yellow viscous oil.

### Step 4. Preparation of 2-[5-(2-Methoxy-phenyl)-[1,3,4]oxadiazol-2-yl]-ethylamine

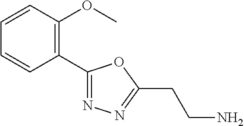

{2-[5-(2-Methoxy-phenyl)-[1,3,4]oxadiazol-2-yl]-ethyl}-carbamic acid tert-butyl ester) was dissolved in absolute methylene chloride (400 mL) and cooled in an ice water bath. Trifluoroacetic acid (140 mL) was added and the reaction mixture was stirred at ambient temperature for 20 hours. The solvent and the most of the trifluoroacetic acid were removed in vacuo, water was added and the resulting mixture was extracted with benzene. The aqueous layer was saturated with potassium carbonate to alkaline pH and extracted three times with chloroform (500 mL). The combined organic phases were dried over anhydrous sodium sulfate, concentrated in vacuo and purified by column chromatography, eluting with chloroform-methanol-triethylamine, 10:1:1, to give 30.0 g (60%) of the title compound as a free base.

LCMS (ES): observeds 220.2 (M+1), calculated 220.25 [M+1].

¹H NMR (400 MHz d₆-DMSO) δ ppm 2.92-2.93 (m, 4H), 3.87 (s, 3H), 7.09-7.14 (m, 1H), 7.24-7.27 (m, 1H), 7.56-7.61 (m, 1H), 7.78-7.81 (m, 1H).

### Preparation 16

### 2-(2-Aminoethyl)-1-ethyl-N-(2-methoxyethyl)-1H-benzimidazole-5-carboxamide

### Step 1. Methyl 3-{[N-(tert-butoxycarbonyl)-beta-alanyl]amino}-4-(ethylamino)benzoate

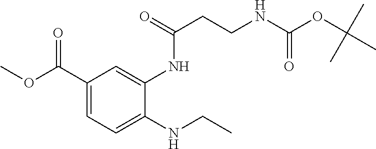

EDC (560 g, 3.61 mol) was added to a mixture of 3-N-tert-butyloxycarbonylaminopropionic acid (487.6 g, 2.58 mol) and HOBt (487 g, 3.61 mol) in CH₂Cl₂ (5 L). The resulting mixture was stirred at room temperature for 1 hour. 3-Amino-4-ethylaminobenzoic acid methyl ester (prepared according to the method of Bioorganic & Medicinal Chemistry, 13(5), 2005, 1587-1597, 500 g, 2.58 mol) was added and the mixture was stirred at room temperature overnight.

The mixture was washed with saturated aq. NH₄Cl (10 L) and brine, dried over Na₂SO₄ and concentrated in vacuo to afford the required product, methyl 3-{[N-(tert-butoxycarbonyl)-beta-alanyl]amino}-4-(ethylamino)benzoate (1200 g, 100%) as a grey solid.

### Step 2. Methyl 2-{2-[(tert-butoxycarbonyl)amino]ethyl}-1-ethyl-1H-benzimidazole-5-carboxylate

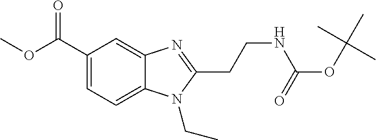

para-Toluene sulfonic acid (471 g, 2.74 mol) was added to a mixture of methyl 3-{[N-(tert-butoxycarbonyl)-beta-alanyl]amino}-4-(ethylamino)benzoate (1000 g, 2.74 mol) and MeOH (15 L). The resulting mixture was heated to reflux for 4 hours. Most of the solvent was removed in vacuo and the residue was poured into saturated aqueous Na₂CO₃ (40 L). The resulting mixture was filtered and the filter cake was washed with petroleum ether to give methyl 2-{2[(tert-butoxycarbonyl)amino]ethyl}-1-ethyl-1H-benzimidazole-5-carboxylate (700 g, 73.6%) as a grey solid.

### Step 3. 2-{2-[(tert-Butoxycarbonyl)amino]ethyl}-1-ethyl-1H-benzimidazole-5-carboxylic acid

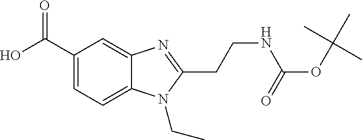

A solution of LiOH (51.9 g, 2.16 mol) in water (3 L) was added to a solution of methyl 2-{2[(tert-butoxycarbonyl)amino]ethyl}-1-ethyl-1H-benzimidazole-5-carboxylate (500 g, 1.44 mol) in MeOH (7 L). The resulting mixture was stirred at room temperature overnight. The mixture was then evaporated in vacuo and the residue was neutralized with concentrated hydrochloric acid. The mixture was then filtered and the filter cake was washed with water and dried in vacuo to give 2-{2-[(tert-butoxycarbonyl)amino]ethyl}-1-ethyl-1H-benzimidazole-5-carboxylic acid (450 g, 87.5%) as a grey solid.

### Step 4. tert-Butyl (2-{1-ethyl-5-[(2-methoxyethyl)carbamoyl]-1H-benzimidazol-2-yl}ethyl)carbamate

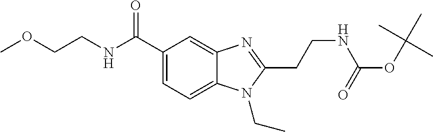

EDC (177.7 g, 1.26 mol) was added to a mixture of 2-{2-[(tert-butoxycarbonyl)amino]ethyl}-1-ethyl-1H-benzimidazole-5-carboxylic acid (300 g, 0.90 mol) and HOBt (170 g, 1.26 mol) in CH₂Cl₂ (4 L). The resulting mixture was stirred at room temperature for 1 hour. 2-Methoxy-ethylamine (189 g, 2.52 mol) was added and the mixture was stirred at room temperature for 3 hours. TLC (ethyl acetate) indicated that the reaction was complete. The mixture was washed with saturated aqueous NH₄Cl (2 L), aqueous NaOH (2 L, 0.5 mol/L) and brine, dried over Na₂SO₄ and concentrated in vacuo to afford tert-butyl (2-{1-ethyl-5-[{(2-methoxyethyl)carbamoyl]-1H-benzimidazol-2-yl}ethyl)carbamate (280 g, 80.0%) as a white solid.

### Step 5. 2-(2-Aminoethyl)-1-ethyl-N-(2-methoxyethyl)-1H-benzimidazole-5-carboxamide

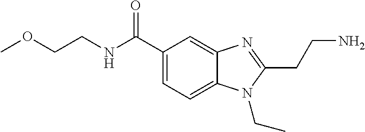

Methanol saturated with hydrogen chloride gas (1 L) was added dropwise to a mixture of tert-butyl (2-{1-ethyl-5-[{(2-methoxyethyl)carbamoyl]-1H-benzimidazol-2-yl}ethyl)carbamate (120 g, 0.308 mol) and MeOH (1.5 L). After the addition, the resulting mixture was allowed to stir at room temperature for 3 hours. The mixture was then evaporated in vacuo and the residue was dissolved in H₂O (1 L) and extracted with CH₂Cl₂ (400 mL×3). The aqueous layer was basified to pH 11 with aqueous NaOH (2 N), and extracted with CH₂Cl₂ (200 mL×3). The combined organic layers were concentrated in vacuo to give 2-(2-aminoethyl)-1-ethyl-N-(2-methoxyethyl)-1H-benzimidazole-5-carboxamide (60 g, 67.2%) as a grey oil. MS: observed [M+1] 291.2, calculated [M+1] 291.17.

### Preparation 17

### 1-(6-Methyl-5,6,7,8-tetrahydropyrido[4,3-d]pyrimidin-2-yl)pyrrolidin-3-amine Trihydrochloride

### Step 1. Preparation of tert-butyl {1-[amino(imino)methyl]pyrrolidin-3-yl}carbamate hydrochloride

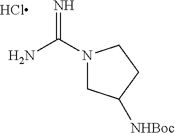

Pyrazolecarboxamidine (7.66 g, 53.8 mmol) was added in one portion to tert-butylpyrrolidin-2-yl carbamate (10 g, 53.8 mmol) in dimethylformamide (50 mL). Diisopropylamine (9.4 mL, 53.8 mmol) was then added dropwise and the reaction mixture was stirred at room temperature overnight. The dimethylformamide was evaporated, and dry diethyl ether (150 mL) was added to the oily residue which was stirred until a fine white precipitate formed. The precipitate was separated by filtration to give the title compound in 100% yield.

### Step 2. Preparation of 3-[(dimethylamino)methylene]-1-methylpiperidin-4-one

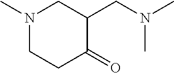

To a solution of 1-methylpiperidin-4-one (10 g, 88 mmol) in toluene (100 mL) was added 1,1-dimethoxy-N,N-dimethylmethanamine (52.7 g, 0.442 mol). The solution was heated to reflux overnight. The solvents were evaporated in vacuo, heptane (100 ml) was added and the solvents evaporated again to give the desired product. NMR indicated that the product was 70-80% pure and it was used in the next step without further purification.

### Step 3. Preparation of tert-butyl 1-(6-methyl-5,6,7,8-tetrahydropyrido[4,3-d]pyrimidin-2-yl)pyrrolidin-3-yl]carbamate

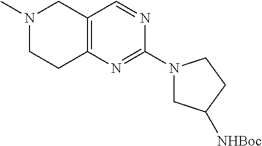

3-[(Dimethylamino)methylene]-1-methylpiperidin-4-one (45.4 g, 0.27 mol) and tert-butyl-1-[amino(imino)methyl]pyrrolidin-3-yl}carbamate hydrochloride (66.1 g, 0.25 mol) were dissolved in ethanol (600 mL) and to this was added sodium methoxide (13.5 g, 0.25 mol) dropwise. The reaction mixture was refluxed for 6 hours and then cooled to room temperature. The reaction mixture was then evaporated to dryness, and the residue was treated with water (500 mL). The precipitate was separated by filtration, washed with water (250 mL) and diethyl ether (500 mL) and dried to give the title compound 59.0 g (yield 70.8%).

### Step 4. Preparation of 1-(6-methyl-5,6,7,8-tetrahydropyrido[4,3-d]pyrimidin-2-yl)pyrrolidin-3-amine trihydrochloride

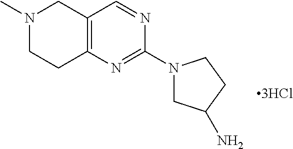

tert-Butyl-1-(6-methyl-5,6,7,8-tetrahydropyrido[4,3-d]pyrimidin-2-yl)pyrrolidin-3-yl]carbamate (59.0 g, 0.177 mol) was dissolved in methanol (200 mL) and cooled to 0° C. To this was added a solution of 4 M hydrogen chloride in dioxane (500 mL). The mixture was allowed to warm to room temperature, stirred at room temperature for 1 hour and then evaporated to dryness. The residue was boiled with ethanol (200 mL), then cooled to 0° C. and the resulting precipitate was filtered off. This gave the title compound (54.9 g, yield 90%) as a solid. ¹H NMR (DMSO-d₆) δ ppm 2.12 (m, 1H) 2.30 (m, 1H) 2.86-2.94 (s+m, 4H) 3.14-3.24 (m, 1H) 3.37-3.46 (m, 1H) 3.56-3.77 (br m, 6H) 3.78 (br m, 1H) 4.13 (dd, J=14.6, 8.3 Hz, 1H) 4.35 (d, J=14.0 Hz, 1H) 8.28 (s, 1H) 8.52 (br s, 3H) 11.71 (br s, 1H). LRMS [M+H] 234.

### Biological Data

Fluorescence Intensity h-PGDSTBA Enzyme Assay

Prostaglandin D Synthase (PGDS) converts the substrate prostaglandin H₂ (PGH₂) to prostaglandin D₂. The depletion of PGH₂ was measured via an Fe(II) reduction of the remaining PGH₂ to malondialdehyde (MDA) and 12-HHT. The enzyme assay is based on the quantitative formation of a fluorescent complex from the non-fluorescent compounds MDA and 2-thiobarbituric acid (TBA), substantially as described in U.S. patent application publication US-2004/152148 by Lombardt.

The enzyme assay (31 μls) contained 100 mM Tris base pH 8.0, 100 μM MgCl₂, 0.1 mg/ml IgG Rabbit serum, 5.0 μM PGH2 (Cayman; ethanol solution, #17020), 2.5 mM L-Glutathione (Sigma; reduced form #G4251), 1:175,000 human recombinant H-PGDS (from 1 mg/ml), 0.5% DMSO and inhibitor (varying concentration). Three μls of diluted inhibitor (dissolved in DMSO) was plated into a 384-well assay plate followed by a 25 μl addition of an enzyme solution containing h-PGDS, Tris, MgCl₂, IgG and L-Glutathione. After preincubation of inhibitor and enzyme solution for 10 minutes at room temperature, the reaction was initiated with a 3 μl addition of substrate solution in 10 mM HCl. The reaction was terminated after 42 second by the addition (3 μl) of stop buffer containing FeCl₂ and citric acid. After addition of 45.5 μls of TBA plates were heated for one hour in a 70 C oven. Plates were cooled at room temperature overnight and read on a plate reader the next day with excitation @ 530 nm and emission @ 565 nm.

IC₅₀'s of inhibitors were calculated with a 4-parameter fit using 11 inhibitor concentrations in duplicate with 3-fold serial dilutions. Controls on each plate included no inhibitor (zero % effect) and an inhibitor 10-fold in excess of its' IC₅₀ (100% effect). The highest inhibitor concentration tested was typically 1 μM.

Examples 529, 565, 566, 574-588 and 591 were tested in a slightly modified assay: The enzyme assay (30 μls during biological process) contained 100 mM Trizma pH 8.0, 100 μM MgCl₂, 0.1 mg/ml IgG Rabbit serum, 5.0 μM PGH2 (Cayman; ethanol solution, #17020), 2.5 mM L-Glutathione (Sigma; reduced form #G4251), 1:40,000 human recombinant H-PGDS (from 1 mg/ml), 0.5% DMSO and inhibitor (varying concentration). 3 μls of diluted inhibitor (dissolved in DMSO) was plated into a 384-well assay plate followed by a 24 μl addition of an enzyme solution containing h-PGDS, Trizma, MgCl₂, IgG and L-Glutathione. After pre-incubation of inhibitor and enzyme solution for 10 minutes at room temperature, the reaction was initiated with a 3 μl addition of substrate solution in 10 mM HCl. The reaction was terminated after 40 second by the addition of 3 μl stop buffer containing FeCl₂ and citric acid. After addition of 45 μls of TBA plates were heated for one hour in a 70° C. oven. Plates were cooled at room temperature overnight and read on a plate reader the next day with excitation @ 530 nm and emission @ 560 nm. IC₅₀'s of inhibitors were calculated with a 4-parameter fit using 11 inhibitor concentrations in duplicate with ½ log serial dilutions. Controls on each plate included no inhibitor (zero % effect) and an inhibitor 500-fold in excess of its' IC₅₀ (100% effect). The highest inhibitor concentration tested was typically 10 μM.

The following table shows the IC₅₀ values thus obtained.

| | IC₅₀ |
|---|---|
| Example | (nM) |
| 1 | 3.54 |
| 2 | 3.53 |
| 3 | 2.89 |
| 4 | 59.9 |
| 5 | 4.86 |
| 6 | 13.0 |
| 7 | 38.8 |
| 8 | 25.7 |
| 9 | 83.6 |
| 10 | 26.9 |
| 11 | 18.9 |
| 12 | 117 |
| 13 | 20.4 |
| 14 | 9.18 |
| 15 | 70.9 |
| 16 | 4.10 |
| 17 | 112 |
| 18 | 31.1 |
| 19 | 117 |
| 20 | 35.1 |
| 21 | 4.94 |
| 22 | 13.8 |
| 23 | 106 |
| 24 | 20.2 |
| 25 | 399 |
| 26 | 41.8 |
| 27 | 4.29 |
| 28 | 47.1 |
| 29 | 17.6 |
| 30 | 41.0 |
| 31 | 35.0 |
| 32 | 31.1 |
| 33 | 2.72 |
| 34 | 73.4 |
| 35 | 50.8 |
| 36 | 27.7 |
| 37 | 15.8 |
| 38 | 14.8 |
| 39 | 42.8 |
| 40 | 7.14 |
| 41 | 18.2 |
| 42 | 12.6 |
| 43 | 6.64 |
| 44 | 13.2 |
| 45 | 30.7 |
| 46 | 17.3 |
| 47 | 21.7 |
| 48 | 10.7 |
| 49 | 10.8 |
| 50 | 8.14 |
| 51 | 25.4 |
| 52 | 77.6 |
| 53 | 19.2 |
| 54 | 13.5 |
| 55 | 11.1 |
| 56 | 19.8 |
| 57 | 32.8 |
| 58 | 9.46 |
| 59 | 10.6 |
| 60 | 38.9 |
| 61 | 5.48 |
| 62 | 17.2 |
| 63 | 5.32 |
| 64 | 12.9 |
| 65 | 60.3 |
| 66 | 95.2 |
| 67 | 31.9 |
| 68 | 75.8 |
| 69 | 23.9 |
| 70 | 34.2 |
| 71 | 61.8 |
| 72 | 51.0 |
| 73 | 92.9 |
| 74 | 42.3 |
| 75 | 48.0 |
| 76 | 34.7 |
| 77 | 90.3 |
| 78 | 45.0 |
| 79 | 10.3 |
| 80 | 33.3 |
| 81 | 41.2 |
| 82 | 21.5 |
| 83 | 72.8 |
| 84 | 13.8 |
| 85 | 4.88 |
| 86 | 14.8 |
| 87 | 35.8 |
| 88 | 9.06 |
| 89 | 6.13 |
| 90 | 0.852 |
| 91 | 6.44 |
| 92 | 18.7 |
| 93 | 12.5 |
| 94 | 14.8 |
| 95 | 95.7 |
| 96 | 175 |
| 97 | 153 |
| 98 | 146 |
| 99 | 31.5 |
| 100 | 12.8 |
| 101 | 17.4 |
| 102 | 86.3 |
| 103 | 316 |
| 104 | 3.43 |
| 105 | 58.6 |
| 106 | 34.3 |
| 107 | 15.4 |
| 108 | 29.6 |
| 109 | 34.5 |
| 110 | 87.1 |
| 111 | 108 |
| 112 | 29.2 |
| 113 | 149 |
| 114 | 236 |
| 115 | 95.0 |
| 116 | 163 |
| 117 | 108 |
| 118 | 5.30 |
| 119 | 59.8 |
| 120 | 67.3 |
| 121 | 129 |
| 122 | >1000 |
| 123 | 15.6 |
| 124 | 18.3 |
| 125 | 23.9 |
| 126 | 33.8 |
| 127 | 30.3 |
| 128 | 28.3 |
| 129 | 92.0 |
| 130 | 39.4 |
| 131 | 27.2 |
| 132 | 6.36 |
| 133 | 59.2 |
| 134 | 47.7 |
| 135 | 46.5 |
| 136 | 3.51 |
| 137 | 11.2 |
| 138 | 287 |
| 139 | 39.0 |
| 140 | 32.0 |
| 141 | 34.5 |
| 142 | 25.2 |
| 143 | 11.0 |
| 144 | 109 |
| 145 | 223 |
| 146 | 34.0 |
| 147 | 381 |
| 148 | 32.0 |
| 149 | 20.1 |
| 150 | 2.88 |
| 151 | 1.88 |
| 152 | 4.49 |
| 153 | 2.99 |
| 154 | 43.7 |
| 155 | 5.9 |
| 156 | 15.7 |
| 157 | 8.79 |
| 158 | 337 |
| 159 | 391 |
| 160 | 40.8 |
| 161 | 88 |
| 162 | 19.6 |
| 163 | 146 |
| 164 | 9.13 |
| 165 | 20.8 |
| 166 | 2.57 |
| 167 | 285 |
| 168 | 175 |
| 169 | 43.5 |
| 170 | 26.1 |
| 171 | 52.7 |
| 172 | 5.74 |
| 173 | 82 |
| 174 | 601 |
| 175 | 47.1 |
| 176 | 33.4 |
| 177 | 8.19 |
| 178 | 6.88 |
| 179 | 20.5 |
| 180 | 23.1 |
| 181 | 44.6 |
| 182 | 79.2 |
| 183 | 17.4 |
| 184 | 49.5 |
| 185 | 8.99 |
| 186 | 34.6 |
| 187 | 14.5 |
| 188 | 16.3 |
| 189 | 6.74 |
| 190 | 1330 |
| 191 | 2.33 |
| 192 | 9.04 |
| 193 | 2.23 |
| 194 | 376 |
| 195 | 12.1 |
| 196 | 12.4 |
| 197 | 34 |
| 198 | 38.7 |
| 199 | 622 |
| 200 | 447 |
| 201 | 59.2 |
| 202 | 99.9 |
| 203 | 150 |
| 204 | 125 |
| 205 | 29.9 |
| 206 | 16.6 |
| 207 | 27.1 |
| 208 | 18.3 |
| 209 | 13.1 |
| 210 | 39.2 |
| 211 | 362 |
| 212 | 297 |
| 213 | 28.3 |
| 214 | 20.7 |
| 215 | 101 |
| 216 | 128 |
| 217 | 39.9 |
| 218 | 27.6 |
| 219 | 89.4 |
| 220 | 93.4 |
| 221 | 55.9 |
| 222 | 351 |
| 223 | 44.1 |
| 224 | 56.4 |
| 225 | 265 |
| 226 | 12.4 |
| 227 | 62.2 |
| 228 | 6.51 |
| 229 | 125 |
| 230 | 47.2 |
| 231 | 7.86 |
| 232 | 137 |
| 233 | 85.5 |
| 234 | 66.2 |
| 235 | 38.8 |
| 236 | 109 |
| 237 | 504 |
| 238 | 38.9 |
| 239 | 67.6 |
| 240 | 10.7 |
| 241 | 150 |
| 242 | 300 |
| 243 | 124 |
| 244 | 230 |
| 245 | 11.5 |
| 246 | 29.2 |
| 247 | 18.1 |
| 248 | 73.3 |
| 249 | 29.8 |
| 250 | 74.5 |
| 251 | 105 |
| 252 | 12.9 |
| 253 | 100 |
| 254 | 28.2 |
| 255 | 38.7 |
| 256 | 145 |
| 257 | 777 |
| 258 | 715 |
| 259 | 280 |
| 260 | 316 |
| 261 | 91.8 |
| 262 | 992 |
| 263 | 825 |
| 264 | 238 |
| 265 | 152 |
| 266 | 1000 |
| 267 | 1000 |
| 268 | 286 |
| 269 | 672 |
| 270 | 216 |
| 271 | 219 |
| 272 | 474 |
| 273 | 559 |
| 274 | 106 |
| 275 | 179 |
| 276 | 252 |
| 277 | 274 |
| 278 | 324 |
| 279 | 211 |
| 280 | 62.4 |
| 281 | 561 |
| 282 | 959 |
| 283 | 826 |
| 284 | 519 |
| 285 | 1000 |
| 286 | 536 |
| 287 | 816 |
| 288 | 333 |
| 289 | 466 |
| 290 | 627 |
| 291 | 203 |
| 292 | 215 |
| 293 | 508 |
| 294 | 191 |
| 295 | 377 |
| 296 | 209 |
| 297 | 351 |
| 298 | 528 |
| 299 | 578 |
| 300 | 762 |
| 301 | 49.9 |
| 302 | 177 |
| 303 | 331 |
| 304 | 177 |
| 305 | 38.7 |
| 306 | 387 |
| 307 | 187 |
| 308 | 54 |
| 309 | 557 |
| 310 | 19.4 |
| 311 | 368 |
| 312 | 19.9 |
| 313 | 7.62 |
| 314 | 79.5 |
| 315 | 72.5 |
| 316 | 382 |
| 317 | 153 |
| 318 | 49.2 |
| 319 | 140 |
| 320 | 17.4 |
| 321 | 84.5 |
| 322 | 1000 |
| 323 | 1000 |
| 324 | 1000 |
| 325 | 1000 |
| 326 | 414 |
| 327 | 597 |
| 328 | 92 |
| 329 | 552 |
| 330 | 507 |
| 331 | 234 |
| 332 | 326 |
| 333 | 77.1 |
| 334 | 1000 |
| 335 | 352 |
| 336 | 87.3 |
| 337 | 396 |
| 338 | 298 |
| 339 | 266 |
| 340 | 1000 |
| 341 | 131 |
| 342 | 1000 |
| 343 | 975 |
| 344 | 159 |
| 345 | 308 |
| 346 | 4.07 |
| 347 | 4.3 |
| 348 | 24.6 |
| 349 | 47 |
| 350 | 6.8 |
| 351 | 25.4 |
| 352 | 458 |
| 353 | 25.3 |
| 354 | 1.66 |
| 355 | 138 |
| 356 | 11.6 |
| 357 | 1.73 |
| 358 | 199 |
| 359 | 37.1 |
| 360 | 10.3 |
| 361 | 11.8 |
| 362 | 12 |
| 363 | 5.1 |
| 364 | 39.3 |
| 365 | 4.2 |
| 366 | 7.5 |
| 367 | 6.6 |
| 368 | 16.6 |
| 369 | 9.3 |
| 370 | 32.9 |
| 371 | 20.1 |
| 372 | 249 |
| 373 | 39.7 |
| 374 | 92.1 |
| 375 | 25.5 |
| 376 | 55.2 |
| 377 | 11.2 |
| 378 | 21 |
| 379 | 4.79 |
| 380 | 2.1 |
| 381 | 7.5 |
| 382 | 9.1 |
| 383 | 16.5 |
| 384 | 39.6 |
| 385 | 42.7 |
| 386 | 23.2 |
| 387 | 20.9 |
| 388 | 15.7 |
| 389 | 23.9 |
| 390 | 13 |
| 391 | 0.906 |
| 392 | 1.12 |
| 393 | 1.96 |
| 394 | 24.4 |
| 395 | 4.1 |
| 396 | 3 |
| 397 | 18.8 |
| 398 | 3.3 |
| 399 | 27.6 |
| 400 | 86.1 |
| 401 | 31.1 |
| 402 | 8 |
| 403 | 238 |
| 404 | 17.4 |
| 405 | 211 |
| 406 | 101 |
| 407 | 149 |
| 408 | 103 |
| 409 | 233 |
| 410 | 39.8 |
| 411 | 127 |
| 412 | 46.8 |
| 413 | 92.9 |
| 414 | 35.3 |
| 415 | 24.5 |
| 416 | 44.8 |
| 417 | 40.1 |
| 418 | 96.1 |
| 419 | 3.94 |
| 420 | 34.1 |
| 421 | 7.37 |
| 422 | 159 |
| 423 | 8.76 |
| 424 | 67.8 |
| 425 | 52.6 |
| 426 | 111 |
| 427 | 43.8 |
| 428 | 40.9 |
| 429 | 21.8 |
| 430 | 11.7 |
| 431 | 59 |
| 432 | 14.9 |
| 433 | 33.7 |
| 434 | 101 |
| 435 | 49.5 |
| 436 | 6.12 |
| 437 | 54.3 |
| 438 | 374 |
| 439 | 20 |
| 440 | 14.4 |
| 441 | 45.2 |
| 442 | 41.3 |
| 443 | 359 |
| 444 | 95.2 |
| 445 | 589 |
| 446 | 5.33 |
| 447 | 47.3 |
| 448 | 9.13 |
| 449 | 25.9 |
| 450 | 42.5 |
| 451 | 234 |
| 452 | 23.6 |
| 453 | 102 |
| 454 | 149 |
| 455 | 95.1 |
| 456 | 178 |
| 457 | 428 |
| 458 | 697 |
| 459 | 222 |
| 460 | 1000 |
| 461 | 91.5 |
| 462 | 1000 |
| 463 | 653 |
| 464 | 1000 |
| 465 | 345 |
| 466 | 1000 |
| 467 | 1000 |
| 468 | 688 |
| 469 | 1000 |
| 470 | 1000 |
| 471 | 192 |
| 472 | 256 |
| 473 | 381 |
| 474 | 734 |
| 475 | 1000 |
| 476 | 1000 |
| 477 | 895 |
| 478 | 377 |
| 479 | 896 |
| 480 | 327 |
| 481 | 365 |
| 482 | 527 |
| 483 | 151 |
| 484 | 178 |
| 485 | 201 |
| 486 | 204 |
| 487 | 1000 |
| 488 | 11.4 |
| 489 | 214 |
| 490 | 39.9 |
| 491 | 139 |
| 492 | 1000 |
| 493 | 980 |
| 494 | 758 |
| 495 | 1000 |
| 496 | 429 |
| 497 | 1000 |
| 498 | 1000 |
| 499 | 321 |
| 500 | 1000 |
| 501 | 1000 |
| 502 | 1000 |
| 503 | 1000 |
| 504 | 687 |
| 505 | 1000 |
| 506 | 1000 |
| 507 | 578 |
| 508 | 412 |
| 509 | 1000 |
| 510 | 575 |
| 511 | 1000 |
| 512 | 359 |
| 513 | 8.9 |
| 514 | 5.2 |
| 515 | 0.95 |
| 516 | 31.9 |
| 517 | 11.8 |
| 518 | 17.5 |
| 519 | 8.3 |
| 520 | 8.8 |
| 521 | 4.82 |
| 522 | 25 |
| 523 | 14.1 |
| 524 | 9.36 |
| 525 | 68.5 |
| 526 | 1510 |
| 527 | 10.3 |
| 528 | 6.45 |
| 529 | 11.3 |
| 530 | 3.03 |
| 531 | 3.34 |
| 532 | 3.94 |
| 533 | 8.79 |
| 534 | 1.04 |
| 535 | 5.56 |
| 536 | 53.5 |
| 537 | 61.2 |
| 538 | 30.1 |
| 539 | 64.4 |
| 540 | 5.41 |
| 541 | 16.9 |
| 542 | 39.1 |
| 543 | 104 |
| 544 | 12 |
| 545 | 14.2 |
| 546 | 59.6 |
| 547 | 98.4 |
| 548 | 54.7 |
| 549 | 37.9 |
| 550 | 30.4 |
| 551 | 84.7 |
| 552 | 60.8 |
| 553 | 31.8 |
| 554 | 223 |
| 555 | 174 |
| 556 | 90 |
| 557 | 89.6 |
| 558 | 119 |
| 559 | 440 |
| 560 | 129 |
| 561 | 19.8 |
| 562 | 4.89 |
| 563 | 5.15 |
| 564 | 61.6 |
| 565 | 114 |
| 566 | 349 |
| 567 | 10.6 |
| 568 | 2.63 |
| 569 | 25.1 |
| 570 | 32.5 |
| 571 | 62.1 |
| 572 | 0.886 |
| 573 | 3.24 |
| 574 | 1200 |
| 575 | 162 |
| 576 | 93.4 |
| 577 | 275 |
| 578 | 758 |
| 579 | 128 |
| 580 | 222 |
| 581 | 197 |
| 582 | 34.2 |
| 583 | 277 |
| 584 | 196 |
| 585 | 176 |
| 586 | 8.78 |
| 587 | 39.2 |
| 588 | 15.4 |
| 589 | |
| 590 | |
| 591 | 3.76 |
| 592 | |
| 593 | |
| 594 | 17 |
| 595 | 3 |
| 596 | 11 |
| 597 | 35 |

1. A compound of formula (I): 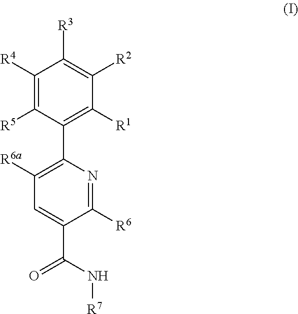
or a pharmaceutically acceptable salt thereof, wherein:
R¹, R², R³, R⁴ and R⁵ are each independently H, F, Cl, —CN, —NH₂, —CH₃, —CH₂F, —CHF₂, —CF₃, —OH, —OCH₃, —OCH₂F, —OCHF₂ or —OCF₃;
R⁶ is H, —NH₂, —OH or —CH₃;
R^{6a} is H, For Cl;
R⁷ is (1) C₁-C₃ alkyl optionally substituted by 1-3 substituents selected from phenyl, —CN, —OH, —NH₂, oxo, —COO(C₁-C₆ alkyl), C₃-C₈ cycloalkyl, —COO—(C₁-C₆ alkylene)-NHHet⁷, —NHHet⁸, —O—(C₁-C₆ alkylene)-Het⁸, —O—(C₁-C₆ alkylene)-phenyl, —CONH₂, —CONH—(C₁-C₆ alkylene)-Het⁹, —NH(phenyl), phenyl, —N(C₁-C₆ alkyl)(C₁-C₆ alkyl), —O(phenyl), —NHCOO—(C₁-C₆ alkylene)-phenyl, Het⁵, Het⁶, Het⁷ and Het⁸, said phenyl, C₃-C₈ cycloalkyl, Het⁵, Het⁶, Het⁷ and Het⁸ being optionally substituted by 1-3 substituents selected from C₁-C₆ alkyl, C₃-C₈ cycloalkyl —CO(C₁-C₆ alkyl), C₁-C₆ alkoxy, (C₁-C₆ alkoxy)C₁-C₆ alkyl, hydroxyl(C₁-C₆ alkyl), hydroxylphenyl(C₁-C₆ alkyl), halophenyl, (C₁-C₆ alkyl)phenyl, halo, C₁-C₆ haloalkyl, —S(C₁-C₆ alkyl), —SO₂NH₂, —COO(C₁-C₆ alkyl), —SO₂(C₁-C₆ alkyl), phenyl, phenyl(C₁-C₆ alkyl), (C₁-C₆ alkoxyphenyl), ((C₁-C₆ alkoxy)phenyl)C₁-C₆ alkyl, —(C₁-C₆ alkylene)-SO₂(C₁-C₆ alkyl), halophenyl, Het⁹, Het¹⁰, Het¹¹, —COHet⁹, —(C₁-C₆ alkylene)-Het⁹, —(C₁-C₆ alkylene)-Het¹¹, —SO₂NH(C₁-C₆ alkyl), —(C₁-C₆ alkylene)-COO(C₁-C₆ alkyl), —OH and oxo, said Het⁹, Het¹⁰ and Het¹¹ being optionally substituted by 1-3 substituents selected from C₁-C₆ alkyl, C₃-C₈ cycloalkyl, C₁-C₆ haloalkyl, C₁-C₆ alkoxy(C₁-C₆ alkyl), —OH and oxo;
(2) phenyl, said phenyl being (a) optionally substituted by 1-3 substituents selected from R^{a}, —OR^{b}, —S(O)ₙR^{b}, —COR^{b}, —NR^{x}R^{b}, —OCOR^{b}, —COOR^{b}, —NR^{x}COR^{b}, —CONR^{x}R^{b} —NR^{x}SO₂R^{b}, —SO₂NR^{x}R^{b}, —NR^{x}SO₂NR^{x}R^{b}, —NR^{x}COOR^{b}, —NR^{x}CONR^{x}R^{b}, —OCONR^{x}R^{b}, —OCOOR^{b}, —CONR^{x}SO₂R^{b}, oxo and —CN, and (b) optionally substituted by one or more halo atoms;
(3) Het¹, said Het¹ being (a) optionally substituted by 1-3 substituents selected from R^{a}, —OR^{b}, —S(O)ₙR^{b}, —COR^{b}, —NR^{x}R^{b}, —OCOR^{b}, —COOR^{b}, —NR^{x}COR^{b}, —CONR^{x}R^{b} —NR^{x}SO₂R^{b}, —SO₂NR^{x}R^{b}, —NR^{x}SO₂NR^{x}R^{b}, —NR^{x}COOR^{b}, —NR^{x}CONR^{x}R^{b}, —OCONR^{x}R^{b}, —OCOOR^{b}, —CONR^{x}SO₂R^{b}, oxo and —CN, and (b) optionally substituted by one or more halo atoms;
(4) 8-azabicyclo[3.2.1]octyl, 3,4-dihydro-2H-chromenyl, azabicyclo[3.1.0]hex-6-yl] or 1-oxa-8-azaspiro[4.5]decyl, each being optionally substituted by 1-3 substituents selected from C₁-C₆ alkyl, —COO(C₁-C₆ alkyl), —SO₂(C₁-C₆ alkyl), —CO(C₁-C₆ alkyl), Het⁷, Het⁸, —(C₁-C₆ alkylene)-Het⁷, (C₁-C₆ alkoxy)C₁-C₆ alkyl and oxo, wherein Het⁷ and Het⁸ may optionally be substituted by a C₁-C₆ alkyl, hydroxyl(C₁-C₆ alkyl) or morpholinylcarbonyl group;
(5) Het³, said Het³ being (a) optionally substituted by 1-3 substituents selected from R^{a}, —OR^{b}, —S(O)ₙR^{b}, —COR^{b}, —NR^{x}R^{b}, —OCOR^{b}, —COOR^{b}, —NR^{x}COR^{b}, —CONR^{x}R^{b} —NR^{x}SO₂R^{b}, —SO₂NR^{x}R^{b}, —NR^{x}SO₂NR^{x}R^{b}, —NR^{x}COOR^{b}, —NR^{x}CONR^{x}R^{b}, —OCONR^{x}R^{b}, —OCOOR^{b}, —CONR^{x}SO₂R^{b}, oxo and —CN, and (b) optionally substituted by one or more halo atoms; or
(6) Het⁴ selected from benzofuranyl, benzothienyl, indolyl, indazolyl, benzotriazolyl, pyrrolo[2,3-b]pyridyl, pyrrolo[2,3-c]pyridyl, pyrrolo[3,2-c]pyridyl, pyrrolo[3,2-b]pyridyl, imidazo[4,5-b]pyridyl, imidazo[4,5-c]pyridyl, pyrazolo[4,3-d]pyridyl, pyrazolo[4,3-c]pyridyl, pyrazolo[3,4-c]pyridyl, pyrazolo[3,4-b]pyridyl, isoindolyl, indazolyl, purinyl, indolizinyl, imidazo[1,5-a]pyridyl, pyrazolo[1,5-a]pyridyl, pyrrolo[1,2-b]pyridazinyl, imidazo[1,2-c]pyrimidinyl, isoquinolinyl, cinnolinyl, quinazolinyl, quinoxalinyl, phthalazinyl, 1,6-naphthyridinyl, 1,7-naphthyridinyl, 1,8-naphthyridinyl, 1,5-naphthyridinyl, 2,6-naphthyridinyl, 2,7-naphthyridinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-d]pyrazinyl, pyrido[3,4-b]pyrazinyl, pyrimido[5,4-d]pyrimidinyl, pyrazino[2,3-b]pyrazinyl and pyrimido[4,5-d]pyrimidinyl, said Het⁴ being (a) optionally substituted by 1-3 substituents selected from R^{a}, —OR^{b}, —S(O)ₙR^{b}, —COR^{b}, —NR^{x}R^{b}, —OCOR^{b}, —COOR^{b}, —NR^{x}COR^{b}, —CONR^{x}R^{b} —NR^{x}SO₂R^{b}, —SO₂NR^{x}R^{b}, —NR^{x}SO₂NR^{x}R^{b}, —NR^{x}COOR^{b}, —NR^{x}CONR^{x}R^{b}, —OCONR^{x}R^{b}, —OCOOR^{b}, —CONR^{x}SO₂R^{b}, oxo and —CN, and (b) optionally substituted by one or more halo atoms;
R^{a} is in each instance independently selected from C₁-C₆ alkyl, C₃-C₈ cycloalkyl, C₆-C₁₂ bicycloalkyl, Aryl¹, Het⁵, Het⁶, Het⁷ and Het⁸, said C₁-C₆ alkyl, C₃-C₈ cycloalkyl, C₆-C₁₂ bicycloalkyl, Aryl¹, Het⁵, Het⁶, Het⁷ and Het⁸ each being optionally substituted by 1-3 substituents selected from R^{c}, —OR^{d}, —S(O)ₙR^{d}, —COR^{d}, —NR^{x}R^{d}, —OCOR^{d}, —COOR^{d}, —NR^{x}COR^{d}, —CONR^{x}R^{d} —NR^{x}SO₂R^{d}, —SO₂NR^{x}R^{d}, —NR^{x}SO₂NR^{x}R^{d}, —NR^{x}COOR^{d}, —NR^{x}CONR^{x}R^{d}, —OCONR^{x}R^{d}, —OCOOR^{d}, —CONR^{x}SO₂R^{d}, oxo and —CN and one or more halo atoms;
R^{b} is in each instance independently selected from H, C₁-C₆ alkyl, C₃-C₈ cycloalkyl, C₆-C₁₂ bicycloalkyl, Aryl¹, Het⁵, Het⁶, Het⁷ and Het⁸, said C₁-C₆ alkyl, C₃-C₈ cycloalkyl, C₆-C₁₂ bicycloalkyl, Aryl¹, Het⁵, Het⁶, Het⁷ and Het⁸ each being optionally substituted by 1-3 substituents selected from R^{c}, —OR^{d}, —S(O)ₙR^{d}, —COR^{d}, —NR^{x}R^{d}, —OCOR^{d}, —COOR^{d}, —NR^{x}COR^{d}, —CONR^{x}R^{d} —NR^{x}SO₂R^{d}, —SO₂NR^{x}R^{d}, —NR^{x}SO₂NR^{x}R^{d}, —NR^{x}COOR^{d}, —NR^{x}CONR^{x}R^{d}, —OCONR^{x}R^{d}, —OCOOR^{d}, —CONR^{x}SO₂R^{d}, oxo and —CN and one or more halo atoms;
n is 0, 1 or 2;
R^{x} is in each instance independently H, C₁-C₆ alkyl or C₃-C₈ cycloalkyl, said C₁-C₆ alkyl or C₃-C₈ cycloalkyl being optionally substituted by one or more halo atoms;
Aryl¹ is phenyl or naphthyl;
Het¹ is a 3 to 8-membered saturated or partially unsaturated monocyclic heterocycle, containing 1 or 2 heteroatoms selected from O and N, with the proviso that Het¹ is not piperidinyl, pyrrolidinyl or azetidinyl;
Het² is a 6 to 12-membered saturated or partially unsaturated multicyclic heterocycle containing 1 or 2 heteroatoms selected from O and N, with the proviso that Het² is not a bridged piperidinyl, pyrrolidinyl or azetidinyl ring;
Het³ is (i) a 6-membered aromatic heterocycle containing 1-3 N atoms or (ii) a 5-membered aromatic heterocycle containing either (a) 1-4 N atoms or (b) 1 O or S atom and 0-3 N atoms;
Het⁴ is (i) a 10-membered bicyclic aromatic heterocycle containing 1-4 N atoms or (ii) a 9-membered bicyclic aromatic heterocycle containing either (a) 1-4 N atoms or (b) 1 O or S atom and 0-3 N atoms;
Het⁵ is a 3 to 8-membered saturated or partially unsaturated monocyclic heterocycle, containing 1 or 2 heteroatoms selected from O and N;
Het⁶ is a 6 to 12-membered saturated or partially unsaturated multicyclic heterocycle containing 1 or 2 heteroatoms selected from O and N;
Het⁷ is (i) a 6-membered aromatic heterocycle containing 1-3 N atoms or (ii) a 5-membered aromatic heterocycle containing either (a) 1-4 N atoms or (b) 1 O or S atom and 0-3 N atoms;
Het⁸ is (i) a 10-membered bicyclic aromatic heterocycle containing 1-4 N atoms or (ii) a 9-membered bicyclic aromatic heterocycle containing either (a) 1-4 N atoms or (b) 1 O or S atom and 0-3 N atoms;
R^{c} is in each instance independently selected from C₁-C₆ alkyl, C₃-C₈ cycloalkyl, C₆-C₁₂ bicycloalkyl, Aryl², Het⁹, Het¹⁰, Het¹¹ and Het¹², said C₁-C₆ alkyl, C₃-C₈ cycloalkyl, C₆-C₁₂ bicycloalkyl, Aryl², Het⁹, Het¹⁰, Het¹¹ and Het¹² each being optionally substituted by 1-3 substituents selected from R^{e} and one or more halo atoms;
R^{d} is in each instance independently selected from H, C₁-C₆ alkyl, C₃-C₈ cycloalkyl, C₆-C₁₂ bicycloalkyl, Aryl², Het⁹, Het¹⁰, Het¹¹ and Het¹², said C₁-C₆ alkyl, C₃-C₈ cycloalkyl, C₆-C₁₂ bicycloalkyl, Aryl², Het⁹, Het¹⁰, Het¹¹ and Het¹² each being optionally substituted by 1-3 substituents selected from R^{e} and one or more halo atoms;
Aryl² is phenyl or naphthyl;
Het⁹ is a 3 to 8-membered saturated or partially unsaturated monocyclic heterocycle, containing 1 or 2 heteroatoms selected from O and N;
Het¹⁰ is a 6 to 12-membered saturated or partially unsaturated multicyclic heterocycle containing 1 or 2 heteroatoms selected from O and N;
Het¹¹ is (i) a 6-membered aromatic heterocycle containing 1-3 N atoms or (ii) a 5-membered aromatic heterocycle containing either (a) 1-4 N atoms or (b) 1 O or S atom and 0-3 N atoms;
Het¹² is (i) a 10-membered bicyclic aromatic heterocycle containing 1-4 N atoms or (ii) a 9-membered bicyclic aromatic heterocycle containing either (a) 1-4 N atoms or (b) 1 O or S atom and 0-3 N atoms; and
R^{e} is —OR^{x}, —S(O)ₙR^{x}, —COR^{x}, —NR^{x}R^{x}, —OCOR^{x}, —COOR^{x}, —NR^{x}COR^{x}, —CONR^{x}R^{x} —NR^{x}SO₂R^{x}, —SO₂NR^{x}R^{x}, —NR^{x}SO₂NR^{x}R^{x}, —NR^{x}COOR^{x}, —NR^{x}CONR^{x}R^{x}, —OCONR^{x}, —OCOOR^{x}, —CONR^{x}SO₂R^{x}, oxo or —CN;
with the proviso that the compound of formula (I) is not:
2-hydroxy-N,6-diphenyl-3-pyridinecarboxamide,
N,6-diphenyl-3-pyridinecarboxamide,
6-(2-chlorophenyl)-N-phenyl-3-pyridinecarboxamide,
6-(2-fluorophenyl)-N-phenyl-3-pyridinecarboxamide,
6-(2-methylphenyl)-N-phenyl-3-pyridinecarboxamide,
2-methyl-N,6-diphenyl-3-pyridinecarboxamide,
N-(5-butyl-1,3,4-thiadiazol-2-yl)-2-methyl-6-phenyl-3-pyridinecarboxamide,
N-(4-acetyl-2-thiazolyl)-2-methyl-6-phenyl-3-pyridinecarboxamide,
5-[[(2-methyl-6-phenyl-3-pyridinyl)carbonyl]amino]-2-thiophenecarboxylic acid, methyl ester,
N-[4-(1,1-dimethylethyl)-2-thiazolyl]-2-methyl-6-phenyl-3-pyridinecarboxamide,
N-[4-[5-[(acetylamino)methyl]-2-thienyl]-2-thiazolyl]-2-methyl-6-phenyl-3-pyridinecarboxamide,
N-[4-[4-[(methylsulphonyl)(methyl)amino]phenyl]-2-thiazolyl]-2-methyl-6-phenyl-3-pyridinecarboxamide,
N-[4-[4-(acetylamino)-2-fluorophenyl]-2-thiazolyl]-2-methyl-6-phenyl-3-pyridinecarboxamide,
N-[4-[(2,6-dimethyl-4-morpholinyl)methyl]-2-thiazolyl]-2-methyl-6-phenyl-3-pyridinecarboxamide,
N-[5-[1-(difluoromethyl)-1H-imidazol-2-yl]-4-methyl-2-thiazolyl]-2-methyl-6-phenyl-3-pyridinecarboxamide,
N-[5-(1-ethylpropyl)-1,3,4-thiadiazol-2-yl]-2-methyl-6-phenyl-3-pyridinecarboxamide,
N-(3,5-dimethyl-1-phenyl-1H-pyrazol-4-yl)-2-methyl-6-phenyl-3-pyridinecarboxamide,
N-antipyrinyl-2-methyl-6-phenyl-nicotinamide,
1,2-dihydro-2-oxo-6-phenyl-N-1H-tetrazol-5-yl-3-pyridinecarboxamide,
2-methyl-6-phenyl-N-2-thiazolyl-3-pyridinecarboxamide,
2-methyl-N-(5-methyl-2-thiazolyl)-6-phenyl-3-pyridinecarboxamide,
2-methyl-N-(4-methyl-2-pyridinyl)-6-phenyl-3-pyridinecarboxamide,
N-(5-ethyl-1,3,4-thiadiazol-2-yl)-2-methyl-6-phenyl-3-pyridinecarboxamide,
N-[4-(2-amino-2-oxoethyl)-2-thiazolyl]-2-methyl-6-phenyl-3-pyridinecarboxamide, or
N-[5-(ethylthio)-1,3,4-thiadiazol-2-yl]-2-methyl-6-phenyl-3-pyridinecarboxamide;
6-(2-methylphenyl)-N-[2-[[[1-phenyl-3-(trifluoromethyl)-1H-pyrazol-4-yl]carbonyl]amino]ethyl]-3-pyridinecarboxamide,
N-[2-(5-methoxy-1H-indol-3-yl)ethyl]-6-phenyl-3-pyridinecarboxamide,
N-[4-[4-[1-(2-amino-2-oxoethoxy)-5,6,7,8-tetrahydro-2-naphthalenyl]-1-piperidinyl]butyl]-6-(4-chlorophenyl)-3-pyridinecarboxamide,
N-[4-[4-[1-(2-amino-2-oxoethoxy)-5,6,7,8-tetrahydro-2-naphthalenyl]-1-piperidinyl]butyl]-6-(4-cyanophenyl)-3-pyridinecarboxamide,
6-(4-chlorophenyl)-N-[4-[4-(5,6,7,8-tetrahydro-1-methoxy-2-naphthalenyl]-1-piperidinyl]butyl]-3-pyridinecarboxamide,
6-(4-chlorophenyl)-N-[4-[4-(5,6,7,8-tetrahydro-1-methoxy-2-naphthalenyl]-1-piperidinyl]butyl]-3-pyridinecarboxamide,
6-(2-chlorophenyl)-N-[(1S)-2-[(cyanomethyl)amino]-1-[(2,6-difluorophenyl)methyl]-2-oxoethyl]-3-pyridinecarboxamide,
6-(2-chlorophenyl)-N-[(1S)-2-[(cyanomethyl)amino]-1-[(2,6-difluoro-4-methoxyphenyl)methyl]-2-oxoethyl]-3-pyridinecarboxamide,
6-(2-chlorophenyl)-N-[(1S)-2-[(4-cyano-1-ethyl-4-piperidinyl)amino]-1-[(2,6-difluorophenyl)methyl]-2-oxoethyl]-3-pyridinecarboxamide,
6-(2-chlorophenyl)-N-[(1S)-2-[(cyanomethyl)amino]-2-oxo-1-(2-thiazolylmethyl)ethyl]-3-pyridinecarboxamide,
6-(2-chlorophenyl)-N-[(1S,3S)-1-[[(4-cyano-1-ethyl-4-piperidinyl)amino]carbonyl]-3-phenyl)butyl]-3-pyridinecarboxamide,
N-[[6-(2-chlorophenyl)-3-pyridinyl]carbonyl]-2,6-difluoro-L-phenylalanine,
6-(2-chlorophenyl)-N-[(1S)-2-[(cyanomethyl)amino]-1-[(2,6-difluorophenyl)methyl]-2-oxoethyl]-3-pyridinecarboxamide,
6-(2-chlorophenyl)-N-[(1S)-1-[[(cyanomethyl)amino]carbonyl]-3-methylbutyl]-3-pyridinecarboxamide,
6-(4-methoxyphenyl)-N-[2-[4-(1-pyrrolidinylmethyl)phenyl]ethyl]-3-pyridinecarboxamide,
6-(4-fluorophenyl)-N-[2-[4-(1-pyrrolidinylmethyl)phenyl]ethyl]-3-pyridinecarboxamide,
□-[[[6-(3,4-dimethoxyphenyl)-1,2-dihydro-2-oxo-3-pyridinyl]carbonyl]amino]-4-hydroxybenzeneacetic acid,
N-[4-[4-(2,4-dimethoxyphenyl)-1-piperazinyl]butyl]-6-phenyl-3-pyridinecarboxamide,
5-[[2-(4-fluorophenyl)-1,1-dimethylethylamino]-4-[[[6-(3-methoxyphenyl)-3-pyridinyl]carbonyl]amino]-5-oxo-pentanoic acid,
5-[[2-(4-fluorophenyl)-1,1-dimethylethyl]amino]-5-oxa-4-[[(6-phenyl)-3-pyridinyl)carbonyl]amino]-(4S)-pentanoic acid,
5-[(1,1-dimethyl-2-phenylethyl)amino]-5-oxo-4-[[(6-phenyl)-3-pyridinyl)carbonyl]amino]-pentanoic acid,
5-[[2-(4-chlorophenyl)-1,1-dimethylethyl]amino]-5-oxo-4-[[(6-phenyl-3-pyridinyl)carbonyl]amino]-(4S)-pentanoic acid,
5-oxo-5-[(phenylmethyl)amino]-4-[[(6-phenyl-3-pyridinyl)carbonyl]amino]-(4S)-pentanoic acid 1,1-dimethylethyl ester,
5-oxo-5-[(phenylmethyl)amino]-4-[[(6-phenyl-3-pyridinyl)carbonyl]amino]-pentanoic acid,
5-[[(3-methoxyphenyl)methyl]amino]-5-oxo-4-[[(6-phenyl-3-pyridinyl)carbonyl]amino]-(4S)-pentanoic acid 1,1-dimethylethyl ester,
5-[[(3-methoxyphenyl)methyl]amino]-5-oxo-4-[[(6-phenyl-3-pyridinyl)carbonyl]amino]-(4S)-pentanoic acid,
N-(2-furanylmethyl)-2-methyl-6-phenyl-3-pyridinecarboxamide,
N-methyl-6-phenyl-3-pyridinecarboxamide, or
6-(4-methoxyphenyl)-N-[[3-[(8-methyl-8-azabicyclo[3.2.1]oct-3-yl)phenyl]methyl]-3-pyridinecarboxamide;
and with the proviso that when R¹, R², R³, R⁴ and R⁵ are each H, and R⁷ is optionally substituted C₁-C₆ alkyl, R⁶ is not CH₃ or OH;
and with the proviso that when R¹, R², R⁴ and R⁵ are each H, R³ is trifluoromethyl, R⁶ is CH₃ and R⁷ is methyl or ethyl substituted by R^{a}, R^{a} is not an optionally substituted phenyl ring or an optionally substituted phenyoxy group;
and with the proviso that when R¹, R², R⁴ and R⁵ are each H, R³ is F, R⁶ is H and R⁷ is methyl substituted by R^{a}, R^{a} is not an optionally substituted quinolinyl group;
and with the proviso that when one of R¹ and R⁵ is Cl and the other of R¹ and R⁵ is H, R² is H, R³ is H, R⁴ is H, R⁷ is methyl substituted by —CONR^{x}R^{b} and R^{b} is propyl, R^{b} is not substituted by —COHet³ or —COHet⁴;
and with the proviso that when R⁶ is H, R^{6a} is H, and R⁷ is methyl substituted by R^{a}, R^{a} is not a substituted phenyl group;
and with the proviso that when R⁶ is H and R^{6a} is H, R⁷ is not (CH₃)₂CHCH₂CH₂—.

2. A compound of claim 1, or a pharmaceutically acceptable salt thereof, wherein R¹, R², R³, R⁴ and R⁵ are each independently H, F, —CH₃, —OH or —OCH₃.

3. A compound of claim 1, or a pharmaceutically acceptable salt thereof, wherein R¹ is H, R², R³, R⁴ and R⁵ are each independently H, F, —CH₃, —OH or —OCH₃.

4. A compound of claim 1, or a pharmaceutically acceptable salt thereof, wherein R¹, R³, R⁴ and R⁵ are H and R² is F; or R¹, R³, R⁴ and R⁵ are H and R² is —CH₃; or R¹, R³, R⁴ and R⁵ are H and R² is —OCH₃; or R¹, R², R⁴ and R⁵ are H and R³ is F; or R¹, R³ and R⁵ are H and R² and R⁴ are both F; or R¹, R², R³, R⁴ and R⁵ are each H; or R¹, R³ and R⁵ are H, R² is F and R⁴ is —OCH₃; or R¹, R³ and R⁴ are H, R² is F and R⁵ is —OH.

5. A compound of claim 1, or a pharmaceutically acceptable salt thereof, wherein R⁶ is H.

6. A compound of claim 1, or a pharmaceutically acceptable salt thereof, wherein R^{6a} is H or Cl.

7. A compound of claim 1, or a pharmaceutically acceptable salt thereof, wherein R⁷ is C₁-C₃ alkyl optionally substituted by 1-3 substituents selected from phenyl, —CN, —OH, —NH₂, oxo, —COO(C₁-C₆ alkyl), C₃-C₈ cycloalkyl, —COO—(C₁-C₆ alkylene)-NHHet⁷, —NHHet⁸, —O—(C₁-C₆ alkylene)-Het⁶, —O—(C₁-C₆ alkylene)-phenyl, —CONH₂, —CONH—(C₁-C₆ alkylene)-Het⁹, —NH(phenyl), phenyl, —N(C₁-C₆ alkyl)(C₁-C₆ alkyl), —O(phenyl), —NHCOO—(C₁-C₆ alkylene)-phenyl, Het⁵, Het⁶, Het⁷ and Het⁸, said phenyl, C₃-C₈ cycloalkyl, Het⁵, Het⁶, Het⁷ and Het⁶ being optionally substituted by 1-3 substituents selected from C₁-C₆ alkyl, C₃-C₈ cycloalkyl —CO(C₁-C₆ alkyl), C₁-C₆ alkoxy, (C₁-C₆ alkoxy)C₁-C₆ alkyl, hydroxyl(C₁-C₆ alkyl), hydroxylphenyl(C₁-C₆ alkyl), halophenyl, (C₁-C₆ alkyl)phenyl, halo, C₁-C₆ haloalkyl, —S(C₁-C₆ alkyl), —SO₂NH₂, —COO(C₁-C₆ alkyl), —SO₂(C₁-C₆ alkyl), phenyl, phenyl(C₁-C₆ alkyl), (C₁-C₆ alkoxyphenyl), ((C₁-C₆ alkoxy)phenyl)C₁-C₆ alkyl, —(C₁-C₆ alkylene)-SO₂(C₁-C₆ alkyl), halophenyl, Het⁹, Het¹⁰, —COHet⁹, —(C₁-C₆ alkylene)-Het⁹, —(C₁-C₆ alkylene)-Het¹¹, —SO₂NH(C₁-C₆ alkyl), —(C₁-C₆ alkylene)-COO(C₁-C₆ alkyl), —OH and oxo, said Het⁹, Het¹⁰ and Het¹¹ being optionally substituted by 1-3 substituents selected from C₁-C₆ alkyl, C₃-C₈ cycloalkyl, C₁-C₆ haloalkyl, C₁-C₆ alkoxy(C₁-C₆ alkyl), —OH and oxo.

8. A compound of claim 1, or a pharmaceutically acceptable salt thereof, wherein R⁷ is phenyl optionally substituted by 1-3 substituents selected from C₁-C₆ alkyl, C₁-C₆ alkoxy and halo.

9. A compound of claim 1, or a pharmaceutically acceptable salt thereof, wherein R⁷ is a 5- or 6-membered saturated heterocycle comprising one O or N atom, said heterocycle being optionally substituted by 1-3 substituents selected from R^{a}, —OR^{b}, —COOR^{b}, oxo, —NR^{x}R^{b}.

10. A compound of claim 1, or a pharmaceutically acceptable salt thereof, wherein R⁷ is 8-azabicyclo[3.2.1]octyl, 3,4-dihydro-2H-chromenyl, azabicyclo[3.1.0]hex-6-yl] or 1-oxa-8-azaspiro[4.5]decyl, each being optionally substituted by 1-3 substituents selected from C₁-C₆ alkyl, —COO(C₁-C₆ alkyl), —SO₂(C₁-C₆ alkyl), —CO(C₁-C₆ alkyl), Het⁷, Het⁸, —(C₁-C₆ alkylene)-Het⁷, (C₁-C₆ alkoxy)C₁-C₆ alkyl and oxo, wherein Het⁷ and Het⁸ may optionally be substituted by a C₁-C₆ alkyl, hydroxyl(C₁-C₆ alkyl) or morpholinylcarbonyl group.

11. A compound of claim 1, or a pharmaceutically acceptable salt thereof, wherein R⁷ is Het³ optionally substituted by 1-3 substituents R^{a} and optionally substituted by one or more halo atoms.

12. A pharmaceutical composition comprising a compound of claim 1, or a pharmaceutically acceptable salt thereof, and a pharmaceutically acceptable excipient.

13. A method of treating an allergic or respiratory condition, in a subject in need of such treatment, comprising administering to the subject a therapeutically effective amount of a compound of formula (I): 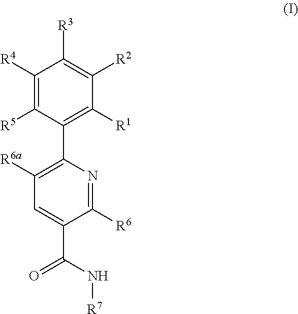
or a pharmaceutically acceptable salt thereof, wherein:
R¹, R², R³, R⁴ and R⁵ are each independently H, F, Cl, —CN, —NH₂, —CH₃, —CH₂F, —CHF₂, —CF₃, —OH, —OCH₃, —OCH₂F, —OCHF₂ or —OCF₃;
R⁶ is H, —NH₂, —OH or —CH₃;
R^{6a} is H, For Cl;
R⁷ is (1) C₁-C₃ alkyl optionally substituted by 1-3 substituents selected from phenyl, —CN, —OH, —NH₂, oxo, —COO(C₁-C₆ alkyl), C₃-C₈ cycloalkyl, —COO—(C₁-C₆ alkylene)-NHHet⁷, —NHHet⁸, —O—(C₁-C₆ alkylene)-Het⁸, —O—(C₁-C₆ alkylene)-phenyl, —CONH₂, —CONH—(C₁-C₆ alkylene)-Het⁹, —NH(phenyl), phenyl, —N(C₁-C₆ alkyl)(C₁-C₆ alkyl), —O(phenyl), —NHCOO—(C₁-C₆ alkylene)-phenyl, Het⁵, Het⁶, Het⁷ and Het⁸, said phenyl, C₃-C₈ cycloalkyl, Het⁵, Het⁶, Het⁷ and Het⁸ being optionally substituted by 1-3 substituents selected from C₁-C₆ alkyl, C₃-C₈ cycloalkyl —CO(C₁-C₆ alkyl), C₁-C₆ alkoxy, (C₁-C₆ alkoxy)C₁-C₆ alkyl, hydroxyl(C₁-C₆ alkyl), hydroxylphenyl(C₁-C₆ alkyl), halophenyl, (C₁-C₆ alkyl)phenyl, halo, C₁-C₆ haloalkyl, —S(C₁-C₆ alkyl), —SO₂NH₂, —COO(C₁-C₆ alkyl), —SO₂(C₁-C₆ alkyl), phenyl, phenyl(C₁-C₆ alkyl), (C₁-C₆ alkoxyphenyl), ((C₁-C₆ alkoxy)phenyl)C₁-C₆ alkyl, —(C₁-C₆ alkylene)-SO₂(C₁-C₆ alkyl), halophenyl, Het⁹, Het¹⁰, Het¹¹, —COHet⁹, —(C₁-C₆ alkylene)-Het⁹, —(C₁-C₆ alkylene)-Het¹¹, —SO₂NH(C₁-C₆ alkyl), —(C₁-C₆ alkylene)-COO(C₁-C₆ alkyl), —OH and oxo, said Het⁹, Het¹⁰ and Het¹¹ being optionally substituted by 1-3 substituents selected from C₁-C₆ alkyl, C₃-C₈ cycloalkyl, C₁-C₆ haloalkyl, C₁-C₆ alkoxy(C₁-C₆ alkyl), —OH and oxo;
(2) phenyl, said phenyl being (a) optionally substituted by 1-3 substituents selected from R^{a}, —OR^{b}, —S(O)ₙR^{b}, —COR^{b}, —NR^{x}R^{b}, —OCOR^{b}, —COOR^{b}, —NR^{x}COR^{b}, —CONR^{x}R^{b} —NR^{x}SO₂R^{b}, —SO₂NR^{x}R^{b}, —NR^{x}SO₂NR^{x}R^{b}, —NR^{x}COOR^{b}, —NR^{x}CONR^{x}R^{b}, —OCONR^{x}R^{b}, —OCOOR^{b}, —CONR^{x}SO₂R^{b}, oxo and —CN, and (b) optionally substituted by one or more halo atoms;
(3) Het¹, said Het¹ being (a) optionally substituted by 1-3 substituents selected from R^{a}, —OR^{b}, —S(O)ₙR^{b}, —COR^{b}, —NR^{x}R^{b}, —OCOR^{b}, —COOR^{b}, —NR^{x}COR^{b}, —CONR^{x}R^{b} —NR^{x}SO₂R^{b}, —SO₂NR^{x}R^{b}, —NR^{x}SO₂NR^{x}R^{b}, —NR^{x}COOR^{b}, —NR^{x}CONR^{x}R^{b}, —OCONR^{x}R^{b}, —OCOOR^{b}, —CONR^{x}SO₂R^{b}, oxo and —CN, and (b) optionally substituted by one or more halo atoms;
(4) an 8- to 11-membered saturated or partially unsaturated heterocycle containing 1 oxygen atom, 1 nitrogen atom or 1 oxygen and 1 nitrogen atom, said heterocycle being optionally substituted by 1-3 substituents selected from R^{a}, —COOR^{b}, —SO₂R^{b}, —COR^{b} and oxo;
(5) Het³, said Het³ being (a) optionally substituted by 1-3 substituents selected from R^{a}, —OR^{b}, —S(O)ₙR^{b}, —COR^{b}, —NR^{x}R^{b}, —OCOR^{b}, —COOR^{b}, —NR^{x}COR^{b}, —CONR^{x}R^{b} —NR^{x}SO₂R^{b}, —SO₂NR^{x}R^{b}, —NR^{x}SO₂NR^{x}R^{b}, —NR^{x}COOR^{b}, —NR^{x}CONR^{x}R^{b}, —OCONR^{x}R^{b}, —OCOOR^{b}, —CONR^{x}SO₂R^{b}, oxo and —CN, and (b) optionally substituted by one or more halo atoms; or
(6) Het⁴ selected from benzofuranyl, benzothienyl, benzimidazolyl, indolyl, indazolyl, benzotriazolyl, pyrrolo[2,3-b]pyridyl, pyrrolo[2,3-c]pyridyl, pyrrolo[3,2-c]pyridyl, pyrrolo[3,2-b]pyridyl, imidazo[4,5-b]pyridyl, imidazo[4,5-c]pyridyl, pyrazolo[4,3-d]pyridyl, pyrazolo[4,3-c]pyridyl, pyrazolo[3,4-c]pyridyl, pyrazolo[3,4-b]pyridyl, isoindolyl, indazolyl, purinyl, indolizinyl, imidazo[1,5-a]pyridyl, pyrazolo[1,5-a]pyridyl, pyrrolo[1,2-b]pyridazinyl, imidazo[1,2-c]pyrimidinyl, isoquinolinyl, cinnolinyl, quinazolinyl, quinoxalinyl, phthalazinyl, 1,6-naphthyridinyl, 1,7-naphthyridinyl, 1,8-naphthyridinyl, 1,5-naphthyridinyl, 2,6-naphthyridinyl, 2,7-naphthyridinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-d]pyrazinyl, pyrido[3,4-b]pyrazinyl, pyrimido[5,4-d]pyrimidinyl, pyrazino[2,3-b]pyrazinyl and pyrimido[4,5-d]pyrimidinyl, said Het⁴ being (a) optionally substituted by 1-3 substituents selected from R^{a}, —OR^{b}, —S(O)ₙR^{b}, —COR^{b}, —NR^{x}R^{b}, —OCOR^{b}, —COOR^{b}, —NR^{x}COR^{b}, —CONR^{x}R^{b} —NR^{x}SO₂R^{b}, —SO₂NR^{x}R^{b}, —NR^{x}SO₂NR^{x}R^{b}, —NR^{x}COOR^{b}, —NR^{x}CONR^{x}R^{b}, —OCONR^{x}R^{b}, —OCOOR^{b}, —CONR^{x}SO₂R^{b}, oxo and —CN, and (b) optionally substituted by one or more halo atoms;
R^{a} is in each instance independently selected from C₁-C₆ alkyl, C₃-C₈ cycloalkyl, C₆-C₁₂ bicycloalkyl, Aryl¹, Het⁵, Het⁶, Het⁷ and Het⁸, said C₁-C₆ alkyl, C₃-C₈ cycloalkyl, C₆-C₁₂ bicycloalkyl, Aryl¹, Het⁵, Het⁶, Het⁷ and Het⁸ each being optionally substituted by 1-3 substituents selected from R^{c}, —OR^{d}, —S(O)ₙR^{d}, —COR^{d}, —NR^{x}R^{d}, —OCOR^{d}, —COOR^{d}, —NR^{x}COR^{d}, —CONR^{x}R^{d} —NR^{x}SO₂R^{d}, —SO₂NR^{x}R^{d}, —NR^{x}SO₂NR^{x}R^{d}, —NR^{x}COOR^{d}, —NR^{x}CONR^{x}R^{d}, —OCONR^{x}R^{d}, —OCOOR^{d}, —CONR^{x}SO₂R^{d}, oxo and —CN and one or more halo atoms;
R^{b} is in each instance independently selected from H, C₁-C₆ alkyl, C₃-C₈ cycloalkyl, C₆-C₁₂ bicycloalkyl, Aryl¹, Het⁵, Het⁶, Het⁷ and Het⁸, said C₁-C₆ alkyl, C₃-C₈ cycloalkyl, C₆-C₁₂ bicycloalkyl, Aryl¹, Het⁵, Het⁶, Het⁷ and Het⁸ each being optionally substituted by 1-3 substituents selected from R^{c}, —OR^{d}, —S(O)ₙR^{d}, —COR^{d}, —NR^{x}R^{d}, —OCOR^{d}, —COOR^{d}, —NR^{x}COR^{d}, —CONR^{x}R^{d} —NR^{x}SO₂R^{d}, —SO₂NR^{x}R^{d}, —NR^{x}SO₂NR^{x}R^{d}, —NR^{x}COOR^{d}, —NR^{x}CONR^{x}R^{d}, —OCONR^{x}R^{d}, —OCOOR^{d}, —CONR^{x}SO₂R^{d}, oxo and —CN and one or more halo atoms;
n is 0, 1 or 2;
R^{x} is in each instance independently H, C₁-C₆ alkyl or C₃-C₈ cycloalkyl, said C₁-C₆ alkyl or C₃-C₈ cycloalkyl being optionally substituted by one or more halo atoms;
Aryl¹ is phenyl or naphthyl;
Het¹ is a 3 to 8-membered saturated or partially unsaturated monocyclic heterocycle, containing 1 or 2 heteroatoms selected from O and N, with the proviso that Het¹ is not piperidinyl, pyrrolidinyl and azetidinyl;
Het² is a 6 to 12-membered saturated or partially unsaturated multicyclic heterocycle containing 1 or 2 heteroatoms selected from O and N, with the proviso that Het² is not a bridged piperidinyl, pyrrolidinyl or azetidinyl ring;
Het³ is (i) a 6-membered aromatic heterocycle containing 1-3 N atoms or (ii) a 5-membered aromatic heterocycle containing either (a) 1-4 N atoms or (b) 1 O or S atom and 0-3 N atoms;
Het⁴ is (i) a 10-membered bicyclic aromatic heterocycle containing 1-4 N atoms or (ii) a 9-membered bicyclic aromatic heterocycle containing either (a) 1-4 N atoms or (b) 1O or S atom and 0-3 N atoms;
Het⁵ is a 3 to 8-membered saturated or partially unsaturated monocyclic heterocycle, containing 1 or 2 heteroatoms selected from O and N;
Het⁶ is a 6 to 12-membered saturated or partially unsaturated multicyclic heterocycle containing 1 or 2 heteroatoms selected from O and N;
Het⁷ is (i) a 6-membered aromatic heterocycle containing 1-3 N atoms or (ii) a 5-membered aromatic heterocycle containing either (a) 1-4 N atoms or (b) 1 O or S atom and 0-3 N atoms;
Het⁸ is (i) a 10-membered bicyclic aromatic heterocycle containing 1-4 N atoms or (ii) a 9-membered bicyclic aromatic heterocycle containing either (a) 1-4 N atoms or (b) 1O or S atom and 0-3 N atoms;
R^{c} is in each instance independently selected from C₁-C₆ alkyl, C₃-C₈ cycloalkyl, C₆-C₁₂ bicycloalkyl, Aryl², Het⁹, Het¹⁰, Het¹¹ and Het¹², said C₁-C₆ alkyl, C₃-C₈ cycloalkyl, C₆-C₁₂ bicycloalkyl, Aryl², Het⁹, Het¹⁰, Het¹¹ and Het¹² each being optionally substituted by 1-3 substituents selected from R^{e} and one or more halo atoms;
R^{d} is in each instance independently selected from H, C₁-C₆ alkyl, C₃-C₈ cycloalkyl, C₆-C₁₂ bicycloalkyl, Aryl², Het⁹, Het¹⁰, Het¹¹ and Het¹², said C₁-C₆ alkyl, C₃-C₈ cycloalkyl, C₆-C₁₂ bicycloalkyl, Aryl², Het⁹, Het¹⁰, Het¹¹ and Het¹² each being optionally substituted by 1-3 substituents selected from R^{e} and one or more halo atoms;
Aryl² is phenyl or naphthyl;
Het⁹ is a 3 to 8-membered saturated or partially unsaturated monocyclic heterocycle, containing 1 or 2 heteroatoms selected from O and N;
Het¹⁰ is a 6 to 12-membered saturated or partially unsaturated multicyclic heterocycle containing 1 or 2 heteroatoms selected from O and N;
Het¹¹ is (i) a 6-membered aromatic heterocycle containing 1-3 N atoms or (ii) a 5-membered aromatic heterocycle containing either (a) 1-4 N atoms or (b) 1 O or S atom and 0-3 N atoms;
Het¹² is (i) a 10-membered bicyclic aromatic heterocycle containing 1-4 N atoms or (ii) a 9-membered bicyclic aromatic heterocycle containing either (a) 1-4 N atoms or (b) 1 O or S atom and 0-3 N atoms; and
R^{e} is —OR^{x}, —S(O)ₙR^{x}, —COR^{x}, —NR^{x}R^{x}, —OCOR^{x}, —COOR^{x}, —NR^{x}COR^{x}, —CONR^{x}R^{x} —NR^{x}SO₂R^{x}, —SO₂NR^{x}R^{x}, —NR^{x}SO₂NR^{x}NR^{x}, —NR^{x}COOR^{x}, —NR^{x}CONR^{x}R^{x}, —OCONR^{x}R^{x}, —OCOOR^{x}, —CONR^{x}SO₂R^{x}, oxo or —CN.

14. The method of claim 13 wherein the disease or condition is asthma.

15. (canceled)

16. A combination of a compound of claim 1, or a pharmaceutically acceptable salt thereof, and a second pharmacologically active compound.
